(19) United States
(12) Patent Application Publication
(10) Pub. No.: US 2026/0284069 A1
(43) Pub. Date: Sep. 24, 2026

(54) PARG INHIBITORY COMPOUNDS

(71) Applicant: FoRx Therapeutics AG, Basel (CH)

(72) Inventors: Ulrich LÜCKING, Berlin (DE); Oliver QUEROLLE, Saint Louis (FR); Andreas GOUTOPOULOS, Boston, MA (US); Zaixu XU, Hubei (CN); Sotirios SOTIRIOU, Reinach (CH); Luca IACOVINO, Rheinfelden (CH); Alena FREUDENMANN, Riehen (CH)

(21) Appl. No.: 19/472,467

(22) PCT Filed: Apr. 5, 2024
(86) PCT No.: PCT/EP2024/059304

§ 371 Date: Oct. 3, 2025

Related U.S. Application Data
(60) Provisional application No. 63/494,331, filed on Apr. 5, 2023.
(60) Provisional application No. 63/569,973, filed on Mar. 26, 2024.

(51) Int. Cl. A61K 31/541; A61K 31/444; A61K 31/496; A61K 31/499; A61P 35/00; C07D 471/04

(52) U.S. Cl. CPC A61K 31/541; A61K 31/444; A61K 31/496; A61K 31/499; A61P 35/00; C07D 471/04

(57) ABSTRACT

The present invention relates to a compound of formula (I) or a pharmaceutically acceptable salt thereof. The present invention further relates to the compound of the present invention for use in therapy. Instant compounds are particularly useful as PARG inhibitors and can be used in a method of treatment of a proliferative disorder, preferably of cancer.

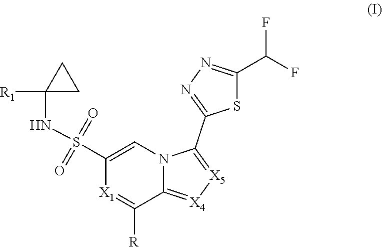

### FIELD OF THE INVENTION

The present invention relates to a compound of formula (I):

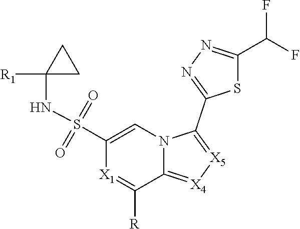

or a pharmaceutically acceptable salt thereof. The present invention further relates to the compound of the present invention for use in therapy. Instant compounds are particularly useful as PARG inhibitors and can be used in a method of treatment of a proliferative disorder, preferably of cancer.

### BACKGROUND OF THE INVENTION

Cancer is a leading cause of death worldwide. Although progression-free survival and overall survival of cancer patients has improved over the past two decades, millions of cancer patients still have few therapeutic options and poor survival outcomes (Jemal et al., J. Natl. Cancer Inst. 2017, 109, 1975).

DNA replication stress (DRS) is a hallmark of cancer cells and a major source of genomic instability (a) Halazonetis et al., Science 2008, 319, 1352; b) Negrini et al., Nat. Rev. Mol. Cell Biol. 2010, 11, 220). In broad terms, DRS refers to the deregulation of DNA replication and cell cycle progression. DRS can be induced from endogenous or exogenous causes such as oncogene activation and chemotherapeutics, respectively (Zeman and Cimprich, Nat. Cell Biol. 2013, 16, 2). At the level of the replication fork, DRS leads to replication fork stalling, disengagement of the replisome and eventually collapse. Several DNA repair proteins are involved in replication fork stability, protection, and restart under DRS conditions (a) Costantino et al., Science 2014, 343, 88; b) Scully et al., Curr. Opin. Genet. Dev. 2021 71, 154).

Poly(ADP)ribosylation (PARylation) is a transient and reversible post-translational modification that occurs at DNA damaged sites and is catalyzed by the poly (ADP-ribose) polymerase (PARP) family of proteins (Cohen and Chang, Nat. Chem. Biol. 2018, 14, 236). PARylation of various DNA repair proteins leads to their activation. Degradation of the poly(ADP) ribose chains is mediated primarily by the poly(ADP-ribose) glycohydrolase (PARG) protein. DNA damage dependent PARylation/dePARylation is a rapid and dynamic process which needs to be well regulated since imbalances between the two processes can lead to DNA damage.

Human PARG encodes a 111 kDa protein of 976 amino acids. It contains a N-terminal regulatory domain, a catalytic domain and an ADP-ribose binding macrodomain. Five human PARG transcripts have been identified. Full length PARG is mostly nuclear; the smaller isoforms localize primarily to the cytoplasm. PARG functions primarily as an exo-hydrolase and it releases mainly mono(ADP-ribose) by hydrolyzing the α-O-glycosidic ribose-ribose bond in PAR. PARG can also act as an endo-hydrolase. PARG preferentially degrades long and linear PAR chains whereas its activity with small and branched PAR chains is significantly reduced (O'Sullivan et al., Nat. Commun. 2019, 10, 1182).

Although PARG is the dominant cellular PAR degrading enzyme, it cannot act on the terminal protein-ribose bond. Additional hydrolases such as terminal ADP-ribose protein glycohydrolase (TARG1) and ADP-ribosylhydrolase 3 (ARH3) are also known to catalyze PAR-degradation. TARG1 and ARH3 complete the reversal of PARylation by removing protein-bound mono(ADP-ribose) moieties (a) Fontana et al., Elife 2017, doi: 10.7554/eLife.28533; b) Rack et al., Genes Dev. 2020, 34, 263). TARG1 is located in the nucleus and cytoplasm. ARH3 is found primarily in the cytoplasm but it can also be found in the mitochondria and in the nucleus (Rack et al., Genes Dev. 2020, 34, 263).

Genomic aberrations targeting tumor suppressor genes or oncogenes, often make cancer cells dependent on specific DNA repair pathways. For instance, it is well known that PARP inhibitors are particularly effective against tumors carrying mutations in the BRCA1 and BRCA2 genes (a) Bryant et al., Nature 2005, 434, 913; b) Farmer et al., Nature 2005, 434, 917). Targeting synthetic lethal interactions like the one between PARP and BRCA is an attractive novel therapeutic approach for cancer treatment.

PARG participates in DNA replication and in various DNA repair mechanisms including single-strand break (SSB) repair and replication fork restart. PARG inhibitors have shown synthetic lethal phenotype in cells with high levels of DRS caused by low expression of genes involved in DNA replication and/or replication fork stability (Pillay et al., Cancer Cell. 2019, 35, 519). Moreover, PARG inactivation, depletion or inhibition sensitizes cells to irradiation and to DNA damaging agents such as alkylating agents (e.g. temozolomide and methyl methanesulfonate) (a) Fujihara et al., Curr. Cancer Drug Targets 2009, 9, 953; b) Gogola et al., Cancer Cell 2018, 33, 1078; c) Houl et al., Nat Commun. 2019, 10, 5654).

Given the therapeutic potential of PARG inhibitors in cancer treatment, there is an increased need for the development of highly potent and selective PARG inhibitors beyond the ones that have already been described (a) James et al., ACS Chem. Biol. 2016, 11, 3179; b) Waszkowycz et al., J. Med. Chem. 2018, 61, 10767).

Certain compounds that are useful as PARG inhibitors are further disclosed in documents WO 2016/092326, WO 2016/097749 and WO 2021/055744.

Document US 2019/233411 discloses certain Gcn2 inhibitors and uses thereof.

Document WO 2009/050183 discloses certain imidazo[1,2-a]pyridine derivatives which are useful for treating diseases mediated by the ALK-5 and/or ALK-4 receptor.

### SUMMARY OF THE INVENTION

It was an objective technical problem of the present invention to provide compounds that are cell-permeable inhibitors of PARG. The technical problem of the present invention is solved by the embodiments described herein and as characterized by the claims.

Accordingly, in a first embodiment, the present invention provides a compound of formula (I):

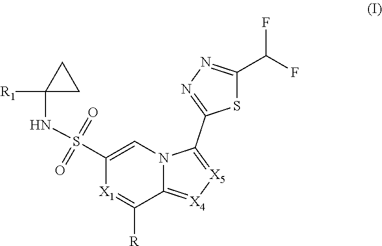

or a pharmaceutically acceptable salt or a prodrug thereof. It is understood that thought the present description the term “a compound of formula (I)” preferably encompasses also a compound of formula (Ia) to (Id), unless indicated to the contrary.

A further embodiment of the present invention relates to a pharmaceutical composition comprising the compound of formula (I) or a pharmaceutically acceptable salt, hydrate or solvate thereof, and a pharmaceutically acceptable carrier.

In a further embodiment, the present invention relates to the compound of formula (I) of the present invention or a pharmaceutically acceptable salt, hydrate or solvate thereof, or a pharmaceutical composition of the present invention, for use in therapy.

The compounds of formula (I) are useful for treating a disease or disorder in which PARG activity is implicated.

The compounds of formula (I) are useful for a method of treating a proliferative disorder. In a preferred embodiment of the present invention, the proliferative disorder is cancer, preferably a human cancer.

### Definitions

The following definitions apply throughout the present specification and the claims, unless specifically indicated otherwise.

The term “hydrogen” is herein used to refer to protium, deuterium and/or tritium, preferably to protium. Accordingly, the term “non-hydrogen atom” refers to any atoms that is not hydrogen, i.e. that is not protium, deuterium or tritium.

The term “hydrocarbon group” refers to a group consisting of carbon atoms and hydrogen atoms.

The term “alicyclic” is used in connection with cyclic groups and denotes that the corresponding cyclic group is non-aromatic.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₅ alkyl” denotes an alkyl group having 1 to 5 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl, and even more preferably to methyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₅ alkenyl” denotes an alkenyl group having 2 to 5 carbon atoms. Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂₋₄ alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more (e.g., one or two) carbon-to-carbon double bonds. The term “C₂₋₅ alkynyl” denotes an alkynyl group having 2 to 5 carbon atoms. Preferred exemplary alkynyl groups are ethynyl, propynyl (e.g., propargyl), or butynyl. Unless defined otherwise, the term “alkynyl” preferably refers to C₂₋₄ alkynyl.

As used herein, the term “alkylene” refers to an alkanediyl group, i.e. a divalent saturated acyclic hydrocarbon group which may be linear or branched. A “C₁₋₅ alkylene” denotes an alkylene group having 1 to 5 carbon atoms, and the term “C₀₋₃ alkylene” indicates that a covalent bond (corresponding to the option “C₀ alkylene”) or a C₁₋₃ alkylene is present. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₁₋₄ alkylene (including, in particular, linear C₁₋₄ alkylene), more preferably to methylene or ethylene, and even more preferably to methylene.

As used herein, the term “alkenylene” refers to an alkenediyl group, i.e. a divalent unsaturated acyclic hydrocarbon group which may be linear or branched and comprises one or more (e.g., one or two) carbon-to-carbon double bonds while it does not comprise any carbon-to-carbon triple bond. A “C₂₋₅ alkenylene” denotes an alkenylene group having 2 to 5 carbon atoms. Unless defined otherwise, the term “alkenylene” preferably refers to C₂₋₄ alkenylene (including, in particular, linear C₂₋₄ alkenylene).

As used herein, the term “alkynylene” refers to an alkynediyl group, i.e. a divalent unsaturated acyclic hydrocarbon group which may be linear or branched and comprises one or more (e.g., one or two) carbon-to-carbon triple bonds and optionally one or more (e.g., one or two) carbon-to-carbon double bonds. A “C₂₋₅ alkynylene” denotes an alkynylene group having 2 to 5 carbon atoms. Unless defined otherwise, the term “alkynylene” preferably refers to C₂₋₄ alkynylene (including, in particular, linear C₂₋₄ alkynylene).

As used herein, the term “carbocyclyl” refers to a hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclyl” preferably refers to aryl, cycloalkyl or cycloalkenyl.

As used herein, the term “heterocyclyl” refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S, N, P and Si, and the remaining ring atoms are carbon atoms, wherein one or more S ring atoms (if present) and/or one or more N ring atoms (if present) and/or one or more P ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. For example, each heteroatom-containing ring comprised in said ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. Unless defined otherwise, “heterocyclyl” preferably refers to heteroaryl, heterocycloalkyl or heterocycloalkenyl.

Preferably, the term “heterocyclyl” refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. For example, each heteroatom-containing ring comprised in said ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. Unless defined otherwise, “heterocyclyl” preferably refers to heteroaryl, heterocycloalkyl or heterocycloalkenyl.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). “Aryl” may, e.g., refer to phenyl, naphthyl, dialinyl (i.e., 1,2-dihydronaphthyl), tetralinyl (i.e., 1,2,3,4-tetrahydronaphthyl), indanyl, indenyl (e.g., 1H-indenyl), anthracenyl, phenanthrenyl, 9H-fluorenyl, or azulenyl. Unless defined otherwise, an “aryl” preferably has 6 to 14 ring atoms, more preferably 6 to 10 ring atoms, even more preferably refers to phenyl or naphthyl, and most preferably refers to phenyl.

As used herein, the term “arylene” refers to an aryl group, as defined herein above, but having two points of attachment, i.e. a divalent aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). “Arylene” may, e.g., refer to phenylene (e.g., phen-1,2-diyl, phen-1,3-diyl, or phen-1,4-diyl), naphthylene (e.g., naphthalen-1,2-diyl, naphthalen-1,3-diyl, naphthalen-1,4-diyl, naphthalen-1,5-diyl, naphthalen-1,6-diyl, naphthalen-1,7-diyl, naphthalen-2,3-diyl, naphthalen-2,5-diyl, naphthalen-2,6-diyl, naphthalen-2,7-diyl, or naphthalen-2,8-diyl), 1,2-dihydronaphthylene, 1,2,3,4-tetrahydronaphthylene, indanylene, indenylene, anthracenylene, phenanthrenylene, 9H-fluorenylene, or azulenylene. Unless defined otherwise, an “arylene” preferably has 6 to 14 ring atoms, more preferably 6 to 10 ring atoms, even more preferably refers to phenylene or naphthylene, and most preferably refers to phenylene (particularly phen-1,4-diyl).

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromanyl, chromenyl (e.g., 2H-1-benzopyranyl or 4H-1-benzopyranyl), isochromenyl (e.g., 1H-2-benzopyranyl), chromonyl, xanthenyl, phenoxathiinyl, pyrrolyl (e.g., 1H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolyl (e.g., 3H-indolyl), isoindolyl, indazolyl, indolizinyl, purinyl, quinolyl, isoquinolyl, phthalazinyl, naphthyridinyl, quinoxalinyl, cinnolinyl, pteridinyl, carbazolyl, @-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, oxadiazolyl (e.g., 1,2,4-oxadiazolyl, 1,2,5-oxadiazolyl (i.e., furazanyl), or 1,3,4-oxadiazolyl), thiadiazolyl (e.g., 1,2,4-thiadiazolyl, 1,2,5-thiadiazolyl, or 1,3,4-thiadiazolyl), phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzothiadiazolyl, benzoxazolyl, benzisoxazolyl, benzimidazolyl, benzo[b]thiophenyl (i.e., benzothienyl), triazolyl (e.g., 1H-1,2,3-triazolyl, 2H-1,2,3-triazolyl, 1H-1,2,4-triazolyl, or 4H-1,2,4-triazolyl), benzotriazolyl, 1H-tetrazolyl, 2H-tetrazolyl, triazinyl (e.g., 1,2,3-triazinyl, 1,2,4-triazinyl, or 1,3,5-triazinyl), furo[2,3-c]pyridinyl, dihydrofuropyridinyl (e.g., 2,3-dihydrofuro[2,3-c]pyridinyl or 1,3-dihydrofuro[3,4-c]pyridinyl), imidazopyridinyl (e.g., imidazo[1,2-a]pyridinyl or imidazo[3,2-a]pyridinyl), quinazolinyl, thienopyridinyl, tetrahydrothienopyridinyl (e.g., 4,5,6,7-tetrahydrothieno[3,2-c]pyridinyl), dibenzofuranyl, 1,3-benzodioxolyl, benzodioxanyl (e.g., 1,3-benzodioxanyl or 1,4-benzodioxanyl), or coumarinyl. Unless defined otherwise, the term “heteroaryl” preferably refers to a 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized.

As used herein, the term “heteroarylene” refers to a heteroaryl group, as defined herein above, but having two points of attachment, i.e. a divalent aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three, or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroarylene” may, e.g., refer to thienylene (i.e., thiophenylene; e.g., thien-2,3-diyl, thien-2,4-diyl, or thien-2,5-diyl), benzo[b]thienylene, naphtho[2,3-b]thienylene, thianthrenylene, furylene (i.e., furanylene; e.g., furan-2,3-diyl, furan-2,4-diyl, or furan-2,5-diyl), benzofuranylene, isobenzofuranylene, chromanylene, chromenylene, isochromenylene, chromonylene, xanthenylene, phenoxathiinylene, pyrrolylene, imidazolylene, pyrazolylene, pyridylene (i.e., pyridinylene), pyrazinylene, pyrimidinylene, pyridazinylene, indolylene, isoindolylene, indazolylene, indolizinylene, purinylene, quinolylene, isoquinolylene, phthalazinylene, naphthyridinylene, quinoxalinylene, cinnolinylene, pteridinylene, carbazolylene, @-carbolinylene, phenanthridinylene, acridinylene, perimidinylene, phenanthrolinylene, phenazinylene, thiazolylene (e.g., thiazol-2,4-diyl, thiazol-2,5-diyl, or thiazol-4,5-diyl), isothiazolylene (e.g., isothiazol-3,4-diyl, isothiazol-3,5-diyl, or isothiazol-4,5-diyl), phenothiazinylene, oxazolylene (e.g., oxazol-2,4-diyl, oxazol-2,5-diyl, or oxazol-4,5-diyl), isoxazolylene (e.g., isoxazol-3,4-diyl, isoxazol-3,5-diyl, or isoxazol-4,5-diyl), oxadiazolylene (e.g., 1,2,4-oxadiazol-3,5-diyl, 1,2,5-oxadiazol-3,4-diyl, or 1,3,4-oxadiazol-2,5-diyl), thiadiazolylene (e.g., 1,2,4-thiadiazol-3,5-diyl, 1,2,5-thiadiazol-3,4-diyl, or 1,3,4-thiadiazol-2,5-diyl), phenoxazinylene, pyrazolo[1,5-a]pyrimidinylene, 1,2-benzoisoxazolylene, benzothiazolylene, benzothiadiazolylene, benzoxazolylene, benzisoxazolylene, benzimidazolylene, benzo[b]thiophenylene (i.e., benzothienylene), triazolylene (e.g., 1H-1,2,3-triazolylene, 2H-1,2,3-triazolylene, 1H-1,2,4-triazolylene, or 4H-1,2,4-triazolylene), benzotriazolylene, 1H-tetrazolylene, 2H-tetrazolylene, triazinylene (e.g., 1,2,3-triazinylene, 1,2,4-triazinylene, or 1,3,5-triazinylene), furo[2,3-c]pyridinylene, dihydrofuropyridinylene (e.g., 2,3-dihydrofuro[2,3-c]pyridinylene or 1,3-dihydrofuro[3,4-c]pyridinylene), imidazopyridinylene (e.g., imidazo[1,2-a]pyridinylene or imidazo[3,2-a]pyridinylene), quinazolinylene, thienopyridinylene, tetrahydrothienopyridinylene (e.g., 4,5,6,7-tetrahydrothieno[3,2-c]pyridinylene), dibenzofuranylene, 1,3-benzodioxolylene, benzodioxanylene (e.g., 1,3-benzodioxanylene or 1,4-benzodioxanylene), or coumarinylene. Unless defined otherwise, the term “heteroarylene” preferably refers to a divalent 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroarylene” refers to a divalent 5 or 6 membered monocyclic ring comprising one or more (e.g., one, two or three) ring heteroatoms independently selected from O, S, and N, wherein one or more S ring atoms (if present) and/or one or more N ring atoms (if present) are optionally oxidized, and wherein one or more carbon ring atoms are optionally oxidized. A “heteroarylene”, including any of the specific heteroarylene groups described herein, may be attached through two carbon ring atoms, particularly through those two carbon ring atoms that have the greatest distance from one another (in terms of the number of ring atoms separating them by the shortest possible connection) within one single ring or within the entire ring system of the corresponding heteroarylene.

As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, decalinyl (i.e., decahydronaphthyl), or adamantyl. Unless defined otherwise, “cycloalkyl” preferably refers to a C₃₋₁₁ cycloalkyl, and more preferably refers to a C₃₋₇ cycloalkyl. A particularly preferred “cycloalkyl” is a monocyclic saturated hydrocarbon ring having 3 to 7 ring members (e.g., cyclopropyl or cyclohexyl).

As used herein, the term “cycloalkylene” refers to a cycloalkyl group, as defined herein above, but having two points of attachment, i.e. a divalent saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkylene” may, e.g., refer to cyclopropylene (e.g., cyclopropan-1,1-diyl or cyclopropan-1,2-diyl), cyclobutylene (e.g., cyclobutan-1,1-diyl, cyclobutan-1,2-diyl, or cyclobutan-1,3-diyl), cyclopentylene (e.g., cyclopentan-1,1-diyl, cyclopentan-1,2-diyl, or cyclopentan-1,3-diyl), cyclohexylene (e.g., cyclohexan-1,1-diyl, cyclohexan-1,2-diyl, cyclohexan-1,3-diyl, or cyclohexan-1,4-diyl), cycloheptylene, decalinylene (i.e., decahydronaphthylene), or adamantylene. Unless defined otherwise, “cycloalkylene” preferably refers to a C₃₋₁₁ cycloalkylene, and more preferably refers to a C₃₋₇ cycloalkylene. A particularly preferred “cycloalkylene” is a divalent monocyclic saturated hydrocarbon ring having 3 to 7 ring members (e.g., cyclopropylene or cyclohexylene).

As used herein, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S, N, P and Si, and the remaining ring atoms are carbon atoms, wherein one or more S ring atoms (if present) and/or one or more N ring atoms (if present) and/or one or more P ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkyl” may, e.g., refer to aziridinyl, azetidinyl, pyrrolidinyl, imidazolidinyl, pyrazolidinyl, piperidinyl, piperazinyl, azepanyl, diazepanyl (e.g., 1,4-diazepanyl), oxazolidinyl, isoxazolidinyl, thiazolidinyl, isothiazolidinyl, morpholinyl (e.g., morpholin-4-yl), thiomorpholinyl (e.g., thiomorpholin-4-yl), oxazepanyl, oxiranyl, oxetanyl, tetrahydrofuranyl, 1,3-dioxolanyl, tetrahydropyranyl, 1,4-dioxanyl, oxepanyl, thiiranyl, thietanyl, tetrahydrothiophenyl (i.e., thiolanyl), 1,3-dithiolanyl, thianyl, 1,1-dioxothianyl, thiepanyl, decahydroquinolinyl, decahydroisoquinolinyl, or 2-oxa-5-aza-bicyclo[2.2.1]hept-5-yl. Unless defined otherwise, “heterocycloalkyl” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized.

Preferably, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkyl” may, e.g., refer to aziridinyl, azetidinyl, pyrrolidinyl, imidazolidinyl, pyrazolidinyl, piperidinyl, piperazinyl, azepanyl, diazepanyl (e.g., 1,4-diazepanyl), oxazolidinyl, isoxazolidinyl, thiazolidinyl, isothiazolidinyl, morpholinyl (e.g., morpholin-4-yl), thiomorpholinyl (e.g., thiomorpholin-4-yl), oxazepanyl, oxiranyl, oxetanyl, tetrahydrofuranyl, 1,3-dioxolanyl, tetrahydropyranyl, 1,4-dioxanyl, oxepanyl, thiiranyl, thietanyl, tetrahydrothiophenyl (i.e., thiolanyl), 1,3-dithiolanyl, thianyl, 1,1-dioxothianyl, thiepanyl, decahydroquinolinyl, decahydroisoquinolinyl, or 2-oxa-5-aza-bicyclo[2.2.1]hept-5-yl. Unless defined otherwise, “heterocycloalkyl” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from 0, S and N, wherein one or more S ring atoms (if present) and/or one or more N ring atoms (if present) are optionally oxidized, and wherein one or more carbon ring atoms are optionally oxidized.

As used herein, the term “heterocycloalkylene” refers to a heterocycloalkyl group, as defined herein above, but having two points of attachment, i.e. a divalent saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S, N, P and Si, and the remaining ring atoms are carbon atoms, wherein one or more S ring atoms (if present) and/or one or more N ring atoms (if present) and/or one or more P ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkylene” may, e.g., refer to aziridinylene, azetidinylene, pyrrolidinylene, imidazolidinylene, pyrazolidinylene, piperidinylene, piperazinylene, azepanylene, diazepanylene (e.g., 1,4-diazepanylene), oxazolidinylene, isoxazolidinylene, thiazolidinylene, isothiazolidinylene, morpholinylene, thiomorpholinylene, oxazepanylene, oxiranylene, oxetanylene, tetrahydrofuranylene, 1,3-dioxolanylene, tetrahydropyranylene, 1,4-dioxanylene, oxepanylene, thiiranylene, thietanylene, tetrahydrothiophenylene (i.e., thiolanylene), 1,3-dithiolanylene, thianylene, 1,1-dioxothianylene, thiepanylene, decahydroquinolinylene, decahydroisoquinolinylene, or 2-oxa-5-aza-bicyclo[2.2.1]hept-5-ylene. Unless defined otherwise, “heterocycloalkylene” preferably refers to a divalent 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkylene” refers to a divalent 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized.

Preferably, the term “heterocycloalkylene” refers to a heterocycloalkyl group, as defined herein above, but having two points of attachment, i.e. a divalent saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkylene” may, e.g., refer to aziridinylene, azetidinylene, pyrrolidinylene, imidazolidinylene, pyrazolidinylene, piperidinylene, piperazinylene, azepanylene, diazepanylene (e.g., 1,4-diazepanylene), oxazolidinylene, isoxazolidinylene, thiazolidinylene, isothiazolidinylene, morpholinylene, thiomorpholinylene, oxazepanylene, oxiranylene, oxetanylene, tetrahydrofuranylene, 1,3-dioxolanylene, tetrahydropyranylene, 1,4-dioxanylene, oxepanylene, thiiranylene, thietanylene, tetrahydrothiophenylene (i.e., thiolanylene), 1,3-dithiolanylene, thianylene, 1,1-dioxothianylene, thiepanylene, decahydroquinolinylene, decahydroisoquinolinylene, or 2-oxa-5-aza-bicyclo[2.2.1]hept-5-ylene. Unless defined otherwise, “heterocycloalkylene” preferably refers to a divalent 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkylene” refers to a divalent 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized.

As used herein, the term “N-heterocycloalkyl” refers to the heterocycloalkyl groups as defined hereinabove wherein said heterocycloalkyl includes at least one nitrogen atom which serves as an attachment point of said heterocycloalkyl.

As used herein, the term “cycloalkenyl” refers to an unsaturated alicyclic (non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond. “Cycloalkenyl” may, e.g., refer to cyclopropenyl, cyclobutenyl, cyclopentenyl, cyclohexenyl, cyclohexadienyl, cycloheptenyl, or cycloheptadienyl. Unless defined otherwise, “cycloalkenyl” preferably refers to a C₃₋₁₁ cycloalkenyl, and more preferably refers to a C₃₋₇ cycloalkenyl. A particularly preferred “cycloalkenyl” is a monocyclic unsaturated alicyclic hydrocarbon ring having 3 to 7 ring members and containing one or more (e.g., one or two; preferably one) carbon-to-carbon double bonds.

As used herein, the term “cycloalkenylene” refers to a cycloalkenyl group, as defined hereinabove, but having two points of attachment, i.e. a divalent unsaturated alicyclic (non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond.

As used herein, the term “heterocycloalkenyl” refers to an unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S, N, P and Si, and the remaining ring atoms are carbon atoms, wherein one or more S ring atoms (if present) and/or one or more N ring atoms (if present) and/or one or more P ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkenyl” may, e.g., refer to imidazolinyl (e.g., 2-imidazolinyl (i.e., 4,5-dihydro-1H-imidazolyl), 3-imidazolinyl, or 4-imidazolinyl), tetrahydropyridinyl (e.g., 1,2,3,6-tetrahydropyridinyl), dihydropyridinyl (e.g., 1,2-dihydropyridinyl or 2,3-dihydropyridinyl), pyranyl (e.g., 2H-pyranyl or 4H-pyranyl), thiopyranyl (e.g., 2H-thiopyranyl or 4H-thiopyranyl), dihydropyranyl, dihydrofuranyl, dihydropyrazolyl, dihydropyrazinyl, dihydroisoindolyl, octahydroquinolinyl (e.g., 1,2,3,4,4a,5,6,7-octahydroquinolinyl), or octahydroisoquinolinyl (e.g., 1,2,3,4,5,6,7,8-octahydroisoquinolinyl). Unless defined otherwise, “heterocycloalkenyl” preferably refers to a 3 to 11 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms; more preferably, “heterocycloalkenyl” refers to a 5 to 7 membered monocyclic unsaturated non-aromatic ring group containing one or more (e.g., one, two, or three) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

Preferably, the term “heterocycloalkenyl” refers to an unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkenyl” may, e.g., refer to imidazolinyl (e.g., 2-imidazolinyl (i.e., 4,5-dihydro-1H-imidazolyl), 3-imidazolinyl, or 4-imidazolinyl), tetrahydropyridinyl (e.g., 1,2,3,6-tetrahydropyridinyl), dihydropyridinyl (e.g., 1,2-dihydropyridinyl or 2,3-dihydropyridinyl), pyranyl (e.g., 2H-pyranyl or 4H-pyranyl), thiopyranyl (e.g., 2H-thiopyranyl or 4H-thiopyranyl), dihydropyranyl, dihydrofuranyl, dihydropyrazolyl, dihydropyrazinyl, dihydroisoindolyl, octahydroquinolinyl (e.g., 1,2,3,4,4a,5,6,7-octahydroquinolinyl), or octahydroisoquinolinyl (e.g., 1,2,3,4,5,6,7,8-octahydroisoquinolinyl). Unless defined otherwise, “heterocycloalkenyl” preferably refers to a 3 to 11 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms; more preferably, “heterocycloalkenyl” refers to a 5 to 7 membered monocyclic unsaturated non-aromatic ring group containing one or more (e.g., one, two, or three) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

As used herein, the term “heterocycloalkenylene” refers to a heterocycloalkenyl group, as defined hereinabove, as defined hereinabove, but having two points of attachment, i.e. a divalent unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S, N, P and Si and the remaining ring atoms are carbon atoms, wherein one or more S ring atoms (if present) and/or one or more N ring atoms (if present) and/or one or more P ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring.

Preferably, the term “heterocycloalkenylene” refers to a heterocycloalkenyl group, as defined hereinabove, as defined hereinabove, but having two points of attachment, i.e. a divalent unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring.

As used herein, the term “halogen” refers to fluoro (—F), chloro (—Cl), bromo (—Br), or iodo (—I). As it is to be understood for the skilled person, the terms “halogen” and “halo” may be used interchangeably.

As used herein, the term “haloalkyl” refers to an alkyl group substituted with one or more (preferably 1 to 6, more preferably 1 to 3) halogen atoms which are selected independently from fluoro, chloro, bromo and iodo, and are preferably all fluoro atoms. It will be understood that the maximum number of halogen atoms is limited by the number of available attachment sites and, thus, depends on the number of carbon atoms comprised in the alkyl moiety of the haloalkyl group. “Haloalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CF₂—CF₃, or —CH(CF₃)₂. A particularly preferred “haloalkyl” group is —CF₃.

The terms “bond” and “covalent bond” are used herein synonymously, unless explicitly indicated otherwise or contradicted by context.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

A skilled person will appreciate that the substituent groups comprised in the compounds of the present invention may be attached to the remainder of the respective compound via a number of different positions of the corresponding specific substituent group. Unless defined otherwise, the preferred attachment positions for the various specific substituent groups are as illustrated in the examples.

As used herein, unless explicitly indicated otherwise or contradicted by context, the terms “a”, “an” and “the” are used interchangeably with “one or more” and “at least one”. Thus, for example, a composition comprising “a” compound of formula (I) can be interpreted as referring to a composition comprising “one or more” compounds of formula (I).

It is to be understood that wherever numerical ranges are provided/disclosed herein, all values and subranges encompassed by the respective numerical range are meant to be encompassed within the scope of the invention. Accordingly, the present invention specifically and individually relates to each value that falls within a numerical range disclosed herein, as well as each subrange encompassed by a numerical range disclosed herein.

As used herein, the term “about” preferably refers to ±10% of the indicated numerical value, more preferably to ±5% of the indicated numerical value, and in particular to the exact numerical value indicated. If the term “about” is used in connection with the endpoints of a range, it preferably refers to the range from the lower endpoint −10% of its indicated numerical value to the upper endpoint +10% of its indicated numerical value, more preferably to the range from of the lower endpoint −5% to the upper endpoint +5%, and even more preferably to the range defined by the exact numerical values of the lower endpoint and the upper endpoint.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

### DETAILED DESCRIPTION OF THE INVENTION

The invention is described in detail in the following. It is to be understood that the present invention specifically relates to each and every combination of features and embodiments described herein, including any combination of general and/or preferred features/embodiments.

In a first embodiment, the present invention relates to a compound of formula (I)

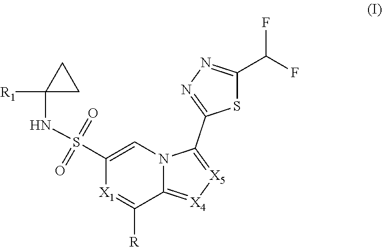

or a pharmaceutically acceptable salt thereof.

In formula (I), X₁, and X₅ are each independently selected from N and CH, and X₄ is selected from N and C—R₂, wherein R₂ is selected from hydrogen, C₁₋₂ alkyl, C₂ alkenyl, C₂ alkynyl, C₁₋₂ haloalkyl, cyclopropyl, cyclobutyl, oxetanyl, halogen, —SO₂(C₁₋₂ alkyl), —SO(NH)(C₁₋₂ alkyl), —SO(NC₁₋₂ alkyl)(C₁₋₂ alkyl), —CONH(C₁₋₂ alkyl), —O(C₁₋₂ alkyl), —NH₂, —NH(C₁₋₂ alkyl) and —N(C₁₋₂ alkyl)(C₁₋₂ alkyl), preferably wherein R₂ is selected from hydrogen, C₁₋₂ alkyl, C₂ alkenyl, C₂ alkynyl, C₁₋₂ haloalkyl, cyclopropyl and halogen, more preferably wherein R₂ is selected from hydrogen, —CH₃, —CF₃, cyclopropyl and —Cl, even more preferably wherein R₂ is hydrogen.

Preferably, X₁ is CH.

Preferably, X₄ is CH and X₅ is N, or X₄ is N and X₅ is CH, or X₄ is N and X₅ is N. However, in one embodiment, X₄ is CH and X₅ is CH.

In formula (I), R is selected from

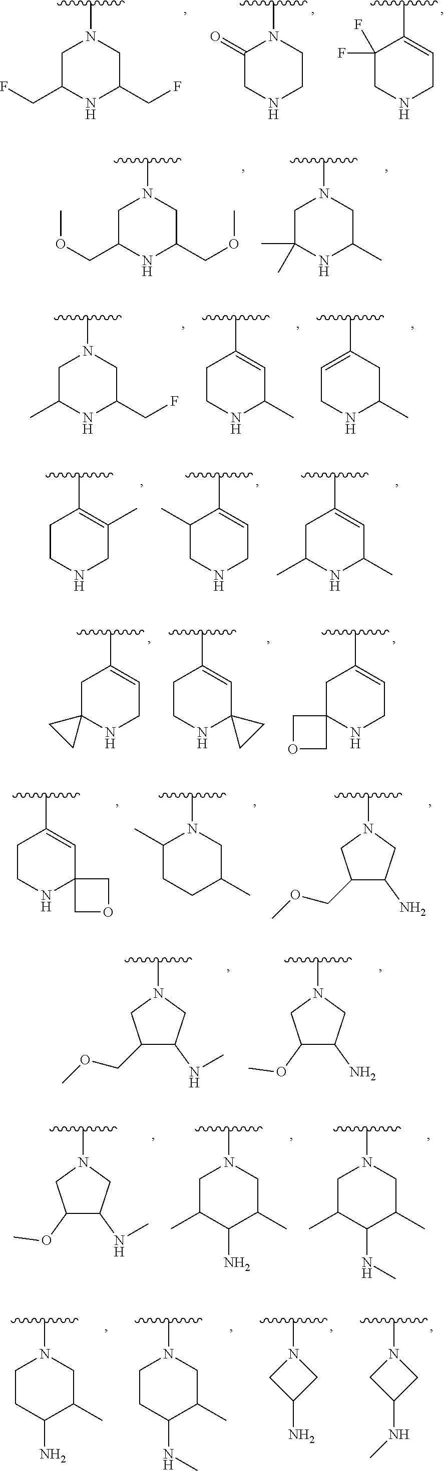

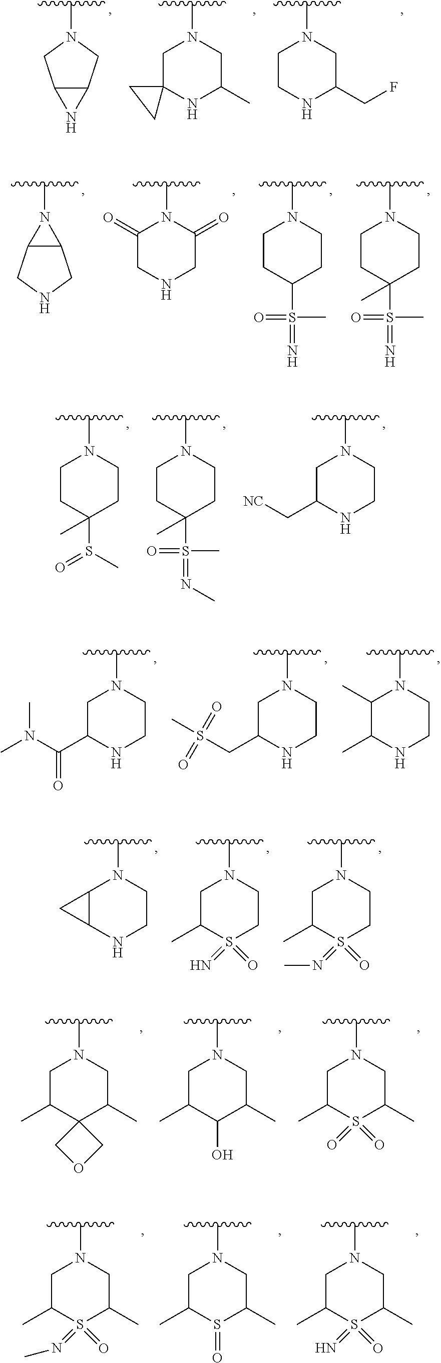

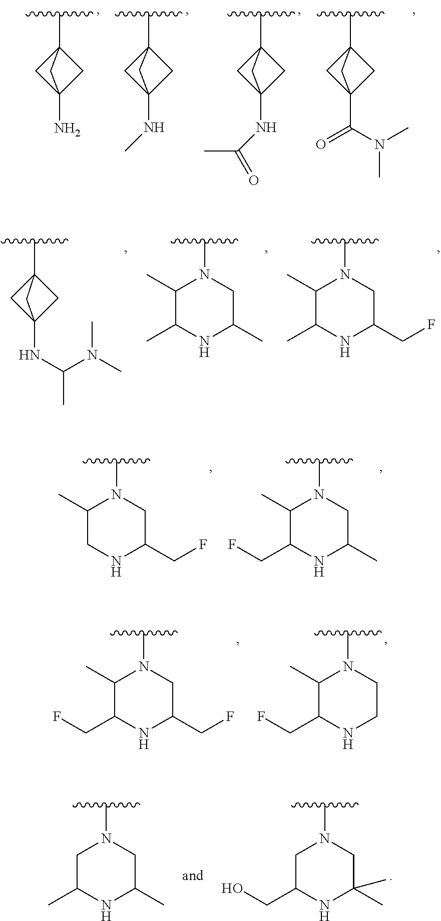

Preferably, R is selected from

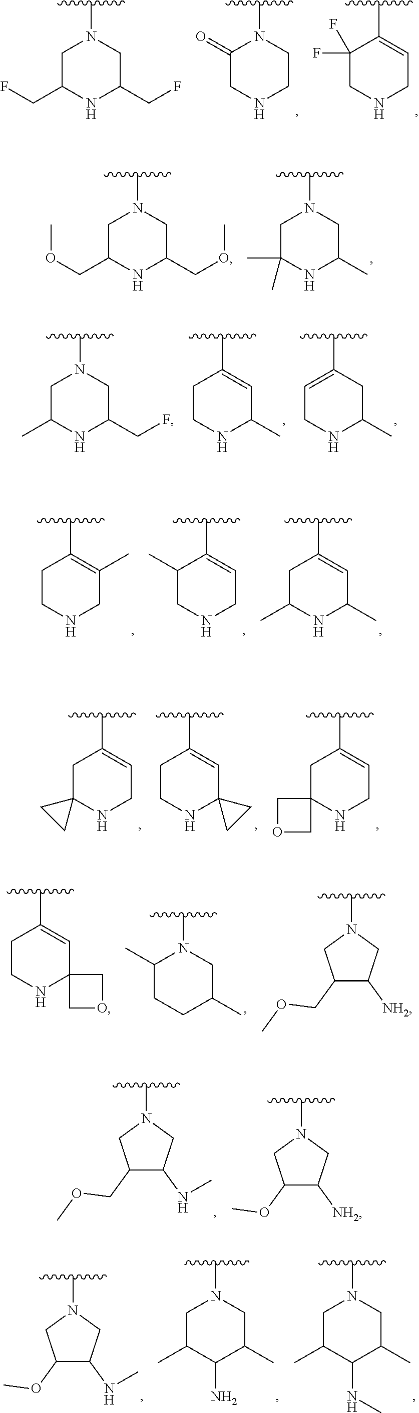

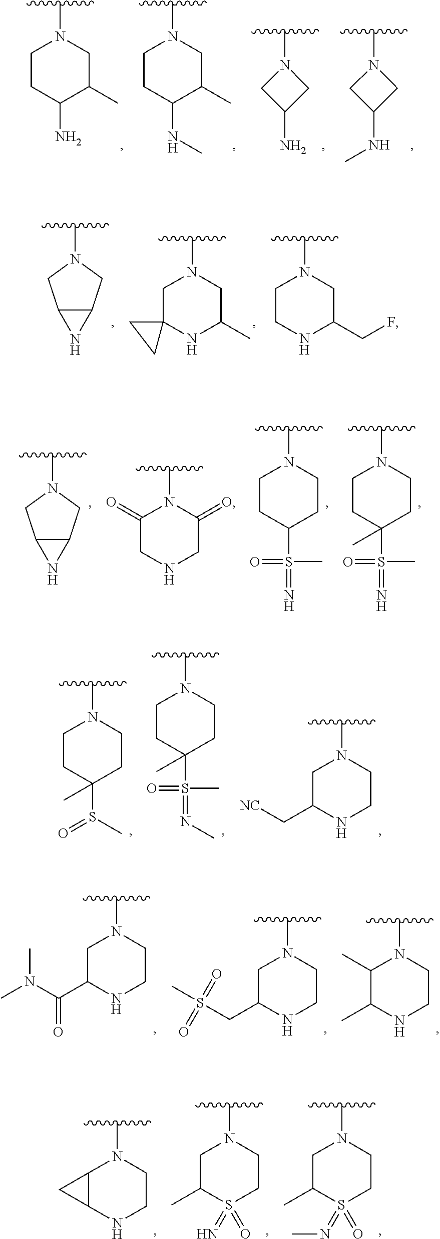

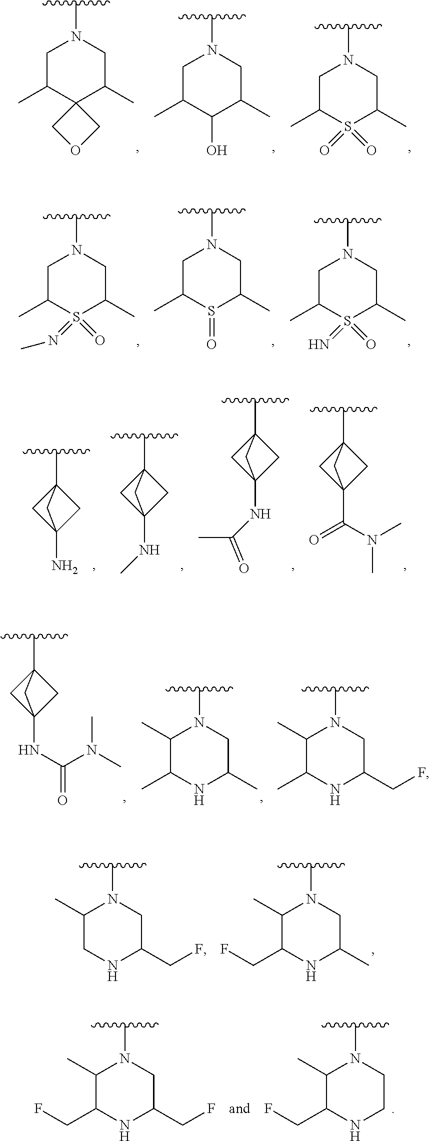

R₁ is selected from the group consisting of cyano, formyl, (C₁₋₂)alkyl, (C₂)alkenyl, (C₂)alkynyl, (C₁₋₂)haloalkyl, —(C₁₋₂ alkylene)-OH and —(C₁₋₂ alkylene)-O—(C₁₋₂ alkyl)). Preferably, R₁ is selected from the group consisting of cyano, formyl, (C₁₋₂)alkyl, (C₂)alkenyl, (C₂)alkynyl and (C₁₋₂)haloalkyl, preferably from the group consisting of cyano, formyl, (C₁₋₂)alkyl, (C₂)alkenyl, (C₂)alkynyl and (C₁₋₂)haloalkyl. More preferably, R₁ is selected from the group consisting of cyano, (C₁₋₂)alkyl, and (C₁₋₂)haloalkyl. Preferably, (C₁₋₂)alkyl as discussed herein is methyl. Preferably, (C₁₋₂)haloalkyl as discussed herein is fluoromethyl. Thus, preferably R₁ is selected from the group consisting of cyano, methyl and fluoromethyl. More preferably, R₁ is methyl, in a particularly preferred embodiment wherein R₁ is methyl, R₁ is CD₃. However, in an alternative preferred embodiment, R₁ is cyano, In again an alternative preferred embodiment, R₁ is fluoromethyl.

In a first specific embodiment of the compound of formula (I), R is

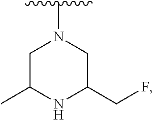 [SMILES: *N1CC(C)NC(CF)C1]

preferably R is selected from

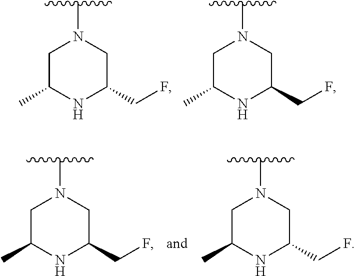

In a second specific embodiment of the compound of formula (I), R is selected from

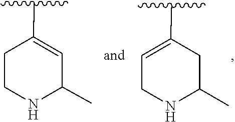

preferably R is selected from

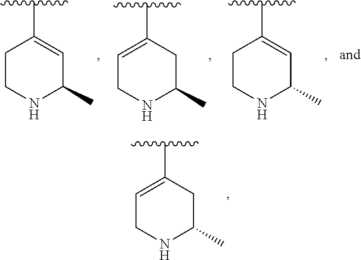

or R is selected from

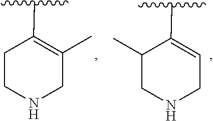

(preferably selected from

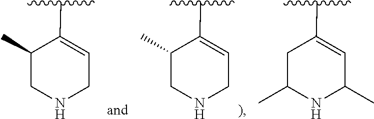

(preferably selected from

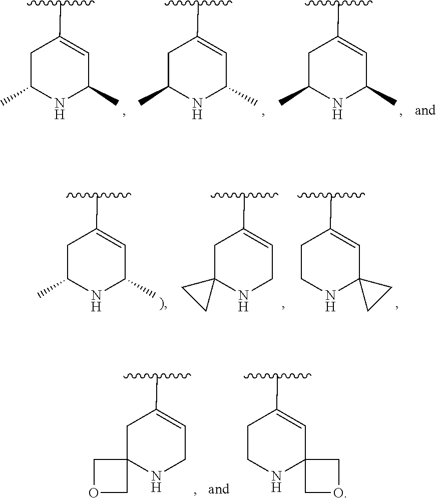

In a third specific embodiment, R is

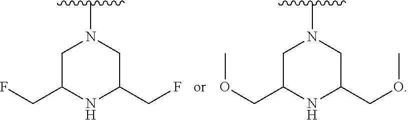 [SMILES: *N1CC(CF)NC(CF)C1 ; *N1CC(COC)NC(COC)C1]

### Preferably, if R is

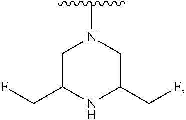 [SMILES: *N1CC(CF)NC(CF)C1]

R is selected from

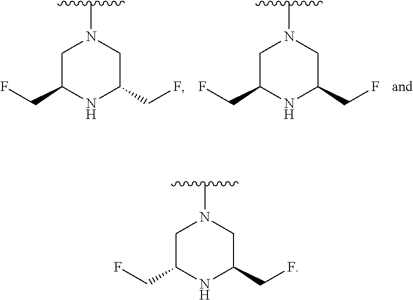

### Preferably, if R is

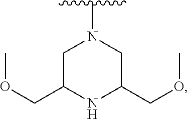 [SMILES: *N1CC(COC)NC(COC)C1]

R is selected from

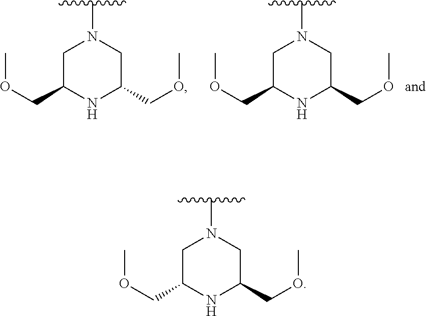

In a fourth specific embodiment of the compound of formula (I), R is selected from

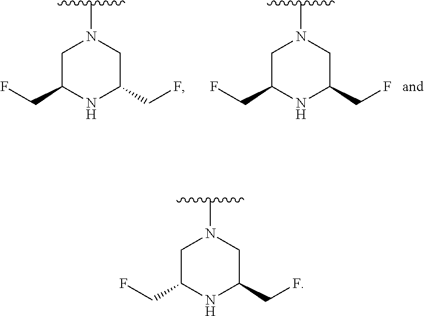

In a fifth specific embodiment of the compound of formula (I), R is selected from

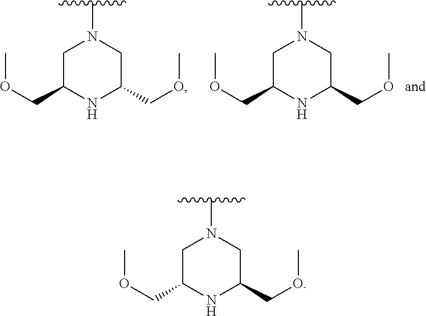

In a sixth specific embodiment of the compound of formula (I), R is

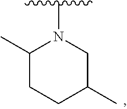

preferably selected from

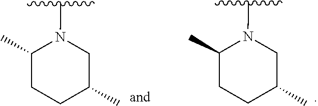

In a seventh specific embodiment of the compound of formula (I), R is selected from

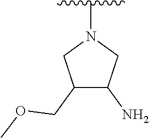 [SMILES: *N1CC(N)C(COC)C1]

(preferably selected from

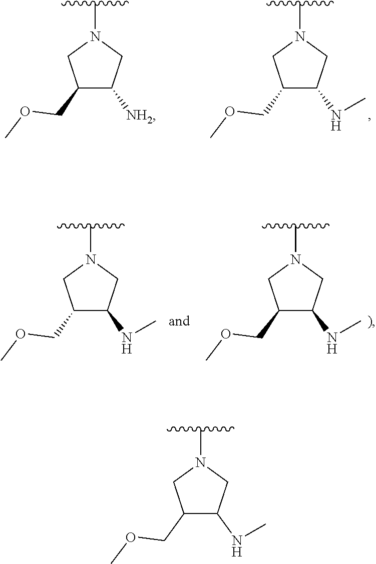

(preferably selected from

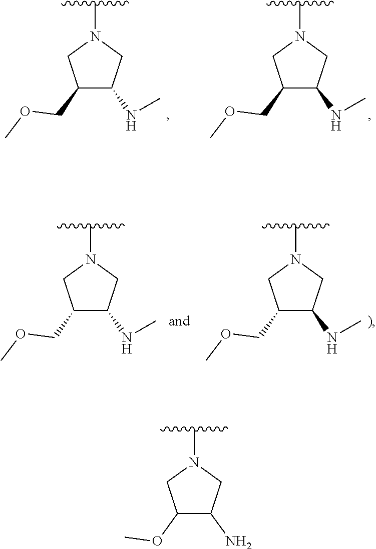

(preferably selected from

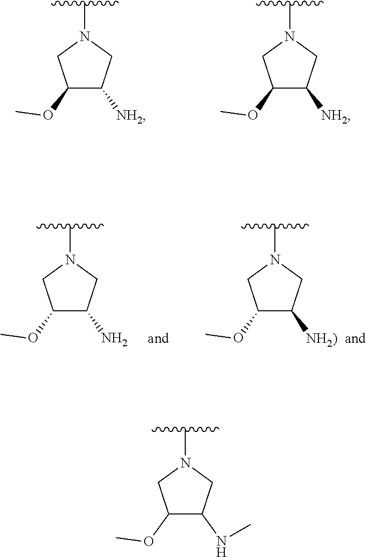

(preferably selected from

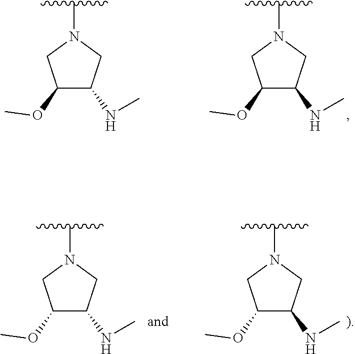

In an eighth specific embodiment of the compound of formula (I), R is selected from

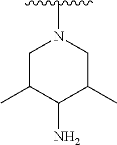

(preferably

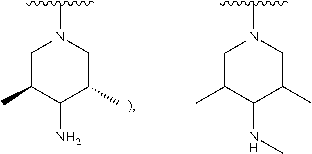

(preferably

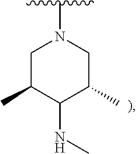

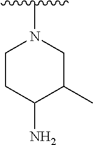 [SMILES: *N1CCC(N)C(C)C1]

(preferably

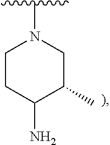 [SMILES: *N1CCC(N)[C@@H](C)C1]

and

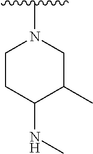 [SMILES: *N1CCC(NC)C(C)C1]

(preferably

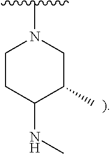 [SMILES: *N1CCC(NC)[C@@H](C)C1]

In a ninth specific embodiment of the compound of formula (I), R is selected from

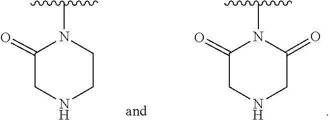 [SMILES: *N1CCNCC1=O ; *N1C(=O)CNCC1=O]

In a tenth specific embodiment of the compound of formula (I), R is

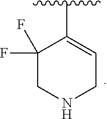 [SMILES: *C1=CCNCC1(F)F]

In an eleventh specific embodiment of the compound of formula (I), R is selected from

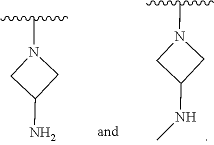

In a twelfth specific embodiment of the compound of formula (I), R is selected from

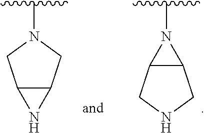 [SMILES: *N1CC2NC2C1 ; *N1C2CNCC21]

In a thirteenth specific embodiment of the compound of formula (I), R is

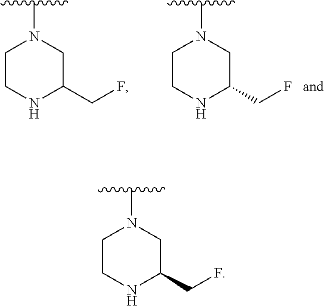

preferably selected from

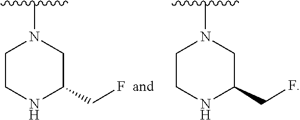

In a fourteenth specific embodiment of the compound of formula (I), R is selected from

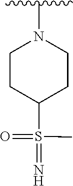 [SMILES: *N1CCC(S(C)(=N)=O)CC1]

(preferably selected from

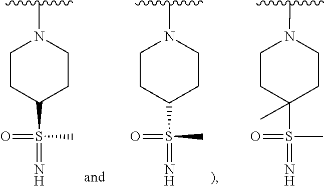 [SMILES: *N1CCC([S@@](C)(=N)=O)CC1 ; *N1CCC([S@](C)(=N)=O)CC1 ; *N1CCC(C)(S(C)(=N)=O)CC1]

(preferably selected from

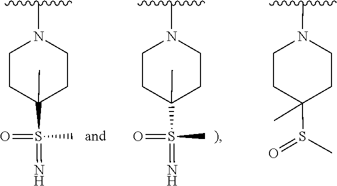

(preferably selected from

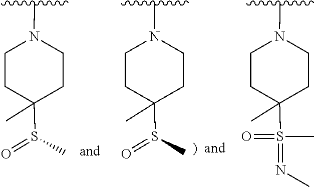 [SMILES: *N1CCC(C)([S@](C)=O)CC1 ; *N1CCC(C)([S@@](C)=O)CC1 ; *N1CCC(C)(S(C)(=O)=NC)CC1]

(preferably selected from

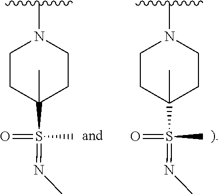

In a fifteenth specific embodiment of the compound of formula (I), R is selected from

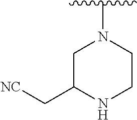 [SMILES: *N1CCNC(CC#N)C1]

(preferably selected from

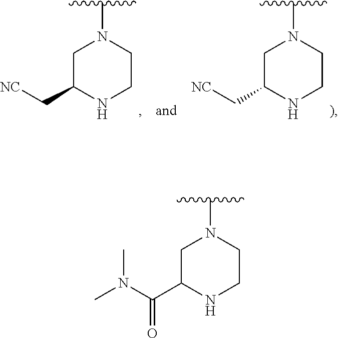 [SMILES: *N1CCN[C@@H](CC#N)C1 ; *N1CCN[C@H](CC#N)C1 ; *N1CCNC(C(=O)N(C)C)C1]

(preferably selected from

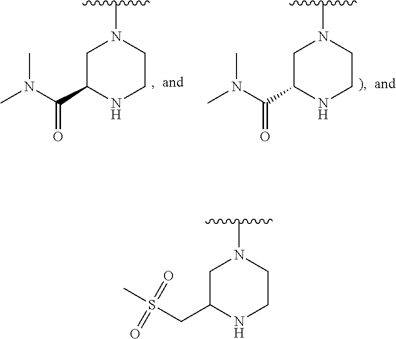 [SMILES: *N1CCN[C@@H](C(=O)N(C)C)C1 ; *N1CCN[C@H](C(=O)N(C)C)C1 ; *N1CCNC(CS(C)(=O)=O)C1]

(preferably selected from

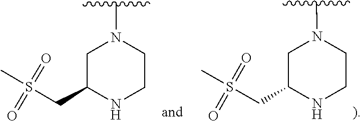 [SMILES: *N1CCN[C@@H](CS(C)(=O)=O)C1 ; *N1CCN[C@H](CS(C)(=O)=O)C1]

In a sixteenth specific embodiment of the compound of formula (I), R is

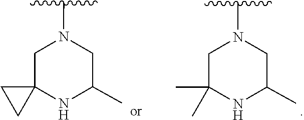

Preferably, if R is, then R is selected from

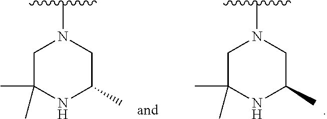

Preferably, if R is

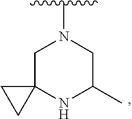 [SMILES: *N1CC(C)NC2(CC2)C1]

then R is selected from

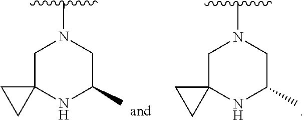

In a seventeenth specific embodiment of the compound of formula (I), R is

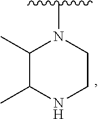 [SMILES: *N1CCNC(C)C1C]

preferably selected from,

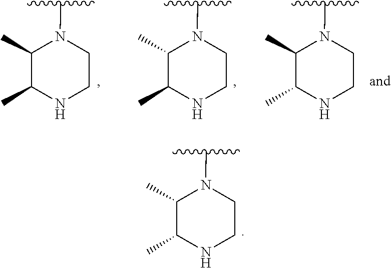

In an eighteenth specific embodiment of the compound of formula (I), R is

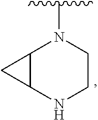 [SMILES: *N1CCNC2CC21]

preferably selected from

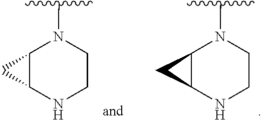 [SMILES: *N1CCN[C@@H]2C[C@@H]21 ; *N1CCN[C@H]2C[C@H]21]

In a nineteenth specific embodiment of the compound of formula (I), R is selected from

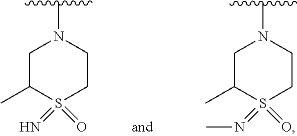

preferably R is selected from

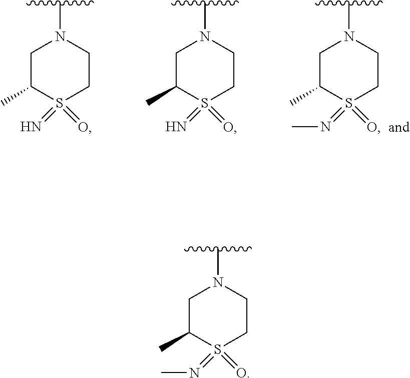

more preferably R is selected from

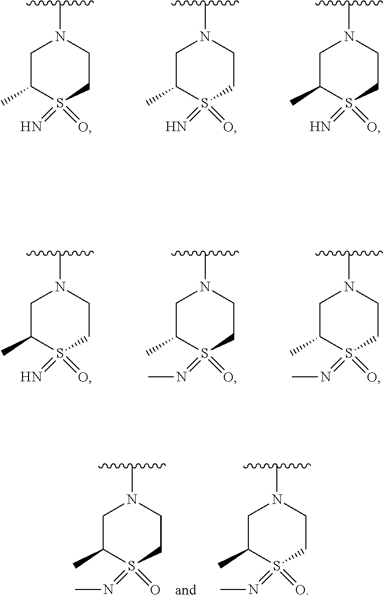

In a twentieth specific embodiment of the compound of formula (I), R is

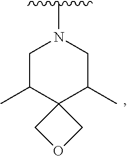

preferably R is selected from

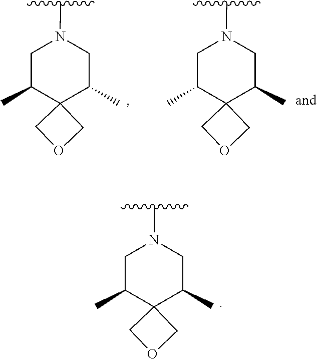

In a twenty-first specific embodiment of the compound of formula (I), R is

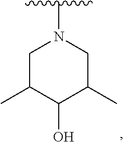

preferably R is selected from

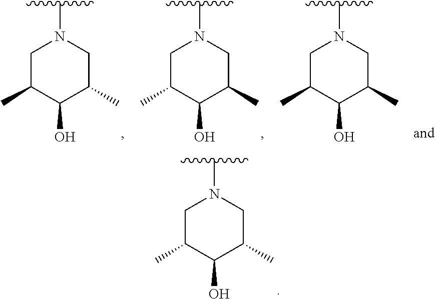

In a twenty-second specific embodiment of the compound of formula (I), R is

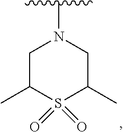

preferably R is selected from

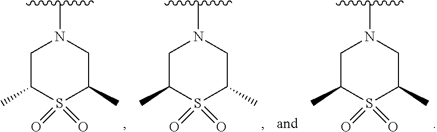

In a twenty-third specific embodiment of the compound of formula (I), R is

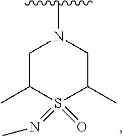

preferably R is selected from

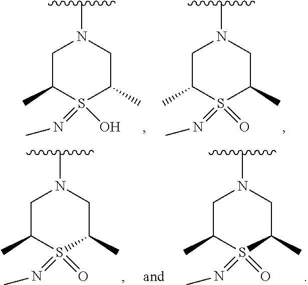

In a twenty-forth specific embodiment of the compound of formula (I), R is

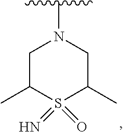

preferably R is selected from

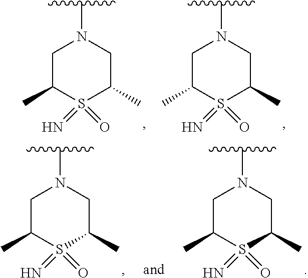

In a twenty-fifth specific embodiment of the compound of formula (I), R is

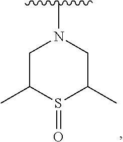

preferably R is selected from

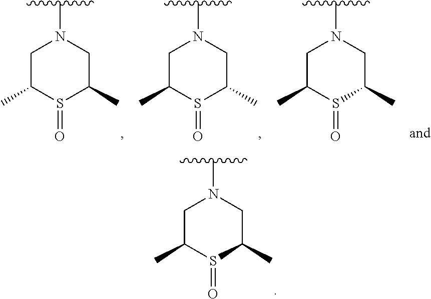

In a twenty-sixth specific embodiment of the compound of formula (I), R is selected from

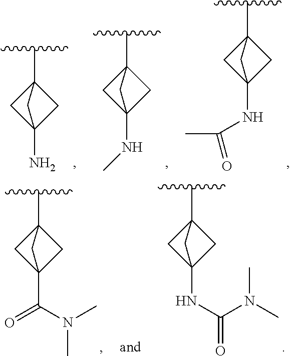

It is to be understood that in any one of first to twenty-sixth specific embodiment, R₁ is as defined for formula (I), or as defined in any one of twenty-seventh to thirty-sixth specific embodiment.

In a twenty-seventh specific embodiment of the compound of formula (I), R₁ is —CH₃. In this twenty-seventh specific embodiment, R is as in formula (I) or any one of first to twenty-sixth specific embodiments of the compound of formula (I).

In a twenty-eighth specific embodiment of the compound of formula (I), R₁ is —OH. In this twenty-eighth specific embodiment, R is as in formula (I) or any one of first to twenty-sixth specific embodiments of the compound of formula (I).

In a twenty-ninth specific embodiment of the compound of formula (I), R₁ is —OCH₃. In this twenty-ninth specific embodiment, R is as in formula (I) or any one of first to twenty-sixth specific embodiments of the compound of formula (I).

In a thirtieth specific embodiment of the compound of formula (I), R₁ is —CH₂OH. In this thirtieth specific embodiment, R is as in formula (I) or any one of first to twenty-sixth specific embodiments of the compound of formula (I).

In a thirty-first specific embodiment of the compound of formula (I), R₁ is —CH₂OCH₃. In this thirty-first specific embodiment, R is as in formula (I) or any one of first to twenty-sixth specific embodiments of the compound of formula (I).

In a thirty-second specific embodiment of the compound of formula (I), R₁ is —CH═CH₂. In this thirty-second specific embodiment, R is as in formula (I) or any one of first to twenty-sixth specific embodiments of the compound of formula (I).

In a thirty-third specific embodiment of the compound of formula (I), R₁ is —C≡CH. In this thirty-third specific embodiment, R is as in formula (I) or any one of first to twenty-sixth specific embodiments of the compound of formula (I).

In a thirty-third specific embodiment of the compound of formula (I), R₁ is —CHF₂. In this twenty-seventh specific embodiment, R is as in formula (I) or any one of first to twenty-sixth specific embodiments of the compound of formula (I).

In a thirty-forth specific embodiment of the compound of formula (I), R₁ is —CF₃. In this thirty-forth specific embodiment, R is as in formula (I) or any one of first to twenty-sixth specific embodiments of the compound of formula (I).

In a thirty-fifth specific embodiment of the compound of formula (I), R₁ is —CH₂CH₃. In this thirty-fifth specific embodiment, R is as in formula (I) or any one of first to twenty-sixth specific embodiments of the compound of formula (I).

In a thirty-sixth specific embodiment of the compound of formula (I), R₁ is —CN, In this thirty-sixth specific embodiment, R is as in formula (I) or any one of first to twenty-sixth specific embodiments of the compound of formula (I).

In a thirty seventh specific embodiment, R is

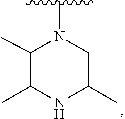

preferably R is selected from the following groups:

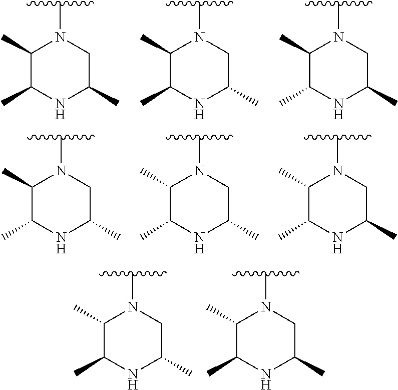

In a thirty-eighth specific embodiment, R is

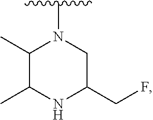 [SMILES: *N1CC(CF)NC(C)C1C]

preferably R is selected from the

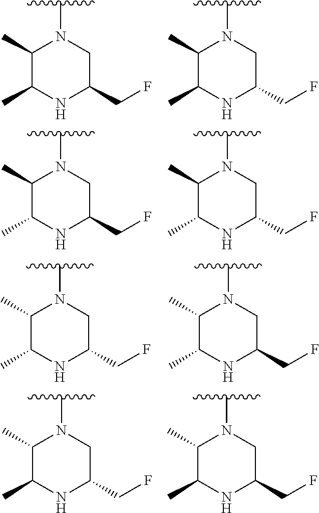

In a thirty ninth specific embodiment, R is

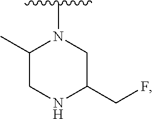 [SMILES: *N1CC(CF)NCC1C]

preferably R is selected from the following groups:

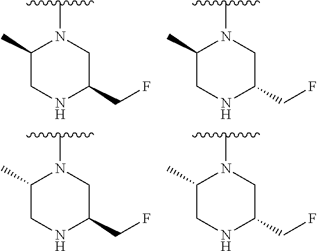

In a fortieth specific embodiment, R is

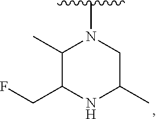 [SMILES: *N1CC(C)NC(CF)C1C]

preferably R is selected from the following groups:

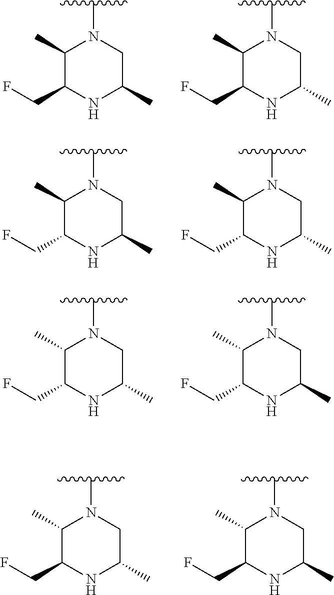

In a forty-first specific embodiment R is

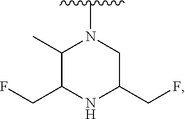 [SMILES: *N1CC(CF)NC(CF)C1C]

preferably R is selected from the following groups:

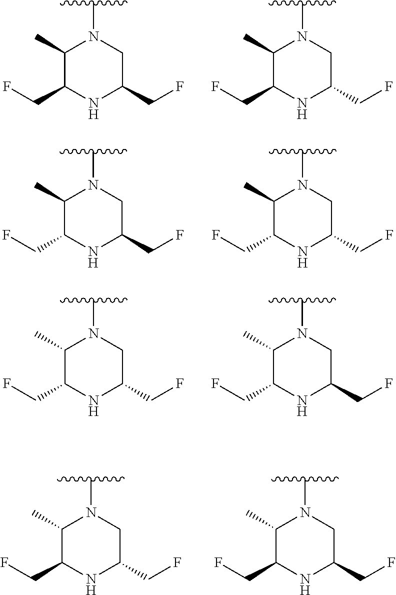

In a forty-second specific embodiment R is

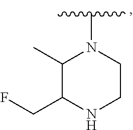 [SMILES: *N1CCNC(CF)C1C]

preferably R is selected from the following groups:

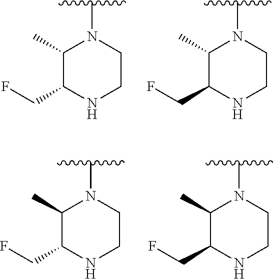

It is to be understood that in any one of thirty-seventh to forty-second specific embodiment, R₁ is as defined for formula (I), or as defined in any one of twenty-seventh to thirty-sixth specific embodiment.

In a forty-third specific embodiment, R is

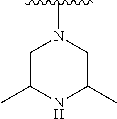

H Preferably, in this specific embodiment, R is selected from

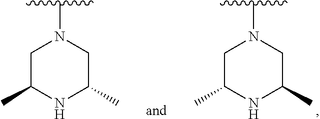

more preferably R is

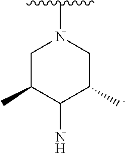

However, alternatively, in this specific embodiment R may also be

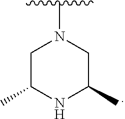

In a forty-fourth specific embodiment, R is

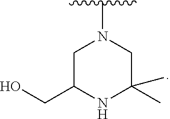 [SMILES: *N1CC(CO)NC(C)(C)C1]

Preferably, in this specific embodiment, R is selected from

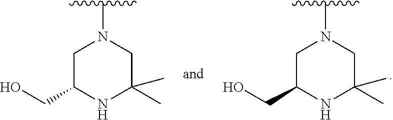 [SMILES: *N1C[C@@H](CO)NC(C)(C)C1 ; *N1C[C@H](CO)NC(C)(C)C1]

In one embodiment, preferably R is

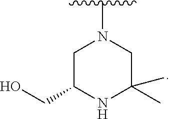 [SMILES: *N1C[C@@H](CO)NC(C)(C)C1]

### Alternatively, R is

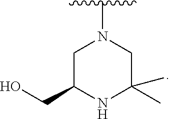 [SMILES: *N1C[C@H](CO)NC(C)(C)C1]

It is to be understood that in forty-third or forty-fourth specific embodiment, R₁ is as defined for formula (I), or as defined in any one of twenty-seventh to thirty-sixth specific embodiment.

In anyone of first to forty-fourth specific embodiments of the compound of formula (I) as described hereinabove, X₁, X₄ and X₅ are as in formula (I).

In one embodiment, the compound of formula (I) is a compound of formula (Ia):

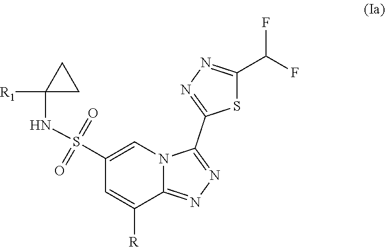

or a pharmaceutically acceptable salt thereof.

In formula (Ia), R and R₁ are as in formula (I), or in any specific embodiment of the compound of formula (I).

In one embodiment, the compound of formula (I) is a compound of formula (Ib):

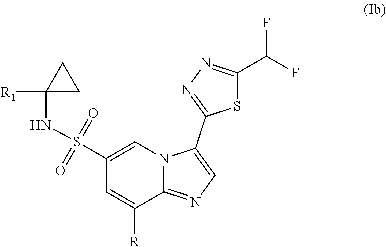

or a pharmaceutically acceptable salt thereof.

In formula (Ib), R and R₁ are as in formula (I), or in any specific embodiment of the compound of formula (I).

In one embodiment, the compound of formula (I) is a compound of formula (Ic):

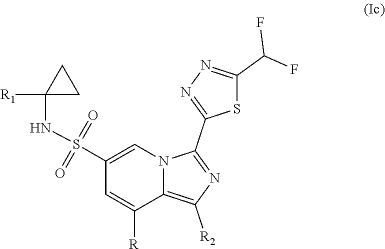

or a pharmaceutically acceptable salt thereof.

In formula (Ic), R, R₁ and R₂ are as in formula (I), or in any specific embodiment of the compound of formula (I).

In one embodiment, the compound of formula (I) is a compound of formula (Id):

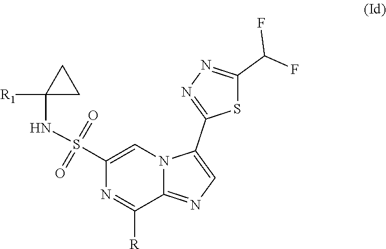

or a pharmaceutically acceptable salt thereof.

In formula (Id), R and R₁ are as in formula (I), or in any specific embodiment of the compound of formula (I).

In one embodiment, the compound of formula (I) is a compound of formula (Ic):

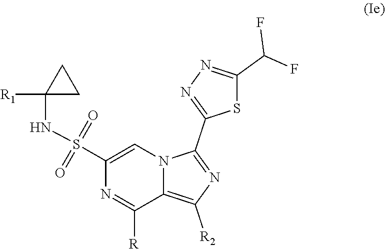

or a pharmaceutically acceptable salt thereof.

In formula (Ie), R, R₁ and R₂ are as in formula (I), or in any specific embodiment of the compound of formula (I). In one specific embodiment, R₁ may be methyl, and R₂ may be —F. Alternatively, R₁ may be methyl, and R₂ may be hydrogen.

In one embodiment, the compound of formula (I) is a compound of formula (If):

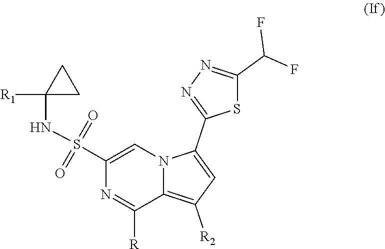

or a pharmaceutically acceptable salt thereof.

In formula (If), R, R₁ and R₂ are as in formula (I), or in any specific embodiment of the compound of formula (I).

In one embodiment, the compound of formula (I) is a compound of formula (Ig):

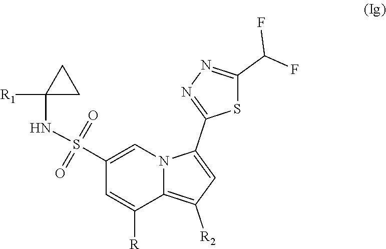

or a pharmaceutically acceptable salt thereof.

In formula (Ig), R, R₁ and R₂ are as in formula (I), or in any specific embodiment of the compound of formula (I).

Particularly preferred compounds of formula (I) are selected from the following compounds or their pharmaceutically acceptable salts:

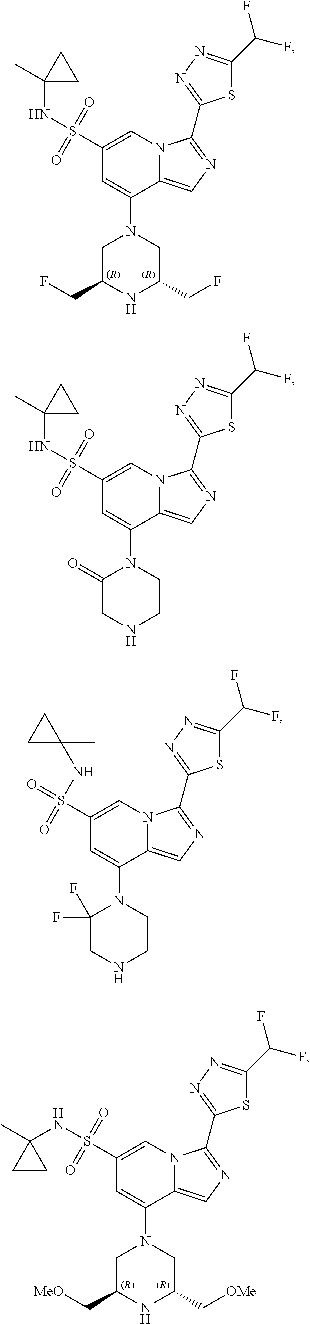

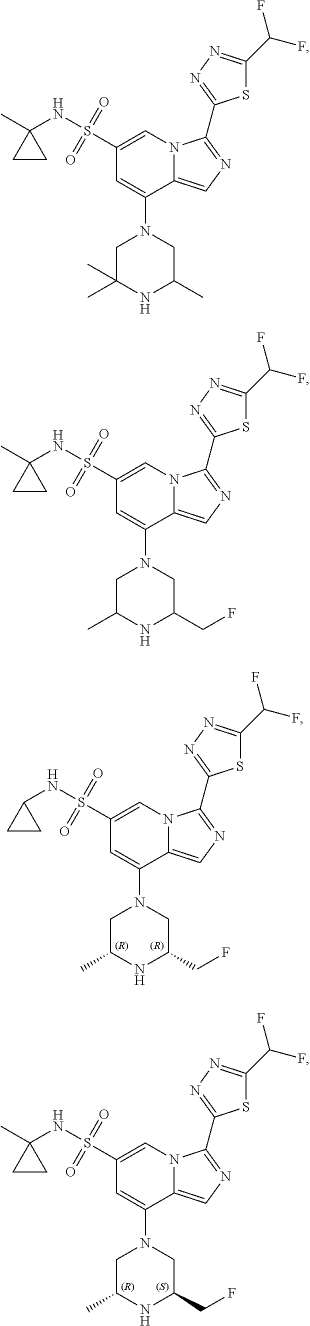

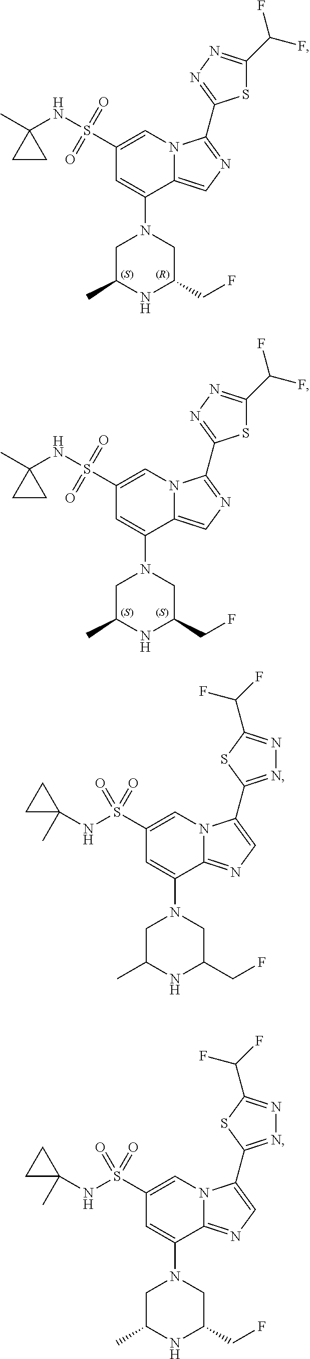

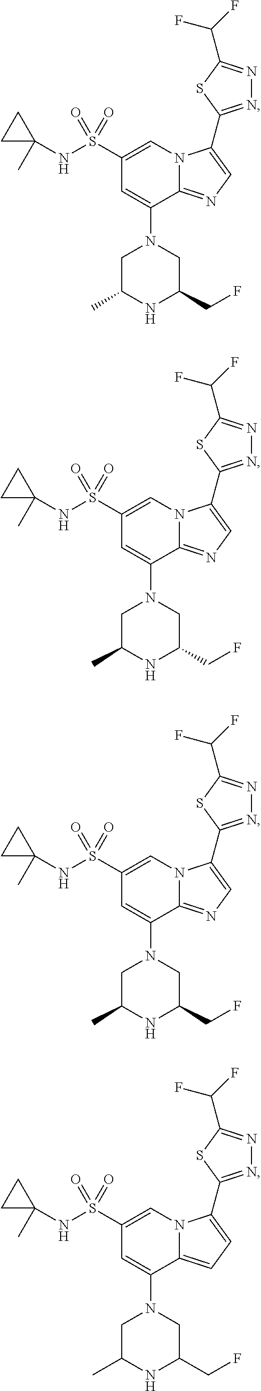 [SMILES: C[C@@H]1CN(c2cc(S(=O)(=O)NC3(C)CC3)cn3c(-c4nnc(C(F)F)s4)cnc23)C[C@@H](CF)N1 ; C[C@H]1CN(c2cc(S(=O)(=O)NC3(C)CC3)cn3c(-c4nnc(C(F)F)s4)cnc23)C[C@H](CF)N1 ; C[C@H]1CN(c2cc(S(=O)(=O)NC3(C)CC3)cn3c(-c4nnc(C(F)F)s4)cnc23)C[C@@H](CF)N1 ; CC1CN(c2cc(S(=O)(=O)NC3(C)CC3)cn3c(-c4nnc(C(F)F)s4)ccc23)CC(CF)N1]

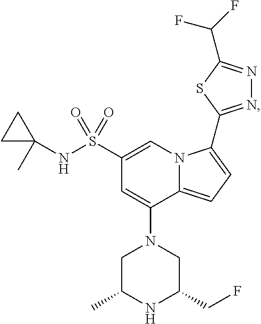 [SMILES: C[C@@H]1CN(c2cc(S(=O)(=O)NC3(C)CC3)cn3c(-c4nnc(C(F)F)s4)ccc23)C[C@H](CF)N1]

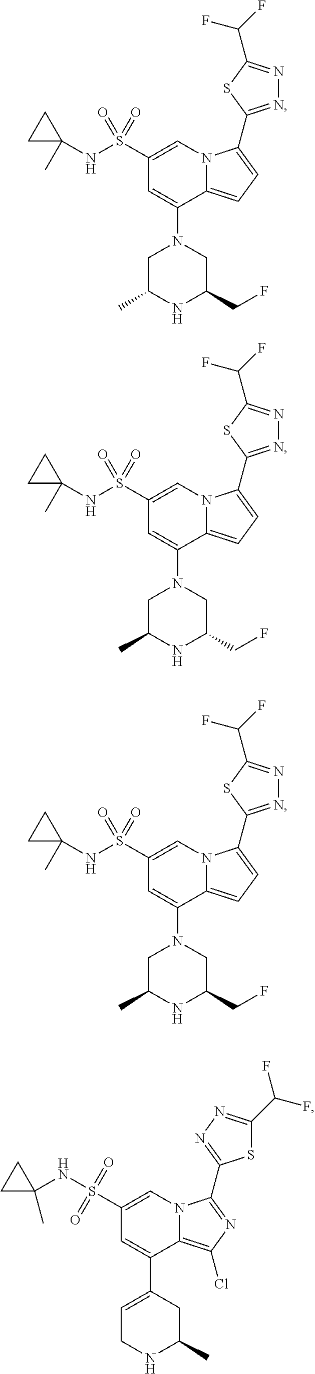 [SMILES: C[C@@H]1CN(c2cc(S(=O)(=O)NC3(C)CC3)cn3c(-c4nnc(C(F)F)s4)ccc23)C[C@@H](CF)N1 ; C[C@H]1CN(c2cc(S(=O)(=O)NC3(C)CC3)cn3c(-c4nnc(C(F)F)s4)ccc23)C[C@H](CF)N1 ; C[C@H]1CN(c2cc(S(=O)(=O)NC3(C)CC3)cn3c(-c4nnc(C(F)F)s4)ccc23)C[C@@H](CF)N1 ; C[C@@H]1CC(c2cc(S(=O)(=O)NC3(C)CC3)cn3c(-c4nnc(C(F)F)s4)nc(Cl)c23)=CCN1]

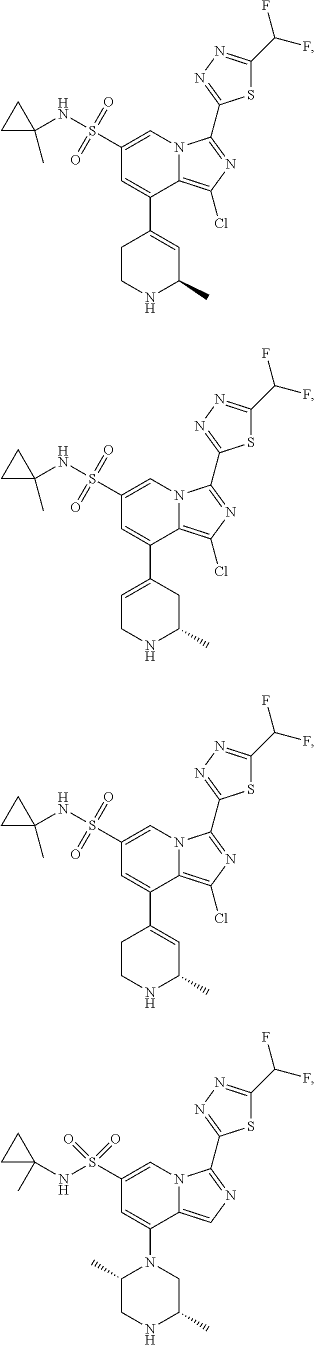 [SMILES: C[C@@H]1C=C(c2cc(S(=O)(=O)NC3(C)CC3)cn3c(-c4nnc(C(F)F)s4)nc(Cl)c23)CCN1 ; C[C@H]1CC(c2cc(S(=O)(=O)NC3(C)CC3)cn3c(-c4nnc(C(F)F)s4)nc(Cl)c23)=CCN1 ; C[C@H]1C=C(c2cc(S(=O)(=O)NC3(C)CC3)cn3c(-c4nnc(C(F)F)s4)nc(Cl)c23)CCN1 ; C[C@H]1CN(c2cc(S(=O)(=O)NC3(C)CC3)cn3c(-c4nnc(C(F)F)s4)ncc23)[C@@H](C)CN1]

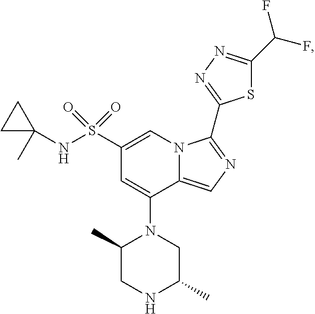 [SMILES: C[C@@H]1CN[C@@H](C)CN1c1cc(S(=O)(=O)NC2(C)CC2)cn2c(-c3nnc(C(F)F)s3)ncc12]

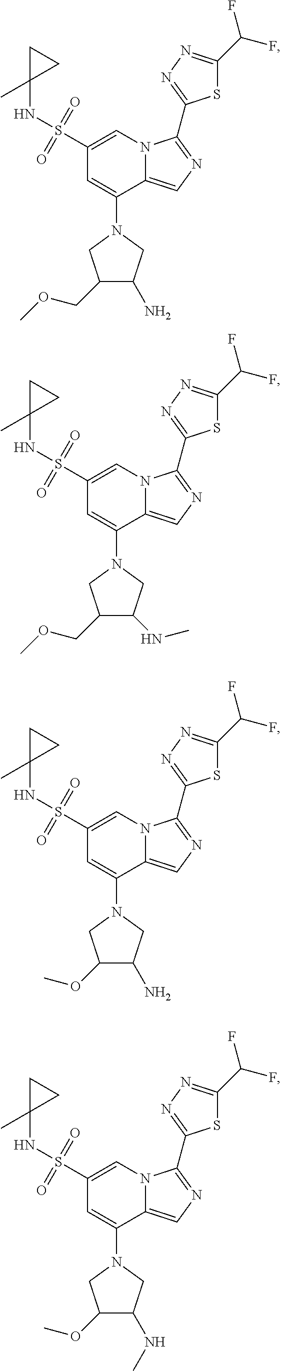 [SMILES: COCC1CN(c2cc(S(=O)(=O)NC3(C)CC3)cn3c(-c4nnc(C(F)F)s4)ncc23)CC1N ; CNC1CN(c2cc(S(=O)(=O)NC3(C)CC3)cn3c(-c4nnc(C(F)F)s4)ncc23)CC1COC ; COC1CN(c2cc(S(=O)(=O)NC3(C)CC3)cn3c(-c4nnc(C(F)F)s4)ncc23)CC1N ; CNC1CN(c2cc(S(=O)(=O)NC3(C)CC3)cn3c(-c4nnc(C(F)F)s4)ncc23)CC1OC]

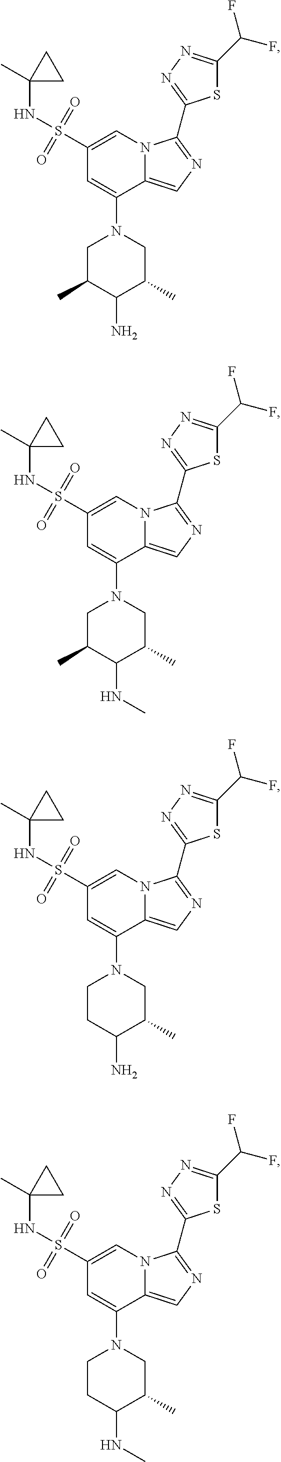 [SMILES: C[C@H]1CN(c2cc(S(=O)(=O)NC3(C)CC3)cn3c(-c4nnc(C(F)F)s4)ncc23)C[C@H](C)C1N ; CNC1[C@@H](C)CN(c2cc(S(=O)(=O)NC3(C)CC3)cn3c(-c4nnc(C(F)F)s4)ncc23)C[C@@H]1C ; C[C@H]1CN(c2cc(S(=O)(=O)NC3(C)CC3)cn3c(-c4nnc(C(F)F)s4)ncc23)CCC1N ; CNC1CCN(c2cc(S(=O)(=O)NC3(C)CC3)cn3c(-c4nnc(C(F)F)s4)ncc23)C[C@@H]1C]

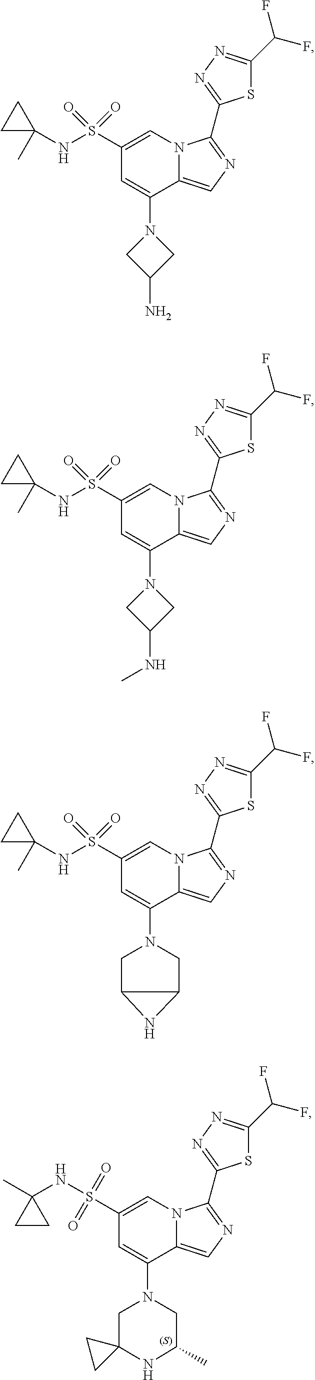

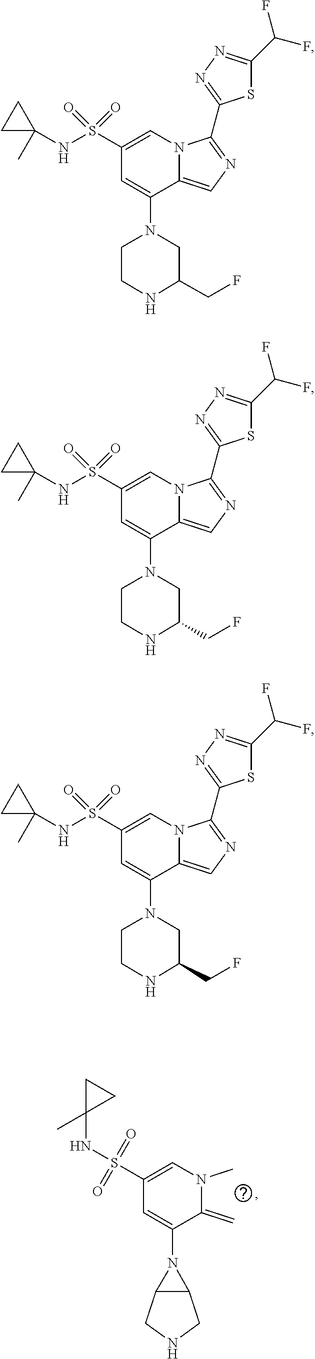

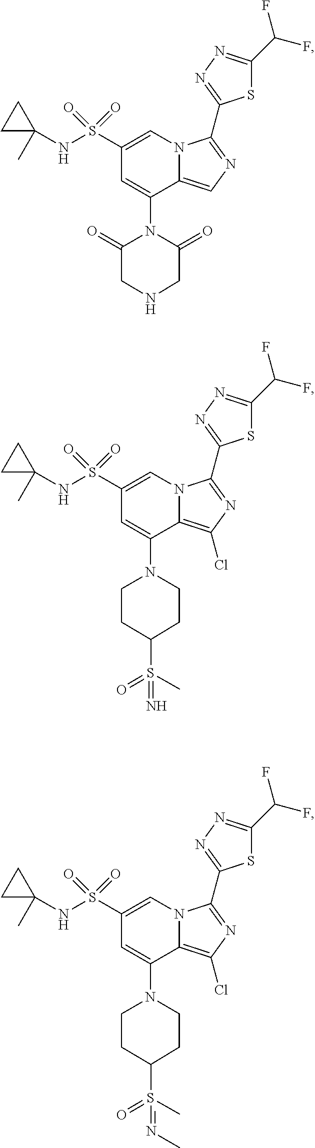 [SMILES: CC1(NS(=O)(=O)c2cc(N3C(=O)CNCC3=O)c3cnc(-c4nnc(C(F)F)s4)n3c2)CC1 ; CC1(NS(=O)(=O)c2cc(N3CCC(S(C)(=N)=O)CC3)c3c(Cl)nc(-c4nnc(C(F)F)s4)n3c2)CC1 ; CN=S(C)(=O)C1CCN(c2cc(S(=O)(=O)NC3(C)CC3)cn3c(-c4nnc(C(F)F)s4)nc(Cl)c23)CC1]

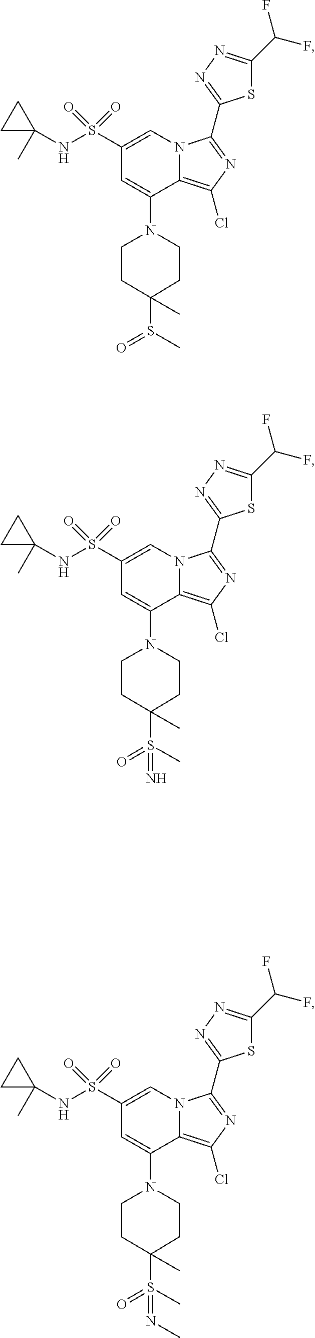 [SMILES: CS(=O)C1(C)CCN(c2cc(S(=O)(=O)NC3(C)CC3)cn3c(-c4nnc(C(F)F)s4)nc(Cl)c23)CC1 ; CC1(NS(=O)(=O)c2cc(N3CCC(C)(S(C)(=N)=O)CC3)c3c(Cl)nc(-c4nnc(C(F)F)s4)n3c2)CC1 ; CN=S(C)(=O)C1(C)CCN(c2cc(S(=O)(=O)NC3(C)CC3)cn3c(-c4nnc(C(F)F)s4)nc(Cl)c23)CC1]

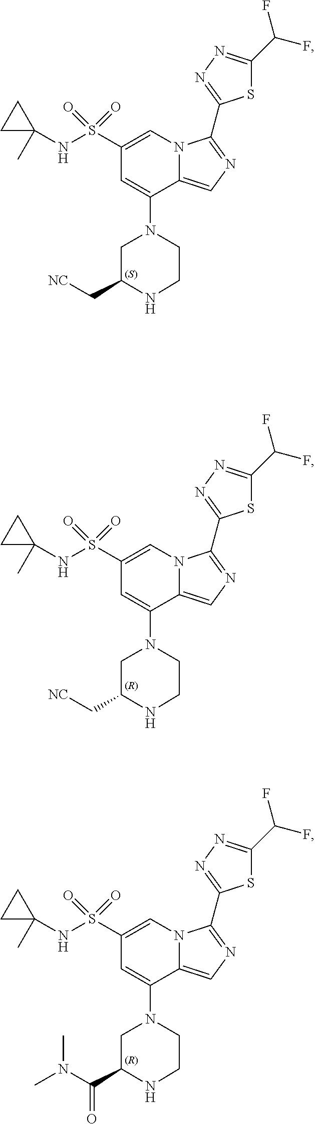

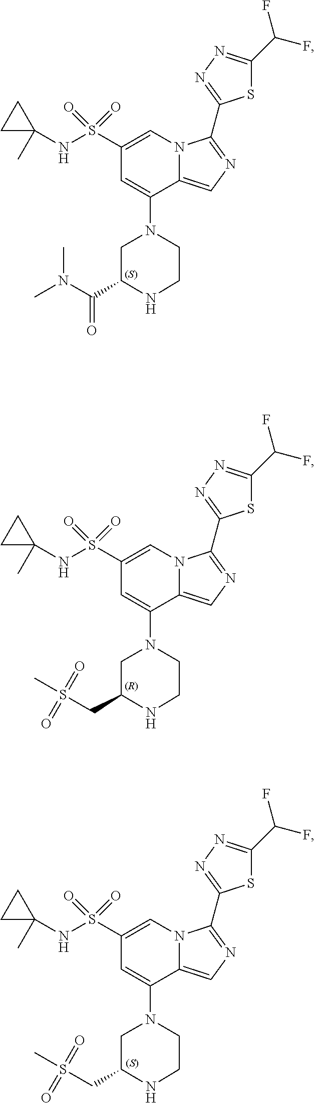

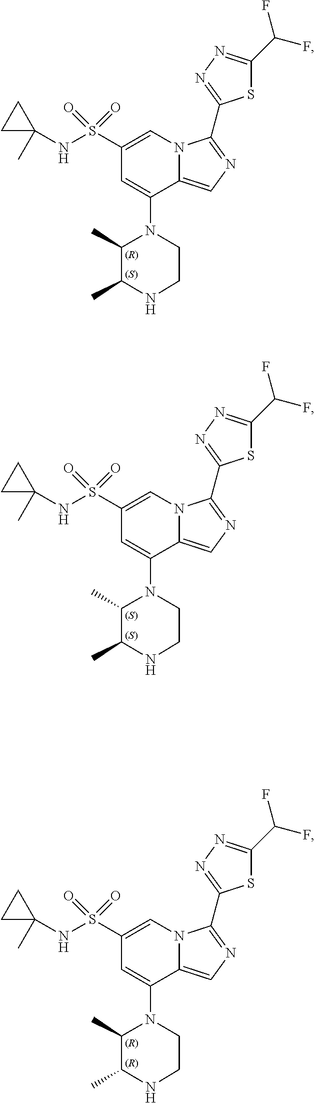

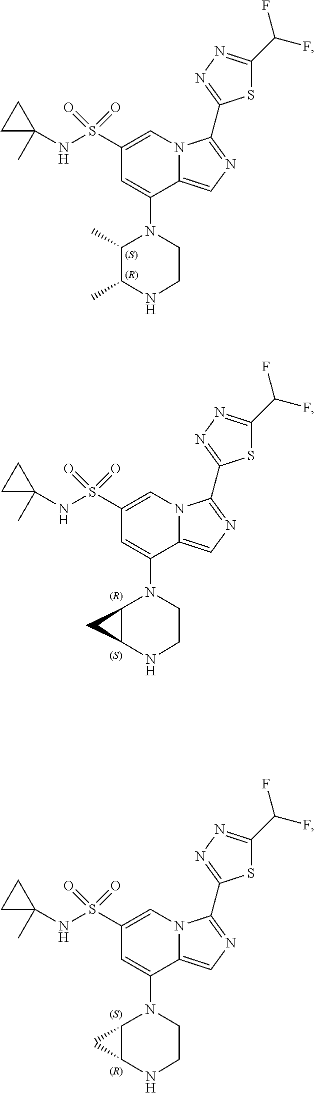

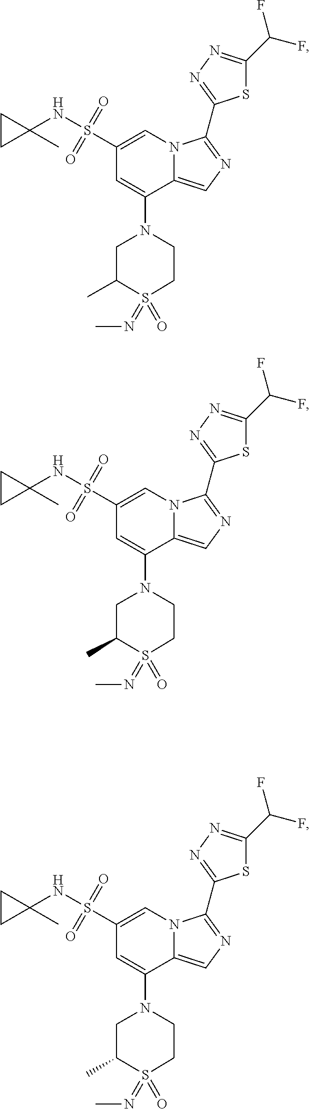 [SMILES: CN=S1(=O)CCN(c2cc(S(=O)(=O)NC3(C)CC3)cn3c(-c4nnc(C(F)F)s4)ncc23)CC1C ; CN=S1(=O)CCN(c2cc(S(=O)(=O)NC3(C)CC3)cn3c(-c4nnc(C(F)F)s4)ncc23)C[C@@H]1C ; CN=S1(=O)CCN(c2cc(S(=O)(=O)NC3(C)CC3)cn3c(-c4nnc(C(F)F)s4)ncc23)C[C@H]1C]

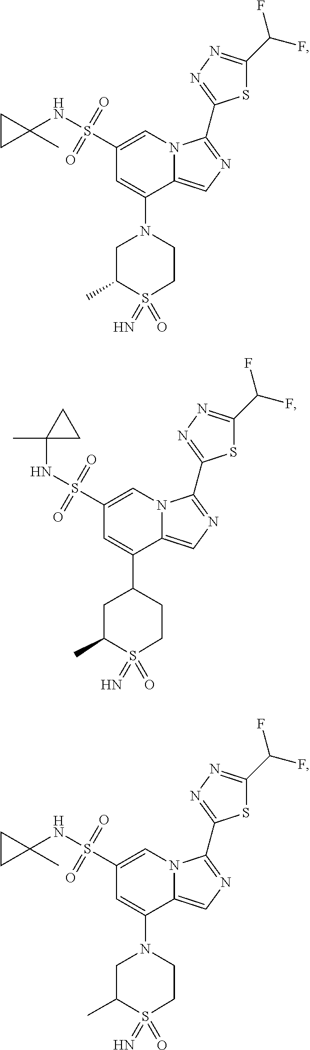 [SMILES: C[C@@H]1CN(c2cc(S(=O)(=O)NC3(C)CC3)cn3c(-c4nnc(C(F)F)s4)ncc23)CCS1(=N)=O ; C[C@H]1CC(c2cc(S(=O)(=O)NC3(C)CC3)cn3c(-c4nnc(C(F)F)s4)ncc23)CCS1(=N)=O ; CC1CN(c2cc(S(=O)(=O)NC3(C)CC3)cn3c(-c4nnc(C(F)F)s4)ncc23)CCS1(=N)=O]

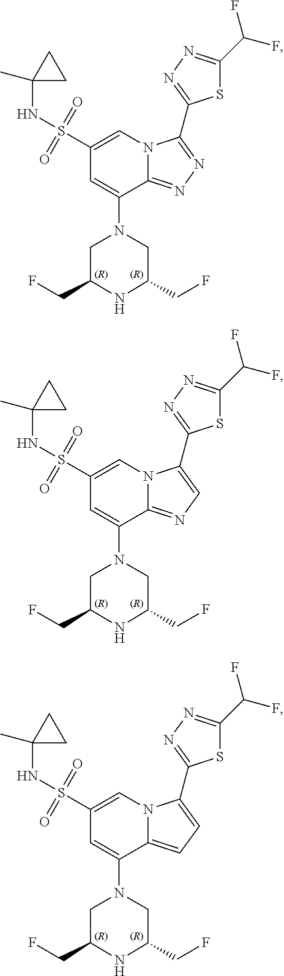

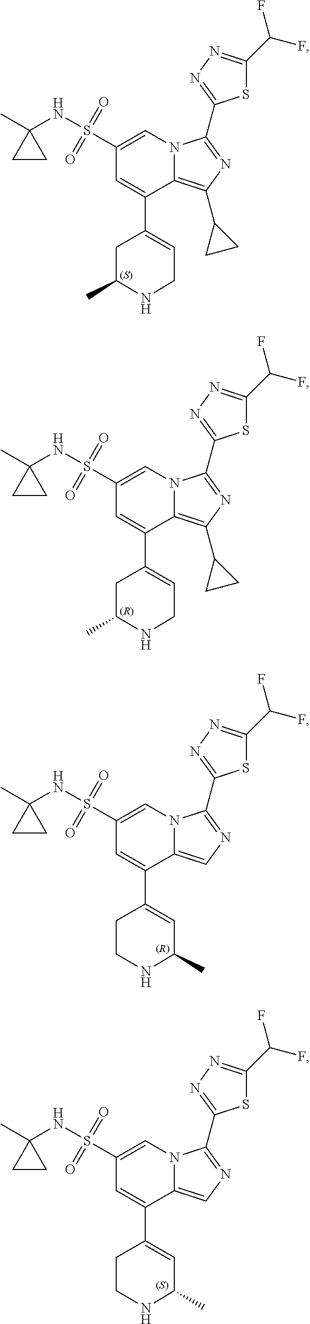

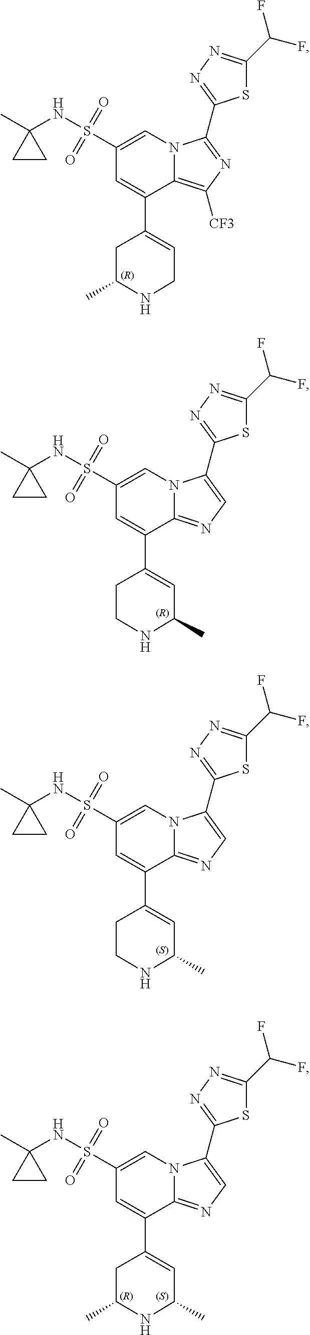

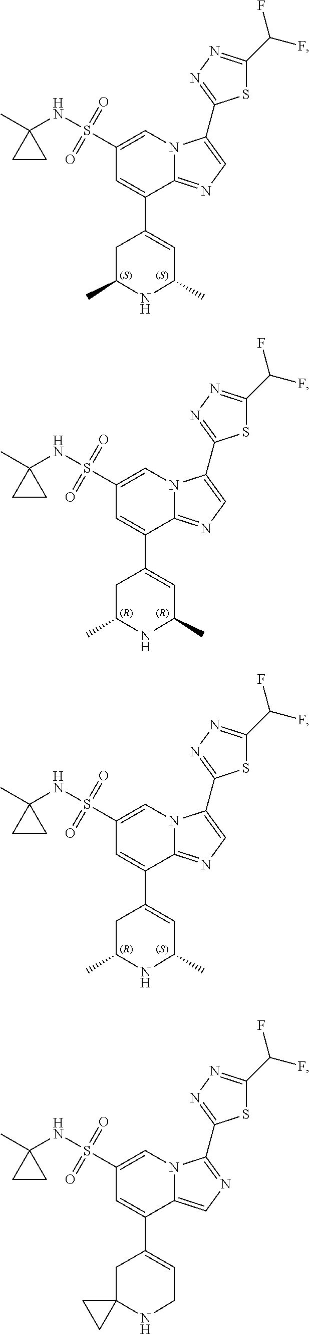

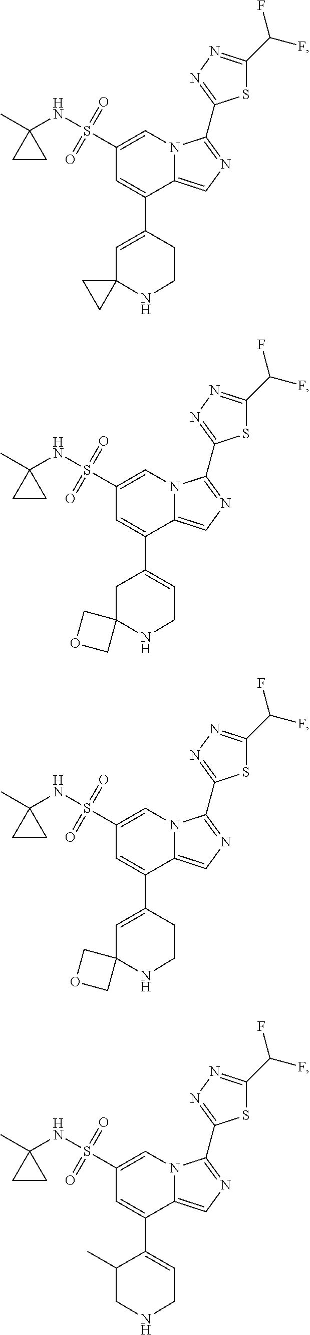 [SMILES: CC1(NS(=O)(=O)c2cc(C3=CC4(CC4)NCC3)c3cnc(-c4nnc(C(F)F)s4)n3c2)CC1 ; CC1(NS(=O)(=O)c2cc(C3=CCNC4(COC4)C3)c3cnc(-c4nnc(C(F)F)s4)n3c2)CC1 ; CC1(NS(=O)(=O)c2cc(C3=CC4(COC4)NCC3)c3cnc(-c4nnc(C(F)F)s4)n3c2)CC1 ; CC1CNCC=C1c1cc(S(=O)(=O)NC2(C)CC2)cn2c(-c3nnc(C(F)F)s3)ncc12]

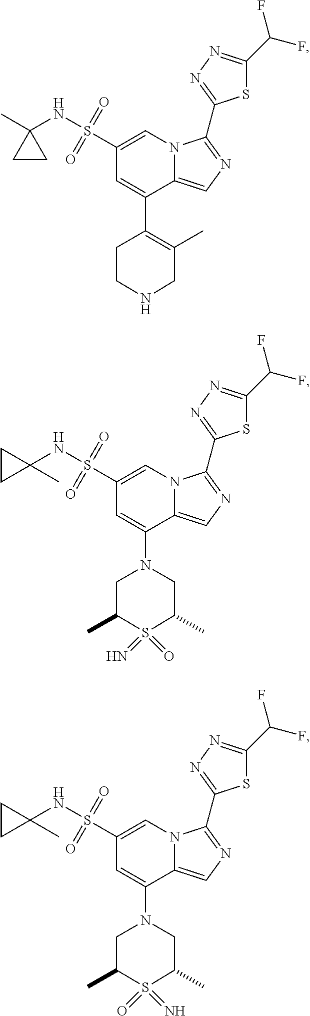 [SMILES: CC1=C(c2cc(S(=O)(=O)NC3(C)CC3)cn3c(-c4nnc(C(F)F)s4)ncc23)CCNC1 ; C[C@H]1CN(c2cc(S(=O)(=O)NC3(C)CC3)cn3c(-c4nnc(C(F)F)s4)ncc23)C[C@H](C)S1(=N)=O ; C[C@H]1CN(c2cc(S(=O)(=O)NC3(C)CC3)cn3c(-c4nnc(C(F)F)s4)ncc23)C[C@H](C)S1(=N)=O]

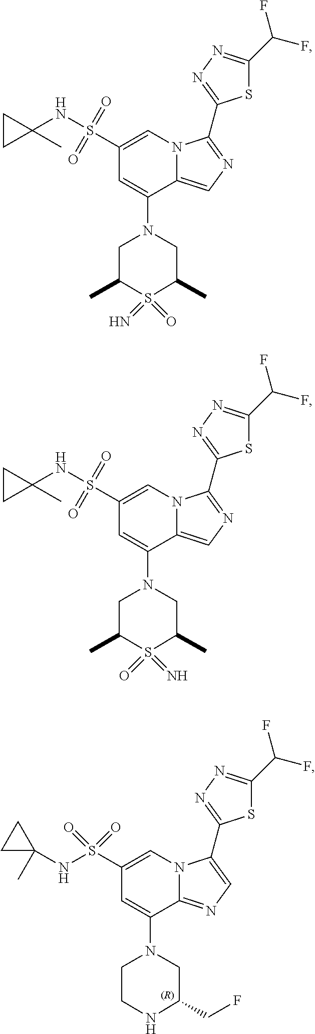

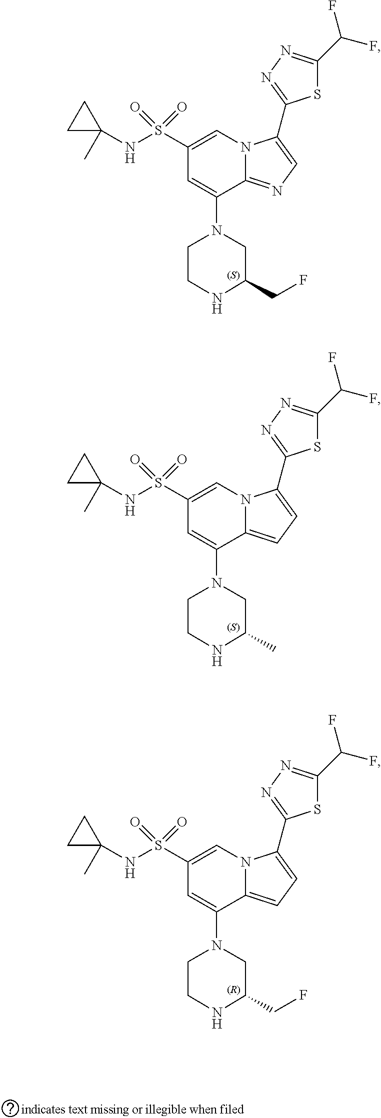

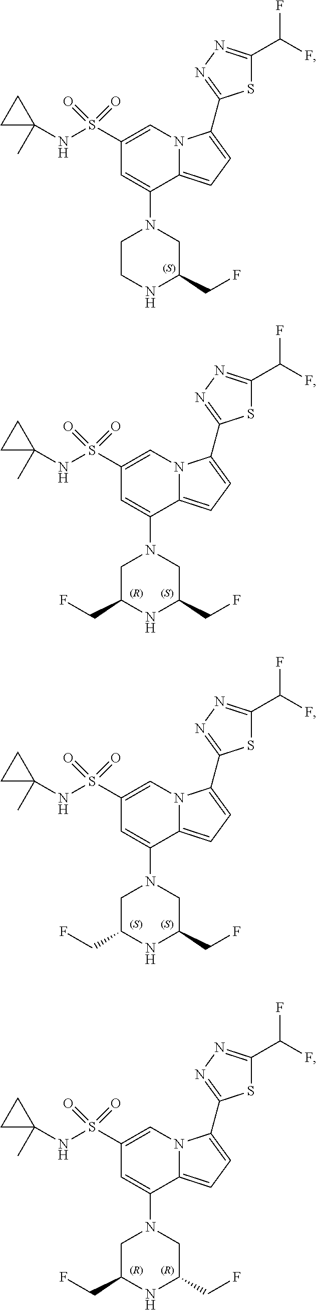

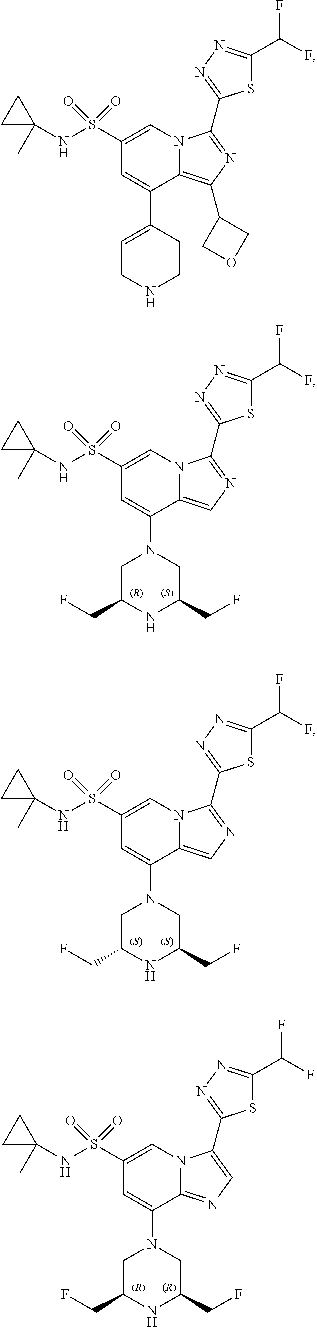

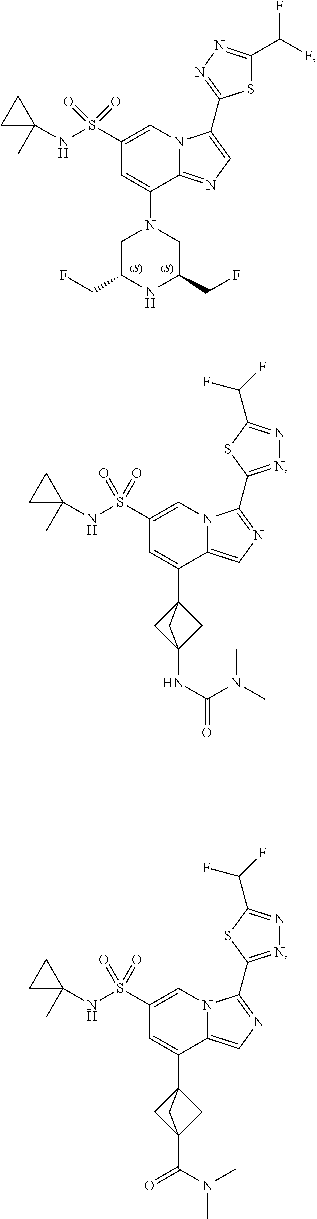

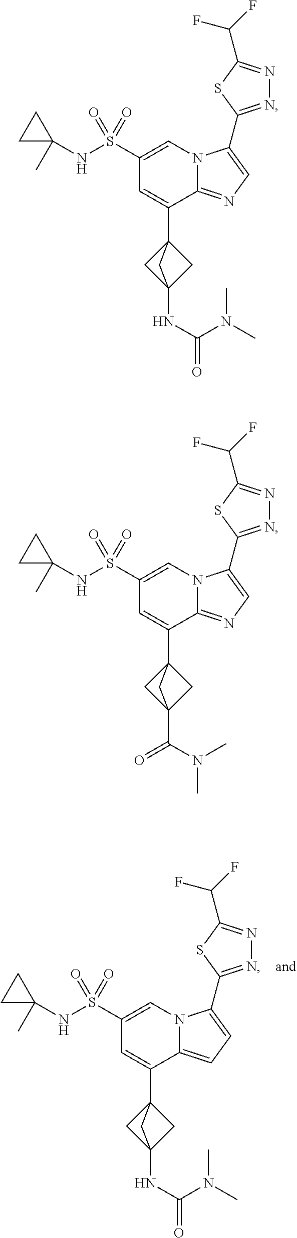 [SMILES: CN(C)C(=O)NC12CC(c3cc(S(=O)(=O)NC4(C)CC4)cn4c(-c5nnc(C(F)F)s5)cnc34)(C1)C2 ; CN(C)C(=O)C12CC(c3cc(S(=O)(=O)NC4(C)CC4)cn4c(-c5nnc(C(F)F)s5)cnc34)(C1)C2 ; CN(C)C(=O)NC12CC(c3cc(S(=O)(=O)NC4(C)CC4)cn4c(-c5nnc(C(F)F)s5)ccc34)(C1)C2]

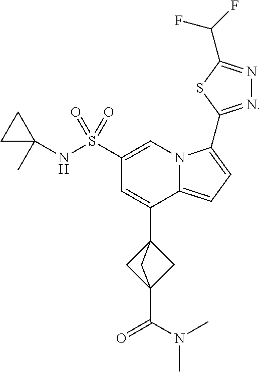 [SMILES: CN(C)C(=O)C12CC(c3cc(S(=O)(=O)NC4(C)CC4)cn4c(-c5nnc(C(F)F)s5)ccc34)(C1)C2]

Further particularly preferred compounds of formula (I) are selected from the following compounds or their pharmaceutically acceptable salts:

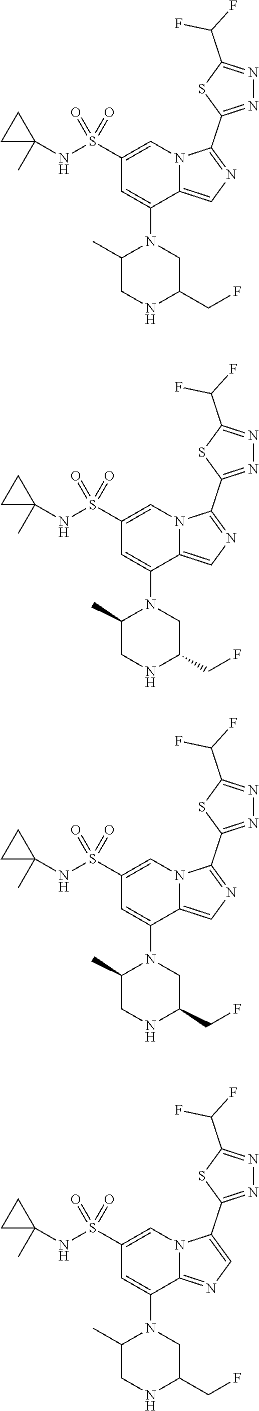 [SMILES: CC1CNC(CF)CN1c1cc(S(=O)(=O)NC2(C)CC2)cn2c(-c3nnc(C(F)F)s3)ncc12 ; C[C@@H]1CN[C@@H](CF)CN1c1cc(S(=O)(=O)NC2(C)CC2)cn2c(-c3nnc(C(F)F)s3)ncc12 ; C[C@@H]1CN[C@H](CF)CN1c1cc(S(=O)(=O)NC2(C)CC2)cn2c(-c3nnc(C(F)F)s3)ncc12 ; CC1CNC(CF)CN1c1cc(S(=O)(=O)NC2(C)CC2)cn2c(-c3nnc(C(F)F)s3)cnc12]

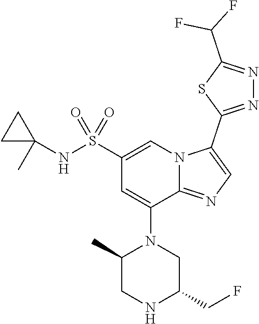 [SMILES: C[C@@H]1CN[C@@H](CF)CN1c1cc(S(=O)(=O)NC2(C)CC2)cn2c(-c3nnc(C(F)F)s3)cnc12]

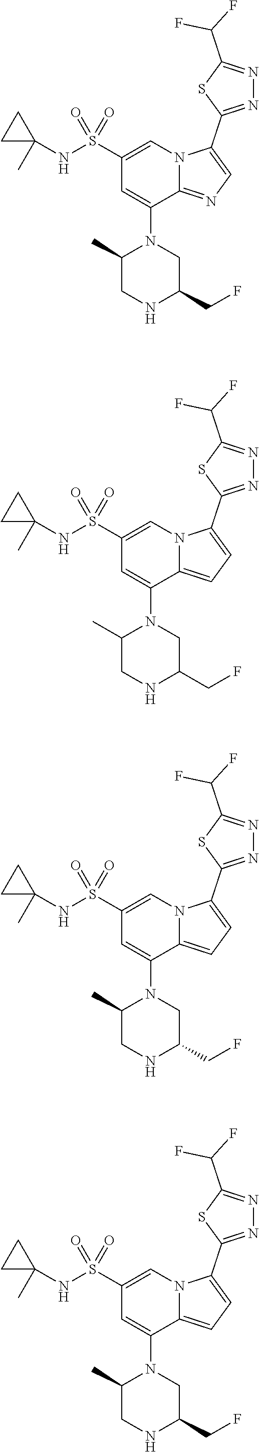 [SMILES: C[C@@H]1CN[C@H](CF)CN1c1cc(S(=O)(=O)NC2(C)CC2)cn2c(-c3nnc(C(F)F)s3)cnc12 ; CC1CNC(CF)CN1c1cc(S(=O)(=O)NC2(C)CC2)cn2c(-c3nnc(C(F)F)s3)ccc12 ; C[C@@H]1CN[C@@H](CF)CN1c1cc(S(=O)(=O)NC2(C)CC2)cn2c(-c3nnc(C(F)F)s3)ccc12 ; C[C@@H]1CN[C@H](CF)CN1c1cc(S(=O)(=O)NC2(C)CC2)cn2c(-c3nnc(C(F)F)s3)ccc12]

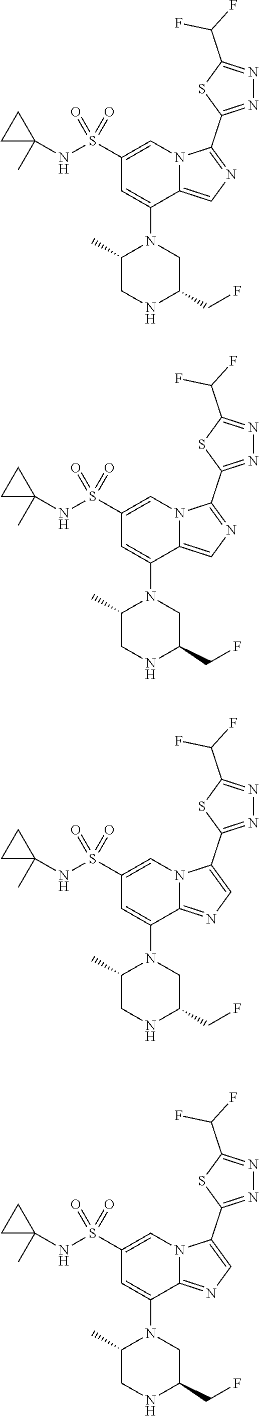 [SMILES: C[C@H]1CN[C@@H](CF)CN1c1cc(S(=O)(=O)NC2(C)CC2)cn2c(-c3nnc(C(F)F)s3)ncc12 ; C[C@H]1CN[C@H](CF)CN1c1cc(S(=O)(=O)NC2(C)CC2)cn2c(-c3nnc(C(F)F)s3)ncc12 ; C[C@H]1CN[C@@H](CF)CN1c1cc(S(=O)(=O)NC2(C)CC2)cn2c(-c3nnc(C(F)F)s3)cnc12 ; C[C@H]1CN[C@H](CF)CN1c1cc(S(=O)(=O)NC2(C)CC2)cn2c(-c3nnc(C(F)F)s3)cnc12]

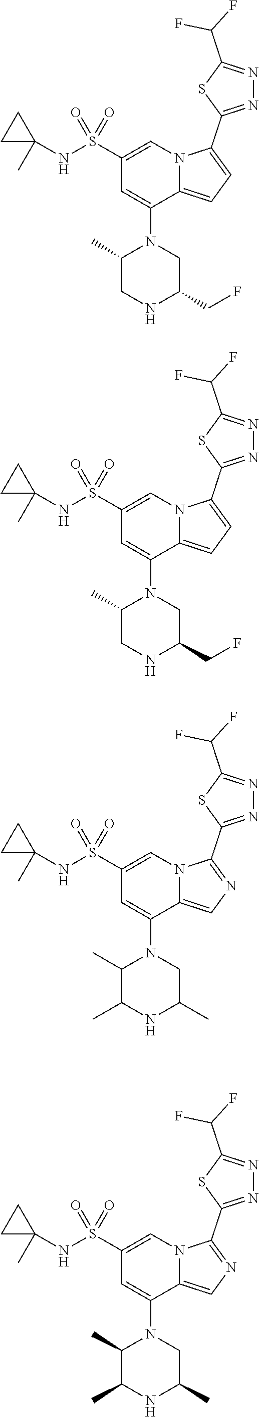 [SMILES: C[C@H]1CN[C@@H](CF)CN1c1cc(S(=O)(=O)NC2(C)CC2)cn2c(-c3nnc(C(F)F)s3)ccc12 ; C[C@H]1CN[C@H](CF)CN1c1cc(S(=O)(=O)NC2(C)CC2)cn2c(-c3nnc(C(F)F)s3)ccc12 ; CC1CN(c2cc(S(=O)(=O)NC3(C)CC3)cn3c(-c4nnc(C(F)F)s4)ncc23)C(C)C(C)N1 ; C[C@@H]1CN(c2cc(S(=O)(=O)NC3(C)CC3)cn3c(-c4nnc(C(F)F)s4)ncc23)[C@H](C)[C@H](C)N1]

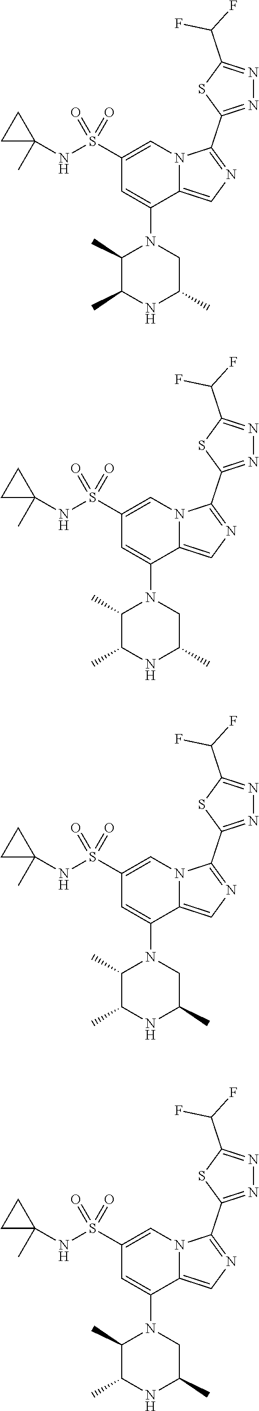 [SMILES: C[C@@H]1N[C@@H](C)CN(c2cc(S(=O)(=O)NC3(C)CC3)cn3c(-c4nnc(C(F)F)s4)ncc23)[C@@H]1C ; C[C@H]1CN(c2cc(S(=O)(=O)NC3(C)CC3)cn3c(-c4nnc(C(F)F)s4)ncc23)[C@@H](C)[C@@H](C)N1 ; C[C@@H]1CN(c2cc(S(=O)(=O)NC3(C)CC3)cn3c(-c4nnc(C(F)F)s4)ncc23)[C@@H](C)[C@@H](C)N1 ; C[C@@H]1CN(c2cc(S(=O)(=O)NC3(C)CC3)cn3c(-c4nnc(C(F)F)s4)ncc23)[C@H](C)[C@@H](C)N1]

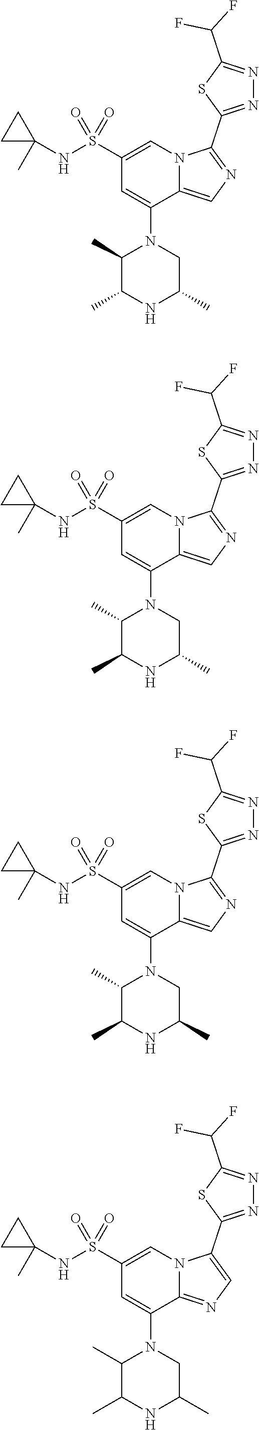 [SMILES: C[C@@H]1[C@@H](C)N[C@@H](C)CN1c1cc(S(=O)(=O)NC2(C)CC2)cn2c(-c3nnc(C(F)F)s3)ncc12 ; C[C@@H]1N[C@@H](C)CN(c2cc(S(=O)(=O)NC3(C)CC3)cn3c(-c4nnc(C(F)F)s4)ncc23)[C@H]1C ; C[C@@H]1CN(c2cc(S(=O)(=O)NC3(C)CC3)cn3c(-c4nnc(C(F)F)s4)ncc23)[C@@H](C)[C@H](C)N1 ; CC1CN(c2cc(S(=O)(=O)NC3(C)CC3)cn3c(-c4nnc(C(F)F)s4)cnc23)C(C)C(C)N1]

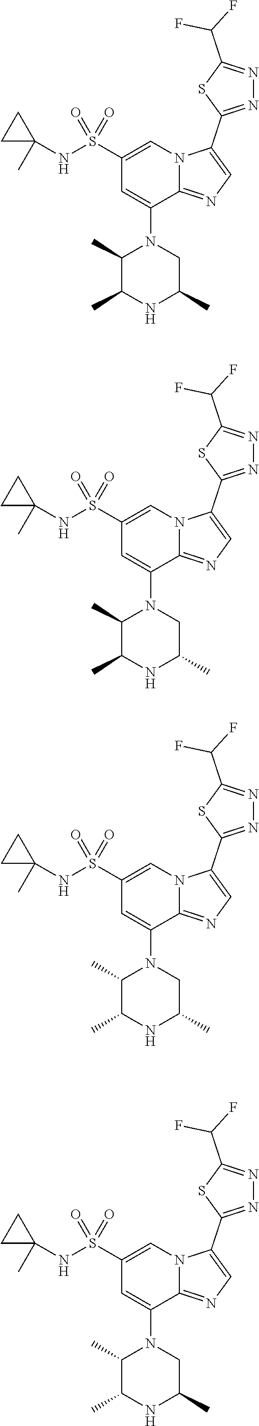 [SMILES: C[C@@H]1CN(c2cc(S(=O)(=O)NC3(C)CC3)cn3c(-c4nnc(C(F)F)s4)cnc23)[C@H](C)[C@H](C)N1 ; C[C@@H]1N[C@@H](C)CN(c2cc(S(=O)(=O)NC3(C)CC3)cn3c(-c4nnc(C(F)F)s4)cnc23)[C@@H]1C ; C[C@H]1CN(c2cc(S(=O)(=O)NC3(C)CC3)cn3c(-c4nnc(C(F)F)s4)cnc23)[C@@H](C)[C@@H](C)N1 ; C[C@@H]1CN(c2cc(S(=O)(=O)NC3(C)CC3)cn3c(-c4nnc(C(F)F)s4)cnc23)[C@@H](C)[C@@H](C)N1]

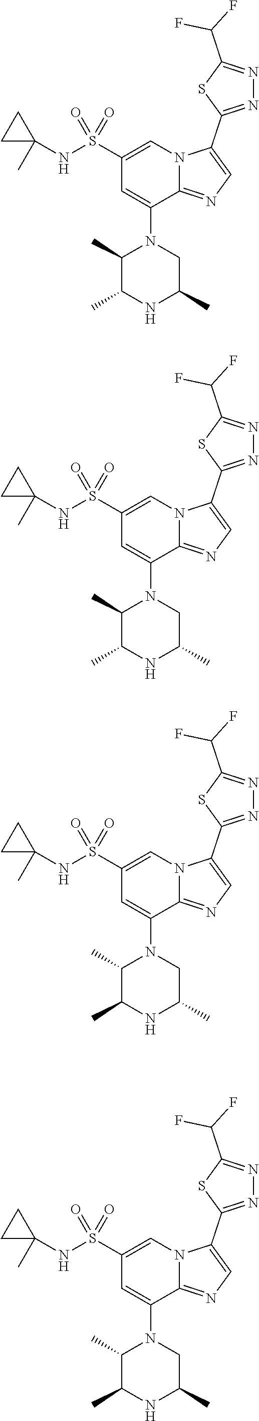 [SMILES: C[C@@H]1CN(c2cc(S(=O)(=O)NC3(C)CC3)cn3c(-c4nnc(C(F)F)s4)cnc23)[C@H](C)[C@@H](C)N1 ; C[C@@H]1[C@@H](C)N[C@@H](C)CN1c1cc(S(=O)(=O)NC2(C)CC2)cn2c(-c3nnc(C(F)F)s3)cnc12 ; C[C@@H]1N[C@@H](C)CN(c2cc(S(=O)(=O)NC3(C)CC3)cn3c(-c4nnc(C(F)F)s4)cnc23)[C@H]1C ; C[C@@H]1CN(c2cc(S(=O)(=O)NC3(C)CC3)cn3c(-c4nnc(C(F)F)s4)cnc23)[C@@H](C)[C@H](C)N1]

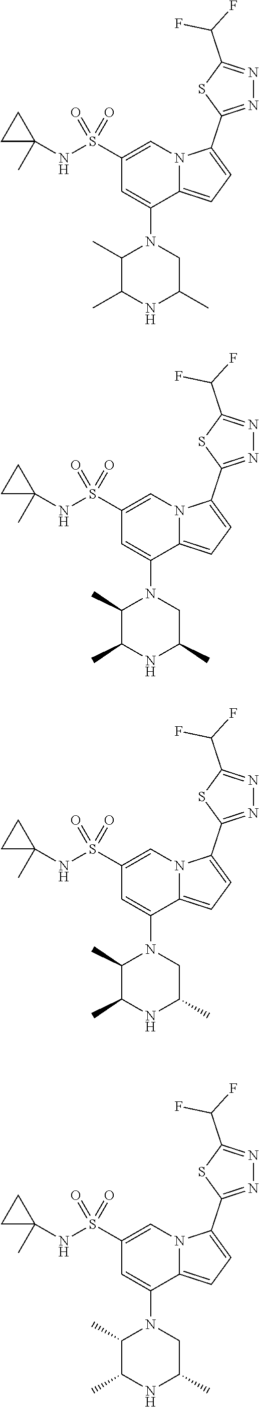 [SMILES: CC1CN(c2cc(S(=O)(=O)NC3(C)CC3)cn3c(-c4nnc(C(F)F)s4)ccc23)C(C)C(C)N1 ; C[C@@H]1CN(c2cc(S(=O)(=O)NC3(C)CC3)cn3c(-c4nnc(C(F)F)s4)ccc23)[C@H](C)[C@H](C)N1 ; C[C@@H]1N[C@@H](C)CN(c2cc(S(=O)(=O)NC3(C)CC3)cn3c(-c4nnc(C(F)F)s4)ccc23)[C@@H]1C ; C[C@H]1CN(c2cc(S(=O)(=O)NC3(C)CC3)cn3c(-c4nnc(C(F)F)s4)ccc23)[C@@H](C)[C@@H](C)N1]

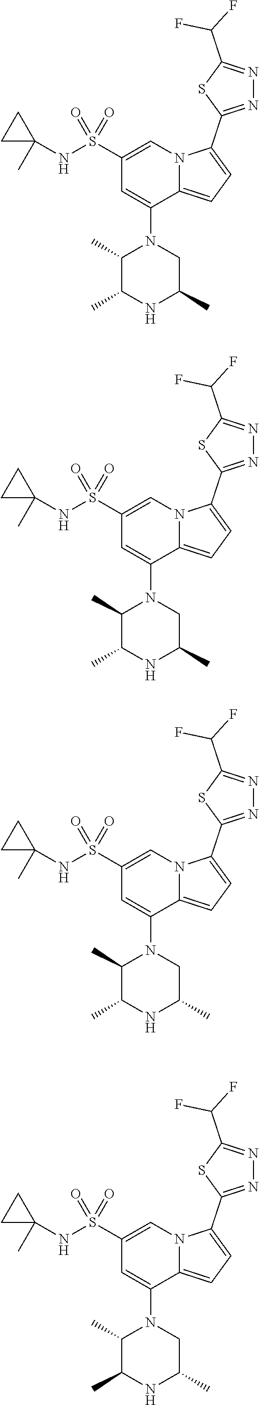 [SMILES: C[C@@H]1CN(c2cc(S(=O)(=O)NC3(C)CC3)cn3c(-c4nnc(C(F)F)s4)ccc23)[C@@H](C)[C@@H](C)N1 ; C[C@@H]1CN(c2cc(S(=O)(=O)NC3(C)CC3)cn3c(-c4nnc(C(F)F)s4)ccc23)[C@H](C)[C@@H](C)N1 ; C[C@@H]1[C@@H](C)N[C@@H](C)CN1c1cc(S(=O)(=O)NC2(C)CC2)cn2c(-c3nnc(C(F)F)s3)ccc12 ; C[C@@H]1N[C@@H](C)CN(c2cc(S(=O)(=O)NC3(C)CC3)cn3c(-c4nnc(C(F)F)s4)ccc23)[C@H]1C]

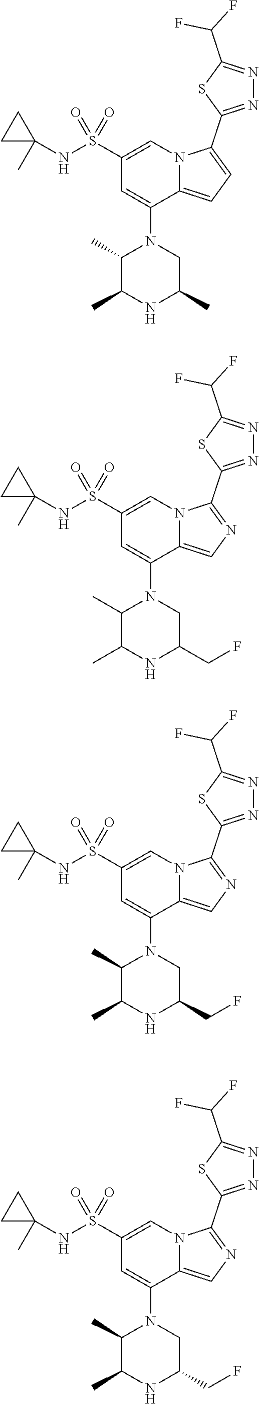 [SMILES: C[C@@H]1CN(c2cc(S(=O)(=O)NC3(C)CC3)cn3c(-c4nnc(C(F)F)s4)ccc23)[C@@H](C)[C@H](C)N1 ; CC1NC(CF)CN(c2cc(S(=O)(=O)NC3(C)CC3)cn3c(-c4nnc(C(F)F)s4)ncc23)C1C ; C[C@@H]1N[C@H](CF)CN(c2cc(S(=O)(=O)NC3(C)CC3)cn3c(-c4nnc(C(F)F)s4)ncc23)[C@@H]1C ; C[C@@H]1N[C@@H](CF)CN(c2cc(S(=O)(=O)NC3(C)CC3)cn3c(-c4nnc(C(F)F)s4)ncc23)[C@@H]1C]

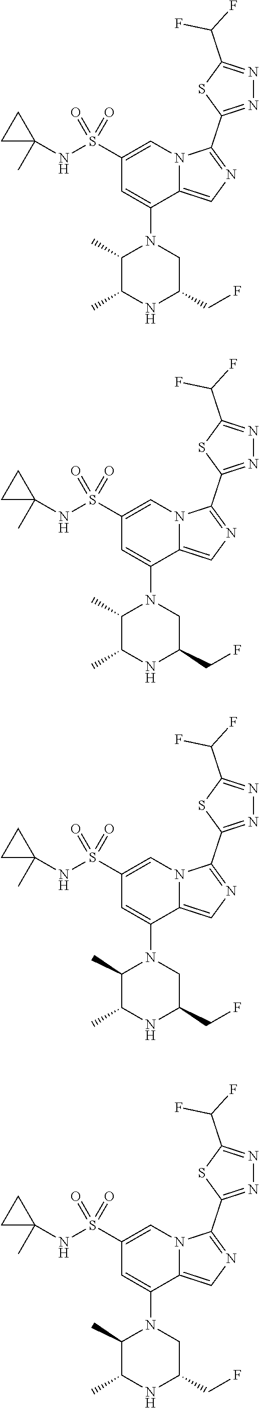 [SMILES: C[C@H]1N[C@@H](CF)CN(c2cc(S(=O)(=O)NC3(C)CC3)cn3c(-c4nnc(C(F)F)s4)ncc23)[C@H]1C ; C[C@H]1N[C@H](CF)CN(c2cc(S(=O)(=O)NC3(C)CC3)cn3c(-c4nnc(C(F)F)s4)ncc23)[C@H]1C ; C[C@@H]1[C@@H](C)N[C@H](CF)CN1c1cc(S(=O)(=O)NC2(C)CC2)cn2c(-c3nnc(C(F)F)s3)ncc12 ; C[C@@H]1[C@@H](C)N[C@@H](CF)CN1c1cc(S(=O)(=O)NC2(C)CC2)cn2c(-c3nnc(C(F)F)s3)ncc12]

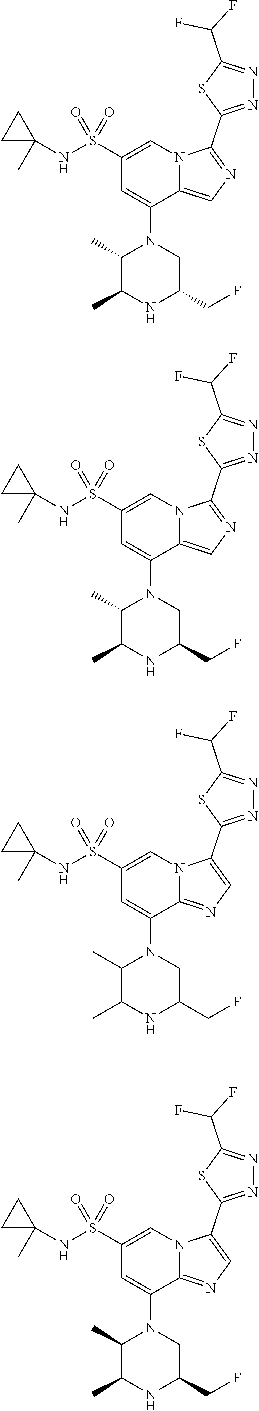 [SMILES: C[C@@H]1N[C@@H](CF)CN(c2cc(S(=O)(=O)NC3(C)CC3)cn3c(-c4nnc(C(F)F)s4)ncc23)[C@H]1C ; C[C@@H]1N[C@H](CF)CN(c2cc(S(=O)(=O)NC3(C)CC3)cn3c(-c4nnc(C(F)F)s4)ncc23)[C@H]1C ; CC1NC(CF)CN(c2cc(S(=O)(=O)NC3(C)CC3)cn3c(-c4nnc(C(F)F)s4)cnc23)C1C ; C[C@@H]1N[C@H](CF)CN(c2cc(S(=O)(=O)NC3(C)CC3)cn3c(-c4nnc(C(F)F)s4)cnc23)[C@@H]1C]

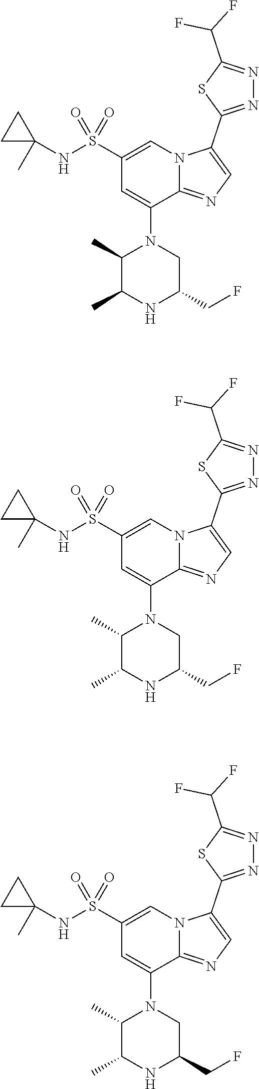 [SMILES: C[C@@H]1N[C@@H](CF)CN(c2cc(S(=O)(=O)NC3(C)CC3)cn3c(-c4nnc(C(F)F)s4)cnc23)[C@@H]1C ; C[C@H]1N[C@@H](CF)CN(c2cc(S(=O)(=O)NC3(C)CC3)cn3c(-c4nnc(C(F)F)s4)cnc23)[C@H]1C ; C[C@H]1N[C@H](CF)CN(c2cc(S(=O)(=O)NC3(C)CC3)cn3c(-c4nnc(C(F)F)s4)cnc23)[C@H]1C]

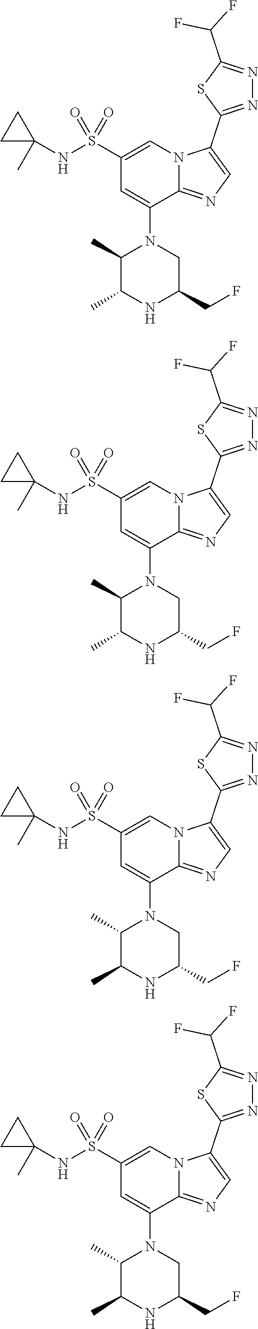

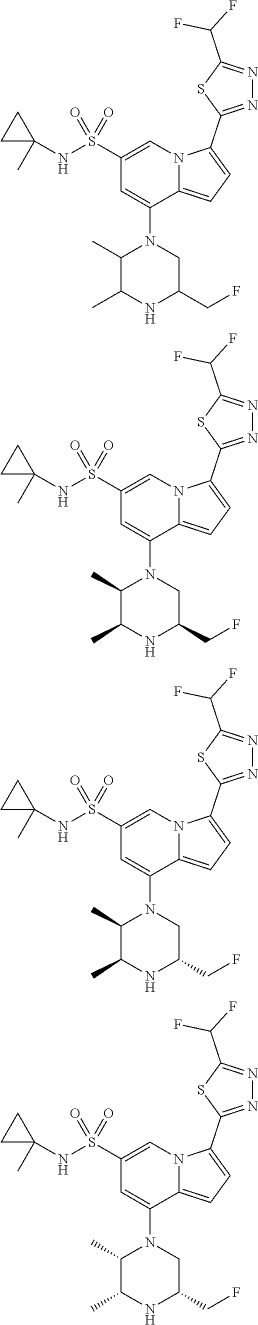

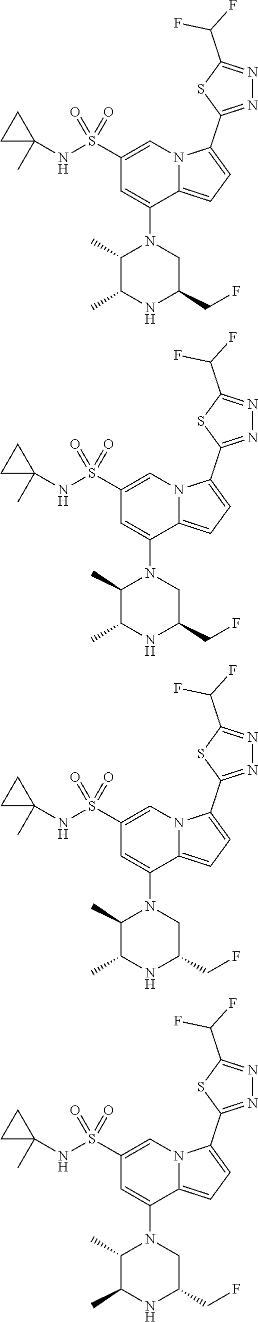

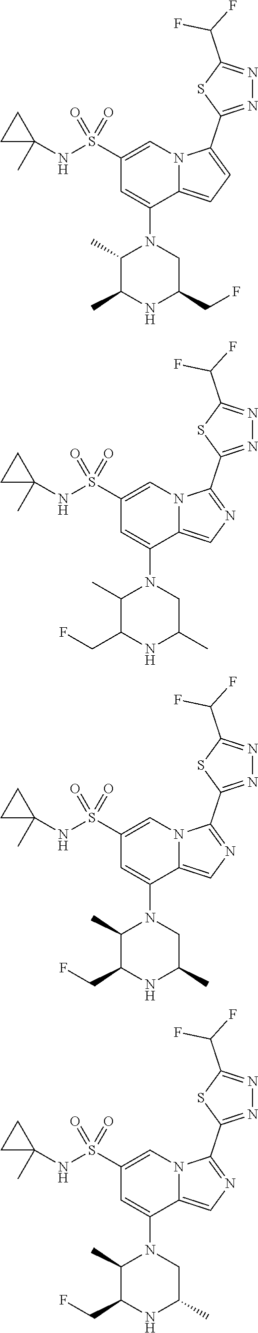

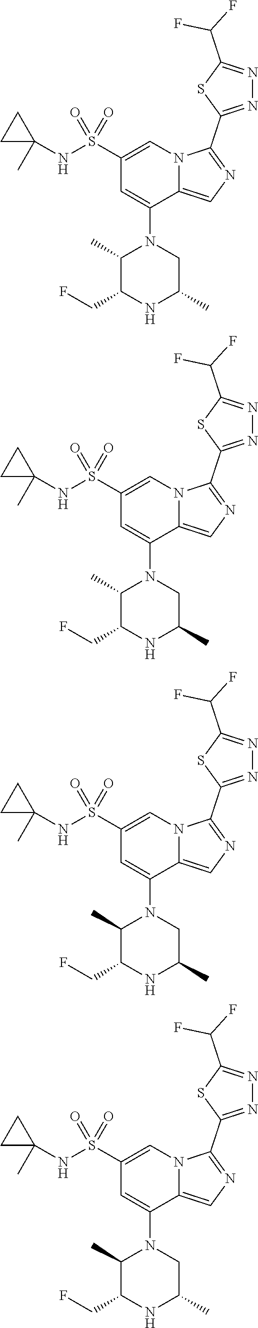

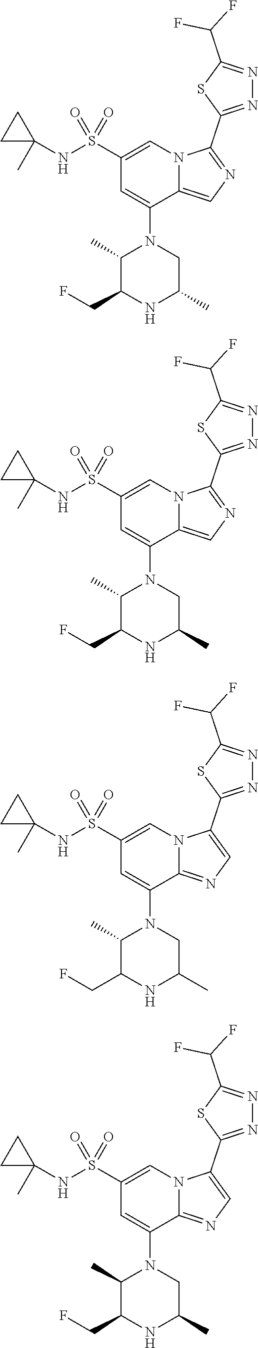

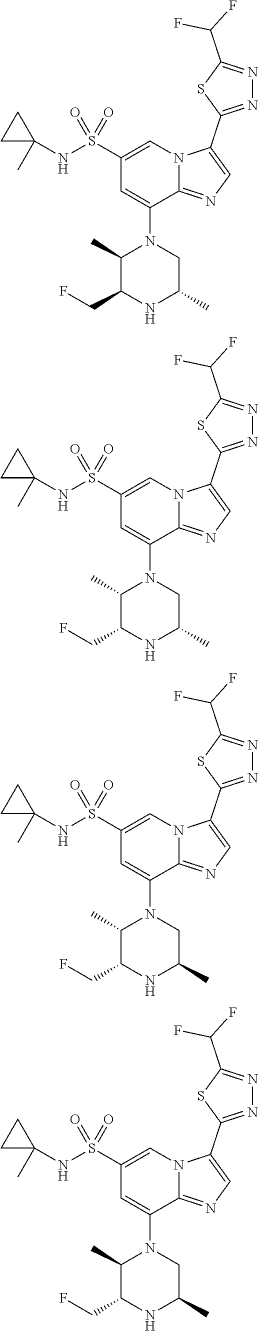

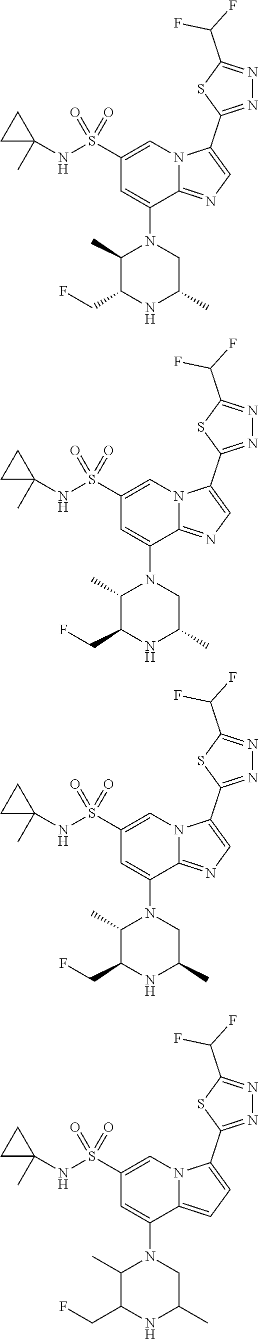

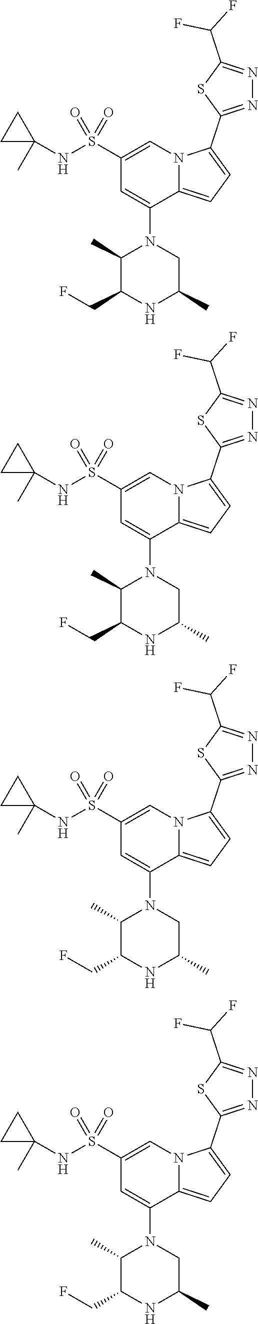

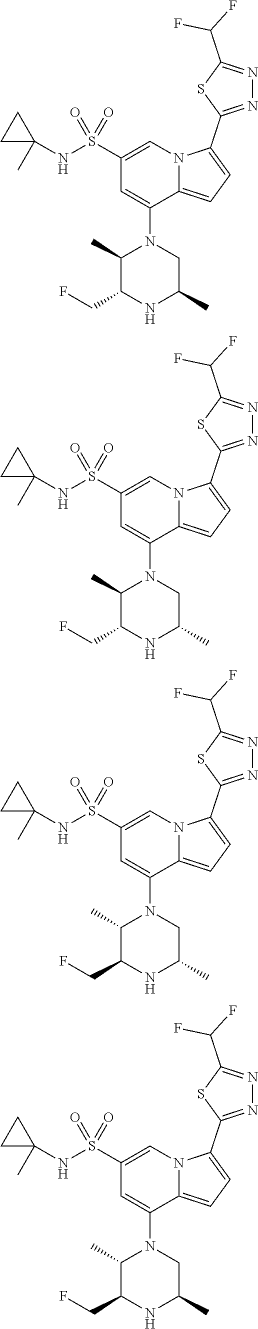

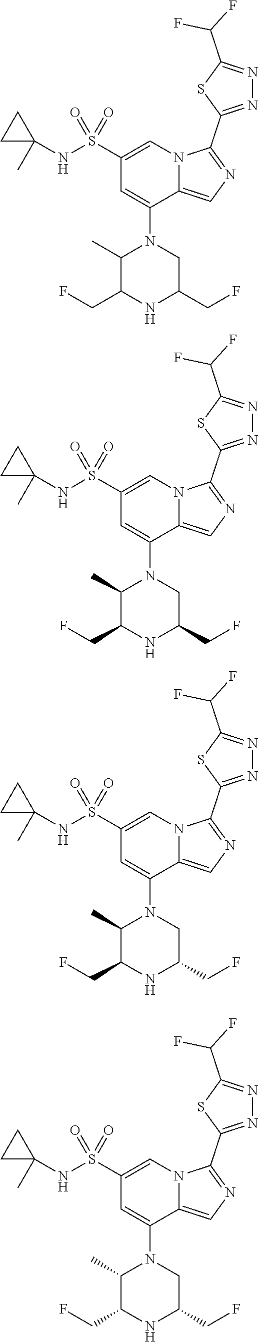

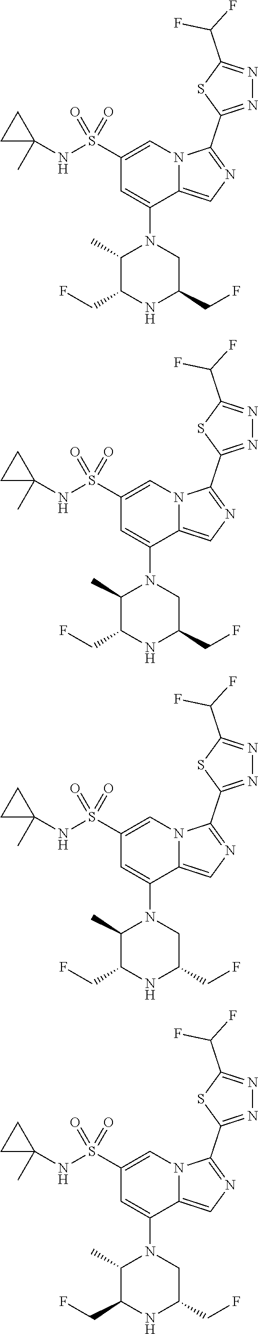

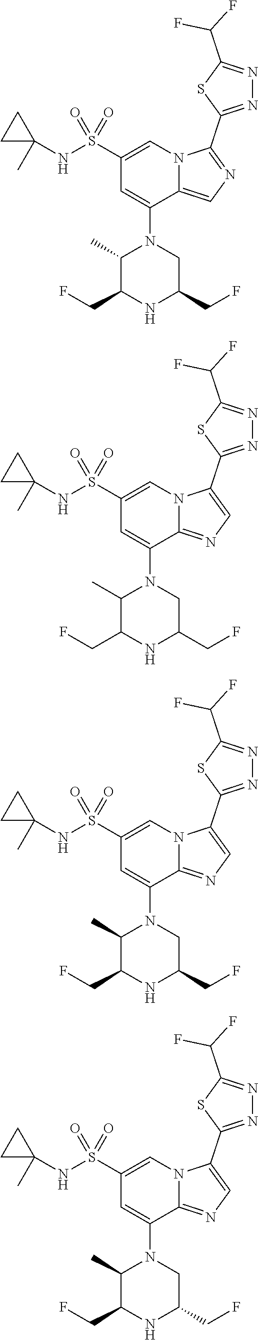

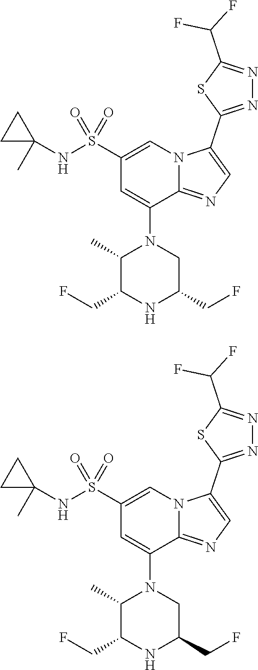 [SMILES: C[C@H]1[C@@H](CF)N[C@@H](CF)CN1c1cc(S(=O)(=O)NC2(C)CC2)cn2c(-c3nnc(C(F)F)s3)cnc12 ; C[C@H]1[C@@H](CF)N[C@H](CF)CN1c1cc(S(=O)(=O)NC2(C)CC2)cn2c(-c3nnc(C(F)F)s3)cnc12]

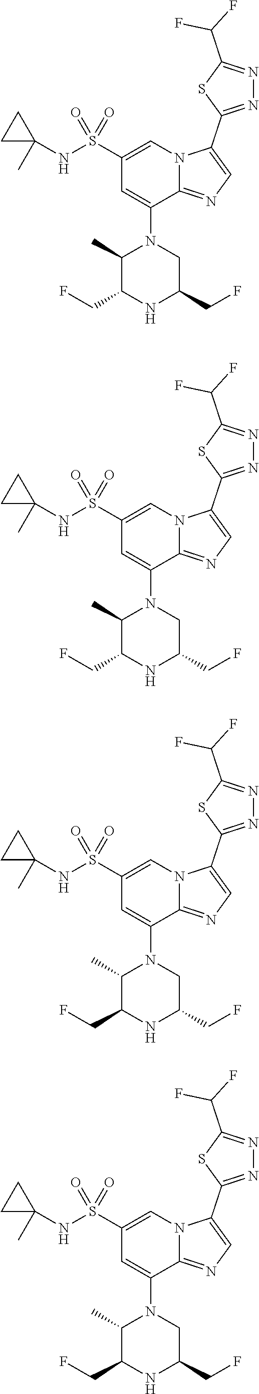 [SMILES: C[C@@H]1[C@@H](CF)N[C@H](CF)CN1c1cc(S(=O)(=O)NC2(C)CC2)cn2c(-c3nnc(C(F)F)s3)cnc12 ; C[C@@H]1[C@@H](CF)N[C@@H](CF)CN1c1cc(S(=O)(=O)NC2(C)CC2)cn2c(-c3nnc(C(F)F)s3)cnc12 ; C[C@H]1[C@H](CF)N[C@@H](CF)CN1c1cc(S(=O)(=O)NC2(C)CC2)cn2c(-c3nnc(C(F)F)s3)cnc12 ; C[C@H]1[C@H](CF)N[C@H](CF)CN1c1cc(S(=O)(=O)NC2(C)CC2)cn2c(-c3nnc(C(F)F)s3)cnc12]

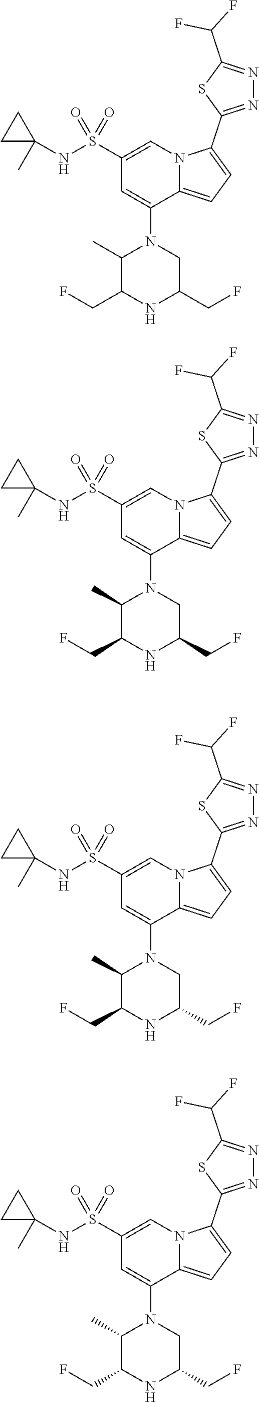 [SMILES: CC1C(CF)NC(CF)CN1c1cc(S(=O)(=O)NC2(C)CC2)cn2c(-c3nnc(C(F)F)s3)ccc12 ; C[C@@H]1[C@H](CF)N[C@H](CF)CN1c1cc(S(=O)(=O)NC2(C)CC2)cn2c(-c3nnc(C(F)F)s3)ccc12 ; C[C@@H]1[C@H](CF)N[C@@H](CF)CN1c1cc(S(=O)(=O)NC2(C)CC2)cn2c(-c3nnc(C(F)F)s3)ccc12 ; C[C@H]1[C@@H](CF)N[C@@H](CF)CN1c1cc(S(=O)(=O)NC2(C)CC2)cn2c(-c3nnc(C(F)F)s3)ccc12]

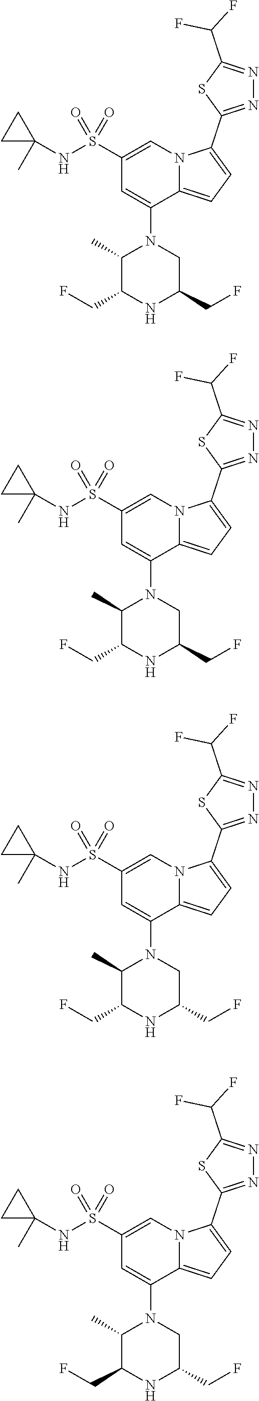 [SMILES: C[C@H]1[C@@H](CF)N[C@H](CF)CN1c1cc(S(=O)(=O)NC2(C)CC2)cn2c(-c3nnc(C(F)F)s3)ccc12 ; C[C@@H]1[C@@H](CF)N[C@H](CF)CN1c1cc(S(=O)(=O)NC2(C)CC2)cn2c(-c3nnc(C(F)F)s3)ccc12 ; C[C@@H]1[C@@H](CF)N[C@@H](CF)CN1c1cc(S(=O)(=O)NC2(C)CC2)cn2c(-c3nnc(C(F)F)s3)ccc12 ; C[C@H]1[C@H](CF)N[C@@H](CF)CN1c1cc(S(=O)(=O)NC2(C)CC2)cn2c(-c3nnc(C(F)F)s3)ccc12]

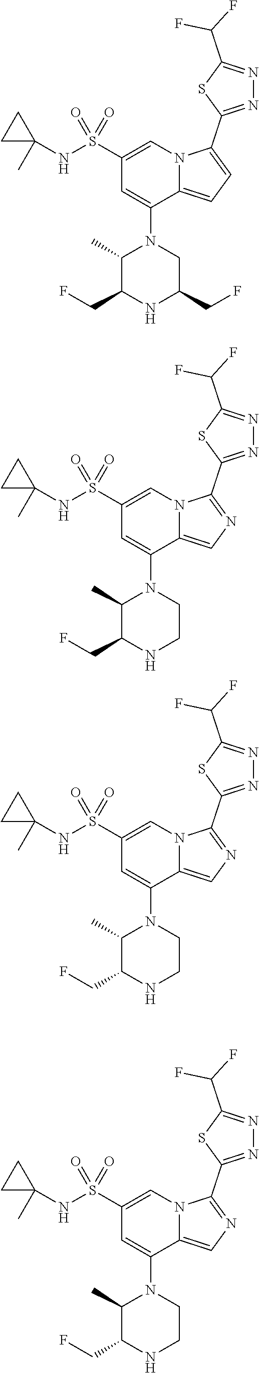 [SMILES: C[C@H]1[C@H](CF)N[C@H](CF)CN1c1cc(S(=O)(=O)NC2(C)CC2)cn2c(-c3nnc(C(F)F)s3)ccc12 ; C[C@@H]1[C@H](CF)NCCN1c1cc(S(=O)(=O)NC2(C)CC2)cn2c(-c3nnc(C(F)F)s3)ncc12 ; C[C@H]1[C@@H](CF)NCCN1c1cc(S(=O)(=O)NC2(C)CC2)cn2c(-c3nnc(C(F)F)s3)ncc12 ; C[C@@H]1[C@@H](CF)NCCN1c1cc(S(=O)(=O)NC2(C)CC2)cn2c(-c3nnc(C(F)F)s3)ncc12]

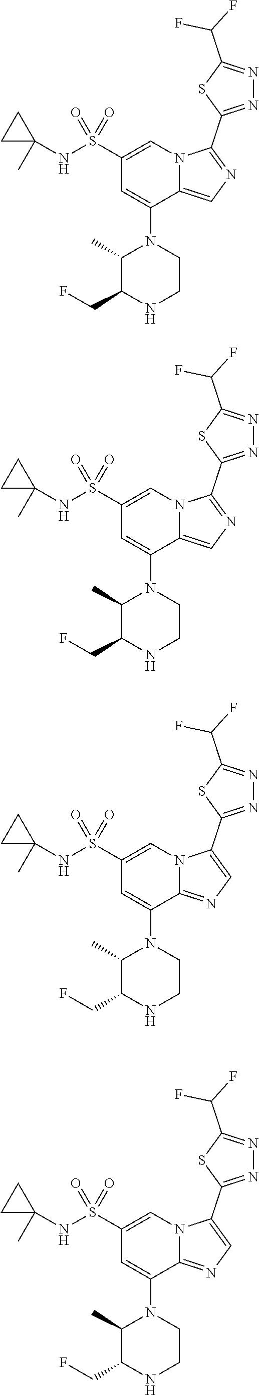 [SMILES: C[C@H]1[C@H](CF)NCCN1c1cc(S(=O)(=O)NC2(C)CC2)cn2c(-c3nnc(C(F)F)s3)ncc12 ; C[C@@H]1[C@H](CF)NCCN1c1cc(S(=O)(=O)NC2(C)CC2)cn2c(-c3nnc(C(F)F)s3)ncc12 ; C[C@H]1[C@@H](CF)NCCN1c1cc(S(=O)(=O)NC2(C)CC2)cn2c(-c3nnc(C(F)F)s3)cnc12 ; C[C@@H]1[C@@H](CF)NCCN1c1cc(S(=O)(=O)NC2(C)CC2)cn2c(-c3nnc(C(F)F)s3)cnc12]

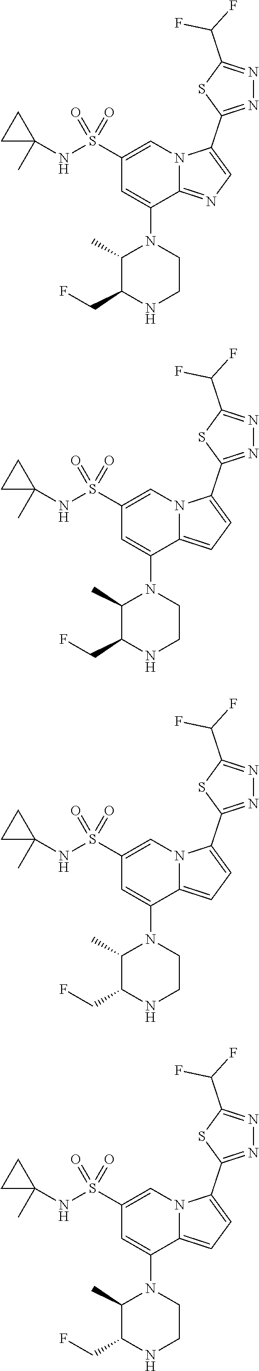 [SMILES: C[C@H]1[C@H](CF)NCCN1c1cc(S(=O)(=O)NC2(C)CC2)cn2c(-c3nnc(C(F)F)s3)cnc12 ; C[C@@H]1[C@H](CF)NCCN1c1cc(S(=O)(=O)NC2(C)CC2)cn2c(-c3nnc(C(F)F)s3)ccc12 ; C[C@H]1[C@@H](CF)NCCN1c1cc(S(=O)(=O)NC2(C)CC2)cn2c(-c3nnc(C(F)F)s3)ccc12 ; C[C@@H]1[C@@H](CF)NCCN1c1cc(S(=O)(=O)NC2(C)CC2)cn2c(-c3nnc(C(F)F)s3)ccc12]

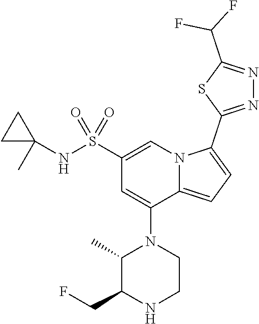 [SMILES: C[C@H]1[C@H](CF)NCCN1c1cc(S(=O)(=O)NC2(C)CC2)cn2c(-c3nnc(C(F)F)s3)ccc12]

The present invention further provides the following compounds or their pharmaceutically acceptable salts:

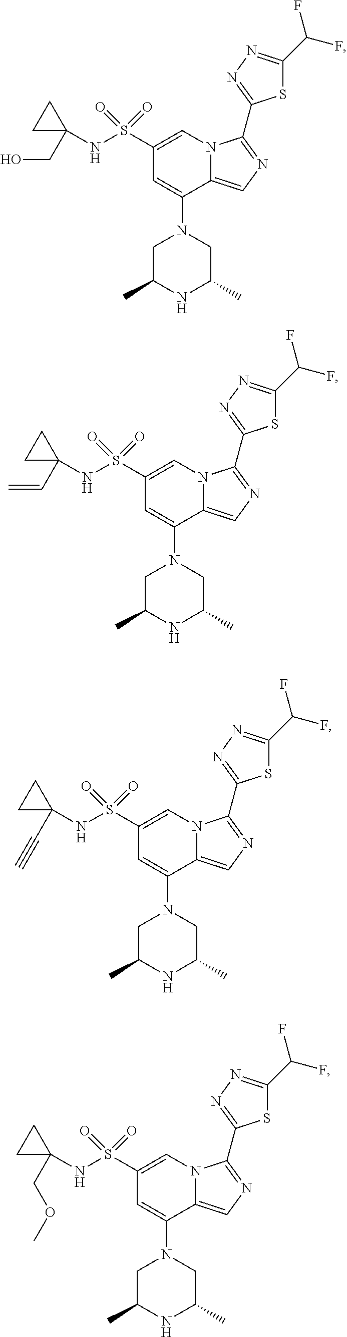 [SMILES: C[C@H]1CN(c2cc(S(=O)(=O)NC3(CO)CC3)cn3c(-c4nnc(C(F)F)s4)ncc23)C[C@H](C)N1 ; C=CC1(NS(=O)(=O)c2cc(N3C[C@H](C)N[C@@H](C)C3)c3cnc(-c4nnc(C(F)F)s4)n3c2)CC1 ; C#CC1(NS(=O)(=O)c2cc(N3C[C@H](C)N[C@@H](C)C3)c3cnc(-c4nnc(C(F)F)s4)n3c2)CC1 ; COCC1(NS(=O)(=O)c2cc(N3C[C@H](C)N[C@@H](C)C3)c3cnc(-c4nnc(C(F)F)s4)n3c2)CC1]

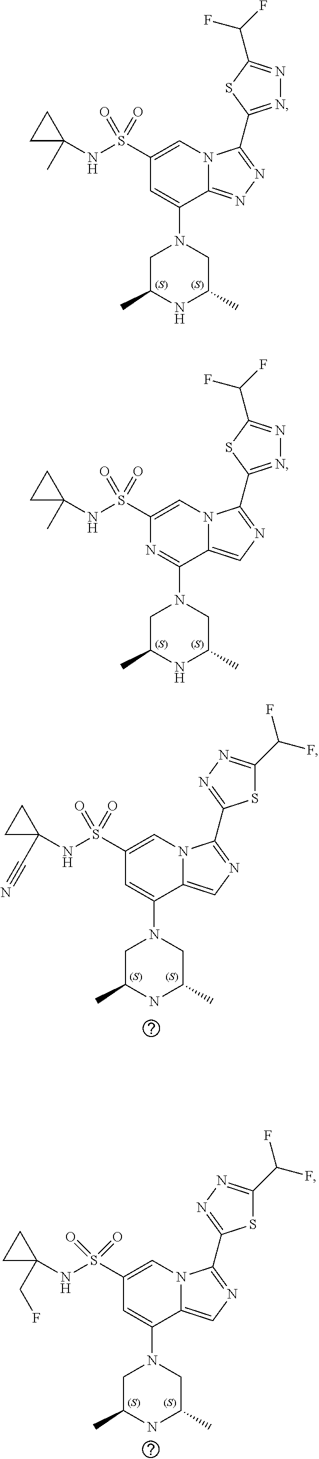

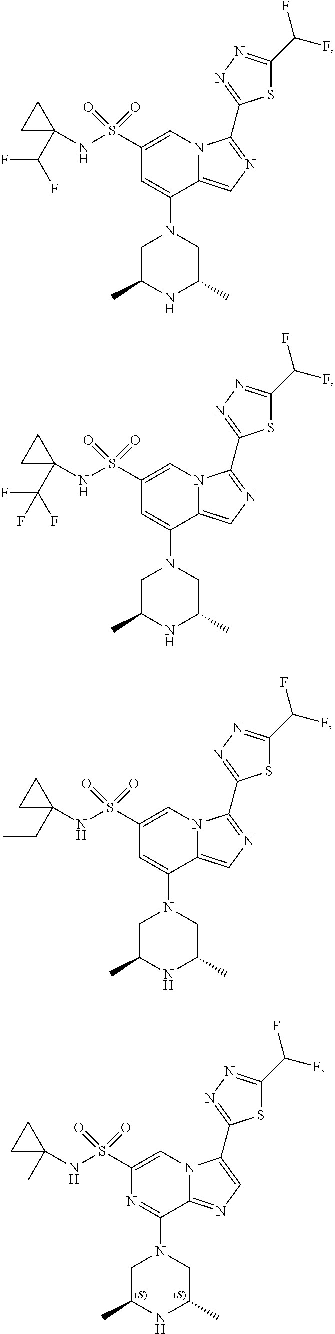

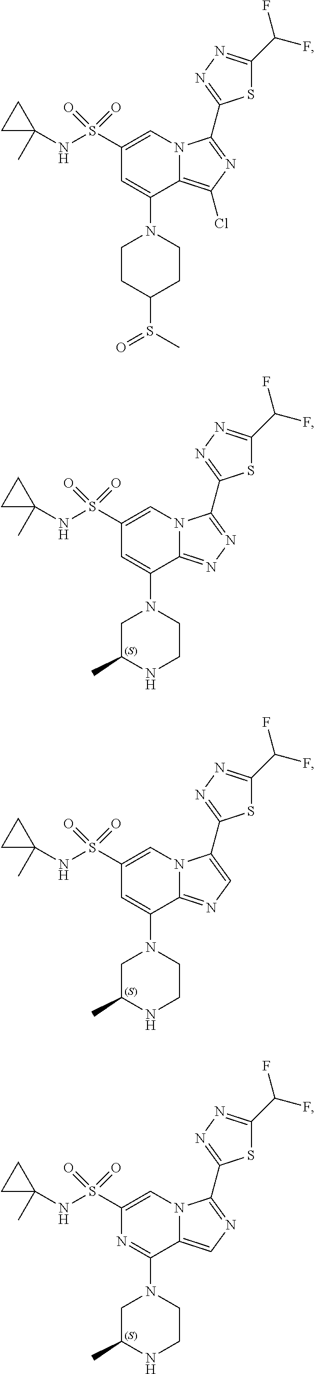

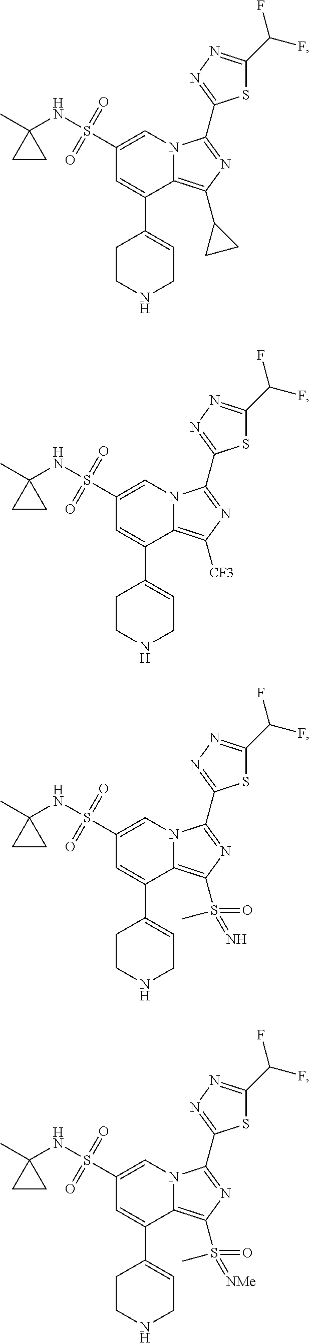

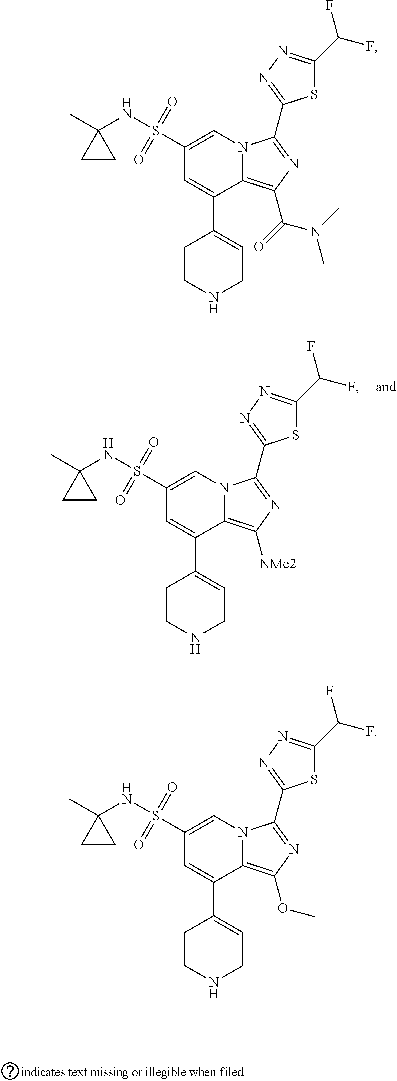 [SMILES: CN(C)C(=O)c1nc(-c2nnc(C(F)F)s2)n2cc(S(=O)(=O)NC3(C)CC3)cc(C3=CCNCC3)c12 ; CN(C)c1nc(-c2nnc(C(F)F)s2)n2cc(S(=O)(=O)NC3(C)CC3)cc(C3=CCNCC3)c12 ; COc1nc(-c2nnc(C(F)F)s2)n2cc(S(=O)(=O)NC3(C)CC3)cc(C3=CCNCC3)c12]

Particularly preferred compound of formula (I) is:

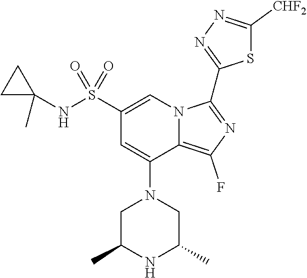 [SMILES: C[C@H]1CN(c2cc(S(=O)(=O)NC3(C)CC3)cn3c(-c4nnc(C(F)F)s4)nc(F)c23)C[C@H](C)N1]

or pharmaceutically acceptable salt thereof.

Further particularly preferred compound of formula (I) is:

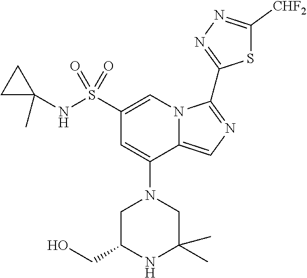 [SMILES: CC1(C)CN(c2cc(S(=O)(=O)NC3(C)CC3)cn3c(-c4nnc(C(F)F)s4)ncc23)C[C@@H](CO)N1]

or pharmaceutically acceptable salt thereof.

Further particularly preferred compound of formula (I) is:

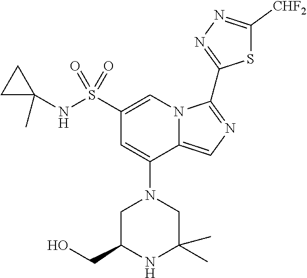 [SMILES: CC1(C)CN(c2cc(S(=O)(=O)NC3(C)CC3)cn3c(-c4nnc(C(F)F)s4)ncc23)C[C@H](CO)N1]

or a pharmaceutically acceptable salt thereof.

Particularly preferred compound of formula (I) is

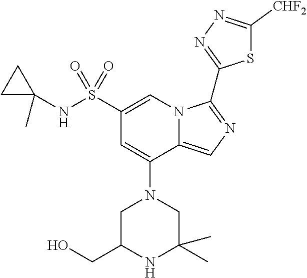 [SMILES: CC1(C)CN(c2cc(S(=O)(=O)NC3(C)CC3)cn3c(-c4nnc(C(F)F)s4)ncc23)CC(CO)N1]

or a pharmaceutically acceptable salt thereof. Even more preferred is an enantiomer of the compound of formula:

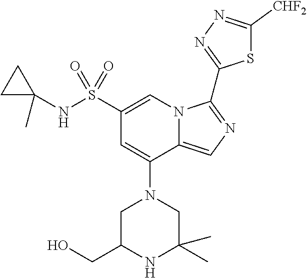 [SMILES: CC1(C)CN(c2cc(S(=O)(=O)NC3(C)CC3)cn3c(-c4nnc(C(F)F)s4)ncc23)CC(CO)N1]

characterized by longer retention time when analyzed using SFC, preferably according to Method 1 as disclosed herein (Column:Chiralpak IH-3 50*4.6 mm I.D., 3 μm Mobile phase:Phase A for CO₂,and Phase B for EtOH(0.05% DEA); Gradient elution:EtOH(0.05% DEA) in CO₂ from 5% to 40%, Flow rate: 3 mL/min; Detector:PDA; Column Temp:35 C; Back Pressure:100 Bar), or a pharmaceutically acceptable salt thereof.

The present invention also relates to each of the intermediates described further below in the examples section of this specification, including any one of these intermediates in non-salt form or in the form of a salt (e.g., a pharmaceutically acceptable salt) of the respective compound. Such intermediates can be used, in particular, in the synthesis of the compounds of formula (I).

The scope of the invention embraces all pharmaceutically acceptable salt forms of the compounds of formula (I) which may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts. Preferred pharmaceutically acceptable salts of the compounds of formula (I) include a hydrochloride salt, a hydrobromide salt, a mesylate salt, a sulfate salt, a tartrate salt, a fumarate salt, an acetate salt, a citrate salt, and a phosphate salt. A particularly preferred pharmaceutically acceptable salt of the compound of formula (I) is a hydrochloride salt. Accordingly, it is preferred that the compound of formula (I), including any one of the specific compounds of formula (I) described herein, is in the form of a hydrochloride salt, a hydrobromide salt, a mesylate salt, a sulfate salt, a tartrate salt, a fumarate salt, an acetate salt, a citrate salt, or a phosphate salt, and it is particularly preferred that the compound of formula (I) is in the form of a hydrochloride salt.

The present invention also specifically relates to the compound of formula (I), including any one of the specific compounds of formula (I) described herein, in non-salt form.

Moreover, the scope of the invention embraces the compounds of formula (I) in any solvated form, including, e.g., solvates with water (i.e., as a hydrate) or solvates with organic solvents such as, e.g., methanol, ethanol, isopropanol, acetic acid, ethyl acetate, ethanolamine, DMSO, or acetonitrile. All physical forms, including any amorphous or crystalline forms (i.e., polymorphs), of the compounds of formula (I) are also encompassed within the scope of the invention. It is to be understood that such solvates and physical forms of pharmaceutically acceptable salts of the compounds of the formula (I) are likewise embraced by the invention.

Furthermore, the compounds of formula (I) may exist in the form of different isomers, in particular stereoisomers (including, e.g., geometric isomers (or cis/trans isomers), enantiomers and diastereomers) or tautomers (including, in particular, prototropic tautomers, such as keto/enol tautomers or thione/thiol tautomers). All such isomers of the compounds of formula (I) are contemplated as being part of the present invention, either in admixture or in pure or substantially pure form. As for stereoisomers, the invention embraces the isolated optical isomers of the compounds according to the invention as well as any mixtures thereof (including, in particular, racemic mixtures/racemates). The racemates can be resolved by physical methods, such as, e.g., fractional crystallization, separation or crystallization of diastereomeric derivatives, or separation by chiral column chromatography. The individual optical isomers can also be obtained from the racemates via salt formation with an optically active acid followed by crystallization. The present invention further encompasses any tautomers of the compounds of formula (I). It will be understood that some compounds may exhibit tautomerism. In such cases, the formulae provided herein expressly depict only one of the possible tautomeric forms. The formulae and chemical names as provided herein are intended to encompass any tautomeric form of the corresponding compound and not to be limited merely to the specific tautomeric form depicted by the drawing or identified by the name of the compound.

The scope of the invention also embraces compounds of formula (I), in which one or more atoms are replaced by a specific isotope of the corresponding atom. For example, the invention encompasses compounds of formula (I), in which one or more hydrogen atoms (or, e.g., all hydrogen atoms) are replaced by deuterium atoms (i.e., ²H; also referred to as “D”). Accordingly, the invention also embraces compounds of formula (I) which are enriched in deuterium. Naturally occurring hydrogen is an isotopic mixture comprising about 99.98 mol-% hydrogen-1 (1H) and about 0.0156 mol-% deuterium (²H or D). The content of deuterium in one or more hydrogen positions in the compounds of formula (I) can be increased using deuteration techniques known in the art. For example, a compound of formula (I) or a reactant or precursor to be used in the synthesis of the compound of formula (I) can be subjected to an H/D exchange reaction using, e.g., heavy water (D₂O). Further suitable deuteration techniques are described in: Atzrodt J et al., Bioorg Med Chem, 20(18), 5658-5667, 2012; William J S et al., Journal of Labelled Compounds and Radiopharmaceuticals, 53(11-12), 635-644, 2010; Modvig A et al., J Org Chem, 79, 5861-5868, 2014. The content of deuterium can be determined, e.g., using mass spectrometry or NMR spectroscopy. Unless specifically indicated otherwise, it is preferred that the compound of formula (I) is not enriched in deuterium. Accordingly, the presence of naturally occurring hydrogen atoms or ¹H hydrogen atoms in the compounds of formula (I) is preferred.

The present invention also embraces compounds of formula (I), in which one or more atoms are replaced by a positron-emitting isotope of the corresponding atom, such as, e.g., ¹⁸F, ¹¹C, ¹³N, ¹⁵O, ⁷⁶Br, ⁷⁷Br, ¹²⁰I and/or ¹²⁴I. Such compounds can be used as tracers, trackers or imaging probes in positron emission tomography (PET). The invention thus includes (i) compounds of formula (I), in which one or more fluorine atoms (or, e.g., all fluorine atoms) are replaced by ¹⁸F atoms, (ii) compounds of formula (I), in which one or more carbon atoms (or, e.g., all carbon atoms) are replaced by ¹¹C atoms, (iii) compounds of formula (I), in which one or more nitrogen atoms (or, e.g., all nitrogen atoms) are replaced by ¹³N atoms, (iv) compounds of formula (I), in which one or more oxygen atoms (or, e.g., all oxygen atoms) are replaced by ¹⁵O atoms, (v) compounds of formula (I), in which one or more bromine atoms (or, e.g., all bromine atoms) are replaced by ⁷⁶Br atoms, (vi) compounds of formula (I), in which one or more bromine atoms (or, e.g., all bromine atoms) are replaced by ⁷⁷Br atoms, (vii) compounds of formula (I), in which one or more iodine atoms (or, e.g., all iodine atoms) are replaced by ¹²⁰I atoms, and (viii) compounds of formula (I), in which one or more iodine atoms (or, e.g., all iodine atoms) are replaced by ¹²⁴I atoms. In general, it is preferred that none of the atoms in the compounds of formula (I) are replaced by specific isotopes.

The present invention further embraces the prodrugs of the compounds of formula (I). As preferably understood herein, the term “prodrug” of the compound of formula (I) refers to a derivative of the compounds of formula (I) that upon administration to a subject becomes metabolized to the said compound of formula (I). Said prodrugs of the compound of formula (I) may include modifications of —OH, —NH₂, or —COOH group if present in the compound of formula (I), which preferably can be hydrolyzed to —OH, —NH₂, or —COOH groups, respectively, e.g. upon administration to the subject. For example, as known to the skilled person, such prodrugs may preferably include for the compounds of formula (I) which comprise —OH moiety derivatives wherein said —OH moiety is turned into an —ORₓ moiety, wherein Rx preferably comprises a moiety selected from —CO—, —CH₂—O—CO, —CH₂—O—CO—O—, and —CH(CH₃)—O—COO—, more preferably wherein Rx is selected from —CO—R_{y}, —CH₂—O—CO—R_{y}, —CH₂—O—CO—O—R_{y}, and —CH(CH₃)—O—COO—R_{y}, wherein R_{y} is preferably carbocyclyl, heterocyclyl, C₁₋₅ alkyl, —NH—(C₁₋₅ alkyl) or —S—(C₁₋₅ alkyl), wherein the said alkyl is optionally substituted with a group selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl), and wherein the said carbocyclyl and heterocyclyl are each optionally substituted with a group selected from halogen, —CN, —OH, C₁₋₅ alkyl, C₁₋₅ haloalkyl, —O(C₁₋₅ alkyl), —O(C₁₋₅ haloalkyl), —SH, —S(C₁₋₅ alkyl), —S(C₁₋₅ haloalkyl), —NH₂, —NH(C₁₋₅ alkyl), —NH(C₁₋₅ haloalkyl), —N(C₁₋₅ alkyl)(C₁₋₅ alkyl), —N(C₁₋₅ haloalkyl)(C₁₋₅ alkyl), —CONH₂, —CONH(C₁₋₅ alkyl), and —CON(C₁₋₅ alkyl)(C₁₋₅ alkyl). Furthermore, for example, as known to the skilled person, such prodrugs may preferably include for the compounds of formula (I) which comprise —NH₂ moiety derivatives wherein said —NH₂ moiety is turned into —NHCOO—R_{y} moiety, wherein R_{y} is as defined hereinabove. Furthermore, for examples, as known to the skilled person, such prodrugs may preferably include for the compounds of formula (I) which comprise —COOH moiety derivatives wherein said —COOH group is turned into —COOR_{y} moiety, wherein R_{y} is as defined hereinabove. Further examples of groups that can be derivatized to yield prodrugs are known to the skilled person.

### Pharmaceutical Compositions

The compounds provided herein may be administered as compounds perse or may be formulated as medicaments. The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, and/or solubility enhancers.

The pharmaceutical compositions may comprise one or more solubility enhancers, such as, e.g., poly(ethylene glycol), including poly(ethylene glycol) having a molecular weight in the range of about 200 to about 5,000 Da (e.g., PEG 200, PEG 300, PEG 400, or PEG 600), ethylene glycol, propylene glycol, glycerol, a non-ionic surfactant, tyloxapol, polysorbate 80, macrogol-15-hydroxystearate (e.g., Kolliphor® HS 15, CAS 70142-34-6), a phospholipid, lecithin, dimyristoyl phosphatidylcholine, dipalmitoyl phosphatidylcholine, distearoyl phosphatidylcholine, a cyclodextrin, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, a carboxyalkyl thioether, hydroxypropyl methylcellulose, hydroxypropylcellulose, polyvinylpyrrolidone, a vinyl acetate copolymer, vinyl pyrrolidone, sodium lauryl sulfate, dioctyl sodium sulfosuccinate, or any combination thereof.

The pharmaceutical compositions may also comprise one or more preservatives, particularly one or more antimicrobial preservatives, such as, e.g., benzyl alcohol, chlorobutanol, 2-ethoxyethanol, m-cresol, chlorocresol (e.g., 2-chloro-3-methyl-phenol or 4-chloro-3-methyl-phenol), benzalkonium chloride, benzethonium chloride, benzoic acid (or a pharmaceutically acceptable salt thereof), sorbic acid (or a pharmaceutically acceptable salt thereof), chlorhexidine, thimerosal, or any combination thereof.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The compounds of formula (I) or the above described pharmaceutical compositions comprising a compound of formula (I) may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, or vaginal administration.

If said compounds or pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously administering the compounds or pharmaceutical compositions, and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Said compounds or pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

For oral administration, the compounds or pharmaceutical compositions are preferably administered by oral ingestion, particularly by swallowing. The compounds or pharmaceutical compositions can thus be administered to pass through the mouth into the gastrointestinal tract, which can also be referred to as “oral-gastrointestinal” administration.

Alternatively, said compounds or pharmaceutical compositions can be administered in the form of a suppository or pessary, or may be applied topically in the form of a gel, hydrogel, lotion, solution, cream, ointment or dusting powder. The compounds of the present invention may also be dermally or transdermally administered, for example, by the use of a skin patch.

Said compounds or pharmaceutical compositions may also be administered by sustained release systems. Suitable examples of sustained-release compositions include semi-permeable polymer matrices in the form of shaped articles, e.g., films, or microcapsules. Sustained-release matrices include, e.g., polylactides, copolymers of L-glutamic acid and gamma-ethyl-L-glutamate, poly(2-hydroxyethyl methacrylate), ethylene vinyl acetate, or poly-D-(−)-3-hydroxybutyric acid. Sustained-release pharmaceutical compositions also include liposomally entrapped compounds. The present invention thus also relates to liposomes containing a compound of the invention.

Said compounds or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

It is also envisaged to prepare dry powder formulations of the compounds of formula (I) for pulmonary administration, particularly inhalation. Such dry powders may be prepared by spray drying under conditions which result in a substantially amorphous glassy or a substantially crystalline bioactive powder. Accordingly, dry powders of the compounds of the present invention can be made according to an emulsification/spray drying process.

For topical application to the skin, said compounds or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

The present invention thus relates to the compounds or the pharmaceutical compositions provided herein, wherein the corresponding compound or pharmaceutical composition is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. Preferred routes of administration are oral administration or parenteral administration. For each of the compounds or pharmaceutical compositions provided herein, it is particularly preferred that the respective compound or pharmaceutical composition is to be administered orally (particularly by oral ingestion).

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy.

A proposed, yet non-limiting dose of the compounds according to the invention for oral administration to a human (of approximately 70 kg body weight) may be 0.05 to 2000 mg, preferably 0.1 mg to 1000 mg, of the active ingredient per unit dose. The unit dose may be administered, e.g., 1 to 3 times per day. The unit dose may also be administered 1 to 7 times per week, e.g., with not more than one administration per day. It will be appreciated that it may be necessary to make routine variations to the dosage depending on the age and weight of the patient/subject as well as the severity of the condition to be treated. The precise dose and also the route of administration will ultimately be at the discretion of the attendant physician or veterinarian.

### Therapeutic Use

In one embodiment, the present invention relates to the compound of formula (I), or a pharmaceutically acceptable salt, hydrate or solvate thereof, or a pharmaceutical composition as defined herein for use in therapy. It is to be understood that preferably the medical uses described herein also apply to the compounds provided in the present application that do not fall under the scope of formula (I).

The present invention provides compounds that function as inhibitors of PARG. Thus, the present invention provides a method of inhibiting PARG enzyme activity in vitro or in vivo, said method comprising contacting a cell with an effective amount of the compound of formula (I), or a pharmaceutically acceptable salt, hydrate or solvate thereof, as defined herein.

The present invention also provides a method of selectively inhibiting PARG enzyme activity over PARP1 or ARH3 enzyme activity in vitro or in vivo. The said method comprises the steps of contacting a cell with an effective amount of a compound, or a pharmaceutically acceptable salt, hydrate or solvate thereof, as defined herein.

In a further embodiment, the present invention relates to the compound of formula (I), as disclosed herein, for use in a method of treating a disease or disorder in which PARG activity is implicated in a subject or patient in need of such treatment. Said method of treatment comprises administering to said subject/patient a therapeutically effective amount of a compound of formula (I), or a pharmaceutically acceptable salt, hydrate or solvate thereof, or a pharmaceutical composition as defined herein. In other words, in one embodiment the present invention relates to the compound of formula (I), as disclosed herein, for use in treating a disease or disorder in which PARG activity is implicated.

In a further embodiment, the present invention relates to a method of inhibiting cell proliferation, in vitro or in vivo, said method comprising contacting a cell with an effective amount of the compound of formula (I), or a pharmaceutically acceptable salt, hydrate or solvate thereof, as defined herein. Thus, the present invention relates to the compound of formula (I) or a pharmaceutically acceptable salt thereof for use in of inhibiting cell proliferation, in vitro or in vivo.

Thus, in a further embodiment, the present invention relates to a method of treating a proliferative disorder in a subject or patient in need of such treatment. The said method of treating a proliferative disorder in a subject or patient in need thereof comprises administering to said subject/patient a therapeutically effective amount of the compound of formula (I), or a pharmaceutically acceptable salt, hydrate or solvate thereof, or a pharmaceutical composition as defined herein. Preferably as disclosed herein, the proliferative disorder is cancer. Thus, the present invention relates to a method of treating cancer in a subject or patient in need thereof. The said method of treating cancer in a subject or patient in need thereof comprises administering to said subject/patient a therapeutically effective amount of the compound of formula (I), or a pharmaceutically acceptable salt, hydrate or solvate thereof, or a pharmaceutical composition as defined herein. In a particular embodiment, the cancer is human cancer.

In one embodiment, the present invention relates to the compound of formula (I) or a pharmaceutically acceptable salt, hydrate or solvate thereof, for use in treating a proliferative disorder. Preferably as disclosed herein, the proliferative disorder is cancer. Therefore, the present invention relates to the compound of formula (I) or a pharmaceutically acceptable salt, hydrate or solvate thereof for use in treating cancer. In a particular embodiment, the cancer is human cancer.

In a further embodiment, the present invention relates to the compound of formula (I), or a pharmaceutically acceptable salt, hydrate or solvate thereof, as defined herein, for use in the manufacture of a medicament for the treatment of a proliferative condition. In a preferred embodiment, the proliferative condition is cancer, more preferably a human cancer. Thus, preferably the present invention relates to the compound of formula (I), or a pharmaceutically acceptable salt, hydrate or solvate thereof, as defined herein, for use in the manufacture of a medicament for the treatment of cancer, preferably for the treatment of human cancer.

In a further embodiment, the present invention relates to the compound of formula (I), or a pharmaceutically acceptable salt, hydrate or solvate thereof, as defined herein, for use in the manufacture of a medicament for the inhibition of PARG enzyme activity. Preferably, the inhibition of PARG enzyme activity is selective inhibition of PARG enzyme activity over PARP1 or ARH3 enzyme activity. Thus, the present invention relates to the compound of formula (I), or a pharmaceutically acceptable salt, hydrate or solvate thereof, as defined herein, for use in the manufacture of a medicament for the selective inhibition of PARG enzyme activity over PARP1 or ARH3 enzyme activity.

The present invention further provides the compound of formula (I), or a pharmaceutically acceptable salt, hydrate or solvate thereof, as defined herein for use in the manufacture of a medicament for the treatment of a disease or disorder in which PARG activity is implicated, as defined herein.

As understood herein, the term “proliferative disorder” are used interchangeably herein and pertain to an unwanted or uncontrolled cellular proliferation of excessive or abnormal cells which is undesired, such as, neoplastic or hyperplastic growth, whether in vitro or in vivo. Examples of proliferative conditions include, but are not limited to, pre-malignant and malignant cellular proliferation, including but not limited to, malignant neoplasms and tumours, cancers, leukemias, psoriasis, bone diseases, fibroproliferative disorders (e.g., of connective tissues), and atherosclerosis. Any type of cell may be treated, including but not limited to, lung, colon, breast, ovarian, prostate, liver, pancreas, brain, and skin.

The anti-proliferative effects of the compound of formula (I) of the present invention have particular application in the treatment of human cancers (by virtue of their inhibition of PARG enzyme activity). The anti-cancer effect may arise through one or more mechanisms, including but not limited to, the regulation of cell proliferation, the inhibition of angiogenesis (the formation of new blood vessels), the inhibition of metastasis (the spread of a tumour from its origin), the inhibition of invasion (the spread of tumour cells into neighbouring normal structures), or the promotion of apoptosis (programmed cell death).

The antiproliferative treatment with the compound of formula (I) or a pharmaceutically acceptable salt, hydrate or solvate thereof, as defined hereinbefore, may be applied as a sole therapy or may involve, in addition to the compound of the invention, conventional surgery or radiotherapy or chemotherapy. Such chemotherapy may include one or more of the following categories of anti-tumour agents:—
(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5oc-reductase such as finasteride;
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
(iv) inhibitors of growth factor function: for example such inhibitors include growth factor antibodies and growth factor receptor antibodies (for example the anti-erbB2 antibody trastuzumab [Herceptin™] the anti-EGFR antibody panitumumab, the anti-erbB1 antibody cetuximab [Erbitux, C225] and any growth factor or growth factor receptor antibodies disclosed by Stern et al. (Critical reviews in oncology/haematology, 2005, Vol. 54, pp1 1-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R1 15777) and lonafarnib (SCH66336)), inhibitors of cell signalling through MEK and/or AKT kinases, c-kit inhibitors, abl kinase inhibitors, PI3 kinase inhibitors, PIt3 kinase inhibitors, CSF-1 R kinase inhibitors, IGF receptor (insulin-like growth factor) kinase inhibitors; aurora kinase inhibitors (for example AZD1 152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU1 1248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213; (vii) an endothelin receptor antagonist, for example zibotentan (ZD4054) or atrasentan;
(viii) antisense therapies, for example those which are directed to the targets listed above, such as ISIS 2503, an anti-ras antisense;
(ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(x) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

In a particular embodiment, the antiproliferative treatment defined hereinbefore may involve, in addition to the compound of formula (I) of the invention, conventional surgery or radiotherapy or chemotherapy. Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect the present invention further relates to the compound of formula (I) or a pharmaceutically acceptable salt, hydrate or solvate thereof, as defined herein, for use in the treatment of a cancer (for example a cancer involving a solid tumour) in combination with another anti-tumour agent. The anti-tumour agent is preferably selected from the anti-tumour agents as listed hereinabove.

As understood herein, the term “combination” refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

### EXAMPLES

The following examples are merely illustrative of the present invention and should not be construed to limit the scope of the invention which is defined by the appended claims.

### Synthesis of the Compounds of Formula (I)

The syntheses of embodiments A, B and C of the compounds of formula (I) according to the present invention are preferably carried out according to the general synthetic sequences as shown in Schemes 1-3. It is to be understood that the compounds provided herein, but not falling under the scope of formula (I), can also be prepared as described herein.

In addition to said routes described below, also other routes may be used to synthesize the target compounds, in accordance with common general knowledge of a person skilled in the art of organic synthesis. The order of transformations exemplified in the following Schemes is therefore not intended to be limiting, and suitable synthesis steps from various schemes can be combined to form additional synthesis sequences. In addition, modification of any of the substituents can be achieved before and/or after the exemplified transformations. These modifications can be such as the introduction of protective groups, cleavage of protective groups, reduction or oxidation of functional groups, halogenation, metallation, metal-catalyzed coupling reactions, substitution or other reactions known to a person skilled in the art. These transformations include those which introduce a functionality allowing for further interconversion of substituents. Appropriate protective groups and their introduction and cleavage are well-known to a person skilled in the art (see for example: Greene's Protective Groups in Organic Synthesis; Editor: P.G.M. Wuts, 5th edition, Wiley 2014). Specific examples are described in the subsequent paragraphs. Further, it is possible that two or more successive steps may be performed without work-up being performed between said steps, e.g. a “one-pot” reaction, as it is well-known to a person skilled in the art. It is further understood to the skilled person that a reaction can lead to side product(s) which, when appropriate, can be used for the preparation of compounds of formula (I) using similar procedures as reported in the general schemes hereinbelow.

Accordingly, the compounds of the present invention may also be prepared in analogy to the specific procedures shown in the Examples section, or could be synthesized according to the general schemes or well known procedures to someone skilled in the art. It is to be understood that certain modifications to these schemes that would be obvious to the skilled person are also covered by the present invention.

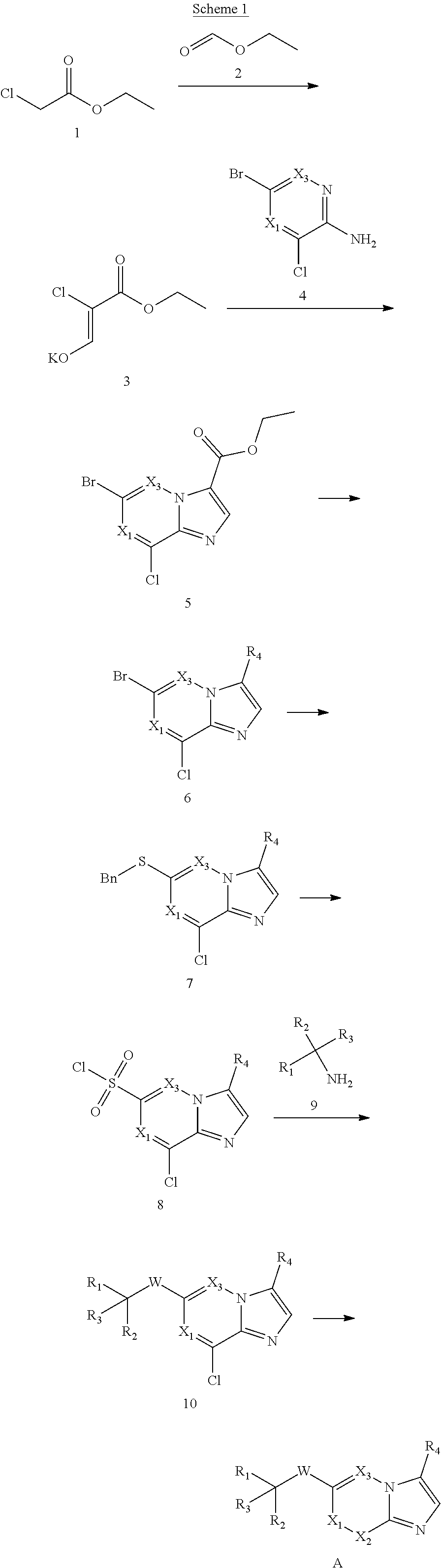

Scheme 1 illustrates a preferred synthetic approach to compounds of the general formula A.

As it is to be understood herein, X₃ is CH. X₂ is C—R. R₄ is 2-(difluoromethyl)-1,3,4-thiadiazol-2-yl. R₂ and R₃ together with a carbon atom that otherwise carries R₂ (not to be confused with R₂ in the definition of X₄) and R₃ form together a cyclopronane ring.

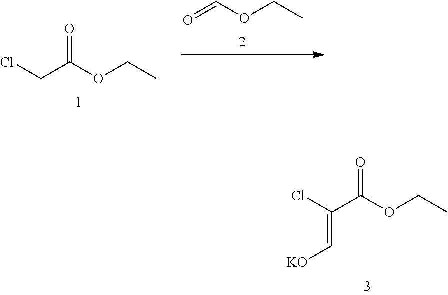

In the first step, ethyl 2-chloroacetate 1 is reacted with ethyl formate 2 under basic condition to provide potassium (Z)-2-chloro-3-ethoxy-3-oxoprop-1-en-1-olate 3. The reaction is preferably carried out in solvents like tert-butyl methyl ether, di-isopropyl ether, diethyl ether, 1,2-dimethoxyethane, dioxane, DMF, DME, THF, or a mixture of toluene, diethyl ether, and EtOH in the presence of a base like sodium ethoxide, sodium methoxide, potassium tert-butylate or sodium tert-butylate. (see for examples: a) Stephen et al, US2017/369489; b) Murar et al, Eu. J. Med. Chem. 2017, 126, 754). The reaction is performed at temperatures ranging from −78° C. to the room temperature. The reaction is preferably completed after 1-24 hours.

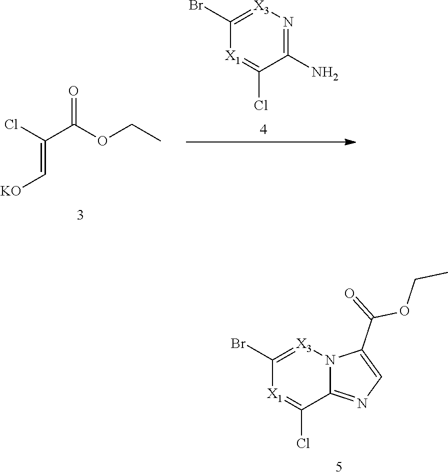

In the second step, a compound of formula 4, in which X₁ is as defined for the compound of formula (I), is reacted with potassium (Z)-2-chloro-3-ethoxy-3-oxoprop-1-en-1-olate 3 to give a compound of formula 5. This cyclization can be carried out under acidic conditions (see for example: Xi et al, WO2019/99311). Preferred is the herein described use of sulfuric acid in EtOH. The reactions are preferably run for 5-24 hours at 70-100° C.

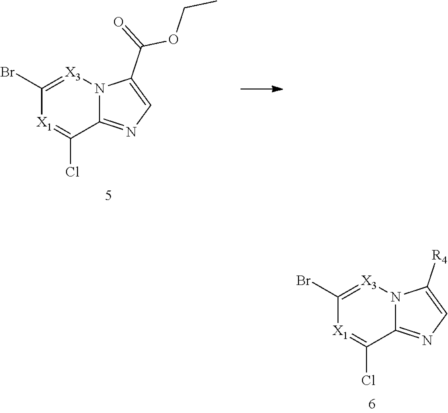

In the third step, a compound of formula 5 in which X₁ is as defined for the compound of formula (I) is converted to a compound of formula 6 in which X₁ is as defined for the compound of formula (I) in several synthetic steps. If R₄ is a 2-(difluoromethyl)-1,3,4-thiadiazole group, a compound of formula 5 is reacted with hydrazine hydrate to produce a hydrazide. This hydrazide formation can be carried out under neutral condition. (see for example: Dong et al, J. Med. Chem. 2020, 63, 3028). The hydrazide formation is preferably performed in EtOH and the reactions are preferably run for 1-24 hours at 50-100° C. with heating or microwave conditions. The hydrazide is then reacted with ethyl 2,2-difluoroacetate to produce a di-acyl hydrazine. This reaction can be carried out under basic condition, preferred is the herein described use of DBU in EtOH, THF, or DMF. The reactions are preferably run for 0.5-24 hours at room temperature to 100° C. in a microwave oven or in an oil bath. Finally, the di-acyl hydrazine is cyclized by treatment with oxygen/sulfur exchange reagents to a compound of formula 6, in which R₄ is 2-(difluoromethyl)-1,3,4-thiadiazole group. (see for example: Brunet et al, WO2020/127974). Preferred is the herein described use of Lawessons reagent in toluene or THF. The reactions are preferably run for 0.5-24 hours at 50-130° C.

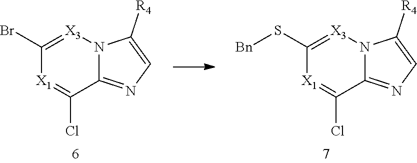

In the fourth step, a compound of formula 6 in which X₁, X₃ and R₄ are as defined for the compound of formula (I) is reacted with benzyl mercaptan to give a compound of formula 7. This coupling reaction can be carried out by a palladium-catalyzed C—S cross-coupling reaction (see for example: Jiang, Buchwald in ‘Metal-Catalyzed Cross-Coupling Reactions’, 2^{nd} edition.: de Meijere, Diederich, Eds.: Wiley-VCH: Weinheim, Germany, 2004). Preferred is the herein described use of tris(dibenzylideneacetone) dipalladium(0), (9,9-dimethyl-9H-xanthene-4,5-diyl)bis(diphenylphosphane) and N-ethyl-N-isopropylpropan-2-amine in dioxane. The reactions are preferably run under an atmosphere of argon for 1-48 hours at 80-100° C. in a microwave oven or in an oil bath.

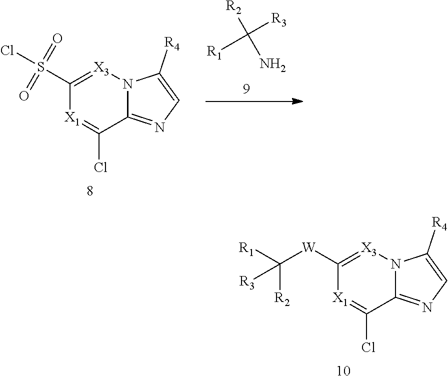

In the fifth step, a compound of formula 7 in which X₁ is as defined for the compound of formula (I) is reacted with chlorination reagent to give a sulfonyl chloride of formula 8. This sulfonyl chloride formation can be carried out by treatment with NCS, sulfonyl chloride, DCDMH, Cl₂ etc., in MeCN with equivalent acetic acid and water. (see for example: Sutton et al, WO 2021/055744). Preferred is the herein described use of DCDMH in MeCN with equivalent acetic acid and water. The reactions are preferably run under an atmosphere of argon for 0.5-5 hours at 0° C. to room temperature.

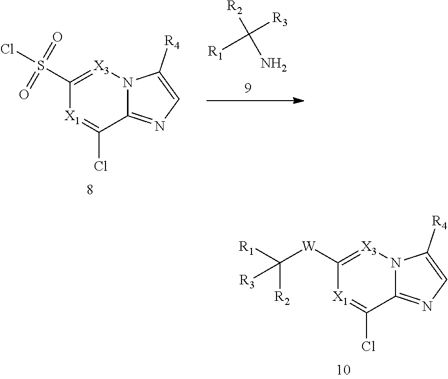

In the sixth step, a compound of formula 8 in which X₁ is as defined for the compound of formula (I) is reacted with an amine of formula 9 in which R₁, is as defined for the compound of formula (I) to give a compound of formula 10. This reaction can be carried out under basic conditions (see for example: Sutton et al, WO 2021/055744). Preferred is the herein described use of trimethylamine, pyridine etc., in DCM, THF or DMF. The reactions are preferably run under an atmosphere of argon for 0.5-24 hours at 0° C. to room temperature.

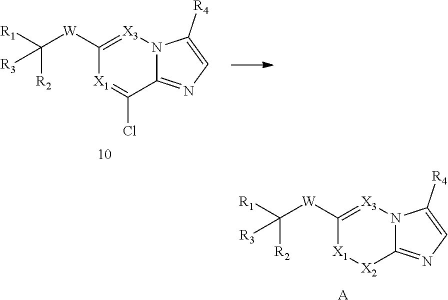

In the final step, a compound of formula 10 in which X₁, X₃, R₁, R₂, R₃ and R₄ are as defined for the compound of formula (I) is coupled with various amines to give a compound of formula A, in which X₂ is defined as for the compound of formula (I). This coupling reaction can be carried out by a palladium-catalyzed C—N cross-coupling reaction (see for example: a) Jiang, Buchwald in ‘Metal-Catalyzed Cross-Coupling Reactions’, 2^{nd} edition.: de Meijere, Diederich, Eds.: Wiley-VCH: Weinheim, Germany, 2004; b) Sutton, et al, WO 2021/055744). Preferred is the herein described use of cesium carbonate and Pd-PEPPSI-IHept Cl in dioxane. The reactions are preferably run under an atmosphere of argon for 1-48 hours at 80-120° C. in a microwave oven or in an oil bath. Preferred is also the herein described use of cesium carbonate RuPhos-Pd-G3, Ruphos in dioxane or palladium acetate, Ruphos, tert-butyl alcohol sodium in THF. The reactions are preferably run under an atmosphere of argon for 1-24 hours at 70-130° C. in a microwave oven or in an oil bath.

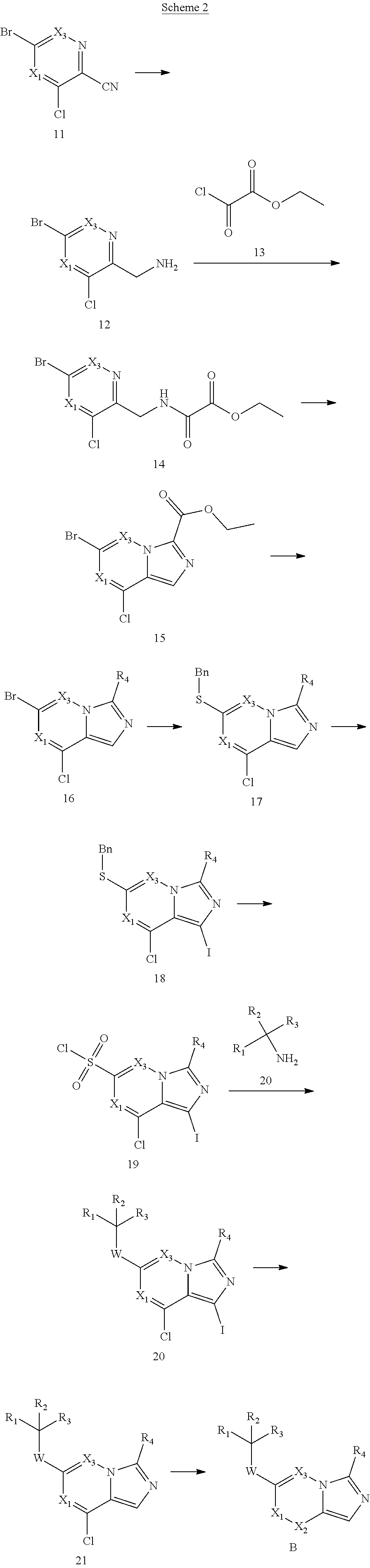

Scheme 2 illustrates a preferred synthetic approach to compounds of the general formula B. As it is to be understandable to the skilled person, the compounds of formula (I) wherein X₄ is C—R₂ other than C—H and X₅ is N are obtainable through functionalization of the C—I position in compound 20, e.g. via palladium-catalyzed cross-coupling reactions.

As it is to be understood herein, X₂ is C—R. X₃ is CH. R₄ is 2-(difluoromethyl)-1,3,4-thiadiazol-2-yl. R₂ and R₃ as shown in scheme 2 and together with a carbon atom that otherwise carries R₂ (not to be confused with R₂ in the definition of X₄) and R₃ form together a cyclopronane ring.

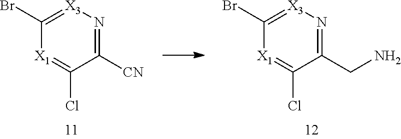

In the first step, the cyano group of a compound of formula 11, in which X₁ is as defined for the compound of formula (I) is reduced to give a compound of formula 12. The reaction is preferably carried out in THF in the presence of a reducing agent like BH₃·THF, BH₃·Me₂S, PtO₂/H₂, sodium tetrahydroborate etc., (see for example: Long et al, WO2018/71535). The reaction is performed at temperatures ranging from 20-40° C. The reaction is preferably completed after 0.5-24 hours.

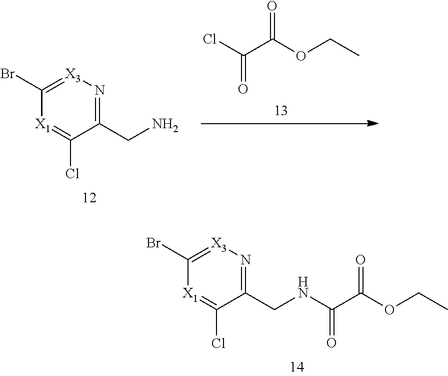

In the second step, a compound of formula 12 in which X₁ is as defined for the compound of formula (I) is reacted with ethyl 2-chloro-2-oxoacetate 13 under basic condition to give a compound of formula 14. The acylation is preferably carried out in a solvent like DCM, dioxane or THF, in the presence of a base like trimethylamine or N-ethyl-N-isopropylpropan-2-amine (see for example: Blaquiere et al, WO2015/25025). The reaction is performed at temperatures ranging from −5° C. to room temperature. The reaction is preferably completed after 1-24 hours.

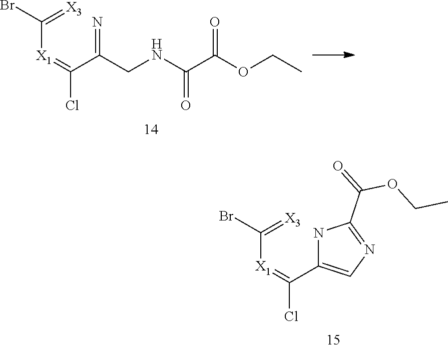

In the third step, a compound of formula 14 in which X₁ is as defined for the compound of formula (I) is converted to a compound of formula 15. The cyclization is preferably carried out in the presence of dehydration reagents like trichlorophosphate, phosphorus pentoxide and trichlorophosphate, pyridine and trifluoroacetic anhydride etc., in 1,2-dichloro-ethane, toluene or neat conditions. The reaction is performed at temperatures ranging from 70-140° C. The reaction is preferably completed after 1-24 hours.

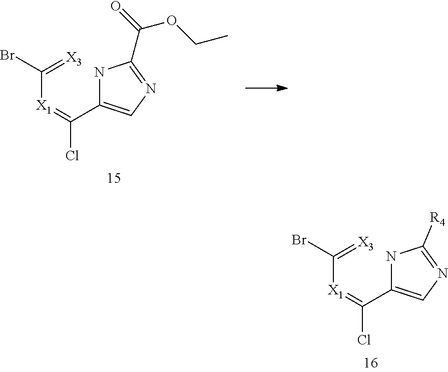

In the fourth step, a compound of formula 15 in which X₁ is as defined for the compound of formula (I) is converted to a compound of formula 16 by several synthetic steps. If R₄ is 2-(difluoromethyl)-1,3,4-thiadiazole, a compound of formula 15 is reacted with hydrazine hydrate to produce a hydrazide. This hydrazide formation can be carried out under neutral conditions (see for example: Dong et al, J. Med. Chem. 2020, 63, 3028). The hydrazide formation is preferably performed in EtOH and the reactions are preferably run for 1-24 hours at 50-100° C. with heating or microwave conditions. The hydrazide is then reacted with ethyl 2,2-difluoroacetate to produce a di-acyl hydrazine. This reaction can be carried out by basic condition, preferred is the herein described use of DBU in EtOH, THF, or DMF. The reactions are preferably run for 0.5-24 hours at room temperature to 100° C. in a microwave oven or in an oil bath. Finally, the di-acyl hydrazine is cyclized by treatment with oxygen/sulfur exchange reagents to a compound of formula 16, in which R₄ is 2-(difluoromethyl)-1,3,4-thiadiazole group. (see for example: Brunet et al, WO2020/127974). Preferred is the herein described use of Lawessons reagent in toluene or THF. The reactions are preferably run for 0.5-24 hours at 50-130° C.

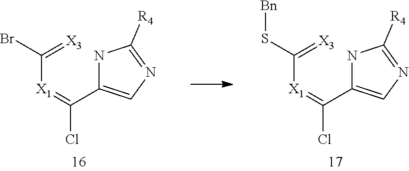

In the fifth step, a compound of formula 16 in which X₁ is as defined for the compound of formula (I) is reacted with benzyl mercaptan to give a compound of formula 17. This coupling reaction can be carried out by a palladium-catalyzed C—S cross-coupling reaction (see for example: Jiang, Buchwald in ‘Metal-Catalyzed Cross-Coupling Reactions’, 2^{nd} edition.: de Meijere, Diederich, Eds.: Wiley-VCH: Weinheim, Germany, 2004). Preferred is the herein described use of tris(dibenzylideneacetone) dipalladium(0), (9,9-dimethyl-9H-xanthene-4,5-diyl)bis(diphenylphosphane) and N-ethyl-N-isopropylpropan-2-amine in dioxane. The reactions are preferably run under an atmosphere of argon for 1-48 hours at 80-100° C. in a microwave oven or in an oil bath.

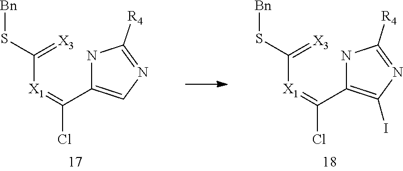

In the sixth step, a compound of formula 17 in which X₁ is as defined for the compound of formula (I) is reacted with an iodide reagent to give a compound of formula 18. This iodization can be carried out by treatment with NIS, 12 etc., in MeCN, THF, dioxane, DMF etc. (see for example: Bentley et al; WO2011/138266). Preferred is the herein described use of NIS in MeCN. The reactions are preferably run under an atmosphere of argon for 0.5-5 hours at 0° C. to room temperature.

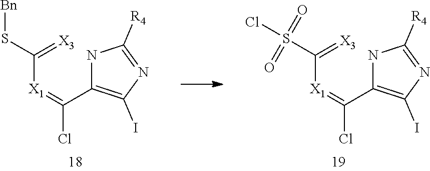

In the seventh step, a compound of formula 18 in which X₁ is as defined for the compound of formula (I) is reacted with chlorination reagent to give a sulfonyl chloride of formula 19. This sulfonyl chloride formation can be carried out by treatment with NCS, sulfonyl chloride, DCDMH, Cl₂ etc., in MeCN with equivalent acetic acid and water. (see for example: Sutton et al, WO 2021/055744). Preferred is the herein described use of DCDMH in MeCN with equivalent acetic acid and water. The reactions are preferably run under an atmosphere of argon for 0.5-5 hours at 0° C. to room temperature.

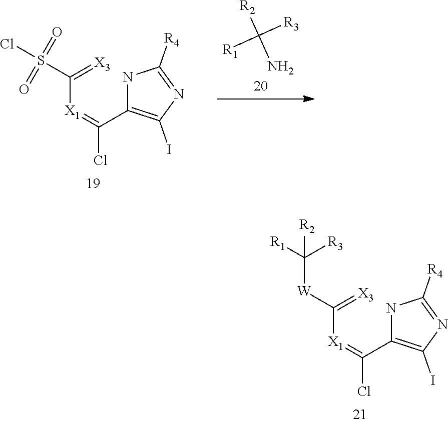

In the eighth step, a compound of formula 19 in which X₁ is as defined for the compound of formula (I) is reacted with an amine of formula 20 in which R₁ is as defined for the compound of formula (I) to give a compound of formula 21. This reaction can be carried out under basic conditions (see for example: Sutton et al, WO 2021/055744). Preferred is the herein described use of trimethylamine, pyridine etc., in DCM, THF or DMF. The reactions are preferably run under an atmosphere of argon for 0.5-24 hours at 0° C. to room temperature.

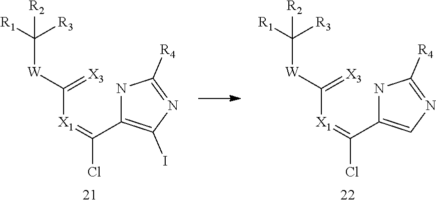

In the ninth step, the iodide of a compound of formula 21 in which X₁ is as defined for the compound of formula (I) is removed by hydrogenation to give a compound of formula 22. The reaction is preferably carried out in THF, MeOH, EtOH, dioxane or DMF in the presence of a hydrogenation catalyst like Pd/C, Pd(OH)₂, Raney Ni, PtO₂ etc. under an atmosphere of hydrogen (see for example: Aissaoui et al, US2011/105514). The reaction is performed at temperatures ranging from 20-80°. The reaction is preferably completed after 0.5-24 hours.

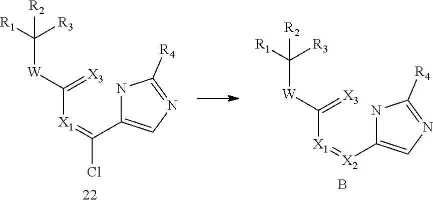

In the final step, a compound of formula 22 in which X₁, R₁, are as defined for the compound of formula (I) is coupled with various amines to give a compound of formula B, in which X₂ is defined as for the compound of formula (I). This coupling reaction can be carried out by a palladium-catalyzed C—N cross-coupling reaction (see for example: a) Jiang, Buchwald in ‘Metal-Catalyzed Cross-Coupling Reactions’, 2^{nd} edition.: de Meijere, Diederich, Eds.: Wiley-VCH: Weinheim, Germany, 2004; b) Sutton et al, WO 2021/055744). Preferred is the herein described use of cesium carbonate and Pd-PEPPSI-IHept Cl in dioxane. The reactions are preferably run under an atmosphere of argon for 1-48 hours at 80-120° C. in a microwave oven or in an oil bath. Preferred is also the herein described use of cesium carbonate RuPhos-Pd-G3, Ruphos in dioxane or palladium acetate, Ruphos, tert-butyl alcohol sodium in THF. The reactions are preferably run under an atmosphere of argon for 1-24 hours at 70-130° C. in a microwave oven or in an oil bath.

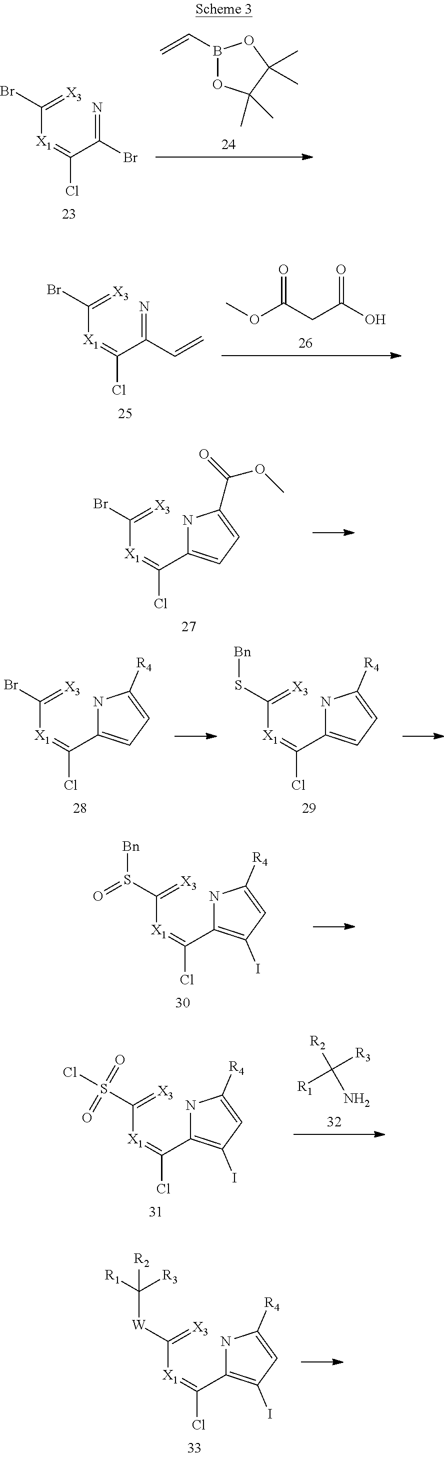

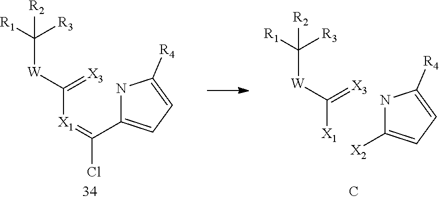

Scheme 3 illustrates a preferred synthetic approach to the compounds of the general formula C. As it is to be understandable to the skilled person, the compounds of formula (I) wherein X₄ is C—R₂ other than C—H, are obtainable through functionalization of C—I position of compound 33, e.g. via palladium-catalyzed cross-coupling reactions.

As it is to be understood herein, X₂ is C—R. X₃ is CH. R₄ is 2-(difluoromethyl)-1,3,4-thiadiazol-2-yl. R₂ and R₃ as shown in scheme 2 and together with a carbon atom that otherwise carries R₂ (not to be confused with R₂ in the definition of X₄) and R₃ form together a cyclopronane ring.

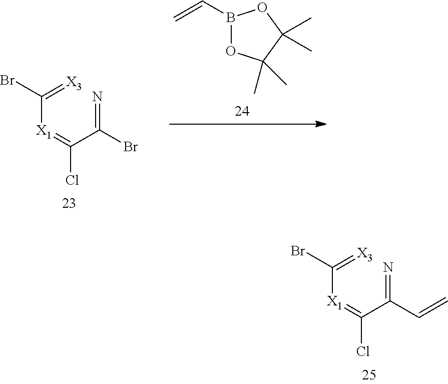

In the first step a compound of formula 23 in which X₁ is as defined for the compound of formula (I) is reacted with 4,4,5,5-tetramethyl-2-vinyl-1,3,2-dioxaborolane 24 to give a compound of formula 25. The coupling reaction is catalyzed by palladium catalysts, e.g. by Pd(0) catalysts like tetrakis(triphenylphosphine) palladium(0) [Pd(PPh₃)₄], tris(dibenzylideneacetone) di-palladium(0) [Pd₂(dba)₃], or by Pd(II) catalysts like dichlorobis(triphenylphosphine)-palladium(II) [Pd(PPh₃)₂Cl₂], palladium(II) acetate and triphenylphosphine or by [1,1′-bis(diphenylphosphino)ferrocene]palladium dichloride. The reaction is preferably carried out in a solvent like 1,2-dimethoxyethane, dioxane, DMF, DME, THF, or isopropanol with water and in the presence of a base like potassium carbonate, sodium carbonate, sodium bicarbonate or potassium phosphate. (see for example: Hall, Boronic Acids, 2005 Wiley VCH Verlag GmbH & Co. KGaA, Weinheim, ISBN 3-527-30991-8 and references cited therein). The reaction is performed at temperatures ranging from room temperature to the boiling point of the respective solvent. Further on, the reaction can be performed at temperatures above the boiling point using pressure tubes and a microwave oven. The reaction is preferably completed after 1 to 36 hours.

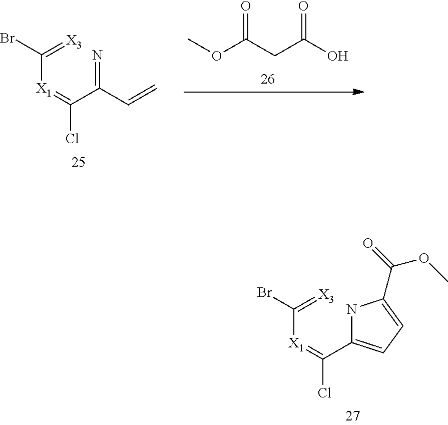

In the second step, a compound of formula 25 in which X₁ is as defined for the compound of formula (I) is reacted with 3-methoxy-3-oxopropanoic acid 26 to give a compound of formula 27. The cyclization is preferably carried out in a solvent like 1,2-dimethoxyethane, dioxane, DMF, DME, THF, or MeCN in the presence of N-iodo-succinimide and sodium acetate. (see for example: Tang et al, Adv. Synth. Catalysis, 2016, 358, 2878). The reaction is performed at temperatures ranging from 80-100° C. in a microwave oven or in an oil bath. The reaction is preferably completed after 1 to 36 hours.

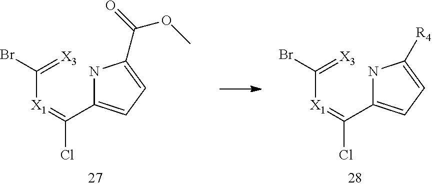

In the third step, a compound of formula 27 in which X₁ is as defined for the compound of formula (I) is converted to a compound of formula 28 by several synthetic steps. If R₄ is 2-(difluoromethyl)-1,3,4-thiadiazole, a compound of formula 27 is reacted with hydrazine hydrate to produce a hydrazide. This hydrazide formation can be carried out under neutral conditions (see for example: Dong et al, J. Med. Chem. 2020, 63, 3028). The hydrazide formation is preferably performed in EtOH and the reactions are preferably run for 1-24 hours at 50-100° C. with heating or microwave conditions. The hydrazide is then reacted with ethyl 2,2-difluoroacetate to produce a di-acyl hydrazine. This reaction can be carried out under basic conditions, preferred is the herein described use of DBU in EtOH, THF, or DMF. The reactions are preferably run for 0.5-24 hours at room temperature to 100° C. in a microwave oven or in an oil bath. Finally, the di-acyl hydrazine is cyclized by treatment with oxygen/sulfur exchange reagents to a compound of formula 28, in which R₄ is 2-(difluoromethyl)-1,3,4-thiadiazole group. (see for example: Brunet et al, WO2020/127974). Preferred is the herein described use of Lawessons reagent in toluene or THF. The reactions are preferably run for 0.5-24 hours at 50-130° C.

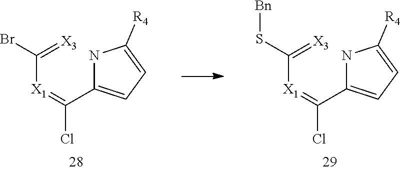

In the fourth step, a compound of formula 28 in which X₁, is as defined for the compound of formula (I) is reacted with benzyl mercaptan to give a compound of formula 29. This coupling reaction can be carried out by a palladium-catalyzed C—S cross-coupling reaction (see for example: Jiang, Buchwald in ‘Metal-Catalyzed Cross-Coupling Reactions’, 2^{nd} edition.: de Meijere, Diederich, Eds.: Wiley-VCH: Weinheim, Germany, 2004). Preferred is the herein described use of tris(dibenzylideneacetone) dipalladium(0), (9,9-dimethyl-9H-xanthene-4,5-diyl)bis(diphenylphosphane) and N-ethyl-N-isopropylpropan-2-amine in dioxane. The reactions are preferably run under an atmosphere of argon for 1-48 hours at 80-100° C. in a microwave oven or in an oil bath.

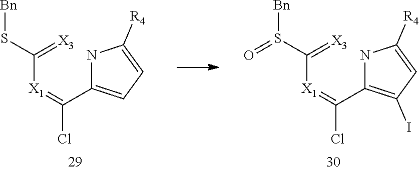

In the fifth step, a compound of formula 29 in which X₁, is as defined for the compound of formula (I) is reacted with an iodide reagent to give a compound of formula 30. This iodization can be carried out by treatment with NIS, 12 etc., in MeCN, THF, dioxane, DMF etc. (see for example: Bentley et al, WO2011/138266). Preferred is the herein described use of NIS in MeCN. The reactions are preferably run under an atmosphere of argon for 0.5-5 hours at 0° C. to room temperature.

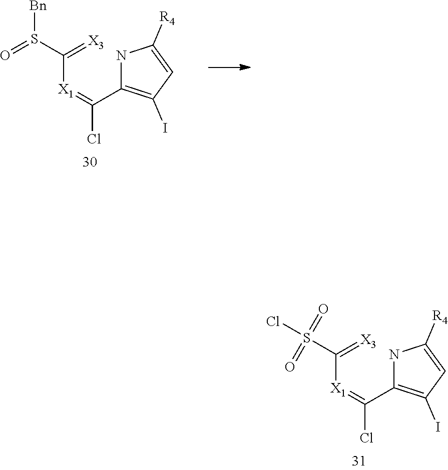

In the six step, a compound of formula 30 in which X₁, is as defined for the compound of formula (I) is reacted with chlorination reagent to give a sulfonyl chloride of formula 31. This sulfonyl chloride formation can be carried out by treatment with NCS, sulfonyl chloride, DCDMH, Cl₂ etc., in MeCN with equivalent acetic acid and water. (see for example: Sutton et al, WO 2021/055744). Preferred is the herein described use of DCDMH in MeCN with equivalent acetic acid and water. The reactions are preferably run under an atmosphere of argon for 0.5-5 hours at 0° C. to room temperature.

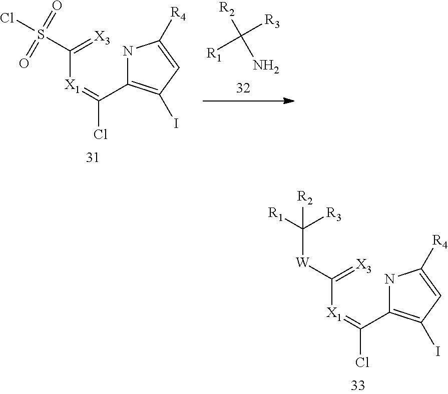

In the seventh step, a compound of formula 31 in which X₁, is as defined for the compound of formula (I) is reacted with an amine of formula 32 in which R₁, R₂ and R₃ are as described hereinabove to give a compound of formula 33. This reaction can be carried out under basic conditions (see for example: Sutton et al, WO 2021/055744). Preferred is the herein described use of trimethylamine, pyridine etc., in DCM, THF or DMF. The reactions are preferably run under an atmosphere of argon for 0.5-24 hours at 0° C. to room temperature.

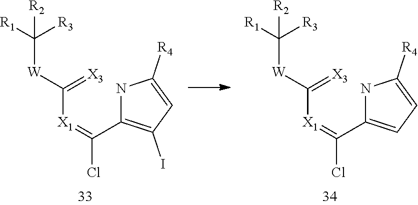

In the eighth step, the iodide of a compound of formula 33 in which X₁ and R₁ are as defined for the compound of formula (I) is removed by hydrogenation to give a compound of formula 34. The reaction is preferably carried out in THF, MeOH, EtOH, dioxane or DMF in the presence of a hydrogenation catalyst like Pd/C, Pd(OH)₂, Raney Ni, PtO₂ etc. under an atmosphere of hydrogen, (see for example: Aissaoui et al, US2011/105514). The reaction is performed at temperatures ranging from 20-80°. The reaction is preferably completed after 0.5-24 hours.

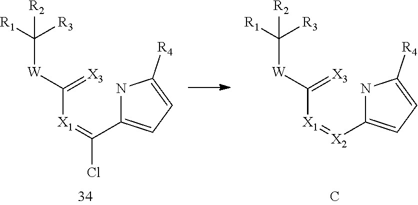

In the final step, a compound of formula 34 in which X₁, and R₁ are as defined for the compound of formula (I) is coupled with various amines to give a compound of formula C. This coupling reaction can be carried out by a palladium-catalyzed C—N cross-coupling reaction (see for example: a) Jiang, Buchwald in ‘Metal-Catalyzed Cross-Coupling Reactions’, 2^{nd} edition.: de Meijere, Diederich, Eds.: Wiley-VCH: Weinheim, Germany, 2004; b) Sutton et al, WO 2021/055744). Preferred is the herein described use of cesium carbonate and Pd-PEPPSI-IHept Cl in dioxane. The reactions are preferably run under an atmosphere of argon for 1-48 hours at 80-120° C. in a microwave oven or in an oil bath. Preferred is also the herein described use of cesium carbonate RuPhos-Pd-G3, Ruphos in dioxane or palladium acetate, Ruphos, tert-butyl alcohol sodium in THF. The reactions are preferably run under an atmosphere of argon for 1-24 hours at 70-130° C. in a microwave oven or in an oil bath.

### PREPARATIVE EXAMPLES

### General Considerations

Abbreviations used in the descriptions that follow are: AcOH (acetic acid); aq. (aqueous); Ar (Argon); Atm (atmosphere); BH₃·THF (boran tetrahydrofuran complex); BH₃·Me₂S (boran dimethylsulfide complex); br. (broad, ¹H NMR signal); Boc₂O (di-tert-butyldicarbonate); (Cataxium APdG₃ (Mesylate[(di(1-adamantyl)-n-butylphosphine)-2-(2′-amino-1,1′-biphenyl)]palladium(II)); (CDCl₃ (deuterated chloroform); cHex (cyclohexane); CMPB (Cyanomethylene trimethylphosphorane); Cs₂CO₃ (cesium carbonate); CuI (copper iodide); DABCO ((1,4-diazabicyclo[2.2.2]octane)); DAST (diethylaminosulfur trifluoride);DBU (1,8-Diazabicyclo(5.4.0)undec-7-ene); DCE (dichloroethane); d (doublet, ¹H NMR signal); DCM (dichloromethane); DIBAL-H (diisobutyl aluminium hydride); DIPEA or DIEA (di-iso-propylethylamine); DMAP (4-N—N-dimethylaminopyridine), DME (1,2-dimethoxyethane), DMEDA (dimethylethylenediamine); DMF (N—N-dimethylformamide); DMSO (dimethyl sulfoxide); DPPA (diphenylphosphoride azide);dtbbpy (Bis(1,1-dimethylethyl)-2,2′-bipyridine); ES (electrospray); EtOAc or EA (ethyl acetate); EtOH (ethanol); h (hour(s)); FA (formic acid); HATU (1-[Bis(dimethylamino)methylene]-1H-1,2,3-triazolo[4,5-b]pyridinium 3-oxide hexafluorophosphate); H₂O (water); HFIP (Hexafluoroisopropanol); ¹H NMR (proton nuclear magnetic resonance spectroscopy); HPLC (High Performance Liquid Chromatography), iPrOH (iso-propanol); K₃PO₄ (tripotassium phosphate); Ir[dF(CF₃)(dtbbpy)PF₆ ((4,4′-Di-t-butyl-2,2′-bipyridine)bis[3,5-difluoro-2-[5-trifluoromethyl-2-pyridinyl-kN)phenyl-kC]iridium(III) hexafluorophosphate); LiHMDS (lithium bis(trimethyl)amide);LiOH (lithium hydroxide); m (multiplet, ¹H NMR signal); mCPBA (meta-chloroperoxybenzoic acid), MeCN (acetonitrile), MeOH (methanol); min (minute(s)); MnO₂ (Manganese (IV) oxide); MS (mass spectrometry); MPLC (medium pressure liquid chromatography); MTBE (methyl tert-butyl ether); NaBH₄ (sodium borohydride); NaBH(OAC)₃ (sodium triacetoxyborohydride); NaHCO₃ (sodium hydrogenocarbonate); Na₂S₂O₃ (sodium thiosulfate); NCS (N-chlorosuccinimide); NH₃ (ammonia); NH₄Cl (ammonium fluoride); NiCl₂ (nickel dichloride); NIS (N-lodosuccinimide); NMP (N-methylpyrrolidone); NMR (nuclear magnetic resonance); Pd/C (palladium on charcoal); Pd₂dba₃ (tris(dibenzylideneacetone)dipalladium); Pd(dppf)Cl₂ (1,1-Bis(diphenylphosphino)ferrocene dichloropalladium); Pd(Ph₃)₂Cl₂ (Bis(triphenylphosphine)palladium(II) dichloride); PE (petroleum ether); Pd-PEPPSI-IPentCl o-picoline ([1,3-bis[2,6-bis(1-ethylpropyl)phenyl]-4,5-dichloro-imidazol-2-ylidene]-dichloro-(2-methylpyridin-1-ium-1-yl)palladium; Pd(OH)₂ (palladium hydroxide); Pd(Ph₃)₄ (Palladium-tetrakis(triphenylphosphine)); Phl(OAc)₂ ((Diacetoxyiodo)benzene)); P(tBu)₃ (Tri-tert-butylphosphine); Py (pyridine); q (quartet, 1H NMR signal); quin (quintet, 1H NMR signal); rac (racemic); RT (retention time); s (singlet, ¹H NMR signal); sat. (saturated); SFC (supercritical fluid chromatography); t (triplet, ¹H NMR signal); TBAF (tetrabutylammonium fluoride); tert-BuBrettPhos-Pd-G3 ([(2-Di-tert-butylphosphino-3,6-dimethoxy-2′,4′,6′-triisopropyl-1,1′-biphenyl)-2-(2′-amino-1,1′-biphenyl)]palladium(II) methanesulfonate); tBuXPhos Pd G3 (Methanesulfonato(2-di-t-butylphosphino-2′,4′,6′-tri-1-propyl-1,1′-biphenyl)(2′-amino-1,1′-biphenyl-2-yl)palladium(II))TBDMSCl orTBSCl (tert-butyldimethylsilyl chloride); tBuOH (tert-butanol); TEA (triethylamine); TFA (trifluoroacetic acid); TFAA (trifluoroacetic anhydride), THF (tetrahydrofuran); TLC (thin layer chromatography); TMSCHN₂ (Trimethylsilyldiazomethane); TMSCN (trimethylsilyl cyanide); TMSOTf (Trimethylsilyl trifluoromethanesulfonate); TTMSS (trimethylsilane); UPLC (Ultra-High Performance Liquid Chromatography), UV (ultraviolet), wt-% (percent by weight); Xantphos (4,5-Bis(diphenylphosphino)-9,9-dimethylxanthene); Xantphos Pd G4 (Methanesulfonato[9,9-dimethyl-4,5-bis(diphenylphosphino)xanthene](2′-methylamino-1,1′-biphenyl-2-yl)palladium(II)).

General Procedure: All starting materials and solvents were obtained either from commercial sources or prepared according to literature references. Commercially available reagents and anhydrous solvents were used as supplied, without further purification. Unless otherwise stated all reactions were stirred. Organic solutions were routinely dried over anhydrous sodium sulfate. Column chromatography was performed on pre-packed silica (100-1000 mesh, 40-63 μm) cartridges using the amount indicated. All air- and moisture-sensitive reactions were carried out in oven-dried (at 120° C.) glassware under an inert atmosphere of nitrogen or argon. Compound names were generated using ChemDraw Professional (Perkin Elmer). In some cases generally accepted names of commercially available reagents were used in place of ChemDraw generated names. Unless specifically indicated to the contrary, for the yield calculation the purity of the obtained product is assumed to be 100%.

### Reversed Phase HPLC m for LCMS Analytical Methods:

Method 1: SHIMADZU LCMS-2020 Kinetex® EVO C18 2.1×30 mm, 5 μm at 50° C.; Mobile Phase: A: 0.0375% TFA in water (v/v); B: 0.01875% TFA in MeCN (v/v); flow rate held at 1.5 mL/min; eluted with the mobile phase over 1.55 min employing UV detection at 220 nm and 254 nm. Gradient information: 0-0.80 min, ramped from 95% A-5% B to 5% A-95% B; 0.80-1.20 min, held at 5% A-95% B; 1.20-1.21 min, returned to 95% A-5% B, 1.21-1.55 min, held at 95% A-5% B.

Method 2: SHIMADZU LCMS-2020 Kinetex® EVO C18 2.1×30 mm, 5 μm at 40° C.; Mobile Phase A: 0.025% NH₃·H₂O in water (v/v); B: MeCN; flow rate held at 1.5 mL/min; eluted with the mobile phase over 1.55 min employing UV detection at 220 nm and 254 nm. Gradient information: 0-0.80 min, ramped from 95% A-5% B to 5% A-95% B; 0.80-1.20 min, held at 5% A-95% B; 1.20-1.21 min, returned to 95% A-5% B, 1.21-1.55 min, held at 95% A-5% B.

Method 3: SHIMADZU LCMS-2020 Kinetex® EVO C18 2.1×30 mm, 5 μm at 50° C.; Mobile Phase: A: 0.0375% TFA in water (v/v); B: 0.01875% TFA in MeCN (v/v); flow rate held at 2.0 mL/min; eluted with the mobile phase over 0.80 min employing UV detection at 220 nm and 254 nm. Gradient information: 0-0.80 min, ramped from 95% A-5% B to 5% A-95% B; 0.80-1.20 min, held at 5% A-95% B; 1.20-1.21 min, returned to 95% A-5% B, 1.21-1.55 min, held at 95% A-5% B.

Method 4: SHIMADZU LCMS-2020 Kinetex® EVO C18 2.1×20 mm 2.6 μm at 50° C.; Mobile Phase: A: 0.0375% TFA in water (v/v); B: 0.01875% TFA in MeCN (v/v); flow rate held at 2.0 mL/min; eluted with the mobile phase over 1.00 min employing UV detection at 220 nm and 254 nm. Gradient information: 0.01-0.60 min, ramped from 95% A-5% B to 5% A-95% B; 0.61-0.78 min, held at 5% A-95% B; 0.78-0.79 min, returned to 95% A-5% B, 0.79-0.80 min, held at 95% A-5% B.

Method 5: SHIMADZU LCMS-2020 Kinetex® EVO C18 2.1×30 mm 5 μm at 50° C. Mobile Phase: A: 0.0375% TFA in water (v/v); B: 0.01875% TFA in MeCN (v/v); flow rate held at 1.5 mL/min; eluted with the mobile phase over 1.00 min employing UV detection at 220 nm and 254 nm. Gradient information: 0.01-0.80 min, ramped from 95% A-5% B to 5% A-95% B; 0.80-0.95 min, held at 5% A-95% B; 0.95-0.96 min, returned to 95% A-5% B, 0.96-1.00 min, held at 95% A-5% B.

Method 6: SHIMADZU LCMS-2020 HALO C18 3.0×30 mm, 5 μm at 50° C.; Mobile Phase: A: 0.0375% TFA in water (v/v); B: 0.01875% TFA in Acetonitrile (v/v); flow rate held at 1.5 mL/min; eluted with the mobile phase over 1.05 min employing UV detection at 220 nm and 254 nm. Gradient information: 0-0.50 min, ramped from 95% A-5% B to 5% A-95% B; 0.50-0.80 min, held at 5% A-95% B; 0.80-0.81 min, returned to 95% A-5% B, 0.81-1.05 min, held at 95% A-5% B.

Method 7: SHIMADZU LCMS-2020 Kinetex® EVO C18 2.1×30 mm 5 μm at 50° C.; Mobile Phase: A: 0.0375% TFA in water (v/v); B: 0.01875% TFA in Acetonitrile (v/v); flow rate held at 2.0 mL/min; eluted with the mobile phase over 0.80 min employing UV detection at 220 nm and 254 nm. Gradient information: 0-0.60 min, ramped from 95% A-5% B to 5% A-95% B; 0.60-0.78 min, held at 5% A-95% B; 0.78-0.79 min, returned to 95% A-5% B, 0.79-0.80 min, held at 95% A-5% B.

### SFC Analytical Method:

SFC Method 1: Column:Chiralpak IH-3 50*4.6 mm I.D., 3 μm Mobile phase:Phase A for CO₂, and Phase B for EtOH(0.05% DEA); Gradient elution:EtOH(0.05% DEA) in CO₂ from 5% to 40%, Flow rate: 3 mL/min; Detector:PDA; Column Temp:35 C; Back Pressure:100 Bar.

### ¹H NMR Spectroscopy:

¹H NMR spectra were acquired on a Bruker Avance III spectrometer at 400 MHz using residual undeuterated solvent as reference. ¹H NMR signals are specified with their multiplicity/combined multiplicities as apparent from the spectrum; possible higher-order effects are not considered. Chemical shifts of the signals (δ) are specified as ppm (parts per million).

### Salt Stoichiometry:

In the present text, in particular in the experimental section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown. Unless specified otherwise, suffixes to chemical names or structural formulae such as “hydrochloride”, “trifluoroacetate”, “sodium salt”, or “×HO”, “×CF₃COOH”, “×Na+”, for example, are to be understood as not a stoichiometric specification, but solely as a salt form. This applies analogously to cases in which synthesis intermediates or example compounds or salts thereof have been obtained, by the preparation and/or purification processes described, as solvates, such as hydrates with (if defined) unknown stoichiometric composition

General procedure 2 (Boc cleavage): To a solution of the Boc-protected compound in DCM (0.1 g/mL) was added TFA (⅕ to ⅓ of the DCM volume) or HCl/Dioxane (4N, ⅕ to ⅓ of the DCM volume) at 0° C. and the mixture was stirred at 0° C. for 2 to 16 h. Then, the mixture was concentrated under reduce pressure. The resulting residue was purified by reverse preparative HPLC (reverse phase) or preparative TLC or SiO₂ column chromatography (normal phase) to give the corresponding product.

### Preparation of Intermediate 1.1

### potassium (Z)-2-chloro-3-ethoxy-3-oxoprop-1-en-1-olate

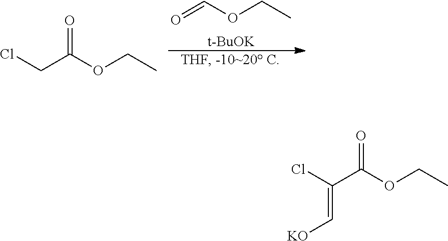

A solution of ethyl 2-chloroacetate (10 g, 81.60 mmol, 8.70 mL) and ethyl formate (6.04 g, 81.60 mmol, 6.56 mL) in tetrahydrofuran (THF) (150 mL) was stirred at −10° C. for 20 min, then t-BuOK (11.90 g, 106.08 mmol) was added in portions so that the temperature of the mixture remained below 0-5° C. The reaction was warmed to 20° C. for 16 hours. The reaction mixture was filtered to give a solid which was triturated with EtOAc (50 mL) for 5 hours at 20° C., filtered and the solid was dried under vacuum to give potassium (Z)-2-chloro-3-ethoxy-3-oxoprop-1-en-1-olate (12 g, 63.61 mmol, 77.95% yield) as a yellow solid.

¹H NMR (400 MHz, DMSO-d₆) δ 8.88-8.24 (m, 1H), 4.16 (q, J=7.2 Hz, 2H), 1.24 (t, J=7.2 Hz, 3H).

### Preparation of Intermediate 1.2

### ethyl 6-bromo-8-chloroimidazo[1,2-a]pyridine-3-carboxylate

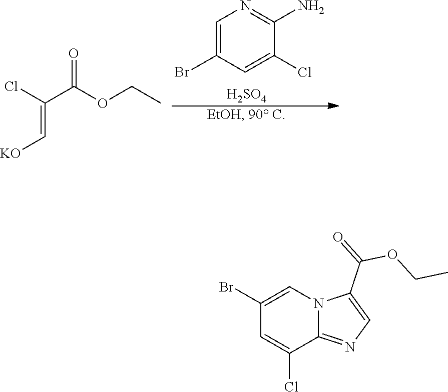

To a solution of 5-bromo-3-chloro-pyridin-2-amine (2 g, 9.64 mmol) and potassium (Z)-2-chloro-3-ethoxy-3-oxoprop-1-en-1-olate (7.27 g, 38.56 mmol) in EtOH (100 mL) at 20° C. was added H₂SO₄ (2.84 g, 28.92 mmol, 1.54 mL). The reaction mixture was heated to 90° C. for 16 hours. The reaction mixture was cooled to 20° C. EtOH was removed under reduced pressure, water (50 mL) was added and the mixture was extracted with EtAOc (3×80 mL). The combined organic layer was washed with brine (50 mL), dried over with Na₂SO₄, filtered and concentrated to give a residue, which was purified by flash silica gel chromatography (ISCO®; 40 g SepaFlash® Silica Flash Column, Eluent of 0-30% Ethyl acetate/Petroleum ether gradient @80 mL/min) to give the product ethyl 6-bromo-8-chloroimidazo[1,2-a]pyridine-3-carboxylate (1.7 g, 5.21 mmol, 54.03% yield) as a white solid.

RT 0.888 min (method 1); m/z 304.9 (M+H)⁺ (ESI⁺); ¹H NMR (DMSO-d₆, 400 MHz): δ 9.24 (s, 1H), 8.301 (s, 1H), 8.05-8.04 (m, 1H), 4.40-4.35 (m, 2H), 1.35 (t, J=7.2 Hz, 3H).

### Preparation of Intermediate 1.3

### 6-bromo-8-chloroimidazo[1,2-a]pyridine-3-carbohydrazide

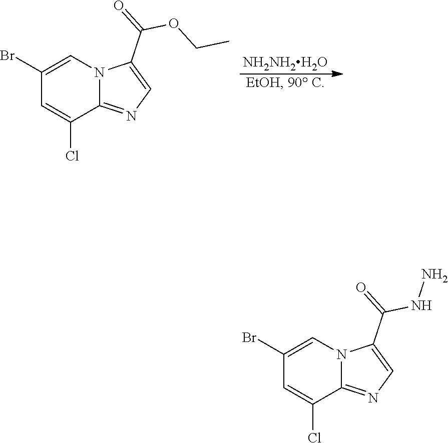

To a solution of ethyl 6-bromo-8-chloroimidazo[1,2-a]pyridine-3-carboxylate (1.7 g, 5.21 mmol, 93% purity) in EtOH (20 mL) at 20° C. was added NH₂NH₂·H₂O (3.26 g, 63.76 mmol, 3.16 mL, 98% purity). The mixture was refluxed for 2 h. and then cooled to 20° C. The precipitated solid was separated off to give the product 6-bromo-8-chloroimidazo[1,2-a]pyridine-3-carbohydrazide (1.5 g, 4.97 mmol, 95.49% yield) as a white solid.

RT 0.487 min (method 1); m/z 290.1 (M+H)⁺ (ESI⁺); ¹H NMR (DMSO-d₆, 400 MHz): 10.68-9.40 (m, 2H), 8.32 (s, 1H), 7.92 (d, J=1.6 Hz, 1H), 4.83-4.27 (m, 2H).

### Preparation of Intermediate 1.4

### 6-bromo-8-chloro-N′-(2,2-difluoroacetyl)imidazo[1,2-a]pyridine-3-carbohydrazide

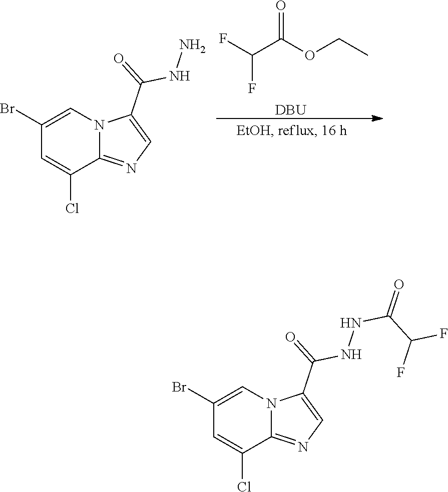

To a mixture of 6-bromo-8-chloroimidazo[1,2-a]pyridine-3-carbohydrazide (1.3 g, 4.31 mmol) and ethyl 2,2-difluoroacetate (5.35 g, 43.11 mmol) in EtOH (110 mL) at 20° C. was added 1,8-diazabicyclo[5.4.0]undec-7-ene (DBU) (1.31 g, 8.62 mmol). The mixture was refluxed for 16 hours before it was cooled to 20° C. and finally concentrated to give a residue, which was purified by flash silica gel chromatography (ISCO®; 20 g SepaFlash® Silica Flash Column, Eluent of 90% Ethyl acetate/Petroleum ether gradient @80 mL/min) to give the product 6-bromo-8-chloro-N′-(2,2-difluoroacetyl) imidazo[1,2-a]pyridine-3-carbohydrazide (0.85 g, 2.17 mmol) as a white solid.

RT 0.770 min (method 1); m/z 368.9 (M+H)⁺ (ESI⁺); ¹H NMR (DMSO-d₆, 400 MHz): 11.03-10.93 (m, 2H), 9.49 (s, 1H), 8.48 (s, 1H), 8.03 (s, 1H), 6.48 (t, J=52.8 Hz, 1H).

### Preparation of Intermediate 1.5

### 2-(6-bromo-8-chloroimidazo[1,2-a]pyridin-3-yl)-5-(difluoromethyl)-1,3,4-thiadiazole

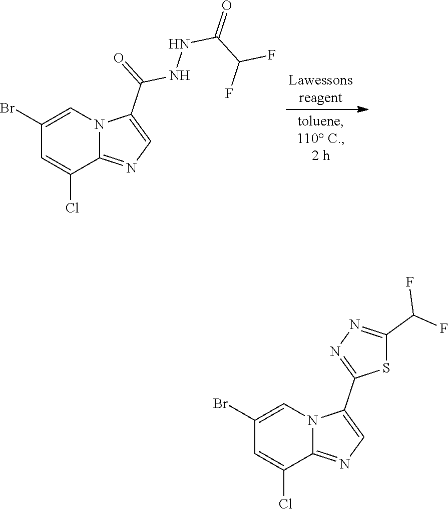 [SMILES: O=C(NNC(=O)C(F)F)c1cnc2c(Cl)cc(Br)cn12 ; FC(F)c1nnc(-c2cnc3c(Cl)cc(Br)cn23)s1]

To a solution of 6-bromo-8-chloro-N′-(2,2-difluoroacetyl)imidazo[1,2-a]pyridine-3-carbohydrazide (200.00 mg, 511.52 μmol) in toluene (4 mL)) at 20° C. was added Lawesson's reagent (227.58 mg, 562.67 μmol. The mixture was stirred at 110° C. for 2 hours. The mixture was cooled to 20° C. and concentrated to give a residue, which was triturated with MeOH (5 mL) for 30 min. after filtration, the cake was collected to give the product 2-(6-bromo-8-chloro-imidazo[1,2-a]pyridin-3-yl)-5-(difluoromethyl)-1,3,4-thiadiazole (140 mg, 382.95 μmol, 74.87% yield) as white solid.

¹H NMR (DMSO-d₆, 400 MHz): 7.54-7.84 (t, J=53.2 Hz, 1H), 8.08 (d, J=1.6 Hz, 1H), 8.64 (s, 1H), 9.61 (d, J=1.6 Hz, 1H).

### Preparation of Intermediate 1.6

### 2-(6-(benzylthio)-8-chloroimidazo[1,2-a]pyridin-3-yl)-5-(difluoromethyl)-1,3,4-thiadiazole

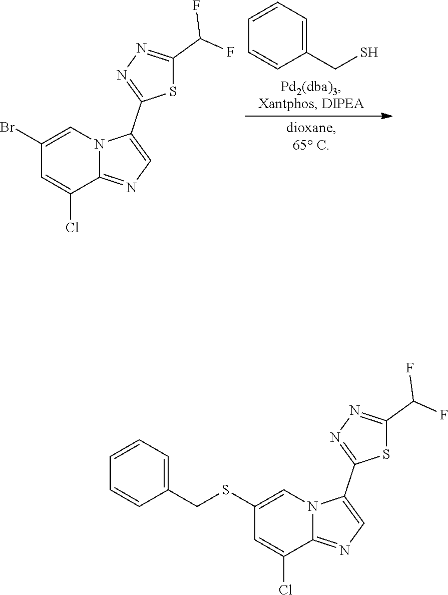

A mixture of 2-(6-bromo-8-chloro-imidazo[1,2-a]pyridin-3-yl)-5-(difluoromethyl)-1,3,4-thiadiazole (100 mg, 273.53 μmol), Xantphos (31.65 mg, 54.71 μmol), N,N-diisopropylethylamine (DIPEA) (70.70 mg, 547.07 μmol) and Pd₂(dba)₃ (tris(dinezylideneacetone)dipalladium(0)) (25.05 mg, 27.35 μmol) in dioxane (2 mL) was stirred at 20° C. N₂ was bubbled through the mixture for 5 min, and finally phenylmethanethiol (33.97 mg, 273.53 μmol, 32.05 μL) was added. The mixture was heated to 65° C. and stirred for 16 h. The reaction mixture was cooled to 20° C. and concentrated to give a residue, which was purified by flash silica gel chromatography (ISCO®; 20 g SepaFlash® Silica Flash Column, Eluent of 0-30% Ethyl acetate/Petroleum ether gradient @30 mL/min) to give the product 2-(6-benzylsulfanyl-8-chloroimidazo[1,2-a]pyridin-3-yl)-5-(difluoromethyl)-1,3,4-thiadiazole (80 mg, 193.70 μmol, 70.81% yield) as a yellow solid.

RT 1.012 min (method 1); m/z 408.9 (M+H)⁺ (ESI⁺).

### Preparation of Intermediate 1.7

### 8-chloro-3-(5-(difluoromethyl)-1,3,4-thiadiazol-2-yl)imidazo[1,2-a]pyridine-6-sulfonyl chloride

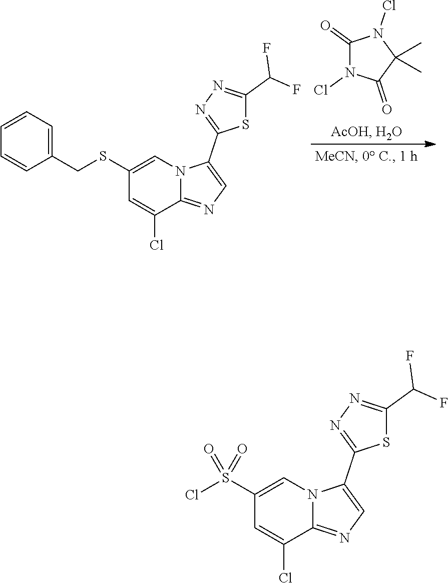

To a mixture of 2-(6-benzylsulfanyl-8-chloroimidazo[1,2-a]pyridin-3-yl)-5-(difluoromethyl)-1,3,4-thiadiazole (20 mg, 48.91 μmol), AcOH (5.29 mg, 88.05 μmol, 5.04 μL) and H₂O (881.21 μg, 48.91 μmol) in MeCN (0.5 mL) at 0° C. was added 1,3-dichloro-5,5-dimethyl-imidazolidine-2,4-dione (17.35 mg, 88.05 μmol). The mixture was stirred at 0° C. for 0.5 h. THF (3 mL) was added and the solution was dried over Na₂SO₄, filtered and concentrated to give the product 8-chloro-3-[5-(difluoromethyl)-1,3,4-thiadiazol-2-yl]imidazo [1,2-a]pyridine-6-sulfonyl chloride (18 mg, 46.73 μmol, 95.53% yield) as a white solid, which was used in the next step without further purification.

### Preparation of intermediate 1.8

### 8-chloro-3-(5-(difluoromethyl)-1,3,4-thiadiazol-2-yl)-N-(1-methylcyclopropyl)imidazo[1,2-a]pyridine-6-sulfonamide

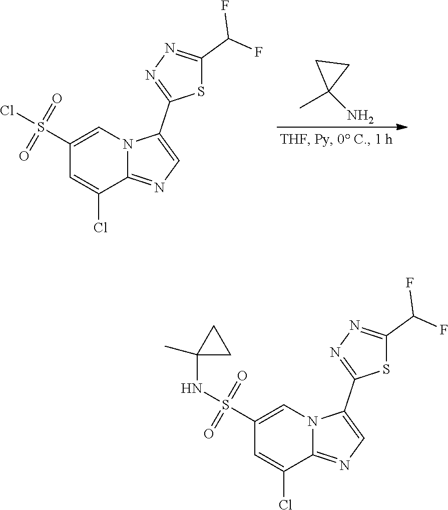

To the mixture of 1-methylcyclopropanamine (78.20 mg, 726.90 μmol, HCl salt) in NaHCO₃ (aq., sat., 3 mL) was added drop-wise 8-chloro-3-(5-(difluoromethyl)-1,3,4-thiadiazol-2-yl)imidazo[1,2-a]pyridine-6-sulfonyl chloride (140 mg, 363.45 μmol) in THF (1.5 mL) at 0° C. The mixture was stirred at 15° C. for 2 h. The reaction mixture was quenched by H₂O (30 mL) and extracted with EtOAc (30 mL, 3×). The combined organic layer was washed with brine (30 mL), dried over Na₂SO₄, filtered and concentrated under reduced pressure. The resulting residue was purified by flash silica gel chromatography (ISCO®; 4 g SepaFlash® Silica Flash Column, Eluent 20-40% Ethyl acetate/Petroleum @75 mL/min) to give the product 8-chloro-3-(5-(difluoromethyl)-1,3,4-thiadiazol-2-yl)-N-(1-methylcyclopropyl)imidazo[1,2-a]pyridine-6-sulfonamide (50 mg, 119.09 μmol, 32.77% yield) as a white solid.

RT 0.809 min (Method 1); m/z 420.1 (M+H)⁺ (ESI⁺); ¹H NMR (DMSO-d₆, 400 MHz) 9.99 (d, J=1.2 Hz, 1H), 8.81 (s, 1H), 8.58 (s, 1H), 7.94 (d, J=1.2 Hz, 1H), 7.73 (t, J=53.2 Hz, 1H), 1.17-1.20 (m, 3H), 0.67-0.76 (m, 2H), 0.45-0.53 (m, 2H).

### Preparation of Intermediate 1.9

### tert-butyl (2S,6S)-4-(3-(5-(difluoromethyl)-1,3,4-thiadiazol-2-yl)-6-(N-(1-methylcyclopropyl)sulfamoyl)imidazo[1,2-a]pyridin-8-yl)-2,6-dimethylpiperazine-1-carboxylate

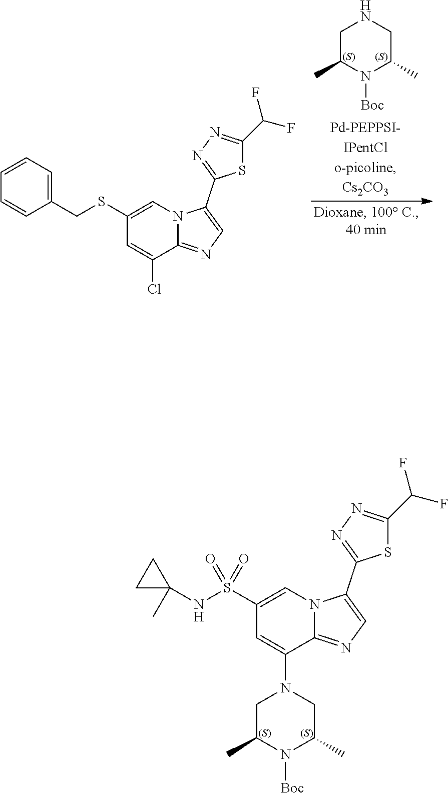

To a solution of 8-chloro-3-(5-(difluoromethyl)-1,3,4-thiadiazol-2-yl)-N-(1-methylcyclopropyl)imidazo[1,2-a]pyridine-6-sulfonamide (50 mg, 0.119 mmol) in dioxane (1 mL) were added tert-butyl (2S,6S)-2,6-dimethylpiperazine-1-carboxylate (51 mg, 0.238 mmol), Pd-PEPPSI-IPentCl o-picoline (12 mg, 0.0119 mmol) and Cs₂CO₃ (116 mg, 0.357 mmol). The mixture was degassed with N₂ (3×) and stirred at 100° C. for 40 min under nitrogen atmosphere. Then, the reaction mixture was cooled to 25° C., filtered and the filtrate was concentrated under vacuum to give. The resulting residue was purified by preparative TLC (petroleum ether:ethyl acetate=0:1) to give the product tert-butyl (2S,6S)-4-(3-(5-(difluoromethyl)-1,3,4-thiadiazol-2-yl)-6-(N-(1-methylcyclopropyl)sulfamoyl)imidazo[1,2-a]pyridin-8-yl)-2,6-dimethylpiperazine-1-carboxylate (40 mg, 0.0669 mmol, 34.22% yield, 61% purity) as a yellow solid.

RT 0.520 min (method 4); m/z 598.4 (M+H)⁺ (ESI⁺)

### Preparation of Example 1

### 3-(5-(difluoromethyl)-1,3,4-thiadiazol-2-yl)-8-((3S,5S)-3,5-dimethylpiperazin-1-yl)-N-(1-methylcyclopropyl)imidazo[1,2-a]pyridine-6-sulfonamide formate

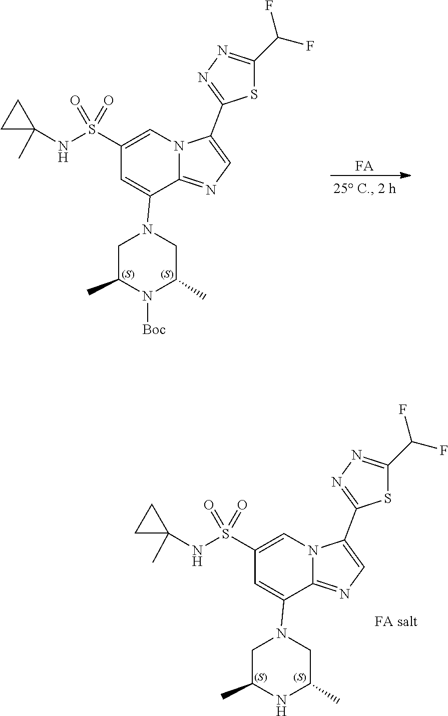

A solution of tert-butyl (2S,6S)-4-(3-(5-(difluoromethyl)-1,3,4-thiadiazol-2-yl)-6-(N-(1-methylcyclopropyl)sulfamoyl)imidazo[1,2-a]pyridin-8-yl)-2,6-dimethylpiperazine-1-carboxylate (38 mg, 0.0636 mmol) in formic acid (1.0 mL) was stirred at 25° C. for 2 hours and then, concentrated under vacuum. The resulting residue was purified by preparative HPLC (column: Phenomenex luna C18 150*25 mm*10 μm; mobile phase: A: 0.225% formic acid in water, B: MeCN; B %: 16%-46%,10 min) and lyophilized directly to give the product 3-(5-(difluoromethyl)-1,3,4-thiadiazol-2-yl)-8-((3S,5S)-3,5-dimethylpiperazin-1-yl)-N-(1-methylcyclopropyl)imidazo[1,2-a]pyridine-6-sulfonamide formate (12.59 mg, 0.0229 mmol, 59.10% yield, 98.80% purity, FA salt) as a yellow solid.

RT 0.362 min (method 4); m/z 498.2 (M+H)⁺ (ESI⁺); ¹H NMR (CD₃CN, 400 MHz): 9.73 (s, 1H), 8.45-8.20 (m, 2H), 7.25 (t, J=53.6 Hz, 1H), 6.91 (s, 1H), 6.40 (br, 1H), 3.72-3.59 (m, 2H), 3.58-3.42 (m, 4H), 1.39-1.23 (m, 6H), 1.21 (s, 3H), 0.85-0.73 (m, 2H), 0.53-0.39 (m, 2H).

### Preparation of Intermediate 2.1

### (5-bromo-3-chloropyridin-2-yl)methanamine

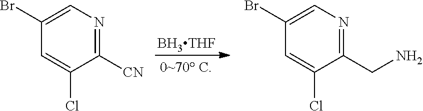 [SMILES: N#Cc1ncc(Br)cc1Cl ; NCc1ncc(Br)cc1Cl ; "BH3·THF"]

To a mixture of 5-bromo-3-chloropicolinonitrile (2.0 g, 9.20 mmol) in THF (10 mL) under ice-water cooling was added BH₃·THF (1 M, 11.04 mL) over 5 min. The mixture was stirred at 0° C. for 30 min before it was warmed to 20° C. and stirred for another 30 min at this temperature. The mixture was cooled to 0° C. and quenched with dropwise addition of MeOH (10 mL) over 5 min. The mixture was heated to 70° C. and stirred for 30 min at this temperature. The reaction was concentrated under vacuum to give the crude product (2.2 g) as a light brown solid. The crude product was dissolved in HCl (aq. 2M, 20 mL), washed with DCM (20 mL; 2×) and the aqueous phase was finally concentrated under vacuum to give the product (5-bromo-3-chloro-2-pyridyl)methanamine (1.5 g, 4.07 mmol, 44.26% yield, 70% purity, HCl salt) as alight brown solid.

RT 0.18 min (method 2); m/z 222.9 (M+H)⁺ (ESI⁺), ¹H NMR (400 MHz, DMSO-d6) δ=8.78 (d, J=2.0 Hz, 1H), 8.69 (br, 3H), 8.47 (d, J=2.0 Hz, 1H), 4.24 (d, J=6.2 Hz, 2H).

### Preparation of Intermediate 2.2

### Ethyl 2-(((5-bromo-3-chloropyridin-2-yl)methyl)amino)-2-oxoacetate

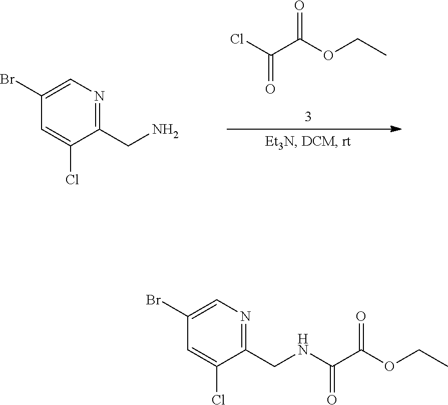

To a mixture of (5-bromo-3-chloro-2-pyridyl)methanamine (1.5 g, 5.82 mmol, HCl salt) in DCM (30 mL) under ice-water cooling was added DIPEA (2.25 g, 17.45 mmol). Then, ethyl 2-chloro-2-oxoacetate (952.77 mg, 6.98 mmol) was added over 5 min and the mixture was stirred at 0° C. for 30 min. The mixture was warmed to 20° C. and stirred for 30 min at this temperature. The mixture was quenched with aqueous NaHCO₃ solution (50 mL) and extracted with DCM (50 mL). The organic phase was separated, dried over Na₂SO₄, filtered and concentrated under vacuum. The residue was purified by column chromatography on silica gel (PE:EtOAc=10:1 to 1:1) to give the product ethyl 2-(((5-bromo-3-chloropyridin-2-yl)methyl)amino)-2-oxoacetate (1300 mg, 3.64 mmol, 62.57% yield, 65.6% purity) as a white solid. RT 0.61 min (method 1); m/z 322.8 (M+H)⁺ (ESI⁺). The product was used without further purification in the next step.

### Preparation of Intermediate 2.3

### Ethyl 6-bromo-8-chloroimidazo[1,5-a]pyridine-3-carboxylate

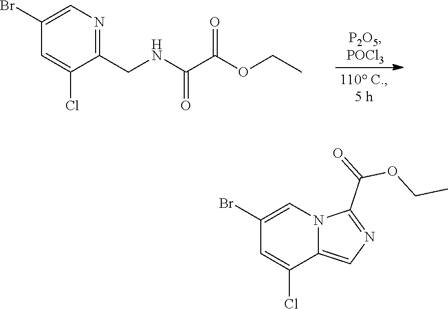 [SMILES: CCOC(=O)C(=O)NCc1ncc(Br)cc1Cl ; CCOC(=O)c1ncc2c(Cl)cc(Br)cn12]

To a mixture of ethyl ethyl 2-(((5-bromo-3-chloropyridin-2-yl)methyl)amino)-2-oxoacetate (1300 mg, 4.04 mmol) in POCl₃ (15 mL) under ice water cooling was added phosphorus pentoxide (2.87 g, 20.21 mmol). The mixture was heated to 110° C. and stirred for 5 h at this temperature. The mixture was cooled to 25° C. and concentrated under vacuum to give a residue. The residue was dissolved in EtOAc (50 mL) and washed with water (30 mL) and an aqueous NaHCO₃ solution (30 mL). Then it was was finally concentrated under vacuum to give a residue. The residue was purified by column chromatography on silica gel (PE:EtOAc=10:1 to 3:1) to give the product ethyl 6-bromo-8-chloroimidazo[1,5-a]pyridine-3-carboxylate (900 mg, 2.97 mmol, 73.34% yield) as a white solid.

RT 0.718 min (method 1), m/z 304.8 (M+H)⁺ (ESI⁺), ¹H NMR (400 MHz, CHLOROFORM-d) δ=9.47 (s, 1H), 7.77 (s, 1H), 7.20 (s, 1H), 4.65-4.42 (m, 2H), 1.57-1.42 (m, 3H)

### Preparation of Intermediate 2.4

### 6-bromo-8-chloroimidazo[1,5-a]pyridine-3-carbohydrazide

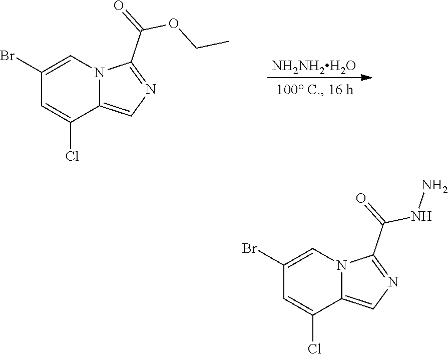

To a mixture of ethyl 6-bromo-8-chloroimidazo[1,5-a]pyridine-3-carboxylate (900 mg, 2.97 mmol) in EtOH (20 mL) was added NH₂NH₂·H₂O (1.48 g, 29.65 mmol, 98%). The mixture was heated to 80° C. and stirred for 2 h at this temperature. The reaction was cooled to 25° C. and the precipitated solid was separated off. The crude product was triturated with EtOH (5 mL) to give 6-bromo-8-chloroimidazo[1,5-a]pyridine-3-carbohydrazide (650 mg, 2.25 mmol, 75.72% yield) as a white solid.

RT 0.56 min (method 1); m/z 290.8 (M+H)⁺ (ESI⁺); ¹H NMR (400 MHz, DMSO-d₆) δ=10.02 (s, 1H), 9.50 (s, 1H), 7.72 (s, 1H), 7.51 (s, 1H), 4.58 (d, J=4.0 Hz, 2H).

### Preparation of Intermediate 4.5

### 6-bromo-8-chloro-N′-(2,2-difluoroacetyl)imidazo[1,5-a]pyridine-3-carbohydrazide

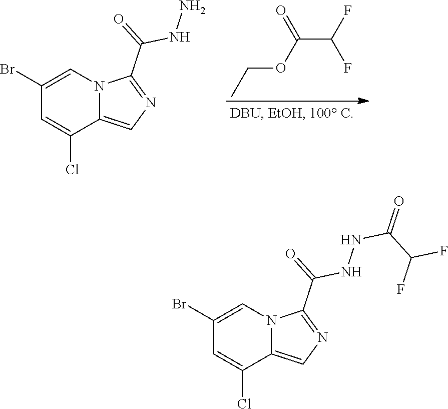

To a mixture of 6-bromo-8-chloroimidazo[1,5-a]pyridine-3-carbohydrazide (650 mg, 2.25 mmol) in EtOH (20 mL) was added ethyl 2,2-difluoroacetate (3.10 g, 22.45 mmol) and DBU (683.58 mg, 4.49 mmol). The mixture was heated to 100° C. stirred for 16 h at this temperature. The mixture was cooled to 25° C. and concentrated under vacuum. The residue was dissolved with DCM (50 mL), washed with an aqueous NH₄Cl solution (30 mL; 2×) and concentrated under vacuum to give the crude product. The crude product was purified by column chromatography on silica gel (PE/EtOAc=1:1 to MeOH:EtOAc=1:10) to give the product 6-bromo-8-chloro-N′-(2,2-difluoroacetyl)imidazo[1,5-a]pyridine-3-carbohydrazide (650 mg, 1.56 mmol, 69.32% yield, 88% purity) as a white solid.

RT 0.62 min (method 1); m/z 368.8 (M+H)⁺ (ESI⁺); ¹H NMR (400 MHz, DMSO-d₆) δ=10.95 (br, 2H), 9.44 (s, 1H), 7.81 (s, 1H), 7.59 (s, 1H), 6.38 (t, J=53.2, 1H).

### Preparation of Intermediate 2.6

### 2-(6-bromo-8-chloroimidazo[1,5-a]pyridin-3-yl)-5-(difluoromethyl)-1,3,4-thiadiazole

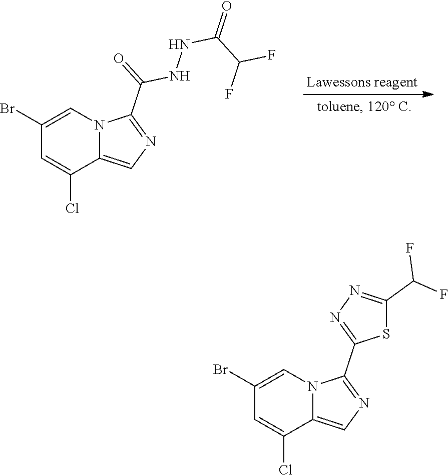 [SMILES: O=C(NNC(=O)C(F)F)c1ncc2c(Cl)cc(Br)cn12 ; FC(F)c1nnc(-c2ncc3c(Cl)cc(Br)cn23)s1]

To a mixture of 6-bromo-8-chloro-N′-(2,2-difluoroacetyl)imidazo[1,5-a]pyridine-3-carbohydrazide (550 mg, 1.50 mmol) in toluene (20 mL) was added Lawessons reagent (665.80 mg, 1.65 mmol) under a N₂ atmosphere. The reaction was heated to 120° C. and stirred for 2 h at this temperature. The reaction was cooled to 25° C. and concentrated under vacuum. The residue was triturated with MeOH (10 mL) at 70° C. for 1 h, filtered and the cake was collected, and dried under vacuum to give the product 2-(6-bromo-8-chloroimidazo[1,5-a]pyridin-3-yl)-5-(difluoromethyl)-1,3,4-thiadiazole (530 mg, 1.45 mmol, 96.88% yield) as a light yellow solid.

RT 0.806 min (method 1); m/z 366.8 (M+H)⁺ (ESI⁺); ¹H NMR (400 MHz, DMSO-d₆) δ=9.62 (s, 1H), 8.64 (s, 1H), 8.09 (s, 1H), 7.70 (t, J=53.2, 1H).

### Preparation of Intermediate 2.7

### 2-(6-(benzylthio)-8-chloroimidazo[1,5-a]pyridin-3-yl)-5-(difluoromethyl)-1,3,4-thiadiazole

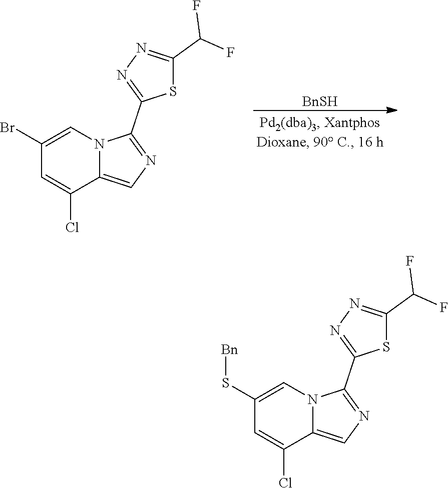

To a mixture of 2-(6-bromo-8-chloroimidazo[1,5-a]pyridin-3-yl)-5-(difluoromethyl)-1,3,4-thiadiazole (450 mg, 1.23 mmol) and phenylmethanethiol (168.17 mg, 1.35 mmol) in dioxane (10 mL) which was degassed with nitrogen for 2 min was added Pd₂(dba)₃ (112.72 mg, 123.09 μmol), Xantphos (71.22 mg, 123.09 μmol) and DIEA (477.26 mg, 3.69 mmol) under nitrogen. The mixture was heated to 90° C. and stirred for 16 h at this temperature. The mixture was filtered and concentrated under vacuum. The residue was purified by column chromatography on silica gel (PE:EtOAc=20:1 to 5:1) to give the product 2-(6-(benzylthio)-8-chloroimidazo[1,5-a]pyridin-3-yl)-5-(difluoromethyl)-1,3,4-thiadiazole (250 mg, 489.15 μmol, 39.74% yield, 80% purity) as a light yellow solid.

RT 0.99 min (method 1); m/z 409.0 (M+H)⁺ (ESI⁺); ¹H NMR (400 MHz, CHLOROFORM-d) δ=9.35 (s, 1H), 7.69-7.67 (m, 1H), 7.39-7.28 (m, 2H), 7.25-7.12 (m, 3H), 7.05 (t, J=53.2, 1H), 7.00 (s, 1H), 6.90 (s, 1H), 4.10 (s, 2H)

### Preparation of Intermediate 2.8

### 2-(6-(benzylthio)-8-chloro-1-iodoimidazo[1,5-a]pyridin-3-yl)-5-(difluoromethyl)-1,3,4-thiadiazole

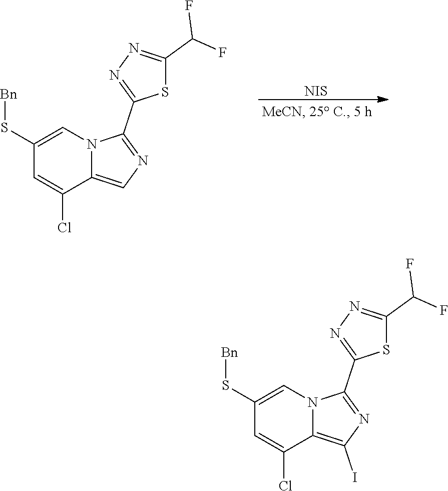 [SMILES: FC(F)c1nnc(-c2ncc3c(Cl)cc(SCc4ccccc4)cn23)s1 ; "NIS" ; FC(F)c1nnc(-c2nc(I)c3c(Cl)cc(SCc4ccccc4)cn23)s1]

To a mixture of 2-(6-(benzylthio)-8-chloroimidazo[1,5-a]pyridin-3-yl)-5-(difluoromethyl)-1,3,4-thiadiazole (130 mg, 317.95 μmol) in MeCN (5 mL) at 0° C. was added NIS (78.68 mg, 349.74 μmol). The mixture was stirred at 25° C. for 5 h. The reaction mixture was used for the next step directly.

RT 0.99 min (method 1); m/z 535.0 (M+H)⁺ (ESI⁺)

### Preparation of Intermediate 2.9

### 8-chloro-3-(5-(difluoromethyl)-1,3,4-thiadiazol-2-yl)-1-iodoimidazo[1,5-a]pyridine-6-sulfonyl chloride

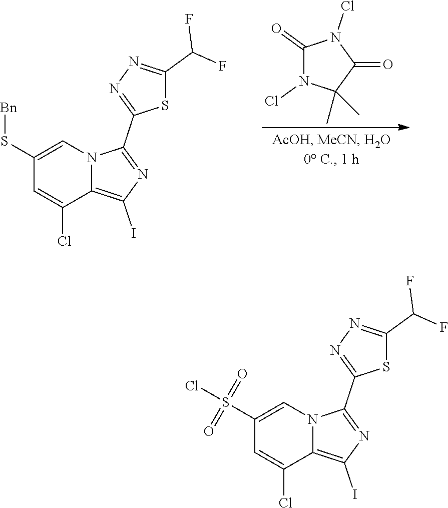 [SMILES: FC(F)c1nnc(-c2nc(I)c3c(Cl)cc(SCc4ccccc4)cn23)s1 ; CC1(C)C(=O)N(Cl)C(=O)N1Cl ; O=S(=O)(Cl)c1cc(Cl)c2c(I)nc(-c3nnc(C(F)F)s3)n2c1]

A mixture of 2-(6-(benzylthio)-8-chloro-1-iodoimidazo[1,5-a]pyridin-3-yl)-5-(difluoromethyl)-1,3,4-thiadiazole (170 mg, 317.95 μmol) in MeCN (5 mL) was cooled to 0° C. before H₂O (5.73 mg, 317.95 μmol), AcOH (38.19 mg, 635.89 μmol) and 1,3-dichloro-5,5-dimethylimidazolidine-2,4-dione (125.28 mg, 635.89 μmol) was added. The mixture was stirred at 0° C. for 2 h. The mixture was diluted with THF (8 mL), dried over Na₂SO₄, filtered and concentrated under vacuum to give the crude product 8-chloro-3-(5-(difluoromethyl)-1,3,4-thiadiazol-2-yl)-1-iodoimidazo[1,5-a]pyridine-6-sulfonyl chloride (160 mg, 219.14 μmol, 68.92% yield, 70% purity) as a light brown oil.

It is noted that it cannot be excluded that the dichloro-compound 1,8-dichloro-3-(5-(difluoromethyl)-1,3,4-thiadiazol-2-yl)imidazo[1,5-a]pyridine-6-sulfonyl chloride was also formed in this process.

### Preparation of Intermediate 2.10

### 8-chloro-3-(5-(difluoromethyl)-1,3,4-thiadiazol-2-yl)-1-iodo-N-(1-methylcyclopropyl)imidazo[1,5-a]pyridine-6-sulfonamide

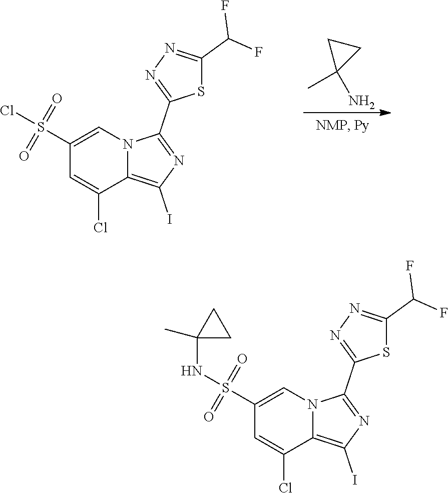

To a mixture of 1-methylcyclopropan-1-amine (37.80 mg, 531.49 μmol) in pyridine (1 mL) and NMP (N-methyl-2-pyrrolidon) (1 mL) at 0° C. was added 8-chloro-3-(5-(difluoromethyl)-1,3,4-thiadiazol-2-yl)-1-iodoimidazo[1,5-a]pyridine-6-sulfonyl chloride (90 mg, 176.09 μmol) in MeCN (2 mL). The reaction was stirred at 0° C. for 50 min. The reaction mixture was quenched with water (10 mL) and extracted with EtOAc (10 mL; 2×). The organic phase was collected, dried over Na₂SO₄, filtered and concentrated under vacuum to give a residue which was purified by preparative TLC (PE:EtOAc=3:1) to give the product 8-chloro-3-(5-(difluoromethyl)-1,3,4-thiadiazol-2-yl)-1-iodo-N-(1-methylcyclopropyl)imidazo[1,5-a]pyridine-6-sulfonamide (25 mg, 45.81 μmol, 26.01% yield) as a light yellow solid.

It is noted that it cannot be excluded that be excluded that the dichloro compound 1,8-dichloro-3-(5-(difluoromethyl)-1,3,4-thiadiazol-2-yl)-N-(1-methylcyclopropyl)imidazo[1,5-a]pyridine-6-sulfonamide was also formed in this process.

RT 0.510 min (method 3); m/z 545.8 (M+H)⁺ (ESI⁺)

### Preparation of Intermediate 2.11

### 8-chloro-3-(5-(difluoromethyl)-1,3,4-thiadiazol-2-yl)-N-(1-methylcyclopropyl)imidazo[1,5-a]pyridine-6-sulfonamide

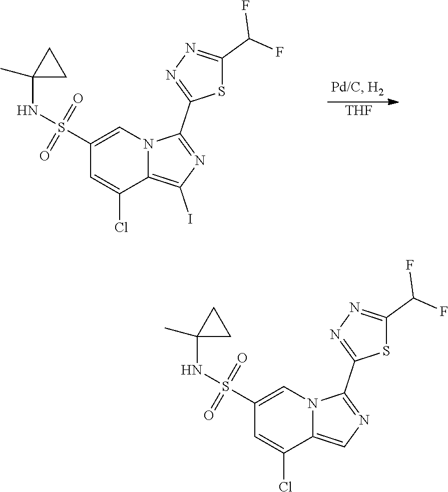 [SMILES: CC1(NS(=O)(=O)c2cc(Cl)c3c(I)nc(-c4nnc(C(F)F)s4)n3c2)CC1 ; CC1(NS(=O)(=O)c2cc(Cl)c3cnc(-c4nnc(C(F)F)s4)n3c2)CC1 ; "THF"]

To a mixture of 8-chloro-3-(5-(difluoromethyl)-1,3,4-thiadiazol-2-yl)-1-iodo-N-(1-methylcyclopropyl)imidazo[1,5-a]pyridine-6-sulfonamide (25 mg, 45.81 μmol) in tetrahydrofuran (3 mL) was added Pd/C (5 mg, 10% purity). The reaction was degassed with H₂ (15 Psi) three times, then the reaction was stirred at 20° C. for 3 h. The reaction mixture was filtered and the filtrate was concentrated under vacuum to give the product 8-chloro-3-(5-(difluoromethyl)-1,3,4-thiadiazol-2-yl)-N-(1-methylcyclopropyl)imidazo[1,5-a]pyridine-6-sulfonamide (20 mg, 30.96 μmol, 67.59% yield, 65% purity) as a brown solid.

It is noted that it cannot be excluded that the dichloro compound 1,8-dichloro-3-(5-(difluoromethyl)-1,3,4-thiadiazol-2-yl)-N-(1-methylcyclopropyl)imidazo[1,5-a]pyridine-6-sulfonamide was also formed in this process.

RT 0.468 min (method 3); m/z 420.0 (M+H)⁺ (ESI⁺)

### Preparation of Intermediate 2.12

### tert-butyl 4-(6-(N-(tert-butoxycarbonyl)-N-(1-methylcyclopropyl)sulfamoyl)-3-(5-(difluoromethyl)-1,3,4-thiadiazol-2-yl)imidazo[1,5-a]pyridin-8-yl)-3,3-difluoro-3,6-dihydropyridine-1(2H)-carboxylate

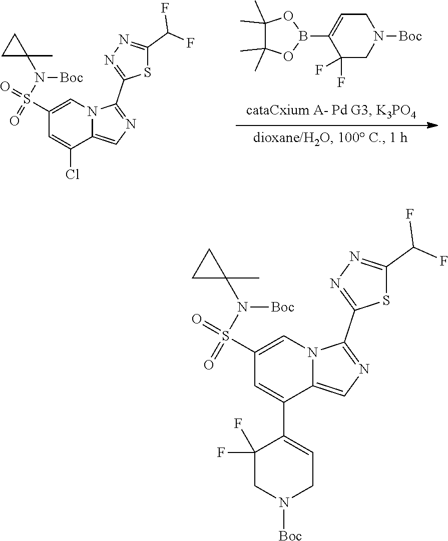 [SMILES: CC(C)(C)OC(=O)N(C1(C)CC1)S(=O)(=O)c1cc(Cl)c2cnc(-c3nnc(C(F)F)s3)n2c1 ; CC(C)(C)OC(=O)N1CC=C(B2OC(C)(C)C(C)(C)O2)C(F)(F)C1 ; CC(C)(C)OC(=O)N1CC=C(c2cc(S(=O)(=O)N(C(=O)OC(C)(C)C)C3(C)CC3)cn3c(-c4nnc(C(F)F)s4)ncc23)C(F)(F)C1]

To a solution of tert-butyl ((8-chloro-3-(5-(difluoromethyl)-1,3,4-thiadiazol-2-yl)imidazo[1,5-a]pyridin-6-yl)sulfonyl)(1-methylcyclopropyl)carbamate (100 mg, 0.192 mmol) and tert-butyl 3,3-difluoro-4-(4,4,5,5-tetramethyl-1,3,2-dioxaborolan-2-yl)-3,6-dihydropyridine-1(2H)-carboxylate (133 mg, 0.385 mmol) in dioxane (1 mL) and water (0.2 mL) were added K₃PO₄ (82 mg, 0.385 mmol) and cataCxium A-Pd G3 (14 mg, 0.0192 mmol). The reaction mixture was degassed with N₂ (3×) and stirred at 100° C. for 1 hr under nitrogen atmosphere. The resulting mixture was cooled to 25° C. and filtered. The filtrate was concentrated under vacuum to give a residue, which was purified by preparative TLC (petroleum ether:ethyl acetate=3:1) to give the product tert-butyl 4-(6-(N-(tert-butoxycarbonyl)-N-(1-methylcyclopropyl)sulfamoyl)-3-(5-(difluoromethyl)-1,3,4-thiadiazol-2-yl)imidazo[1,5-a]pyridin-8-yl)-3,3-difluoro-3,6-dihydropyridine-1(2H)-carboxylate (90 mg, 0.0876 mmol, 45.54% yield, 68.4 purity) as a yellow solid.

RT 0.564 min (method 4), m/z 703.4 (M+H)⁺ (ESI⁺); ¹H NMR (CDCl₃, 400 MHz): 10.24 (s, 1H), 7.80 (s, 1H), 7.42 (s, 1H), 7.09 (t, J=53.6 Hz, 1H), 6.69-6.48 (m, 1H), 4.37-4.27 (m, 2H), 4.03 (t, J=10.0 Hz, 2H), 1.62 (s, 3H), 1.55 (s, 9H), 1.40 (s, 9H), 1.17-1.14 (m, 2H), 1.04-0.99 (m, 2H).

### Preparation of Intermediate 2.13

### tert-butyl 4-(6-(N-(tert-butoxycarbonyl)-N-(1-methylcyclopropyl)sulfamoyl)-1-chloro-3-(5-(difluoromethyl)-1,3,4-thiadiazol-2-yl)imidazo[1,5-a]pyridin-8-yl)-3,3-difluoro-3,6-dihydropyridine-1(2H)-carboxylate

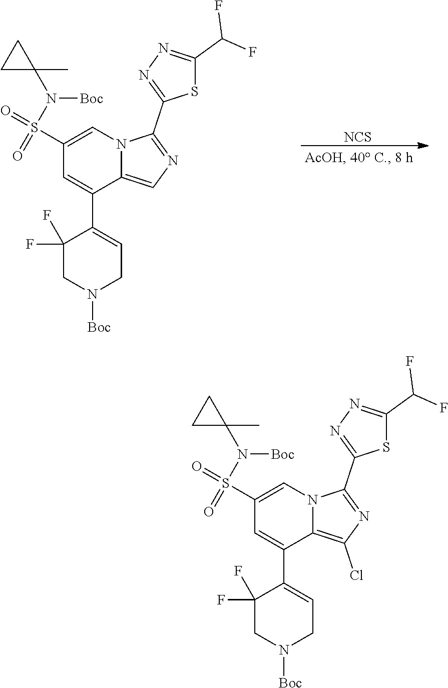 [SMILES: CC(C)(C)OC(=O)N1CC=C(c2cc(S(=O)(=O)N(C(=O)OC(C)(C)C)C3(C)CC3)cn3c(-c4nnc(C(F)F)s4)ncc23)C(F)(F)C1 ; "NCS" ; CC(C)(C)OC(=O)N1CC=C(c2cc(S(=O)(=O)N(C(=O)OC(C)(C)C)C3(C)CC3)cn3c(-c4nnc(C(F)F)s4)nc(Cl)c23)C(F)(F)C1]

To a solution of tert-butyl 4-(6-(N-(tert-butoxycarbonyl)-N-(1-methylcyclopropyl)sulfamoyl)-3-(5-(difluoromethyl)-1,3,4-thiadiazol-2-yl)imidazo[1,5-a]pyridin-8-yl)-3,3-difluoro-3,6-dihydropyridine-1(2H)-carboxylate (60 mg, 0.0584 mmol) in acetic acid (1 mL) was added NCS (23 mg, 0.175 mmol). The mixture was stirred at 40° C. for 8 hr. The resulting mixture was diluted with water (20 mL) and extracted with DCM (20 mL, 3×). The combined organic phase was dried over anhydrous Na₂SO₄, filtered and concentrated under vacuum. The resulting residue was purified by preparative TLC (petroleum ether:ethyl acetate=3:1) to give the product tert-butyl 4-(6-(N-(tert-butoxycarbonyl)-N-(1-methylcyclopropyl)sulfamoyl)-1-chloro-3-(5-(difluoromethyl)-1,3,4-thiadiazol-2-yl)imidazo[1,5-a]pyridin-8-yl)-3,3-difluoro-3,6-dihydropyridine-1(2H)-carboxylate (25 mg, 0.0308 mmol, 52.82% yield, 91% purity) as a yellow solid.

RT 0.598 min (method 4), m/z 737.3 (M+H)⁺ (ESI⁺); ¹H NMR (CDCl₃, 400 MHz): 10.25 (s, 1H), 7.34 (s, 1H), 7.08 (t, J=53.2 Hz, 1H), 6.45-6.21 (m, 1H), 4.37-4.25 (m, 2H), 4.10-3.95 (m, 2H), 1.61 (s, 3H), 1.54 (s, 9H), 1.41 (s, 9H), 1.22-1.09 (m, 2H), 1.04-0.97 (m, 2H).

### Preparation of Example 2

### 1-chloro-8-(3,3-difluoro-1,2,3,6-tetrahydropyridin-4-yl)-3-(5-(difluoromethyl)-1,3,4-thiadiazol-2-yl)-N-(1-methylcyclopropyl)imidazo[1,5-a]pyridine-6-sulfonamide

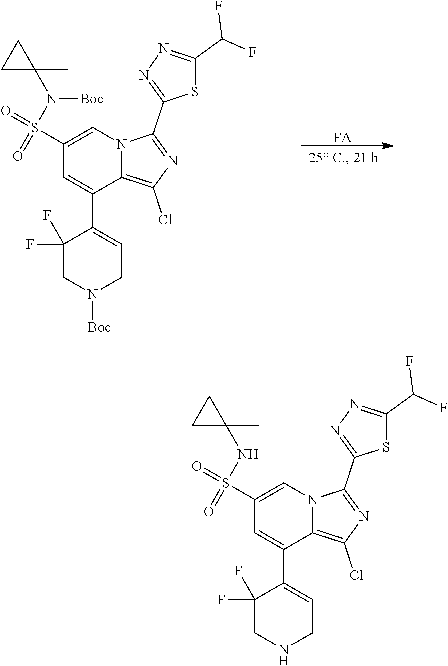 [SMILES: CC(C)(C)OC(=O)N1CC=C(c2cc(S(=O)(=O)N(C(=O)OC(C)(C)C)C3(C)CC3)cn3c(-c4nnc(C(F)F)s4)nc(Cl)c23)C(F)(F)C1 ; "FA" ; CC1(NS(=O)(=O)c2cc(C3=CCNCC3(F)F)c3c(Cl)nc(-c4nnc(C(F)F)s4)n3c2)CC1]

A solution of tert-butyl 4-(6-(N-(tert-butoxycarbonyl)-N-(1-methylcyclopropyl)sulfamoyl)-1-chloro-3-(5-(difluoromethyl)-1,3,4-thiadiazol-2-yl)imidazo[1,5-a]pyridin-8-yl)-3,3-difluoro-3,6-dihydropyridine-1(2H)-carboxylate (25 mg, 0.0308 mmol) in formic acid (1 mL) was stirred at 25° C. for 21 hr and then, concentrated under vacuum. The resulting residue was purified by preparative HPLC (column: Phenomenex luna C18 150*25 mm*10 μm; mobile phase: A: 0.225% formic acid in water, B: MeCN; B %: 20%-50%, 10 min) and lyophilized directly to give the product 1-chloro-8-(3,3-difluoro-1,2,3,6-tetrahydropyridin-4-yl)-3-(5-(difluoromethyl)-1,3,4-thiadiazol-2-yl)-N-(1-methylcyclopropyl)imidazo[1,5-a]pyridine-6-sulfonamide (5.16 mg, 0.0211 mmol, 30.84% yield, 99% purity) as a yellow solid.

RT 0.381 min (method 4); m/z 537.1 (M+H)⁺ (ESI⁺); ¹H NMR (DMSO-d₆, 400 MHz): 9.90 (s, 1H), 8.68 (br, 1H), 7.70 (t, J=52.8 Hz, 1H), 7.22 (s, 1H), 6.56 (s, 1H), 3.52-3.48 (m, 2H), 3.28 (t, J=11.2 Hz, 2H), 1.19 (s, 3H), 0.79-0.65 (m, 2H), 0.55-0.40 (m, 2H).

### Preparation of Intermediate 3.1

### (2S,2'S)-3,3′-(benzylazanediyl)bis(1-methoxypropan-2-ol)

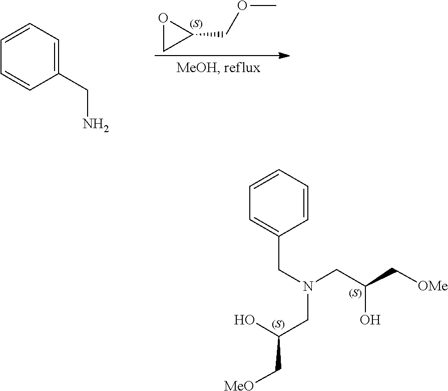

To a mixture of phenylmethanamine (5.00 g, 46.7 mmol) in methanol (50 mL) was added (S)-2-(methoxymethyl)oxirane (12 mL, 140 mmol). The reaction mixture was stirred at 80° C. for 16 h and, then concentrated under vacuum. The residue was dissolved with DCM (8 mL) purified by flash silica gel chromatography (ISCO; 120 g SepaFlash Silica Flash Column, Eluent of 60-68% Ethyl acetate/Petroleum ether, gradient @100 mL/min) to give the product (2S,2'S)-3,3′-(benzylazanediyl)bis(1-methoxypropan-2-ol) (10.00 g, 34.8 mmol, 74.68% yield) as yellow oil.

RT 0.228 min (method 4); m/z 284 (M+H)⁺ (ESI⁺); ¹H NMR (DMSO-d₆, 400 MHz) 7.13-7.37 (m, 5H), 4.59 (d, J=4.4 Hz, 2H), 3.55-3.76 (m, 4H), 3.25-3.31 (m, 2H), 3.11-3.24 (m, 8H), 2.43-2.49 (m, 2H), 2.32-2.42 (m, 2H)

### Preparation of Intermediate 3.2

### (2S,2'S)-(benzylazanediyl)bis(3-methoxypropane-1,2-diyl) diethanesulfonate

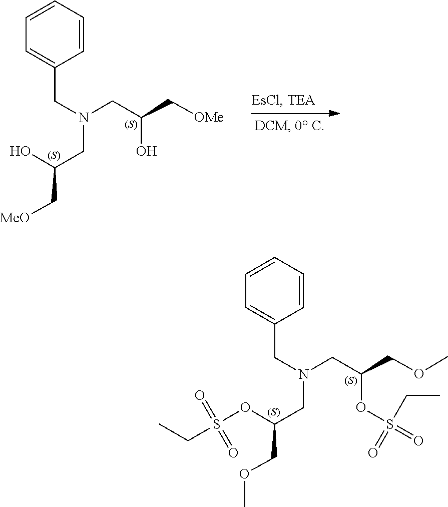

To a solution of (2S,2'S)-3,3′-(benzylazanediyl)bis(1-methoxypropan-2-ol) (2.00 g, 7.06 mmol) in DCM (20 mL) was added TEA (5.3 mL, 21.2 mmol) and ethanesulfonyl chloride (2.72 g, 21.2 mmol) under N₂ at 0° C. The mixture was stirred at 0° C. for 40 min and, then concentrated under vacuum at 25° C. to give the product (2S,2'S)-(benzylazanediyl)bis(3-methoxypropane-1,2-diyl) diethanesulfonate (3.50 g, 5.61 mmol, 79.54% yield) as yellow oil, which was used in the next step without further purification.

RT 0.493 min (method 4); m/z 468.2 (M+H)⁺ (ESI⁺)

### Preparation of Intermediate 3.3

### (2R,6R)-1,4-dibenzyl-2,6-bis(methoxymethyl)piperazine

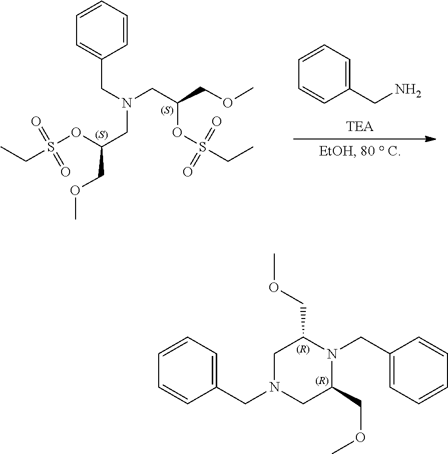

To a solution of (2S,2'S)-(benzylazanediyl)bis(3-methoxypropane-1,2-diyl) diethanesulfonate (3.50 g, 7.49 mmol) in ethanol (50 mL) were added TEA (4.1 mL, 29.9 mmol) and phenylmethanamine (1.20 g, 11.2 mmol). The mixture was heated to 80° C. for 2 h and, then was concentrated under vacuum. The resulting residue was purified by silica gel flash chromatography (ISCO; 120 g SepaFlash Silica Flash Column, Eluent Ethyl acetate/Petroleum ether, gradient from 0 to 26% of Ethyl acetate @80 mL/min) to give the product (2R,6R)-1,4-dibenzyl-2,6-bis(methoxymethyl)piperazine (650 mg, 1.76 mmol, 23.52% yield) as yellow oil.

RT 0.854 min (method 5); m/z 355 (M+H)⁺ (ESI⁺); ¹H NMR (DMSO-d₆, 400 MHz) 7.16-7.35 (m, 10H), 3.64-3.96 (m, 2H), 3.36-3.61 (m, 6H), 3.14 (d, J=7.6 Hz, 6H), 2.81-2.86 (m, 1H), 2.58-2.63 (m, 1H), 2.51-2.55 (m, 1H), 2.24-2.43 (m, 3H)

### Preparation of Intermediate 3.4

### (2R,6R)-2,6-bis(methoxymethyl)piperazine

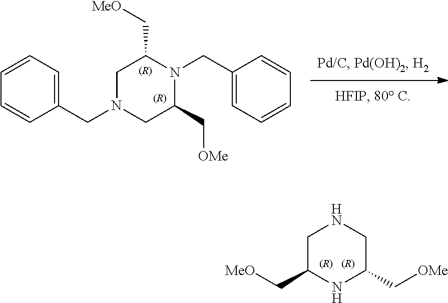

To a mixture of Pd(OH)₂ (250 mg, 0.356 mmol) and Pd/C (250 mg, 0.235 mmol) in 1,1,1,3,3,3-hexafluoropropan-2-ol (8.3 mL, 79.1 mmol) was added (2R,6R)-1,4-dibenzyl-2,6-bis(methoxymethyl)piperazine (500 mg, 1.41 mmol). The reaction mixture was degassed with H₂ (3×), then the heated to 80° C. and stirred for 16 h. The resulting mixture was filtered. The cake was washed with MeOH (12 mL) and the filtrate was concentrated under vacuum to give the product (2R,6R)-2,6-bis(methoxymethyl)piperazine (300 mg, 1.38 mmol, 97.66% yield) as colorless oil, which was used in the next step without further purification.

¹H NMR (DMSO-d₆, 400 MHz) 3.36-3.26 (m, 4H), 3.24 (s, 6H), 2.93-2.85 (m, 1H), 2.72-2.67 (m, 2H), 2.64-2.55 (m, 2H), 2.41-2.36 (m, 1H)

### Preparation of Example 3

### 8-((3R,5R)-3,5-bis(methoxymethyl)piperazin-1-yl)-3-(5-(difluoromethyl)-1,3,4-thiadiazol-2-yl)-N-(1-methylcyclopropyl)imidazo[1,5-a]pyridine-6-sulfonamide formate

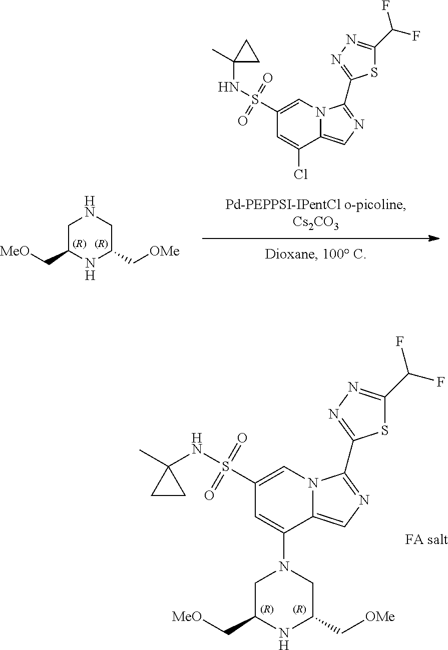

To a mixture of (2R,6R)-2,6-bis(methoxymethyl)piperazine (104 mg, 0.595 mmol) in 1,4-dioxane (1.5 mL) were added Cs₂CO₃ (97 mg, 0.298 mmol), 8-chloro-3-(5-(difluoromethyl)-1,3,4-thiadiazol-2-yl)-N-(1-methylcyclopropyl)imidazo[1,5-a]pyridine-6-sulfonamide (50 mg, 0.119 mmol) and Pd-PEPPSI-IPentCl o-picoline (41 mg, 0.0476 mmol). The reaction mixture was degassed, and purged with N₂ 3 times. Then, the mixture was stirred at 100° C. for 1 h and filtered. The filtrate was concentrated under vacuum to give residue which was purified by preparative HPLC (column: Phenomenex luna C18 150*25 mm*10 μm;mobile phase: A: 0.225% formic acid in water, B: MeCN; B %: 15%-45%, 10 min) and lyophilized to give the product 8-((3R,5R)-3,5-bis(methoxymethyl)piperazin-1-yl)-3-(5-(difluoromethyl)-1,3,4-thiadiazol-2-yl)-N-(1-methylcyclopropyl)imidazo[1,5-a]pyridine-6-sulfonamide (7.4 mg, 0.0131 mmol, 11.03% yield, 99% purity, FA salt) as a yellow solid.

RT 0.358 min (method 4); m/z 558.2 (M+H)⁺ (ESI⁺); ¹H NMR (DMSO-d₆, 400 MHz) 9.58 (s, 1H), 8.46 (s, 1H), 8.20 (s, 1H), 7.92 (s, 1H), 7.68 (t, J=53.2 Hz, 1H), 6.68 (d, J=0.8 Hz, 1H), 3.58-3.66 (m, 2H), 3.40-3.43 (m, 2H), 3.34 (s, 6H), 3.23-3.28 (m, 4H), 3.12-3.18 (m, 2H), 1.15 (s, 3H), 0.65-0.78 (m, 2H), 0.39-0.51 (m, 2H)

### Preparation of Example 4

### (Cis or Trans)-3-(5-(difluoromethyl)-1,3,4-thiadiazol-2-yl)-8-(2-methyl-1-(methylimino)-1-oxido-116-thiomorpholino)-N-(1-methylcyclopropyl)imidazo[1,5-a]pyridine-6-sulfonamide and example 5 (Trans or Cis)-3-(5-(difluoromethyl)-1,3,4-thiadiazol-2-yl)-8-(2-methyl-1-(methylimino)-1-oxido-116-thiomorpholino)-N-(1-methylcyclopropyl)imidazo[1,5-a]pyridine-6-sulfonamide

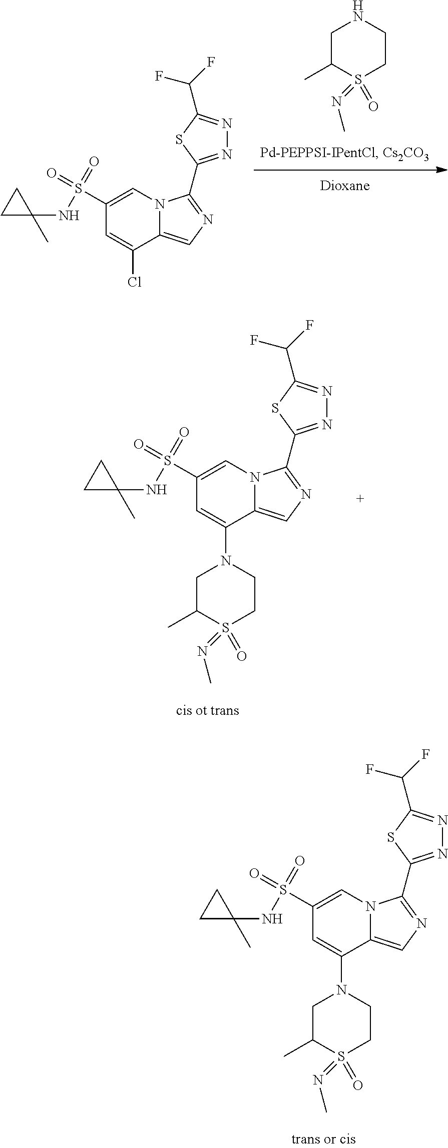

To a mixture of 8-chloro-3-(5-(difluoromethyl)-1,3,4-thiadiazol-2-yl)-N-(1-methylcyclopropyl)imidazo[1,5-a]pyridine-6-sulfonamide (30 mg, 0.0715 mmol) in 1,4-dioxane (0.5 mL) were added 2-methyl-1-(methylimino)-1λ⁶-thiomorpholine 1-oxide (57 mg, 0.286 mmol, HCl salt), Cs₂CO₃ (93 mg, 0.286 mmol) and Pd-PEPPSI-IPentCl o-picoline (3.7 mg, 0.0286 mmol). The reaction mixture was degassed with N₂ (3×), stirred at 100° C. for 16 h, then, cooled to room temperature and concentrated under vacuum. The resulting residue was purified by preparative HPLC (column: Waters Xbridge 150*25 mm*5 μm; mobile phase: A: 10 mM aqueous solution of NH₄HCO₃ in water, B: MeCN; B %: 27%-57%, 10 min) and lyophilized directly to give a crude product, which was further purified by preparative HPLC (column: Phenomenex luna C18 150*25 mm*10 μm; mobile phase: A: 0.225% formic acid in water, B: MeCN; B %: 32%-52%, 15 min) and lyophilized to give the following products.

Example 4 (Cis or Trans)-3-(5-(difluoromethyl)-1,3,4-thiadiazol-2-yl)-8-(2-methyl-1-(methylimino)-1-oxido-116-thiomorpholino)-N-(1-methylcyclopropyl)imidazo[1,5-a]pyridine-6-sulfonamide (1.2 mg, 2.20 μmol, 3.08% yield) as a yellow solid.

RT 0.347 min (method 4); m/z 546.3 (M+H)⁺ (ESI⁺); ¹H NMR (CDCl₃, 400 MHz) 9.91 (s, 1H), 7.68 (s, 1H), 7.09 (t, J=53.2 Hz, 1H), 6.74 (s, 1H), 5.21 (s, 1H), 3.99-3.87 (m, 1H), 3.84-3.69 (m, 2H), 3.59-3.37 (m, 3H), 3.35-3.24 (m, 1H), 2.93 (s, 3H), 1.53 (d, J=6.6 Hz, 3H), 1.40 (s, 3H), 0.97-0.89 (m, 2H), 0.66-0.59 (m, 2H).

Example 5: (Trans or Cis)-3-(5-(difluoromethyl)-1,3,4-thiadiazol-2-yl)-8-(2-methyl-1-(methylimino)-1-oxido-116-thiomorpholino)-N-(1-methylcyclopropyl)imidazo[1,5-a]pyridine-6-sulfonamide (1.2 mg, 2.20 μmol, 3.08% yield) as a yellow solid.

RT 0.358 min (method 4); m/z 546.2 (M+H)⁺ (ESI⁺); ¹H NMR (CDCl₃-d, 400 MHz) 9.92 (s, 1H), 7.68 (s, 1H), 7.09 (t, J=53.2 Hz, 1H), 6.73 (s, 1H), 5.11 (s, 1H), 3.96-3.79 (m, 2H), 3.68-3.46 (m, 3H), 3.45-3.24 (m, 2H), 2.90 (s, 3H), 1.51 (d, J=6.6 Hz, 3H), 1.40 (s, 3H), 0.96-0.90 (m, 2H), 0.65-0.60 (m, 2H)

### Preparation of Intermediate 13.1

### 8-chloro-3-(5-(difluoromethyl)-1,3,4-thiadiazol-2-yl)-1-fluoro-N-(1-methylcyclopropyl)imidazo[1,5-a]pyridine-6-sulfonamide

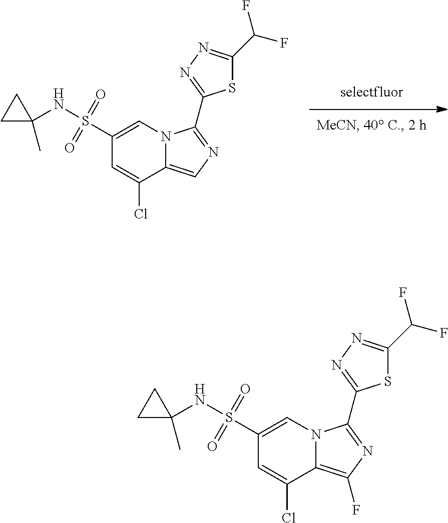 [SMILES: CC1(NS(=O)(=O)c2cc(Cl)c3cnc(-c4nnc(C(F)F)s4)n3c2)CC1 ; CC1(NS(=O)(=O)c2cc(Cl)c3c(F)nc(-c4nnc(C(F)F)s4)n3c2)CC1]

To a solution of 8-chloro-3-(5-(difluoromethyl)-1,3,4-thiadiazol-2-yl)-N-(1-methylcyclopropyl)imidazo[1,5-a]pyridine-6-sulfonamide (2 g, 4.76 mmol) in MeCN (40 mL) was added selectfluor (3 g, 8.47 mmol) at 0° C. The mixture was stirred at 40° C. for 2 h. Thirteen reactions were conducted in parallel on the same scale. The resulting mixtures were combined, cooled down to 20° C. and filtered. The filtrate was concentrated under vacuum. The resulting residue was purified by flash silica gel chromatography (ISCO; 330 g SepaFlash Silica Flash Column, Eluent of 0%-100% Ethyl acetate/Petroleum ether; gradient @200 mL/min) and concentrated under vacuum to give a crude product, which was further purified by flash silica gel chromatography (ISCO; 220 g SepaFlash Silica Flash Column, Eluent of 0%-20% Ethyl acetate/Petroleum ether; gradient @150 mL/min). The fractions containing the compound were mixed and concentrated under vacuum to give the product 8-chloro-3-(5-(difluoromethyl)-1,3,4-thiadiazol-2-yl)-1-fluoro-N-(1-methylcyclopropyl)imidazo[1,5-a]pyridine-6-sulfonamide (12 g, 27.4 mmol, 44.26% yield) as a yellow solid.

RT 0.580 min (method 6); m/z 438.0. (M+H)⁺ (ESI⁺). ¹H NMR (DMSO-d₆, 400 MHz): 9.71 (s, 1H), 8.63 (s, 1H), 7.70 (t, J=53.2 Hz, 1H), 7.40 (s, 1H), 1.22 (s, 3H), 0.76-0.74 (m, 2H), 0.51-0.48 (m, 2H).

### Preparation of Intermediate 13.2

### tert-butyl (2S,6S)-4-(3-(5-(difluoromethyl)-1,3,4-thiadiazol-2-yl)-1-fluoro-6-(N-(1-methylcyclopropyl)sulfamoyl)imidazo[1,5-a]pyridin-8-yl)-2,6-dimethylpiperazine-1-carboxylate

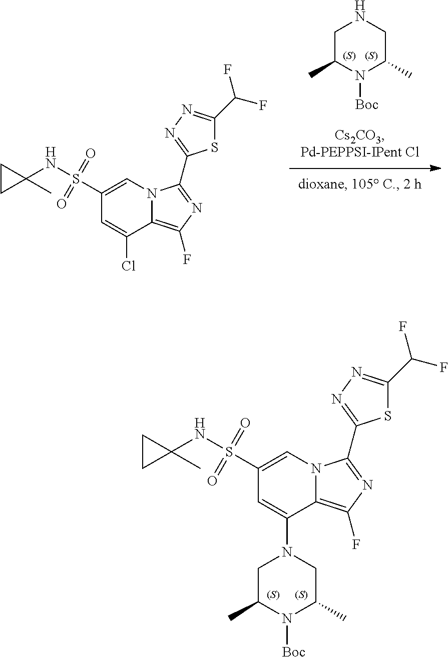

To a mixture of 8-chloro-3-(5-(difluoromethyl)-1,3,4-thiadiazol-2-yl)-1-fluoro-N-(1-methylcyclopropyl)imidazo[1,5-a]pyridine-6-sulfonamide (5 g, 11.42 mmol) in dioxane (50 mL) were added tert-butyl (2S,6S)-2,6-dimethylpiperazine-1-carboxylate (4.89 g, 22.84 mmol), Pd-PEPPSI-IPentCl o-picoline (2-methylpyridine) (0.96 g, 1.14 mmol) and Cs₂CO₃ (3.95 g, 28.6 mmol). The reaction mixture was degassed with N₂ (3×) and stirred at 105° C. for 2 h. In parallel, two reactions were conducted with the same protocol but on different scales of 8-chloro-3-(5-(difluoromethyl)-1,3,4-thiadiazol-2-yl)-1-fluoro-N-(1-methylcyclopropyl)imidazo[1,5-a]pyridine-6-sulfonamide (5 g, 2 g). The combined resulting mixture was cooled down to 20° C. and filtered. The filtrate was diluted with water (400 mL) and extracted with ethyl acetate (400 mL, 2×). The combined organic layer was washed with brine (400 mL, 2×), dried over anhydrous Na₂SO₄, filtered and concentrated under vacuum. The residue was purified by flash silica gel chromatography (ISCO; 220 g SepaFlash Silica Flash Column, Eluent of 0-30% Ethyl acetate/DCM; gradient @200 mL/min) and concentrated under vacuum. The resulting crude product was further purified by Flash silica gel chromatography (ISCO; 330 g SepaFlash Silica Flash Column, Eluent of 0%-25% Ethyl acetate/Petroleum ether; gradient @200 mL/min) and concentrated under vacuum to give an intermediate product (8.50 g, yellow solid) which was dissolved in toluene (60 mL). Sulfhydryl modified silica gel (5.6 g) was then added. The resulting mixture was stirred at 60° C. for 16 h, then cooled down to 20° C. and filtered. The filtrate was concentrated under vacuum to give a crude product (7.8 g) which was dissolved in DMF (100 mL), purified by preparative HPLC (column: Welch Ultimate XB_C18 20-40 μm; mobile phase: A: water, B: MeCN; B %: 0%-100%, 30 min) and lyophilized directly to give the product tert-butyl-(2S,6S)-4-(3-(5-(difluoromethyl)-1,3,4-thiadiazol-2-yl)-1-fluoro-6-(N-(1-methylcyclopropyl)sulfamoyl)imidazo[1,5-a]pyridin-8-yl)-2,6-dimethylpiperazine-1-carboxylate (5.6 g, 9.10 mmol, 33.2% yield) as a yellow solid.

LCMS: RT 0.642 min (method 6); m/z 616.2 (M+H)⁺ (ESI⁺).

### Preparation of Example 13

### 3-(5-(difluoromethyl)-1,3,4-thiadiazol-2-yl)-8-((3S,5S)-3,5-dimethylpiperazin-1-yl)-1-fluoro-N-(1-methylcyclopropyl)imidazo[1,5-a]pyridine-6-sulfonamide

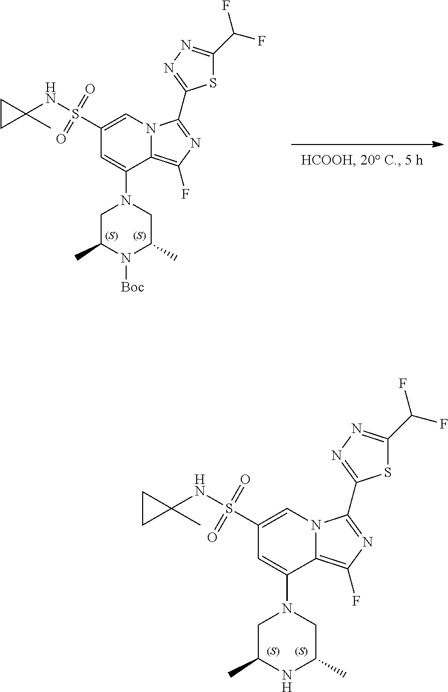

A solution of tert-butyl (2S,6S)-4-(3-(5-(difluoromethyl)-1,3,4-thiadiazol-2-yl)-1-fluoro-6-(N-(1-methylcyclopropyl)sulfamoyl)imidazo[1,5-a]pyridin-8-yl)-2,6-dimethylpiperazine-1-carboxylate (5.6 g, 9.10 mmol) in formic acid (56 mL) was stirred at 50° C. for 1 h. The resulting mixture was concentrated under vacuum directly. The residue was diluted with water (30 mL), basified with NaHCO₃ (aq., sat., 10 mL) until pH ~8-9 and extracted with DCM (200 mL, 3×). The combined organic layer was dried over anhydrous Na₂SO₄, filtered and concentrated under vacuum. The crude product (4.6 g) was dissolved in ethanol (400 mL) and stirred at 80° C. for 20 min. Then, the solution was cooled to room temperature and stirred at 20° C. for 16 h. The precipitate was filtered and the filtrate cake was dried under vacuum to give the product 3-(5-(difluoromethyl)-1,3,4-thiadiazol-2-yl)-8-((3S,5S)-3,5-dimethylpiperazin-1-yl)-1-fluoro-N-(1-methylcyclopropyl)imidazo[1,5-a]pyridine-6-sulfonamide (3.2 g, 6.28 mmol, 69.01% yield) as a yellow solid.

LCMS: RT 0.471 min (method 6); m/z 516.1 (M+H)⁺ (ESI⁺); ¹H NMR (DMSO-d₆, 400 MHz): 9.47 (s, 1H), 8.49 (s, 1H), 7.68 (t, J=52.8 Hz, 1H), 6.61 (s, 1H), 3.28-3.24 (m, 2H), 3.15-3.05 (m, 2H), 2.85-2.75 (m, 2H), 2.12 (s, 1H), 1.17 (s, 3H), 1.15-1.13 (d, J=6.4 Hz, 6H), 0.78-0.69 (m, 2H), 0.50-0.42 (m, 2H)

### Preparation of Intermediate 14.1

### tert-butyl (1-(benzylamino)-2-methylpropan-2-yl)carbamate

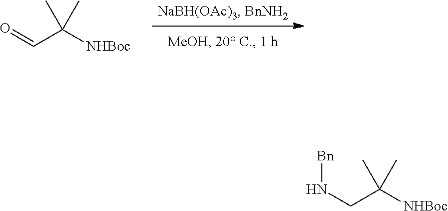 [SMILES: CC(C)(C=O)NC(=O)OC(C)(C)C ; CC(C)(CNCc1ccccc1)NC(=O)OC(C)(C)C]

To a solution of tert-butyl (2-methyl-1-oxopropan-2-yl)carbamate (80.00 g, 427 mmol) in methanol (800 mL) was added phenylmethanamine (45.78 g, 427 mmol) and the mixture was stirred at 20° C. for 10 min. Then, NaBH(OAc)₃ (135.83 g, 641 mmol) was added in batches and the reaction was stirred at 20° C. for 1 h. The resulting mixture was poured into H₂O (1000 mL) and extracted with EtOAc (1000 mL, 2×). The combined organic layer was washed with brine (1000 mL, 2×), dried over anhydrous Na₂SO₄, filtered and concentrated under reduced pressure to give the product tert-butyl (1-(benzylamino)-2-methylpropan-2-yl)carbamate (114.00 g, 404 mmol, 94.55% yield) as a white solid which was directly used in the next step without further purification.

RT 0.412 min (method 6); m/z 279.1. (M+H)⁺ (ESI⁺). ¹H NMR (CDCl₃, 400 MHz): 7.36-7.32 (m, 4H), 7.27-7.24 (m, 1H), 5.05 (br s, 1H), 3.82 (s, 2H), 2.65 (s, 2H), 1.44 (s, 9H), 1.29 (s, 6H).

### Preparation of Intermediate 14.2

### tert-butyl (1-(N-benzyl-2-chloroacetamido)-2-methylpropan-2-yl)carbamate

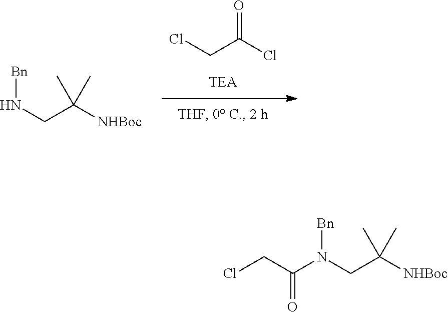 [SMILES: O=C(Cl)CCl ; CC(C)(CNCc1ccccc1)NC(=O)OC(C)(C)C ; CC(C)(CN(Cc1ccccc1)C(=O)CCl)NC(=O)OC(C)(C)C]

To a solution of tert-butyl (1-(benzylamino)-2-methylpropan-2-yl)carbamate (20.00 g, 71.8 mmol) and TEA (20 mL, 144 mmol) in THF (200 mL) at 0° C. was added 2-chloroacetyl chloride (6.3 mL, 79.0 mmol) dropwise over 10 min. The mixture was stirred at 0° C. for 2 h, then poured into H₂O (300 mL) and extracted with EtOAc (300 mL, 2×). The combined organic layer was washed with brine (300 mL, 2×), dried over anhydrous Na₂SO₄, filtered and concentrated under reduced pressure to give the product tert-butyl (1-(N-benzyl-2-chloroacetamido)-2-methylpropan-2-yl)carbamate (26.00 g, 66.3 mmol, 92.34% yield) as brown oil which was directly used in the next step without further purification.

RT 0.565 min (method 6); m/z 255.2. (M-Boc+H)⁺ (ESI⁺). ¹H NMR (CDCl₃, 400 MHz): 7.41-7.28 (m, 5H), 5.05 (br s, 1H), 4.75 (s, 2H), 4.03 (s, 2H), 3.73 (s, 2H), 1.43 (s, 9H), 1.35 (s, 6H)

### Preparation of Intermediate 14.3

### tert-butyl 4-benzyl-2,2-dimethyl-5-oxopiperazine-1-carboxylate

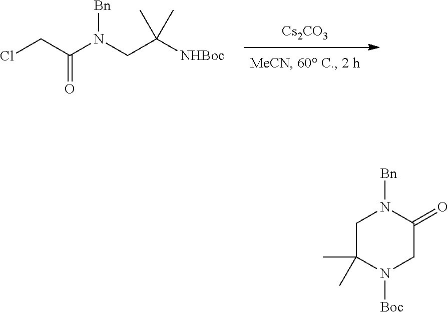 [SMILES: CC(C)(CN(Cc1ccccc1)C(=O)CCl)NC(=O)OC(C)(C)C ; "Cs2CO3" ; CC(C)(C)OC(=O)N1CC(=O)N(Cc2ccccc2)CC1(C)C]

To a solution of tert-butyl (1-(N-benzyl-2-chloroacetamido)-2-methylpropan-2-yl)carbamate (111.00 g, 313 mmol) in MeCN (1500 mL) was added Cs₂CO₃ (203.83 g, 626 mmol) and the mixture was stirred at 60° C. for 2 h. In parallel, four reactions were conducted with the same protocol but on different scales of tert-butyl (1-(N-benzyl-2-chloroacetamido)-2-methylpropan-2-yl)carbamate (26 g, 103 g, 110 g, 110 g). The reaction mixtures were combined, filtered and the filtrate was concentrated under reduced pressure. The resulting residue was purified by flash silica gel chromatography (ISCO; 6000 g SepaFlash, Silica Flash Column, Eluent of 0-50% Ethyl acetate/Petroleum ether gradient @300 mL/min) and concentrated under vacuum to give the product tert-butyl 4-benzyl-2,2-dimethyl-5-oxopiperazine-1-carboxylate (250.00 g, 786 mmol, 60.57% yield) as a white solid.

RT 0.535 min (method 6); m/z 319.1 (M+H)⁺ (ESI⁺). ¹H NMR (CDCl₃, 400 MHz): 7.41-7.28 (m, 5H), 4.60 (s, 2H), 4.10 (s, 2H), 3.10 (s, 2H), 1.46 (s, 9H), 1.30 (s, 6H)

### Preparation of Intermediate 14.4

### tert-butyl 4-benzyl-6-((benzyloxy)methyl)-2,2-dimethyl-5-oxopiperazine-1-carboxylate

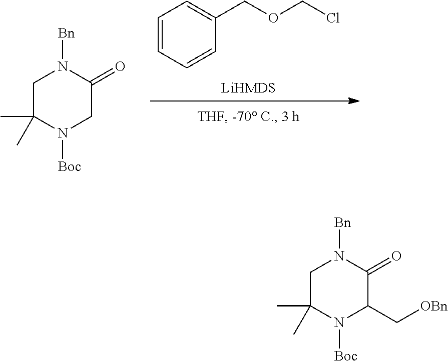

To a solution of tert-butyl 4-benzyl-2,2-dimethyl-5-oxopiperazine-1-carboxylate (60.00 g, 188 mmol) in THF (500 mL) under nitrogen was added LiHMDS (283 mL, 283 mmol) at −70° C. dropwise over a period of 1 h. The mixture was stirred at −70° C. for 0.5 h then ((chloromethoxy)methyl)benzene (44.27 g, 283 mmol) was added dropwise at −70° C. over a period of 0.5 h and the reaction mixture was stirred at −70° C. for 1 h. In parallel, three additional reactions were conducted with the same protocol but on different scales of tert-butyl 4-benzyl-2,2-dimethyl-5-oxopiperazine-1-carboxylate (50 g, 50 g, 60 g). The reaction mixtures were combined, quenched with HCl (aq., 2 L, 1N) and extracted with EtOAc (1 L, 2×). The combined organic layer was washed with brine (1 L, 2×), dried over anhydrous Na₂SO₄, filtered and concentrated under reduced pressure, The residue was purified by reversed-phase MPLC (column: Phenomenex Luna C18 15 μm; 100 A; mobile phase: A: 0.225% formic acid in water, B: MeCN; B %: 10%-80%, 20 min) and lyophilized directly to give the product tert-butyl 4-benzyl-6-((benzyloxy)methyl)-2,2-dimethyl-5-oxopiperazine-1-carboxylate (221.00 g, 497 mmol, LC/MS purity: 98.66%, 71.9% yield) as yellow oil.

RT 0.620 min (method 6); m/z 461.2 (M+Na)⁺ (ESI⁺). ¹H NMR (CDCl₃, 400 MHz): 7.23-7.18 (m, 8H), 7.17-7.15 (m, 2H), 4.81 (d, J=14.8 Hz, 1H), 4.58-4.55 (m, 1H), 4.48 (d, J=12 Hz, 1H), 4.41 (d, J=12 Hz, 1H), 4.30 (d, J=14.8 Hz, 1H), 3.87-3.84 (m, 1H), 3.80-3.77 (m, 2H), 2.50-2.47 (m, 1H), 1.36 (s, 9H), 1.35 (s, 3H), 1.16 (s, 3H)

### Preparation of Intermediate 14.5

### tert-butyl 4-benzyl-6-((benzyloxy)methyl)-2,2-dimethylpiperazine-1-carboxylate

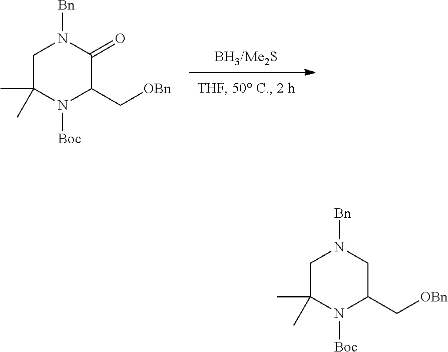 [SMILES: CC(C)(C)OC(=O)N1C(COCc2ccccc2)C(=O)N(Cc2ccccc2)CC1(C)C ; "BH3/Me2S" ; CC(C)(C)OC(=O)N1C(COCc2ccccc2)CN(Cc2ccccc2)CC1(C)C]

To a solution of tert-butyl 4-benzyl-6-((benzyloxy)methyl)-2,2-dimethyl-5-oxopiperazine-1-carboxylate (70.00 g, 160 mmol) in THF (800 mL) was added BH₃/Me₂S (32 mL, 320 mmol, 10 mol/L) at 0° C. The mixture was stirred at 20° C. for 1 h and at 50° C. for 2 h. In parallel, two additional reactions were conducted with the same protocol but on different scales tert-butyl 4-benzyl-6-((benzyloxy)methyl)-2,2-dimethyl-5-oxopiperazine-1-carboxylate (50 g, 70 g). The reaction mixtures were combined, slowly quenched with MeOH (600 mL) at 0° C., stirred at 20° C. for 1 h, then heated to 50° C. and stirred at 50° C. for another 2 h. The resulting mixture was concentrated under vacuum to give a residue which was triturated with petroleum ether (120 mL) for 1 h. The suspension was filtered. The filter cake was collected and dried under vacuum to give the product tert-butyl 4-benzyl-6-((benzyloxy)methyl)-2,2-dimethylpiperazine-1-carboxylate (143.00 g, 331 mmol, LC/MS purity 98.34%, 76.5% yield) as a white solid.

RT 0.527 min (method 6); m/z 425.4 (M+H)⁺ (ESI⁺). ¹H NMR (CDCl₃, 400 MHz): 7.37-7.29 (m, 8H), 7.28-7.26 (m, 2H), 4.54-4.45 (m, 2H), 4.20-4.13 (m, 1H), 3.96-3.88 (m, 1H), 3.54 (d, J=13.2 Hz, 1H), 3.44 (d, J=13.2 Hz, 1H), 3.35-3.30 (m, 1H), 3.10 (d, J=11.6 Hz, 1H), 2.44 (dd, J=2.4, 11.2 Hz, 1H), 2.13 (dd, J=2.4, 11.2 Hz, 1H), 1.94 (d, J=11.6 Hz, 1H), 1.47 (s, 9H), 1.41 (s, 3H), 1.28 (s, 3H).

### Preparation of Intermediate 14.6

### 1-benzyl-5-((benzyloxy)methyl)-3,3-dimethylpiperazine

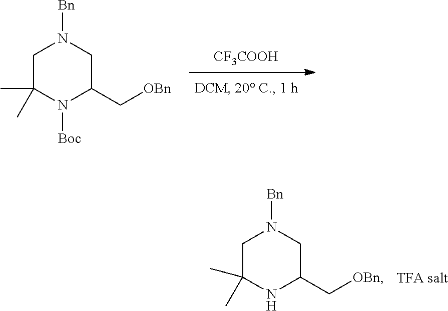 [SMILES: CC(C)(C)OC(=O)N1C(COCc2ccccc2)CN(Cc2ccccc2)CC1(C)C ; "CF3COOH" ; CC1(C)CN(Cc2ccccc2)CC(COCc2ccccc2)N1]

To a solution of tert-butyl 4-benzyl-6-((benzyloxy)methyl)-2,2-dimethylpiperazine-1-carboxylate (58.70 g, 138 mmol) in DCM (640 mL) was added trifluoroacetic acid (160 mL) at room temperature Then, the reaction mixture was stirred at 20° C. for 1 h and, then concentrated under vacuum to give the product 1-benzyl-5-((benzyloxy)methyl)-3,3-dimethylpiperazine 2,2,2-trifluoroacetate (60.00 g, 137 mmol, 98.97% yield) as yellow oil which was directly used in the next step without further purification.

RT 0.438 min (method 6); m/z 325.2. (M+H)⁺ (ESI⁺).

### Preparation of Intermediate 14.7

### (6,6-dimethylpiperazin-2-yl)methanol

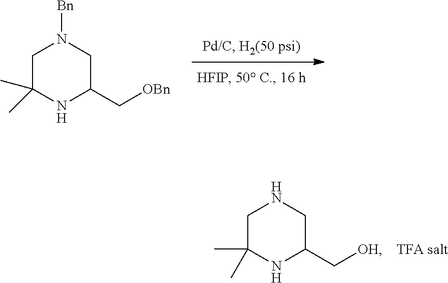

To a solution 1-benzyl-5-((benzyloxy)methyl)-3,3-dimethylpiperazine 2,2,2-trifluoroacetate (50.00 g, 114 mmol) in HFIP (1,1,1,3,3,3-hexafluoropropan-2-ol, 500 mL) was added Pd/C (10.00 g, 41.7 mmol) under N₂. The mixture was degassed with H₂ (3×) and stirred at 50° C. under H₂ (50 psi) for 16 h. In parallel, an additional reaction was conducted with the same protocol but on 60 g scale of 1-benzyl-5-((benzyloxy)methyl)-3,3-dimethylpiperazine 2,2,2-trifluoroacetate. The combined resulting mixture was filtered and concentrated under vacuum to give a residue which was triturated in petroleum ether (50 mL) during 1 h. The suspension was filtered and the filter cake was dried under vacuum to give the product (6,6-dimethylpiperazin-2-yl)methanol; 2,2,2-trifluoroacetate (91.00 g, crude) as a brown solid.

¹H NMR (CDCl₃, 400 MHz): 9.94-9.13 (br s, 2H, CF₃COOH salt), 5.89-5.44 (br s, 1H, OH), 3.74-3.62 (m, 3H), 3.53-3.41 (m, 2H), 3.10-2.94 (m, 2H), 1.48 (s, 3H), 1.44 (s, 3H).

### Preparation of Example 14

### 3-(5-(difluoromethyl)-1,3,4-thiadiazol-2-yl)-8-(5-(hydroxymethyl)-3,3-dimethylpiperazin-1-yl)-N-(1-methylcyclopropyl)imidazo[1,5-a]pyridine-6-sulfonamide

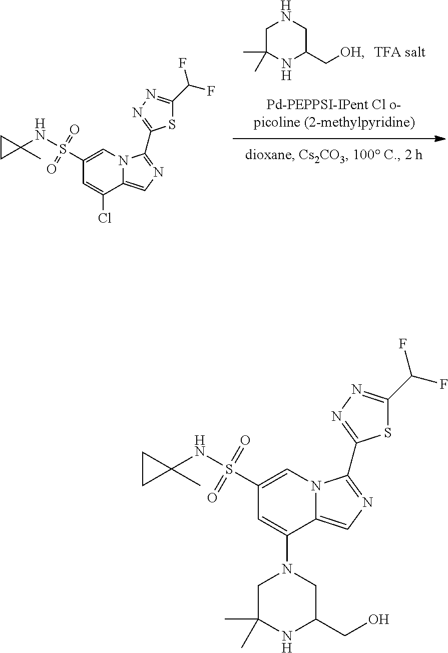

To a solution of 8-chloro-3-(5-(difluoromethyl)-1,3,4-thiadiazol-2-yl)-N-(1-methylcyclopropyl)imidazo[1,5-a]pyridine-6-sulfonamide (6.00 g, 14.3 mmol) and (6,6-dimethylpiperazin-2-yl)methanol 2,2,2-trifluoroacetate (7.381 g, 28.6 mmol) in 1,4-dioxane (60 mL) was added Cs₂CO₃ (9.875 g, 71.5 mmol) and Pd-PEPPSI-IPent Cl o-picoline (2-methylpyridine) (615 mg, 0.715 mmol). The reaction mixture was degassed with N₂ (3×) and stirred at 100° C. for 2 h. In parallel, three reactions were conducted with the same protocol but on different scales of 8-chloro-3-(5-(difluoromethyl)-1,3,4-thiadiazol-2-yl)-N-(1-methylcyclopropyl)imidazo[1,5-a]pyridine-6-sulfonamide (1 g, 5 g, 6 g). The resulting mixtures were concentrated separately under vacuum and the crude compounds were combined. The resulting residue was purified by flash silica gel chromatography (ISCO; 120 g SepaFlash. Silica Flash Column, Eluent of 0-60% Ethyl acetate/Petroleum ether gradient @100 mL/min) to give crude product (10 g) which was dissolved in in MeOH (200 mL). Then, sulfhydryl modified silica gel (20 g) was added and the mixture was stirred at 20° C. for 1 h, followed by removal of sulfhydryl silica gel by filtration. This procedure was repeated four times and the final filtrate was concentrated under vacuum to give a residue, which was purified by reversed-phase flash (ISCO®; 330 g Flash Column Welch Ultimate XB_C18 20-40 μm; 120 A, mobile phase: A: 0.1% formic acid in water, B: MeCN; B %: 0%-40% @100 mL/min). The fractions containing the expected compound were combined and NaHCO₃ (aq., sat., 20 mL) was added until pH=7-8. Then, MeCN was removed under vacuum at 45° C. and the aqueous layer was extracted with EtOAc (500 mL, 3×). The combined organic layer was washed with brine (500 mL), dried over anhydrous Na₂SO₄, filtered and concentrated under vacuum to give the product 3-(5-(difluoromethyl)-1,3,4-thiadiazol-2-yl)-8-(5-(hydroxymethyl)-3,3-dimethylpiperazin-1-yl)-N-(1-methylcyclopropyl)imidazo[1,5-a]pyridine-6-sulfonamide (8.2 g, 15.5 mmol, yield: 36.25%) as a yellow solid.

RT 0.446 min (method 6); m/z 528.1 (M+H)⁺ (ESI⁺). ¹H NMR (DMSO-d₆, 400 MHz): 9.57 (s, 1H), 8.46 (s, 1H), 7.85 (s, 1H), 7.68 (t, J=53.2 Hz, 1H), 6.68 (s, 1H), 4.72 (t, J=5.2 Hz, 1H), 3.63-3.53 (m, 1H), 3.51-3.46 (m, 1H), 3.45-3.36 (m, 2H), 3.30-3.20 (m, 1H), 2.60-2.53 (m, 1H), 2.49-2.43 (m, 1H), 1.89 (br s, 1H), 1.34 (s, 3H), 1.16 (s, 3H), 1.12 (s, 3H), 0.75-0.69 (m, 2H), 0.53-0.39 (m, 2H)

### Preparation of Example 14a

### (R or S)-3-(5-(difluoromethyl)-1,3,4-thiadiazol-2-yl)-8-(5-(hydroxymethyl)-3,3-dimethylpiperazin-1-yl)-N-(1-methylcyclopropyl)imidazo[1,5-a]pyridine-6-sulfonamide and

### Preparation of Example 14b

### (S or R)-3-(5-(difluoromethyl)-1,3,4-thiadiazol-2-yl)-8-(5-(hydroxymethyl)-3,3-dimethylpiperazin-1-yl)-N-(1-methylcyclopropyl)imidazo[1,5-a]pyridine-6-sulfonamide

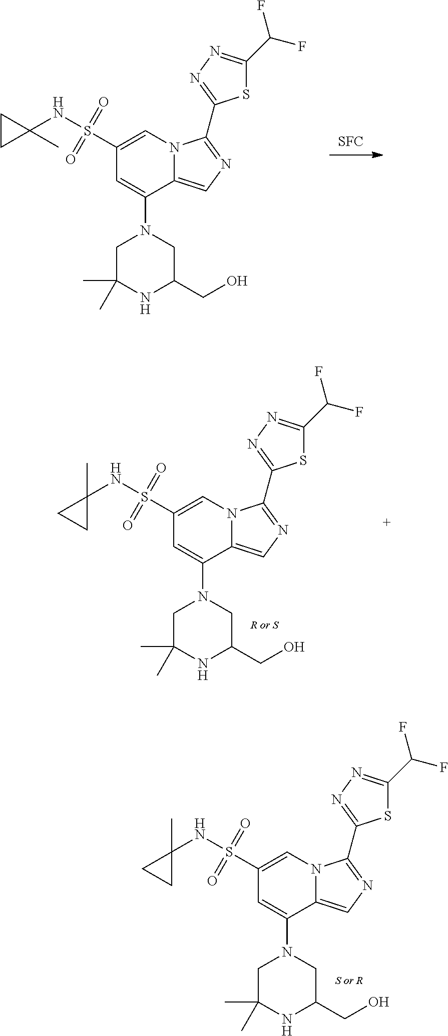 [SMILES: CC1(C)CN(c2cc(S(=O)(=O)NC3(C)CC3)cn3c(-c4nnc(C(F)F)s4)ncc23)CC(CO)N1 ; "SFC" ; CC1(C)CN(c2cc(S(=O)(=O)NC3(C)CC3)cn3c(-c4nnc(C(F)F)s4)ncc23)CC(CO)N1 ; CC1(C)CN(c2cc(S(=O)(=O)NC3(C)CC3)cn3c(-c4nnc(C(F)F)s4)ncc23)CC(CO)N1]

3-(5-(difluoromethyl)-1,3,4-thiadiazol-2-yl)-8-(5-(hydroxymethyl)-3,3-dimethylpiperazin-1-yl)-N-(1-methylcyclopropyl)imidazo[1,5-a]pyridine-6-sulfonamide (8.15 g, 15.4 mmol) was purified by preparative SFC (column: Daicel Chiralpak IH 250×50 mm I.D., 10 μm particle size; Mobile Phase: A: CO₂-ACN, B: EtOH (0.1% NH₃·H₂O): 30% EtOH (0.1% NH₃·H₂O additive) in supercritical CO₂, Gradient information: 10 min, Flow Rate: 150 mL/min). The fractions containing the compounds were concentrated under reduced pressure and lyophilized to give the products (R or S)-3-(5-difluoromethyl)-1,3,4-thiadiazol-2-yl)-8-(5-hydroxymethyl)-3,3-dimethylpiperazin-1-yl)-N-(1-methylcyclopropyl)imidazo[1,5-a]pyridine-6-sulfonamide (3.70 g, 7.01 mmol, 45.4% yield) as a yellow solid.

LCMS: RT 0.443 min (method 6); m/z 528.2 (M+H)⁺ (ESI⁺). SFC: RT 1.63 min (method 1); 100% ee; ¹H NMR (ACN-d₃, 400 MHz): 9.69 (s, 1H), 7.81 (s, 1H), 7.27 (t, J=53.6 Hz, 1H), 6.63 (s, 1H), 6.37 (br s, 1H), 3.68-3.45 (m, 4H), 3.40-3.31 (m, 1H), 2.64-2.54 (m, 2H), 1.43 (s, 3H), 1.25 (s, 3H), 1.17 (s, 3H), 0.89-0.80 (m, 2H), 0.55-0.47 (m, 2H)

and (S or R)-3-(5-(difluoromethyl)-1,3,4-thiadiazol-2-yl)-8-(5-(hydroxymethyl)-3,3-dimethylpiperazin-1-yl)-N-(1-methylcyclopropyl)imidazo[1,5-a]pyridine-6-sulfonamide (3.50 g, 6.63 mmol, 42.94% yield) was obtained as a yellow solid.

LCMS: RT 0.445 min (method 6); m/z 528.2 (M+H) (ES). SFC: RT 1.730 min (method 1); 98.88% ee; ¹H NMR (ACN-d₃, 400 MHz): 9.69 (s, 1H), 7.81 (s, 1H), 7.27 (t, J=53.6 Hz, 1H), 6.63 (s, 1H), 6.38 (br s, 1H), 3.68-3.45 (m, 4H), 3.40-3.31 (m, 1H), 2.64-2.54 (m, 2H), 1.43 (s, 3H), 1.25 (s, 3H), 1.18 (s, 3H), 0.89-0.80 (m, 2H), 0.55-0.47 (m, 2H)

Compounds listed in the table below were prepared according to the corresponding general procedures or when stated in a similar way to related compound and starting from the corresponding intermediates

| Cpd | | Yield | | | |
|---|---|---|---|---|---|
| number | Structure | (%) | Procedure | LC/MS | 1H NMR |
| 10 | 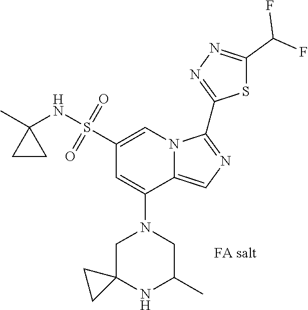 | 30.20 | Procedure 2 | RT 0.461 min (method 4); m/z 510.2 (M + H)⁺ (ESI⁺) | ¹H NMR (CD₃Cl, 400 MHz): 9.84 (s, 1H), 8.25 (br, 1H), 7.67 (s, 1H), 7.08 (t, J = 53.6 Hz, 1H), 6.63 (s, 1H), 5.47-5.08 (m, 1H), 3.69 (d, J = 12.0 Hz, 1H), 3.38 (s, 1H), 3.29 (dd, J = 12.4, 3.2 Hz, 1H), 3.04 (d, J = 12.0 Hz, 1H), 2.69-2.62 (m, 1H), 1.38 (s, 3H), 1.23 (d, J = 6.0 Hz, 3H), 0.94 (s, 2H), 0.86 (s, 2H), 0.62-0.59 (m, 2H), 0.11 (s, 2H). |
| 11 | 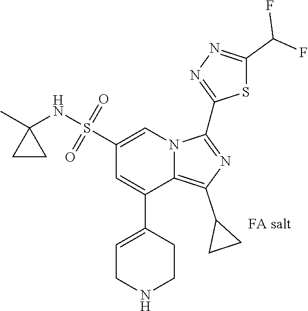 | 28.75 | Procedure 2 | LCMS: RT 0.392 min (method 7); m/z 507.2 (M + H)⁺ (ESI⁺). | ¹H NMR (CD₃CN, 400 MHz): 9.88 (s, 1H), 8.22 (s, 1H), 7.22 (t, J = 53.6 Hz, 1H), 6.96 (s, 1H), 6.42 (br s, 1H), 6.00 (s, 1H), 3.63-3.50 (m, 2H), 3.20-3.15 (m, 2H), 2.57-2.50 (m, 2H), 2.35-2.25 (m, 1H), 1.24 (s, 3H), 1.07- 0.96 (m, 4H), 0.88- 0.78 (m, 2H), 0.53- 0.40 (m, 2H) |
| 12 | 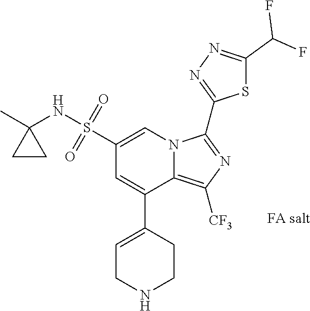 [SMILES: CC1(NS(=O)(=O)c2cc(C3=CCNCC3)c3c(C(F)(F)F)nc(-c4nnc(C(F)F)s4)n3c2)CC1] | 58.70 | Procedure 2 | LCMS: RT 0.470 min (method 6); m/z 535.1 (M + H)⁺ (ESI+). | ¹H NMR (DMSO-d₆, 400 MHz): 9.99 (s, 1H), 8.60 (br s, 1H), 8.26 (s, 1H), 7.72 (t, J = 53.2 Hz, 1H), 7.38 (s, 1H), 5.91 (s, 1H), 3.49-3.37 (m, 2H), 3.10-2.92 (m, 2H), 2.40-2.31 (m, 2H), 1.19 (s, 3H), 0.82- 0.67 (m, 2H), 0.53- 0.44 (m, 2H) |

The following Table 1 provides an overview on the compounds described in the example section:

TABLE 1

| Example | | |
|---|---|---|
| No. | Structure | Name of compound |
| 1 | 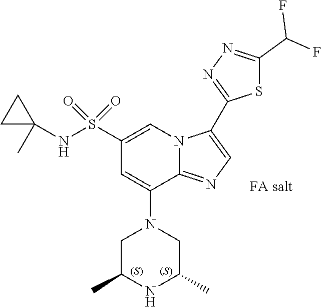 | 3-(5-(difluoromethyl)-1,3,4-thiadiazol-2-yl)- 8-((3S,5S)-3,5-dimethylpiperazin-1-yl)-N-(1- methylcyclopropyl)imidazo[1,2-a]pyridine-6- sulfonamide formate |
| 2 | 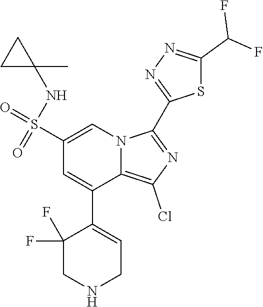 [SMILES: CC1(NS(=O)(=O)c2cc(C3=CCNCC3(F)F)c3c(Cl)nc(-c4nnc(C(F)F)s4)n3c2)CC1] | 1-chloro-8-(3,3-difluoro-1,2,3,6- tetrahydropyridin-4-yl)-3-(5-(difluoromethyl)- 1,3,4-thiadiazol-2-yl)-N-(1- methylcyclopropyl)imidazo[1,5-a]pyridine-6- sulfonamide |
| 3 | 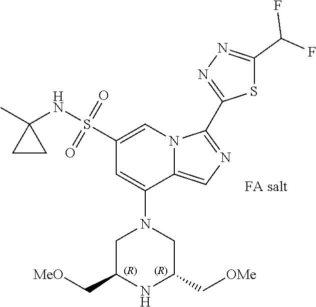 | 8-((3R,5R)-3,5- bis(methoxymethyl)piperazin-1-yl)-3-(5- (difluoromethyl)-1,3,4-thiadiazol-2-yl)-N-(1- methylcyclopropyl)imidazo[1,5-a]pyridine-6- sulfonamide formate |
| 4 | 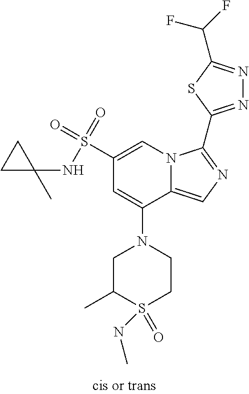 | (Cis or Trans)-3-(5-(difluoromethyl)-1,3,4- thiadiazol-2-yl)-8-(2-methyl-1-(methylimino)-1- oxido-116-thiomorpholino)-N-(1- methylcyclopropyl)imidazo[1,5-a]pyridine-6- sulfonamide |
| 5 | 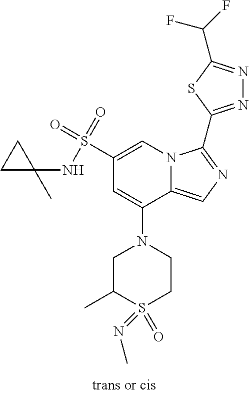 [SMILES: CN=S1(=O)CCN(c2cc(S(=O)(=O)NC3(C)CC3)cn3c(-c4nnc(C(F)F)s4)ncc23)CC1C] | (Trans or cis)-3-(5-(difluoromethyl)-1,3,4- thiadiazol-2-yl)-8-(2-methyl-1-(methylimino)-1- oxido-116-thiomorpholino)-N-(1- methylcyclopropyl)imidazo[1,5-a]pyridine-6- sulfonamide |
| 10 | 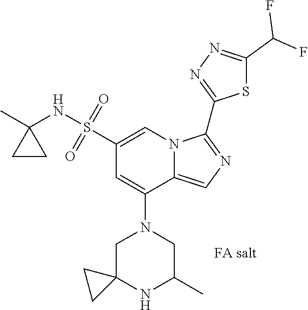 | 3-(5-(difluoromethyl)-1,3,4-thiadiazol-2-yl)- 8-(5-methyl-4,7-diazaspiro[2.5]octan-7-yl)-N-(1- methylcyclopropyl)imidazo[1,5-a]pyridine-6- sulfonamide formate |
| 11 | 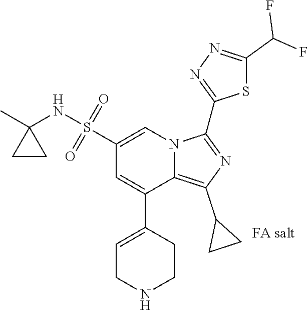 | 1-cyclopropyl-3-(5-(difluoromethyl)-1,3,4- thiadiazol-2-yl)-N-(1-methylcyclopropyl)-8- (1,2,3,6-tetrahydropyridin-4-yl)imidazo[1,5- alpyridine-6-sulfonamide formate |
| 12 | 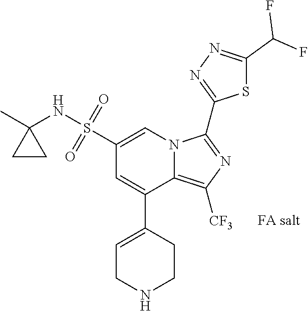 [SMILES: CC1(NS(=O)(=O)c2cc(C3=CCNCC3)c3c(C(F)(F)F)nc(-c4nnc(C(F)F)s4)n3c2)CC1] | 3-(5-(difluoromethyl)-1,3,4-thiadiazol-2-yl)- N-(1-methylcyclopropyl)-8-(1,2,3,6- tetrahydropyridin-4-yl)-1- (trifluoromethyl)imidazo[1,5-a]pyridine-6- sulfonamide formate |
| 13 | 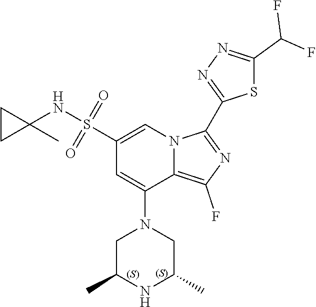 | 3-(5-(difluoromethyl)-1,3,4-thiadiazol-2-yl)- 8-((3S,5S)-3,5-dimethylpiperazin-1-yl)-1-fluoro- N-(1-methylcyclopropyl)imidazo[1,5-a]pyridine-6- sulfonamide |
| 14 | 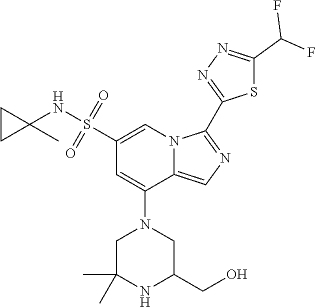 [SMILES: CC1(C)CN(c2cc(S(=O)(=O)NC3(C)CC3)cn3c(-c4nnc(C(F)F)s4)ncc23)CC(CO)N1] | 3-(5-(difluoromethyl)-1,3,4-thiadiazol-2-yl)- 8-(5-(hydroxymethyl)-3,3-dimethylpiperazin-1-yl)- N-(1-methylcyclopropyl)imidazo[1,5-a]pyridine-6- sulfonamide |
| 14a | 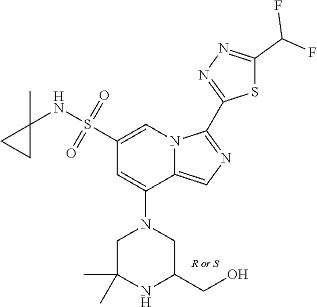 [SMILES: CC1(C)CN(c2cc(S(=O)(=O)NC3(C)CC3)cn3c(-c4nnc(C(F)F)s4)ncc23)CC(CO)N1] | (R or S)-3-(5-(difluoromethyl)-1,3,4- thiadiazol-2-yl)-8-(5-(hydroxymethyl)-3,3- dimethylpiperazin-1-yl)-N-(1- methylcyclopropyl)imidazo[1,5-a]pyridine-6- sulfonamide |
| 14b | 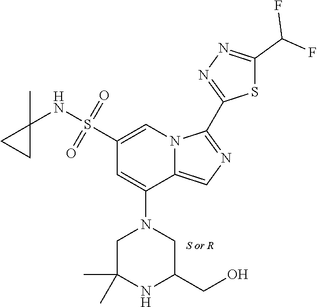 [SMILES: CC1(C)CN(c2cc(S(=O)(=O)NC3(C)CC3)cn3c(-c4nnc(C(F)F)s4)ncc23)CC(CO)N1] | (S or R)-3-(5-(difluoromethyl)-1,3,4- thiadiazol-2-yl)-8-(5-(hydroxymethyl)-3,3- dimethylpiperazin-1-yl)-N-(1- methylcyclopropyl)imidazo[1,5-a]pyridine-6- sulfonamide |

### Biological Evaluation of the Exemplary Compounds

Exemplary compounds of formula (I) were tested in selected biological and/or physicochemical assays one or more times. When tested more than once, data are reported as either average values or as median values, wherein the average value, also referred to as the arithmetic mean value, represents the sum of the values obtained divided by the number of times tested, and the median value represents the middle number of the group of values when ranked in ascending or descending order. If the number of values in the data set is odd, the median value is the middle value. If the number of values in the data set is even, the median is the arithmetic mean of the two middle values. The in vitro pharmacological, pharmacokinetic and physicochemical properties of the compounds can be determined according to the following assays and methods.

### PARG Protein Expression and Purification

A codon optimized gene encoding human PARG (448-976 [H446G, L447S, L473S, N479S, S802A, R811K, M841I, S858P, I916T, T924D, D927K, C963S, A967T]) was synthesized by Genscript, and cloned into pET15b (NcoI/BamHI) with an N-terminal, Thrombin protease cleavable 6His-TwinStrep tag. Expression of the protein in E. coli BL21 (DE3) was induced by addition of 0.2 mM IPTG to a shake flask culture grown to OD600=0.8 at 37° C. Growth was allowed to continue at 30° C. for a further 20 hours before harvesting by centrifugation and storage of the cell pellet at −80° C.

Protein was purified by IMAC and SEC: frozen cell pellets (typically 40 g wet weight) were resuspended by homogenization in 5 volumes buffer A (25 mM Tris/HCl pH 8.0, 200 mM NaCl, 2 mM DTT), supplemented with 1 mg of DNase I from bovine pancreas (Sigma-Aldrich) and protease inhibitors (Roche Complete™ EDTA-free protease inhibitor tablet), and lysed by passage through a Constant Systems BasicZ homogenizer. The lysate was clarified by centrifugation for 60 minutes at 25,000 g, 4° C., and the lysate supernatant was loaded onto 5 ml StrepTrap HP (Cytiva) pre-equilibrated with buffer A. The column was washed with buffer A (~10 CV), then buffer B containing 1M KCl (~5 CV), and then the protein was eluted with buffer A containing 2.5 mM d-Desthiobiotin. Pooled fractions containing 6HisTwinStrep-TEV-hPARG were incubated with TEV protease overnight at 4° C. hPARG was separated from uncleaved material and Thrombin protease through gel filtration with Superdex75 sizing column (GE Healthcare) pre-equilibrated with SEC buffer (15 mM Tris/HCl pH 8.5, 100 mM NaCl, 2 mM DTT). Pooled fractions containing pure hPARG were concentrated using a 10 k MWCO spin concentrator (VivaSpin) to 10 mg/mL, and then either used immediately for crystallisation or snap-frozen in liquid nitrogen for storage at −80° C.

### PARG Enzymatic IC₅₀ Assay

PARG enzyme as incubated with compound or vehicle (DMSO) for 2 hours in a 384 well plate. After adding the PARG substrate ADP-ribose-pNP, the plate was read for absorbance intensity at 405 nm. The vehicle (DMSO) with high absorbance intensity represents no inhibition of enzymatic reaction while the low control (no enzyme) with low absorbance intensity represents full inhibition of enzymatic reaction.

### Materials:

hPARG: Peak Protein, 30 nM
Substrate: ADP-pNP, 800 μM, Jena Bioscience catalog #NU-955
Reaction time: 60 minutes
Assay buffer: 50 mM Tris-HCl pH 8.0, 100 mM NaCl, 2 mM DTT
Temperature: 30° C.
Total volume: 30 μL

### Controls:

0% inhibition control: DMSO
100% inhibition control: No enzyme

The protocol that was used for enzyme reaction and detection is as follows:
1. Transfer 100 nL of the final concentration of test compounds or vehicle (DMSO) to the appropriate wells of a microtiter plate.
2. Centrifuge the plate at 1000 rpm for 1 minute.
3. Transfer 14.6 μL of 2× final concentration of enzyme in assay buffer or assay buffer alone to the appropriate wells.
4. Centrifuge the plate at 1000 rpm for 1 minute.
5. Incubate the plate at room temperature for 15 minutes or 2 hours.
6. Transfer 15.4 μL of 2× substrate in assay buffer to all the test wells.
7. Centrifuge the plate at 1000 rpm for 1 minute.
8. Read the plate on a plate reader (e.g., Spark Tecan).

The Absorbance IC₅₀ value of compounds of Formula (I) in Examples 1 to 12 are provided in Table 1 below.

### Cellular PAR Chain Assay

The ability of compounds to inhibit PARG in response to DNA damage, was assessed with U2OS cells pretreated with the compounds for 1 hour, following a 1-hour treatment with the DNA alkylating agent temozolomide (TMZ). The cells were harvested and fixed in 70% ethanol, rehydrated with glucose and EDTA in PBS and subsequently blocked for 1 hour with PBS 1% BSA and 0.01% Tween-20 (PBT). The cells were incubated for 2 hours at room temperature with a mouse monoclonal antibody against poly (ADP) ribose (PAR) polymer. The cells were washed and incubated with an anti-mouse Alexa-488 conjugated secondary antibody for 1 hour at room temperature. Propidium iodide staining was used to determine DNA content in the cells (staining at 4° C. overnight). The fluorescence intensity of the cells was assessed by flow cytometry (Cytoflex from Beckmann) and the percentage of PAR chain positive cells (gated in relation to TMZ+DMSO treated control) was determined. PAR chain positive cells % were fit against the concentration of the compound using a 4 parameter log-logistic function, generating PAR chain EC₅₀ values:

$$f ( x ) = c + \frac{d - c}{1 + {exp}^{b ( log ( x ) - log ( e ) )}}$$

The PAR chain EC₅₀ value for compounds of Formula (I) in Examples are provided in Table 1 below.

### Cellular Viability Assay

NCIH-460 as a PARG-inhibition sensitive cell line and U2OS as PARG-inhibition insensitive cell line were plated at 1000 cells/well and 2000 cells/well, respectively, in 96-well white plates with clear flat bottom. After 24 hours, the compounds were added with the Tecan digital dispenser (D300e), in duplicates. The outer wells of the plate were excluded. After 96 hours of incubation, 150 μl of the growth medium were removed and 50 μl of Cell Titer-Glo (Promega) were added per well. Following an incubation of 10 minutes, luminescence was read using a plate reader (Tecan). Averaged values of the samples were normalized to DMSO treated control samples. Curves were fit as % of the control vs. log of the compound concentration using a 4 parameter log-logistic function:

$$f ( x ) = c + \frac{d - c}{1 + {exp}^{b ( log ( x ) - log ( e ) )}}$$

The PARGi (NCIH-460 and U2OS) cellular viability EC₅₀ values for compounds of Formula (I) in Examples are provided in Table 2 below.

Table 2: Inhibition of PARG and cellular activity of compounds according to the present invention.

The IC₅₀ (inhibitory concentration at 50% of maximal effect) values are indicated in μM, empty space means that the corresponding compounds have not been tested in the respective assay.
\textcircled{1} Example number
\textcircled{2} IC₅₀ in μM determined in PARG enzymatic assay (PARG protein and 2 hours incubation) described under PARG enzymatic IC₅₀ assay
\textcircled{4} EC₅₀ in μM determined in cellular assay as described under Cellular PAR chain assay (conditions with treatment of TMZ).
\textcircled{6} EC₅₀ in μM determined in NCIH-460 cells as described under Cellular viability assay.
\textcircled{7} EC₅₀ in μM determined in U2OS cells as described under Cellular viability assay.

TABLE 2

| \textcircled{1} | \textcircled{2} | \textcircled{4} | \textcircled{6} | \textcircled{7} |
|---|---|---|---|---|
| 1 | 0.011 | | 0.070 | 12.607 |
| 2 | 0.011 | | 0.199 | 13.312 |
| 3 | 0.012 | | 0.019 | >11.0 |
| 4 | 0.011 | | 0.111 | >20.0 |
| 5 | 0.011 | | 0.208 | >20.0 |
| 10 | 0.011 | | 0.418 | >20 |
| 11 | 0.016 | 0.009 | 0.074 | 1,717 |
| 12 | 0.011 | 0.006 | 0.094 | 3.865 |
| 13 | 0.016 | 0.005 | 0.035 | 3.59 |
| 14 | 0.011 | | 0.094 | 9.515 |
| 14a | 0.015 | | 1.793 | >11.92 |
| 14b | 0.012 | | 0.0774 | >11.394 |

1. A compound of formula (I): 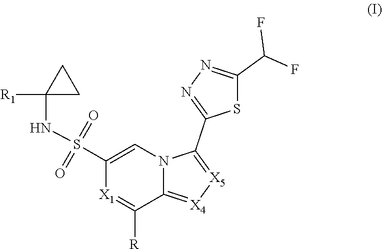
or a pharmaceutically acceptable salt thereof, wherein
X₁, and X₅ are each independently selected from N and CH,
X₄ is selected from N and C—R₂, wherein R₂ is selected from hydrogen, C₁₋₂ alkyl, C₂ alkenyl, C₂ alkynyl, C₁₋₂ haloalkyl, cyclopropyl, cyclobutyl, oxetanyl, halogen, —SO₂(C₁₋₂ alkyl), —SO(NH)(C₁₋₂ alkyl), —SO(NC₁₋₂ alkyl)(C₁₋₂ alkyl), —CONH(C₁₋₂ alkyl), O(C₁₋₂ alkyl), —NH₂, —NH(C₁₋₂ alkyl) and —N(C₁₋₂ alkyl)(C₁₋₂ alkyl);
R is selected from 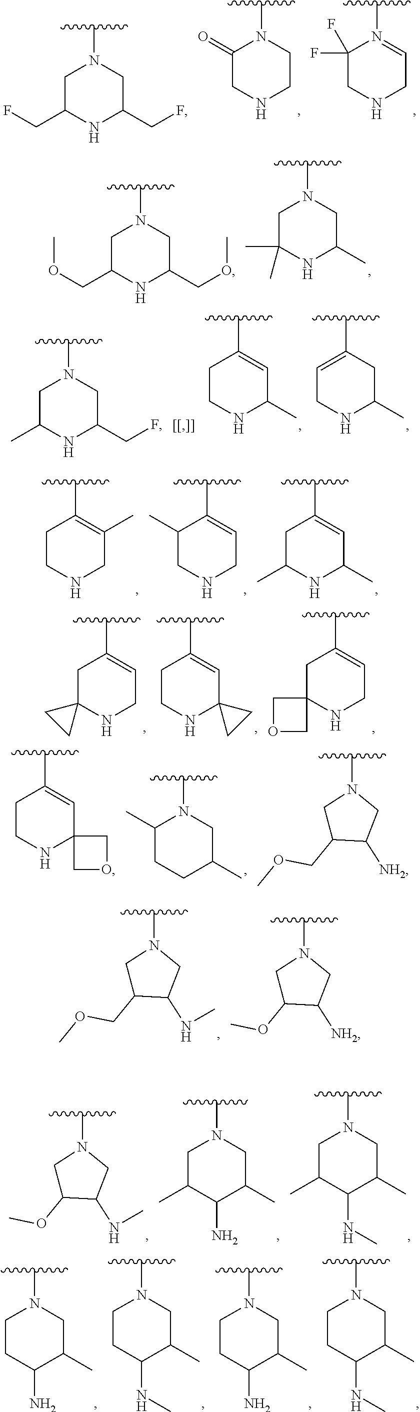 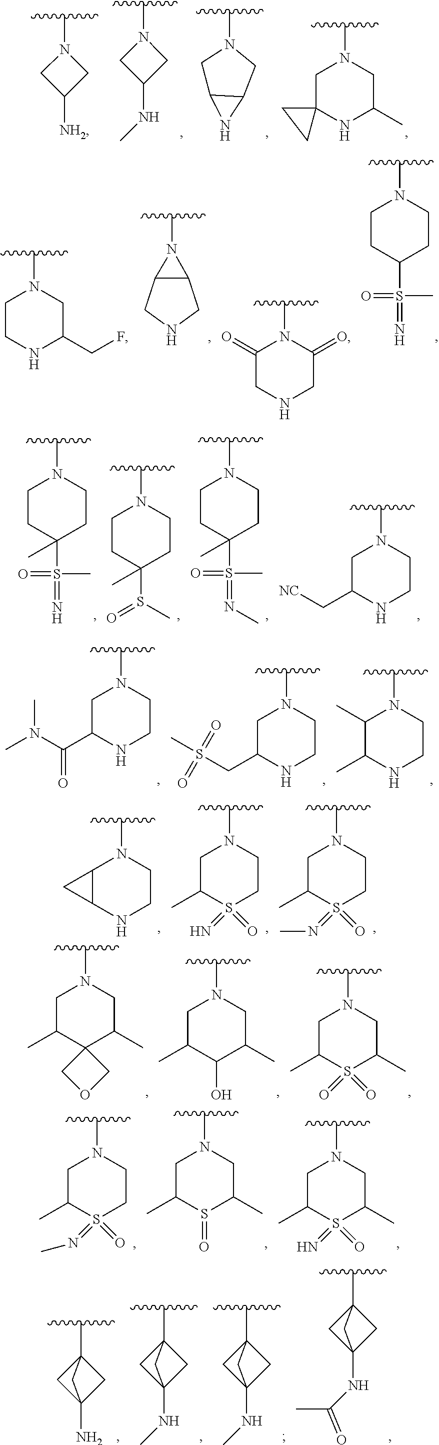 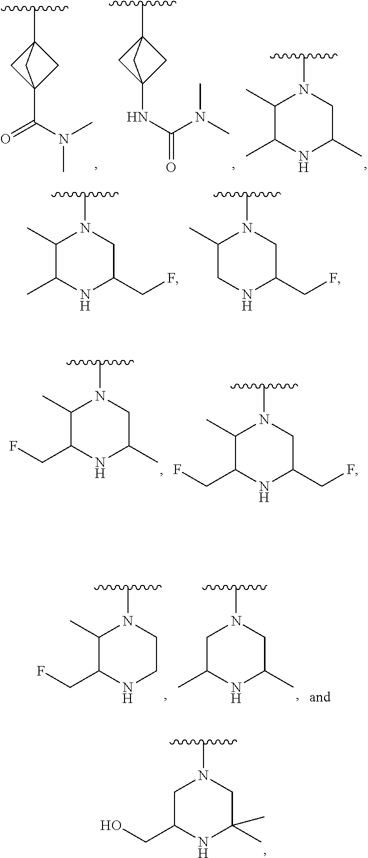
and
R₁ is selected from the group consisting of fluoro, cyano, formyl, (C₁₋₂)alkyl, (C₂)alkenyl, (C₂)alkynyl, (C₁₋₂)haloalkyl, —(C₁₋₂ alkylene)-OH and —(C₁₋₂ alkylene)-O—(C₁₋₂ alkyl).

2. The compound of claim 1, selected from: 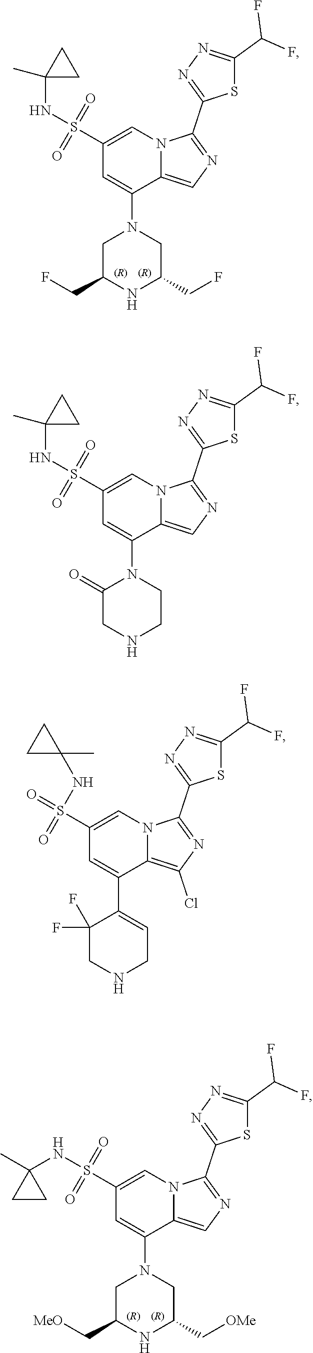 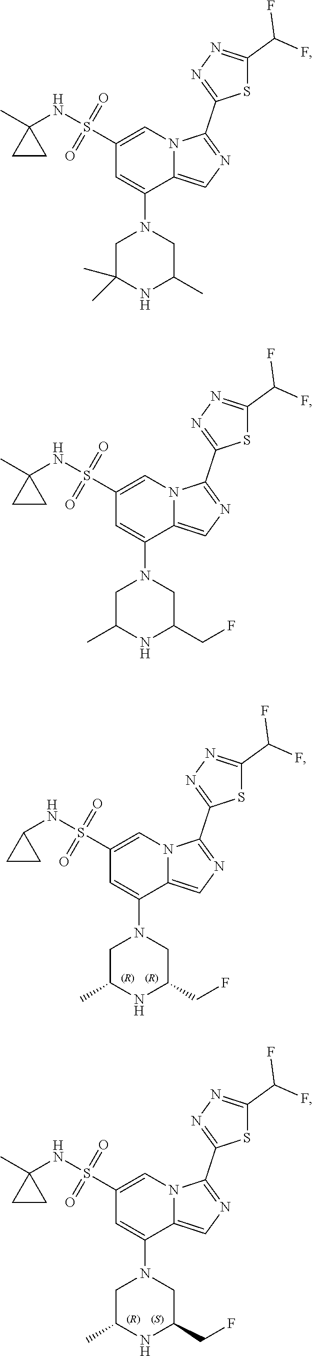 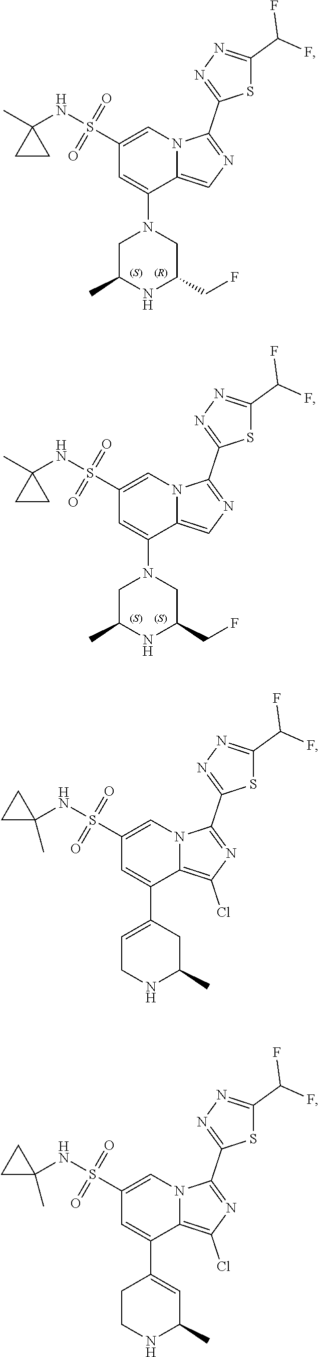 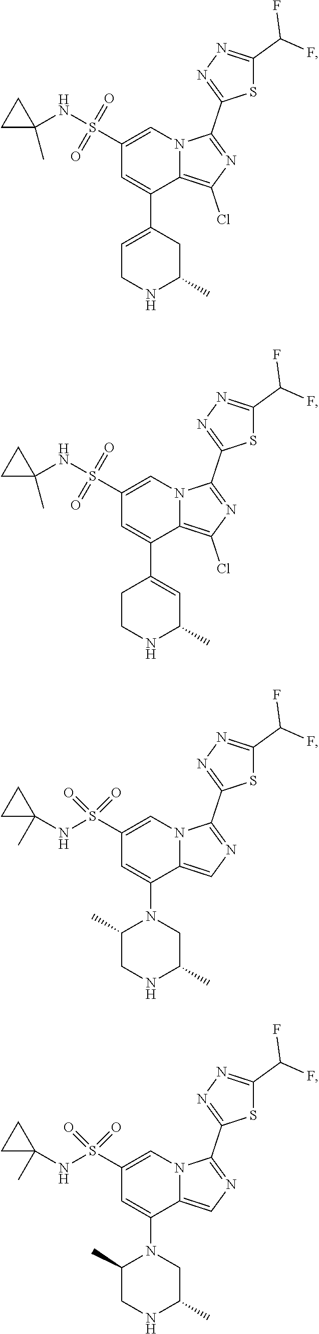 [SMILES: C[C@H]1CC(c2cc(S(=O)(=O)NC3(C)CC3)cn3c(-c4nnc(C(F)F)s4)nc(Cl)c23)=CCN1 ; C[C@H]1C=C(c2cc(S(=O)(=O)NC3(C)CC3)cn3c(-c4nnc(C(F)F)s4)nc(Cl)c23)CCN1 ; C[C@H]1CN(c2cc(S(=O)(=O)NC3(C)CC3)cn3c(-c4nnc(C(F)F)s4)ncc23)[C@@H](C)CN1 ; C[C@@H]1CN[C@@H](C)CN1c1cc(S(=O)(=O)NC2(C)CC2)cn2c(-c3nnc(C(F)F)s3)ncc12] 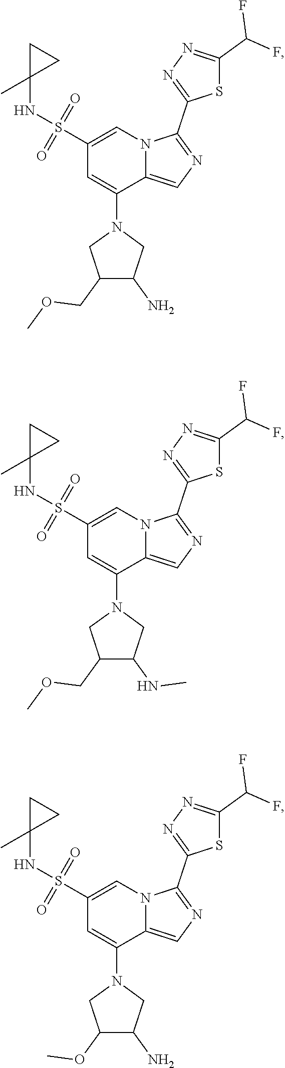 [SMILES: COCC1CN(c2cc(S(=O)(=O)NC3(C)CC3)cn3c(-c4nnc(C(F)F)s4)ncc23)CC1N ; CNC1CN(c2cc(S(=O)(=O)NC3(C)CC3)cn3c(-c4nnc(C(F)F)s4)ncc23)CC1COC ; COC1CN(c2cc(S(=O)(=O)NC3(C)CC3)cn3c(-c4nnc(C(F)F)s4)ncc23)CC1N] 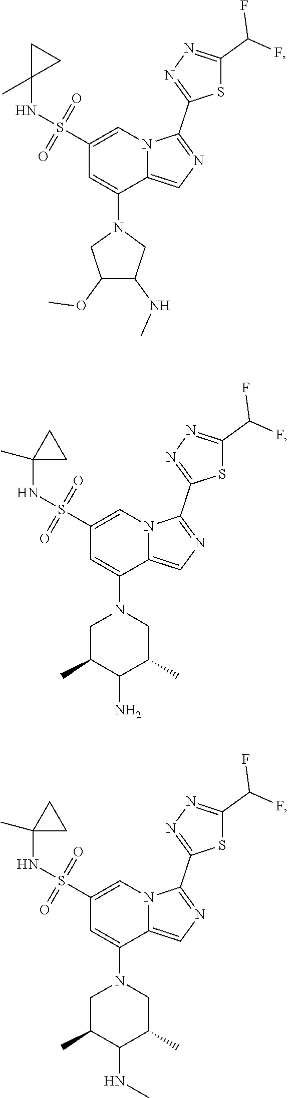 [SMILES: CNC1CN(c2cc(S(=O)(=O)NC3(C)CC3)cn3c(-c4nnc(C(F)F)s4)ncc23)CC1OC ; C[C@H]1CN(c2cc(S(=O)(=O)NC3(C)CC3)cn3c(-c4nnc(C(F)F)s4)ncc23)C[C@H](C)C1N ; CNC1[C@@H](C)CN(c2cc(S(=O)(=O)NC3(C)CC3)cn3c(-c4nnc(C(F)F)s4)ncc23)C[C@@H]1C] 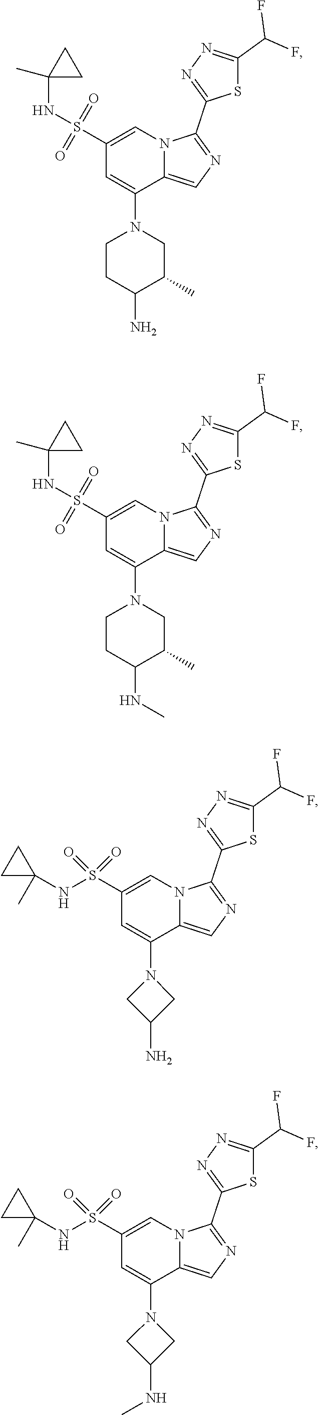 [SMILES: C[C@H]1CN(c2cc(S(=O)(=O)NC3(C)CC3)cn3c(-c4nnc(C(F)F)s4)ncc23)CCC1N ; CNC1CCN(c2cc(S(=O)(=O)NC3(C)CC3)cn3c(-c4nnc(C(F)F)s4)ncc23)C[C@@H]1C ; CC1(NS(=O)(=O)c2cc(N3CC(N)C3)c3cnc(-c4nnc(C(F)F)s4)n3c2)CC1 ; CNC1CN(c2cc(S(=O)(=O)NC3(C)CC3)cn3c(-c4nnc(C(F)F)s4)ncc23)C1] 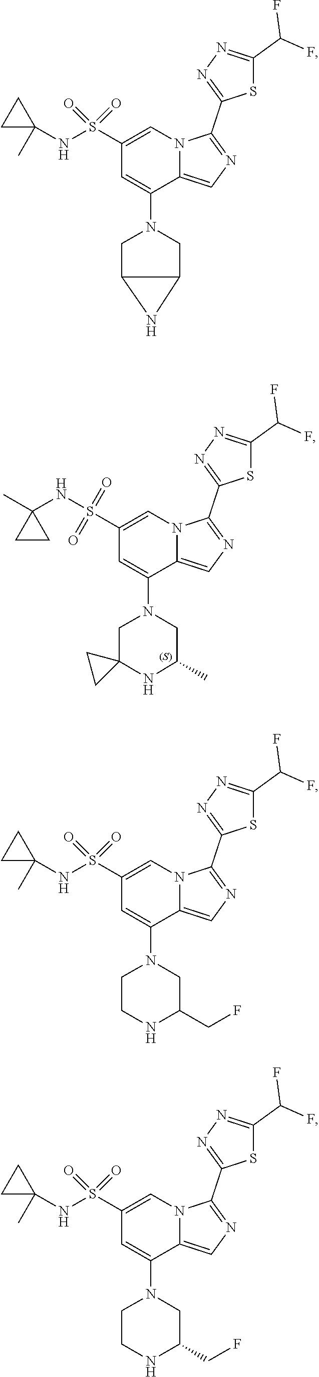 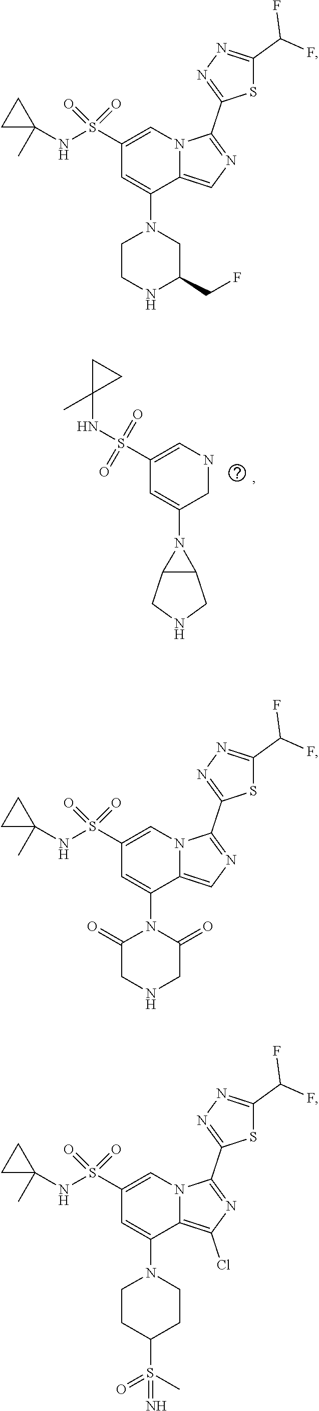 [SMILES: CC1(NS(=O)(=O)c2cc(N3CCN[C@H](CF)C3)c3cnc(-c4nnc(C(F)F)s4)n3c2)CC1 ; CC1(NS(=O)(=O)C2=CNCC(N3C4CNCC43)=C2)CC1 ; CC1(NS(=O)(=O)c2cc(N3C(=O)CNCC3=O)c3cnc(-c4nnc(C(F)F)s4)n3c2)CC1 ; CC1(NS(=O)(=O)c2cc(N3CCC(S(C)(=N)=O)CC3)c3c(Cl)nc(-c4nnc(C(F)F)s4)n3c2)CC1] 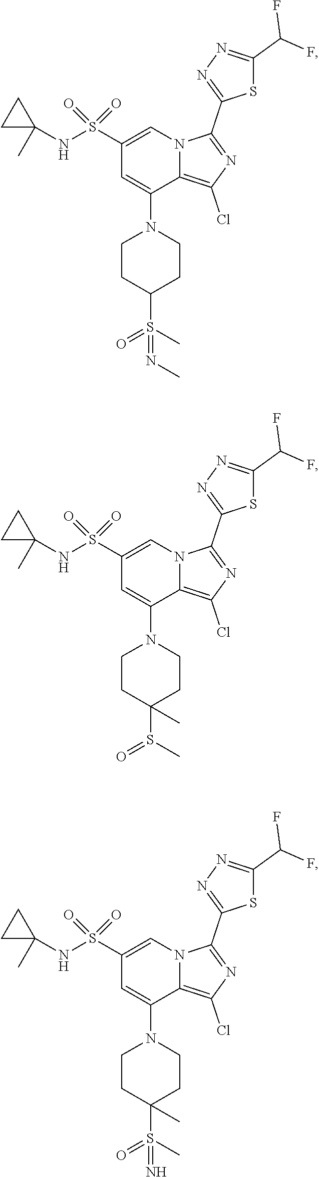 [SMILES: CN=S(C)(=O)C1CCN(c2cc(S(=O)(=O)NC3(C)CC3)cn3c(-c4nnc(C(F)F)s4)nc(Cl)c23)CC1 ; CS(=O)C1(C)CCN(c2cc(S(=O)(=O)NC3(C)CC3)cn3c(-c4nnc(C(F)F)s4)nc(Cl)c23)CC1 ; CC1(NS(=O)(=O)c2cc(N3CCC(C)(S(C)(=N)=O)CC3)c3c(Cl)nc(-c4nnc(C(F)F)s4)n3c2)CC1] 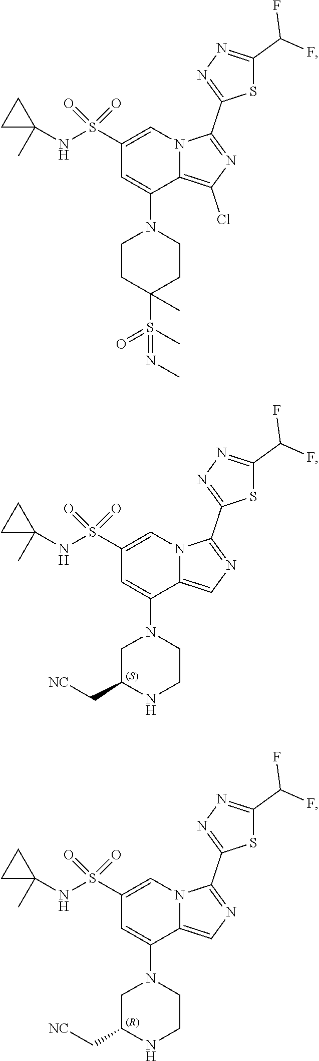 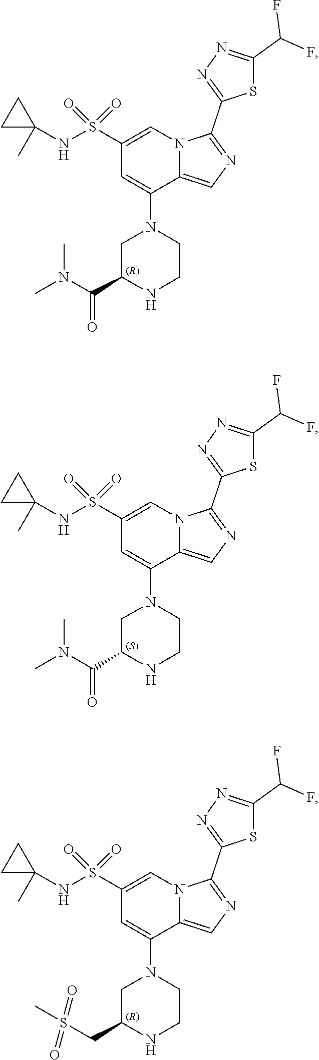 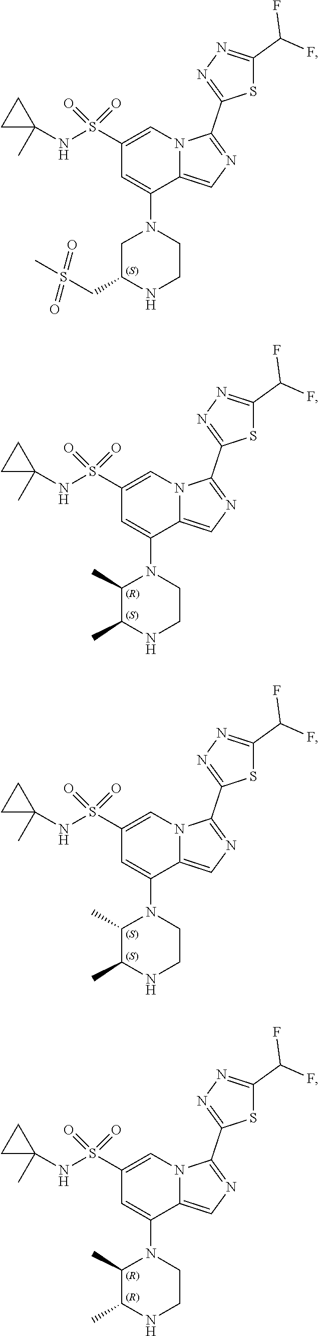 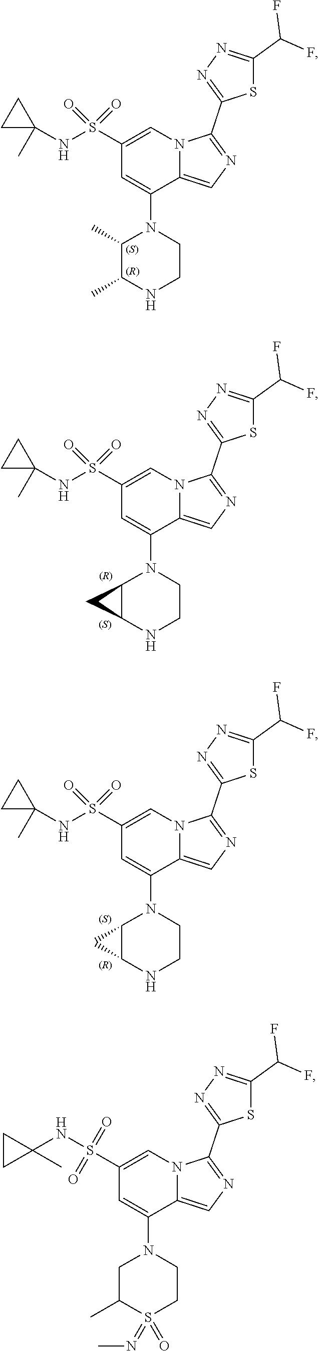 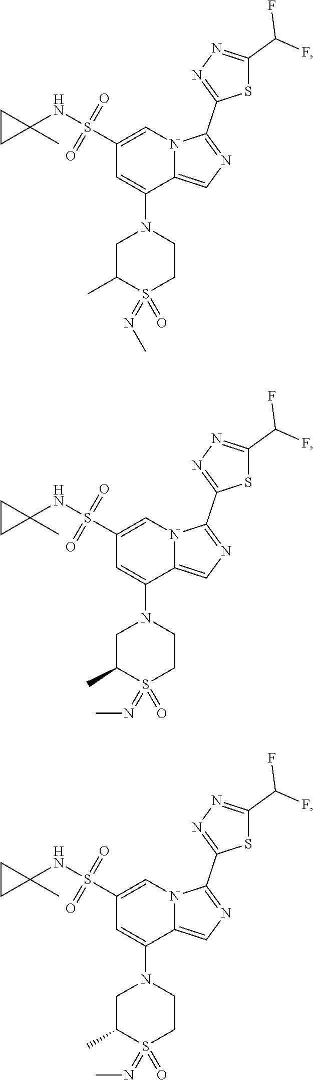 [SMILES: CN=S1(=O)CCN(c2cc(S(=O)(=O)NC3(C)CC3)cn3c(-c4nnc(C(F)F)s4)ncc23)CC1C ; CN=S1(=O)CCN(c2cc(S(=O)(=O)NC3(C)CC3)cn3c(-c4nnc(C(F)F)s4)ncc23)C[C@@H]1C ; CN=S1(=O)CCN(c2cc(S(=O)(=O)NC3(C)CC3)cn3c(-c4nnc(C(F)F)s4)ncc23)C[C@H]1C] 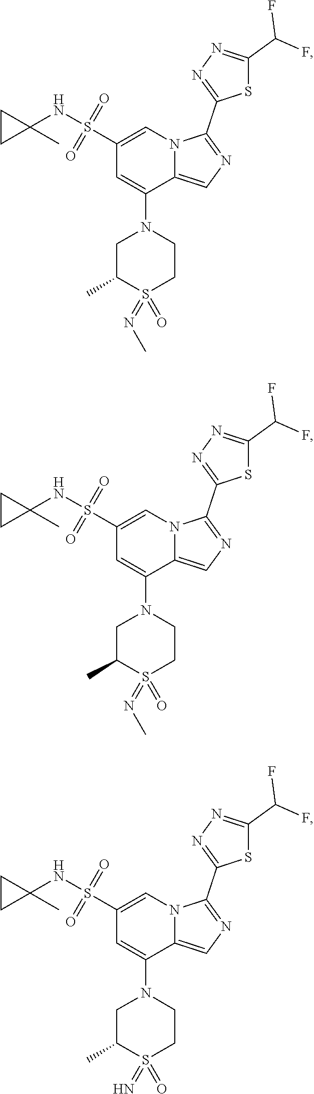 [SMILES: CN=S1(=O)CCN(c2cc(S(=O)(=O)NC3(C)CC3)cn3c(-c4nnc(C(F)F)s4)ncc23)C[C@H]1C ; CN=S1(=O)CCN(c2cc(S(=O)(=O)NC3(C)CC3)cn3c(-c4nnc(C(F)F)s4)ncc23)C[C@@H]1C ; C[C@@H]1CN(c2cc(S(=O)(=O)NC3(C)CC3)cn3c(-c4nnc(C(F)F)s4)ncc23)CCS1(=N)=O] 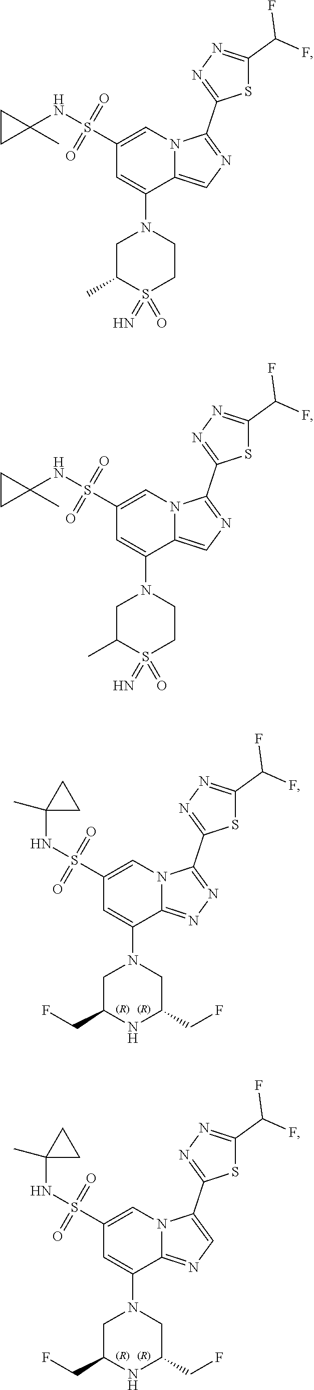 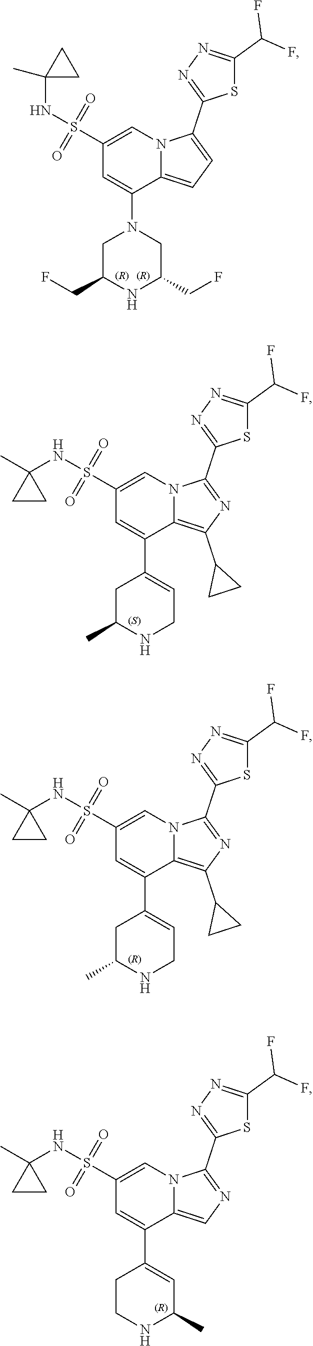 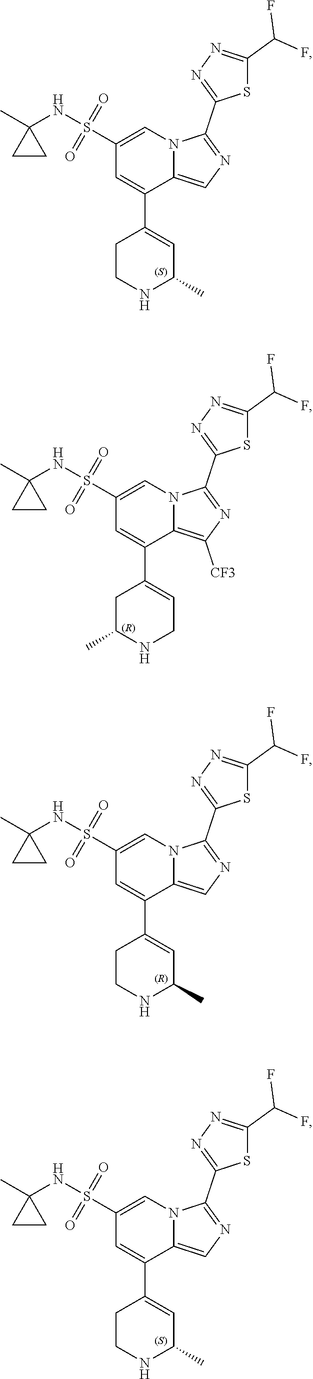 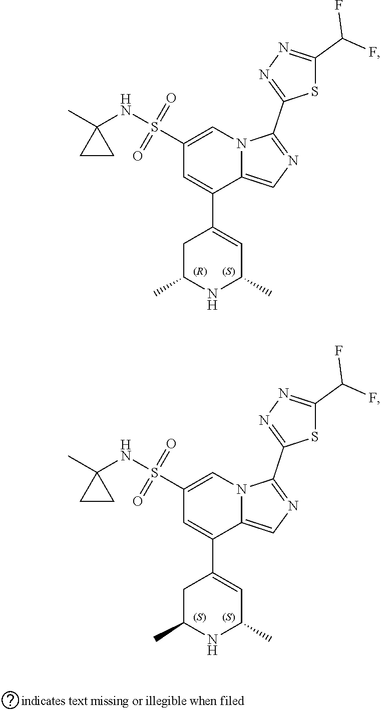 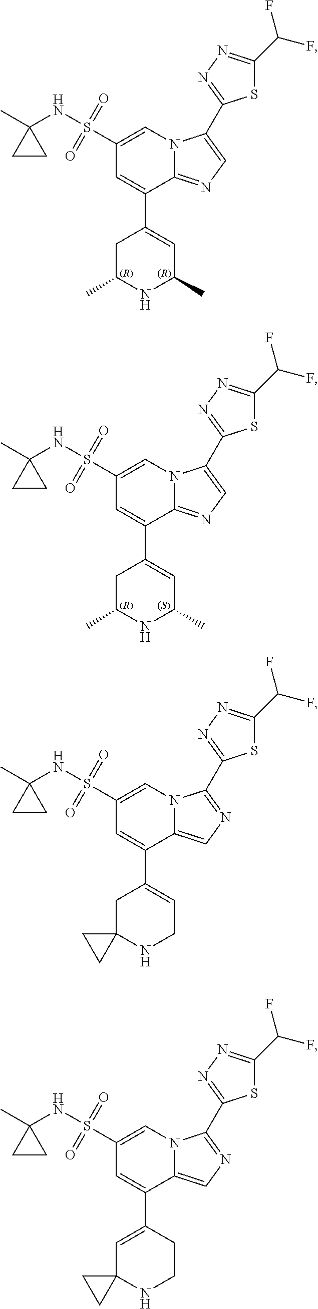 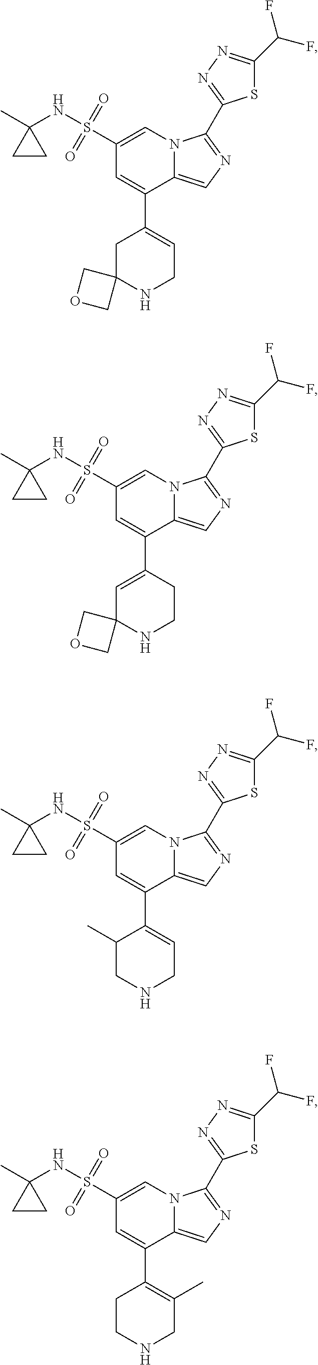 [SMILES: CC1(NS(=O)(=O)c2cc(C3=CCNC4(COC4)C3)c3cnc(-c4nnc(C(F)F)s4)n3c2)CC1 ; CC1(NS(=O)(=O)c2cc(C3=CC4(COC4)NCC3)c3cnc(-c4nnc(C(F)F)s4)n3c2)CC1 ; CC1CNCC=C1c1cc(S(=O)(=O)NC2(C)CC2)cn2c(-c3nnc(C(F)F)s3)ncc12 ; CC1=C(c2cc(S(=O)(=O)NC3(C)CC3)cn3c(-c4nnc(C(F)F)s4)ncc23)CCNC1] 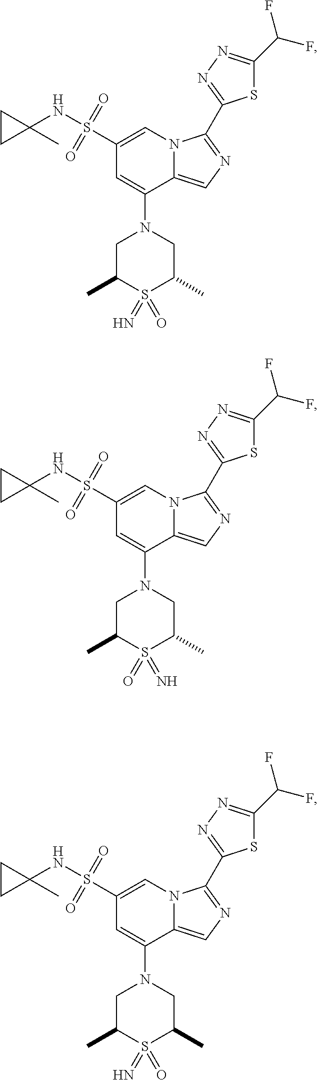 [SMILES: C[C@H]1CN(c2cc(S(=O)(=O)NC3(C)CC3)cn3c(-c4nnc(C(F)F)s4)ncc23)C[C@H](C)S1(=N)=O ; C[C@H]1CN(c2cc(S(=O)(=O)NC3(C)CC3)cn3c(-c4nnc(C(F)F)s4)ncc23)C[C@H](C)S1(=N)=O ; C[C@@H]1CN(c2cc(S(=O)(=O)NC3(C)CC3)cn3c(-c4nnc(C(F)F)s4)ncc23)C[C@H](C)S1(=N)=O] 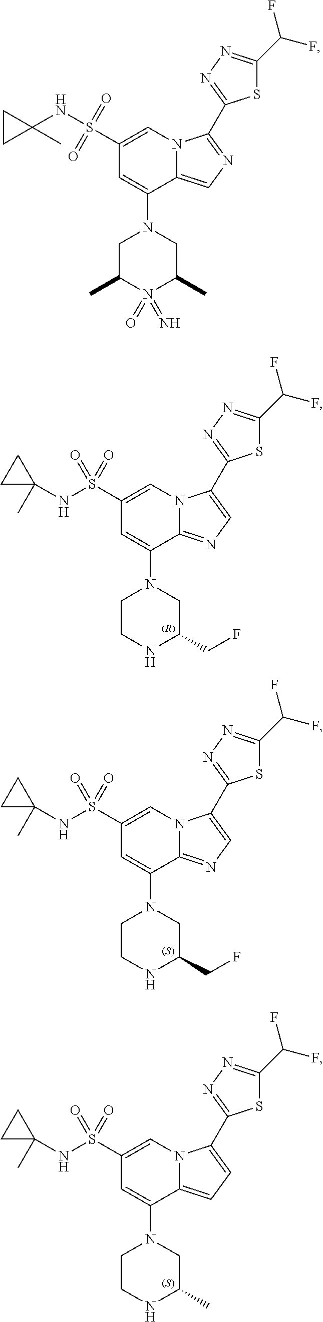 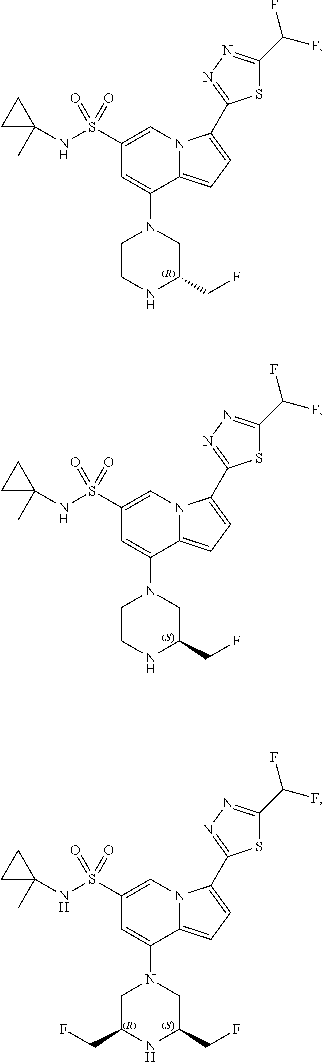 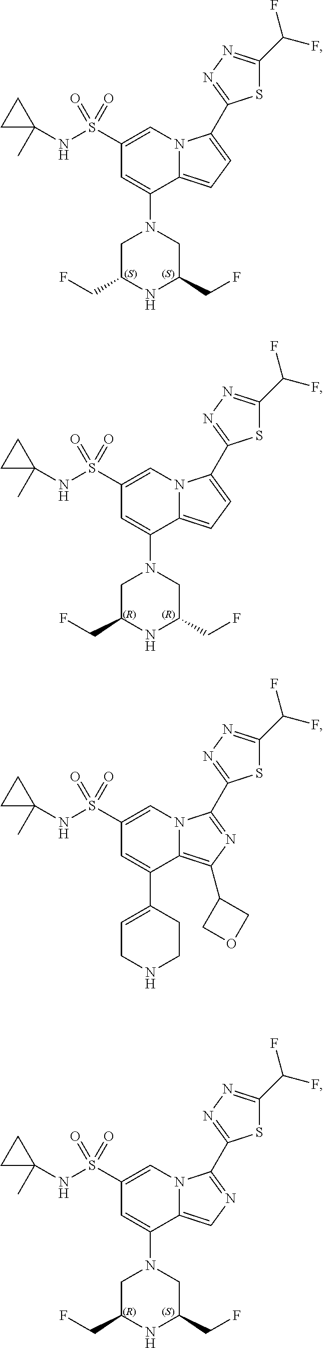 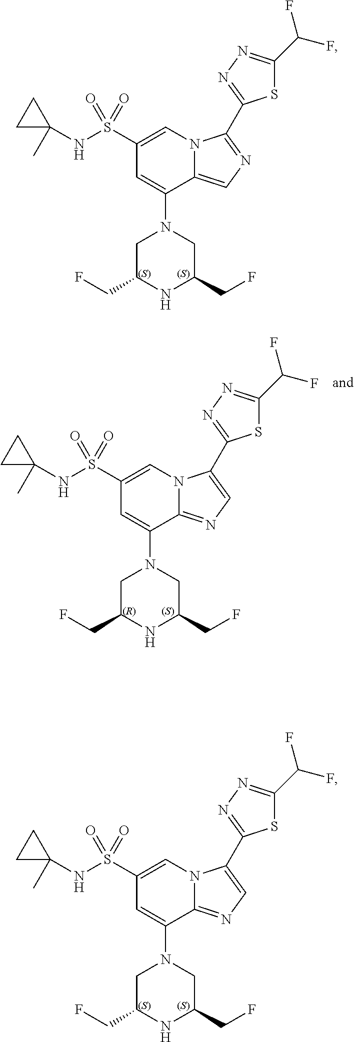
or a pharmaceutically acceptable salt thereof.

3. A compound selected from: 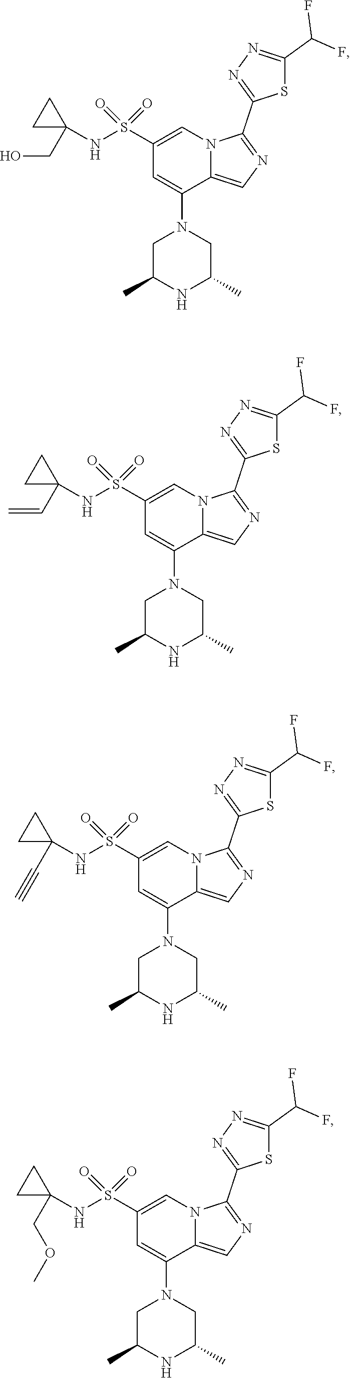 [SMILES: C[C@H]1CN(c2cc(S(=O)(=O)NC3(CO)CC3)cn3c(-c4nnc(C(F)F)s4)ncc23)C[C@H](C)N1 ; C=CC1(NS(=O)(=O)c2cc(N3C[C@H](C)N[C@@H](C)C3)c3cnc(-c4nnc(C(F)F)s4)n3c2)CC1 ; C#CC1(NS(=O)(=O)c2cc(N3C[C@H](C)N[C@@H](C)C3)c3cnc(-c4nnc(C(F)F)s4)n3c2)CC1 ; COCC1(NS(=O)(=O)c2cc(N3C[C@H](C)N[C@@H](C)C3)c3cnc(-c4nnc(C(F)F)s4)n3c2)CC1] 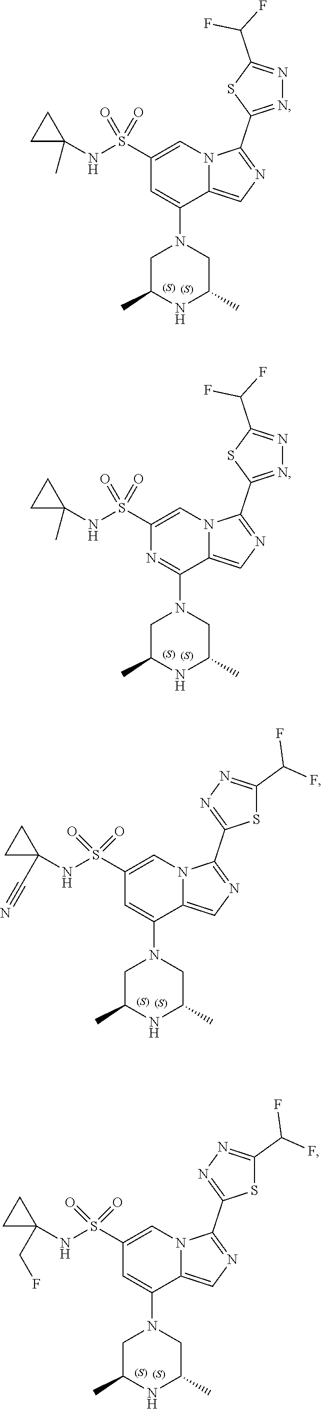 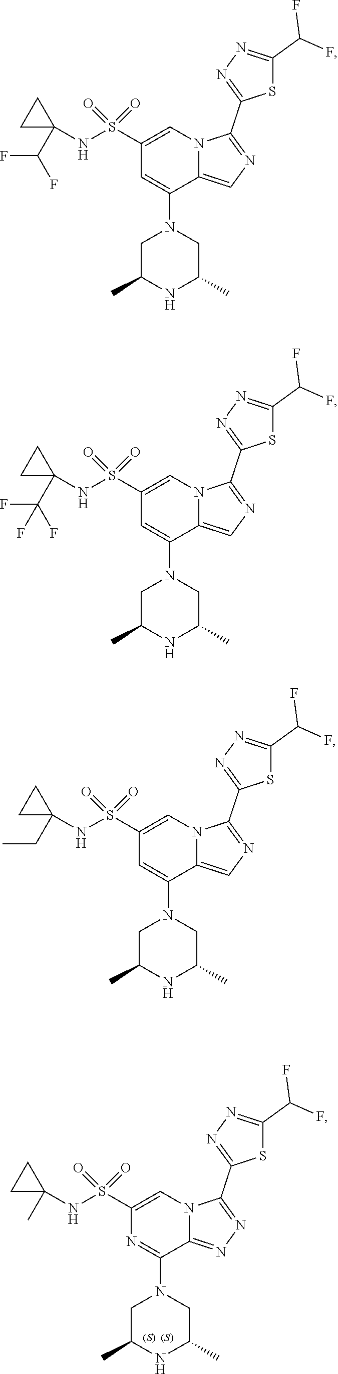 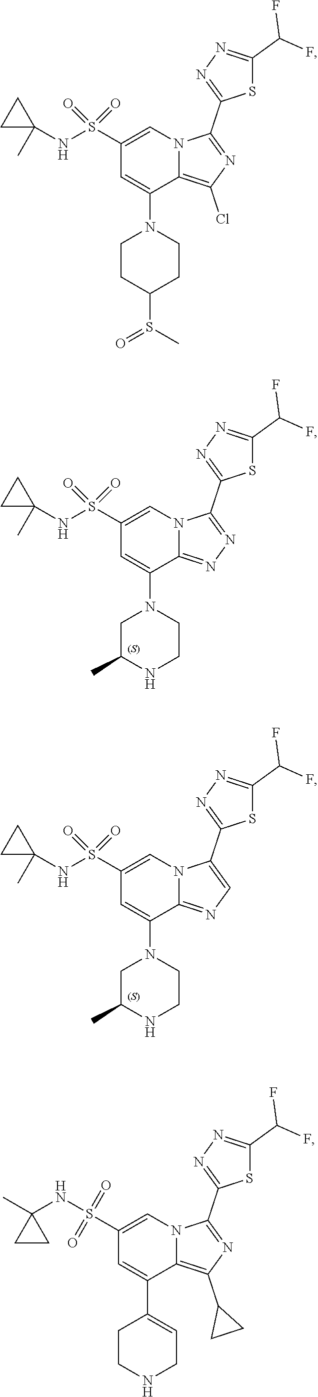 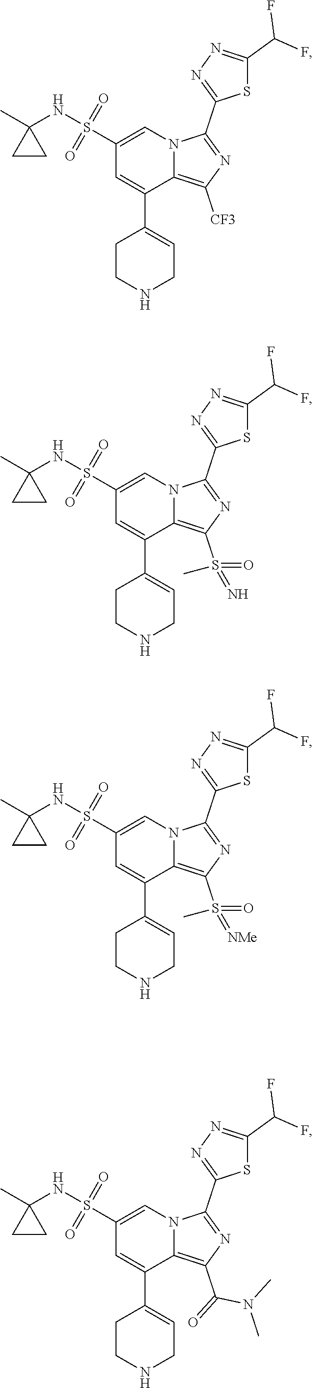 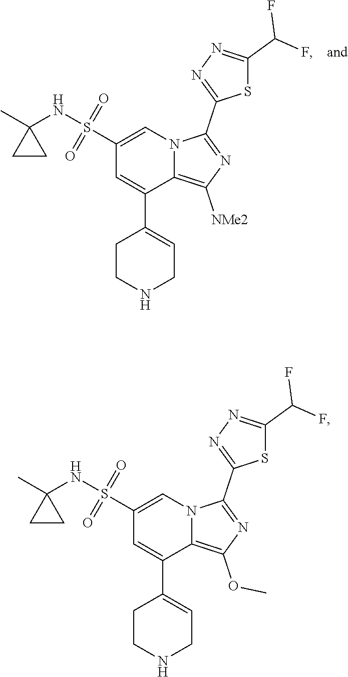 [SMILES: CN(C)c1nc(-c2nnc(C(F)F)s2)n2cc(S(=O)(=O)NC3(C)CC3)cc(C3=CCNCC3)c12 ; COc1nc(-c2nnc(C(F)F)s2)n2cc(S(=O)(=O)NC3(C)CC3)cc(C3=CCNCC3)c12]
or a pharmaceutically acceptable salt thereof.

4. The compound of claim 1, according to formula: 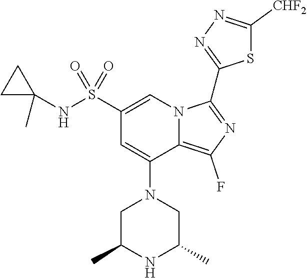 [SMILES: C[C@H]1CN(c2cc(S(=O)(=O)NC3(C)CC3)cn3c(-c4nnc(C(F)F)s4)nc(F)c23)C[C@H](C)N1]
or a pharmaceutically acceptable salt thereof.

5. The compound of claim 1, according to formula 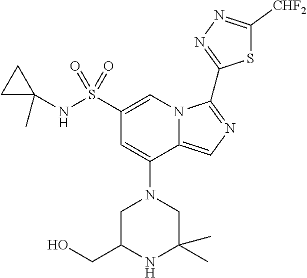 [SMILES: CC1(C)CN(c2cc(S(=O)(=O)NC3(C)CC3)cn3c(-c4nnc(C(F)F)s4)ncc23)CC(CO)N1]
or a pharmaceutically acceptable salt thereof.

6. The compound of claim 1, according to formula: 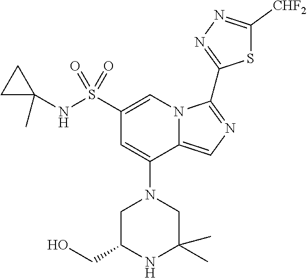 [SMILES: CC1(C)CN(c2cc(S(=O)(=O)NC3(C)CC3)cn3c(-c4nnc(C(F)F)s4)ncc23)C[C@@H](CO)N1]
or a pharmaceutically acceptable salt thereof.

7. The compound of claim 1, according to formula: 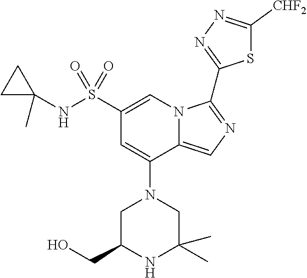 [SMILES: CC1(C)CN(c2cc(S(=O)(=O)NC3(C)CC3)cn3c(-c4nnc(C(F)F)s4)ncc23)C[C@H](CO)N1]
or a pharmaceutically acceptable salt thereof.

8. A pharmaceutical composition comprising the compound of claim 1 or a pharmaceutically acceptable salt, hydrate or solvate thereof, and a pharmaceutically acceptable carrier.

9-12. (canceled)

13. A method of treating a disease or disorder in which PARG activity is implicated, which comprises administering the compound of claim 1 or a pharmaceutically acceptable salt, hydrate, or solvate thereof, to a subject in need thereof.

14. (canceled)

15. (canceled)

16. A method of treating a proliferative disorder, the method comprising the step of administering the compound of claim 1 or a pharmaceutically acceptable salt, hydrate, or solvate thereof, to a subject in need thereof.

17. The method of claim 16, wherein the proliferative disorder is cancer.

18. The method of claim 17, wherein said cancer is a human cancer.

19. The compound of claim 1, wherein R₂ is selected from the group consisting of hydrogen, C₁₋₂ alkyl, C₂ alkenyl, C₂ alkynyl, C₁₋₂ haloalkyl, cyclopropyl and halogen.

20. The compound of claim 19, wherein R₂ is selected from the group consisting of hydrogen, —CH₃, —CF₃, cyclopropyl, and —Cl.

21. The compound of claim 20, wherein R₂ is hydrogen.

22. The compound of claim 1, wherein R₁ is selected from the group consisting of fluoro, cyano, formyl, (C₁₋₂)alkyl, (C₂)alkenyl, (C₂)alkynyl and (C₁₋₂)haloalkyl.

23. The compound of claim 22, wherein R₁ is selected from the group consisting of fluoro, cyano, formyl, (C₁₋₂)alkyl, (C₂)alkenyl, (C₂)alkynyl and (C₁₋₂)haloalkyl.

24. The compound of claim 23, wherein R₁ is selected from the group consisting of cyano, (C₁₋₂)alkyl, and (C₁₋₂)haloalkyl.
